SEQUENCE LISTING (295 sequences)

SEQ ID NO: 1 - DNA, 279 bp, synthetic construct
  source 1..279: mol_type = other DNA
cttcggcctgttcaagcacaagcgcagagtgactgctcttgtagcacggtttcacctggcgtattggccggtattgtaatgggggaccttgtactcacggttctcatagctcttgctgtctattttctcggacgactggtcccacggggacgaggggcagcagaagctgctacacgaaaacagaggattacagagacggagagtccctaccaagaactccaggggcagagaagtgatgtctattctgaccttaacacacaaagaccatactataaatga

SEQ ID NO: 2 - DNA, 189 bp, synthetic construct
  source 1..189: mol_type = other DNA
ggtctggccgtaagtaccatatccagcttttttccgccaggatatcaggtttccttttgtttggtcatggtacttctctttgcggtagacactggtctctattttagtgtcaaaactaatatacgctcctccacgagggattggaaggaccataagttcaaatggaggaaggacccgcaggacaaatga

SEQ ID NO: 3 - DNA, 135 bp, synthetic construct
  source 1..135: mol_type = other DNA
actactactccagctccaaggcctcccacgccagctcccactattgcttctcaaccgttgtcactgcgaccagaggcctgtagacctgcagctggaggcgctgttcacacaaggggtctcgattttgcgtgtgac

SEQ ID NO: 4 - DNA, 285 bp, synthetic construct
  source 1..285: mol_type = other DNA
actactactccagctccaaggcctcccacgccagctcccactattgcttctcaaccgttgtcactgcgaccagaggcctgtagacctgcagctggaggcgctgttcacacaaggggtctcgattttgcgtgtgacggatatcaggtttccttttgtttggtcatggtacttctctttgcggtagacactggtctctattttagtgtcaaaactaatatacgctcctccacgagggattggaaggaccataagttcaaatggaggaaggacccgcaggacaaatga

SEQ ID NO: 5 - DNA, 216 bp, synthetic construct
  source 1..216: mol_type = other DNA
tctggtcaagttctgctggagtcaaacattaaagtgctgcctacttggagcactcctgttcagcctggatatcaggtttccttttgtttggtcatggtacttctctttgcggtagacactggtctctattttagtgtcaaaactaatatacgctcctccacgagggattggaaggaccataagttcaaatggaggaaggacccgcaggacaaatga

SEQ ID NO: 6 - DNA, 846 bp, synthetic construct
  source 1..846: mol_type = other DNA
tatccacctgtcatagttgaaatgaattccagtgttgaggctatcgagggcagtcacgtatcactcctgtgtggtgcagattccaatccaccacccctccttacatggatgcgggatggaactgttctgagagaagcggtggcggaaagtttgctccttgaattggaggaggttactcccgccgaggacggcgtttatgcctgcctggccgagaatgcgtacggacaagacaatcgaacggtcggtttgagcgtgatgtacgcgccttggaaacctacggttaacggcactatggttgcggtagaaggggaaacggtatccatactctgtagtacacaatcaaatcctgatcccatcctcacgatctttaaagagaaacaaatcctttccacagtcatttatgagtctgagcttcagctcgaactgccagcagtctcccctgaggatgatggagaatattggtgcgttgccgaaaaccagtatggccagagagctacagcgttcaatctcagcgtagaatttgctccagttctcttgctggagagtcactgtgcggcggcacgggatactgtccagtgtctttgtgtagtgaaaagcaatcctgagccttctgtagcttttgagttgccttcacgcaacgtgacggtaaatgagagcgaacgcgagttcgtgtatagtgagagaagcggattggtgctgacttcaatcctcacgcttcggggccaggcgcaggcgccacctcgcgtgatttgcactgctcggaacctttacggcgcaaaatccttggagctgccgtttcagggagcccatcggcttatgtgggctaagattggtcctgtgggggct

SEQ ID NO: 7 - DNA, 996 bp, synthetic construct
  source 1..996: mol_type = other DNA
tatccacctgtcatagttgaaatgaattccagtgttgaggctatcgagggcagtcacgtatcactcctgtgtggtgcagattccaatccaccacccctccttacatggatgcgggatggaactgttctgagagaagcggtggcggaaagtttgctccttgaattggaggaggttactcccgccgaggacggcgtttatgcctgcctggccgagaatgcgtacggacaagacaatcgaacggtcggtttgagcgtgatgtacgcgccttggaaacctacggttaacggcactatggttgcggtagaaggggaaacggtatccatactctgtagtacacaatcaaatcctgatcccatcctcacgatctttaaagagaaacaaatcctttccacagtcatttatgagtctgagcttcagctcgaactgccagcagtctcccctgaggatgatggagaatattggtgcgttgccgaaaaccagtatggccagagagctacagcgttcaatctcagcgtagaatttgctccagttctcttgctggagagtcactgtgcggcggcacgggatactgtccagtgtctttgtgtagtgaaaagcaatcctgagccttctgtagcttttgagttgccttcacgcaacgtgacggtaaatgagagcgaacgcgagttcgtgtatagtgagagaagcggattggtgctgacttcaatcctcacgcttcggggccaggcgcaggcgccacctcgcgtgatttgcactgctcggaacctttacggcgcaaaatccttggagctgccgtttcagggagcccatcggcttatgtgggctaagattggtcctgtgggggctggatatcaggtttccttttgtttggtcatggtacttctctttgcggtagacactggtctctattttagtgtcaaaactaatatacgctcctccacgagggattggaaggaccataagttcaaatggaggaaggacccgcaggacaaatga

SEQ ID NO: 8 - DNA, 693 bp, synthetic construct
  source 1..693: mol_type = other DNA
atgaataagcagcgagggaccttttcagaagtctcactcgctcaggatcctaaaagacaacaaagaaagcctaagggaaataagtccagcatatcaggtacggaacaggaaatttttcaagtcgaactgaatttgcagaaccctagcctgaatcaccaaggtatcgacaagatctatgattgtcaagggctcctgccaccgcctgaaaagcttacggcggaggtgctgggcattatttgtatagtcctgatggcaactgtacttaaaactattgtacttatcccgtttctggaacaaaacaatttttctccgaacactcggacacaaaaggcccgacattgtggtcactgtccagaagagtggataacttactctaatagctgttactatatcggaaaagagaggagaacgtgggaagaaagcttgctcgcatgcacttccaaaaactcttcactcttgtccattgataacgaggaggagatgaaatttctggcctcaatcctgccatcatcttggataggcgtattccgcaactcaagtcatcacccttgggtaactataaatggtttggcgttcaagcacaagattaaagactctgataatgccgagttgaactgcgctgttcttcaggtgaaccgcctcaaatctgcccagtgcggaagttctatgatatatcactgcaaacataaactg

SEQ ID NO: 9 - DNA, 216 bp, synthetic construct
  source 1..216: mol_type = other DNA
atggggtggatacgaggcaggaggtctcggcacagctgggagatgtcagagtttcacaactacaacctcgacctgaaaaaatccgacttctctacccgatggcaaaagcagcgatgtccggtagtgaagtcaaaatgtcgggaaaacgcatctccgttttttttttgctgcttcatagccgtcgcgatgggcataagattcatcattatggtgact

SEQ ID NO: 10 - DNA, 552 bp, synthetic construct
  source 1..552: mol_type = other DNA
ctgtgggttagccagccacccgagattcgaaccctcgaaggcagtagtgctttcttgccgtgtagcttcaatgcgtcccaagggcgactggcaatcggttcagtcacatggtttcgcgacgaagttgtacctggcaaggaggtcaggaatgggacaccggaatttcgcggccgactggccccgttggcatcttcccgatttcttcatgatcaccaggcagagcttcacattcgcgacgtacgaggacacgacgcaagcatctatgtatgtagagttgaagttttgggacttggagtaggcacagggaatgggactaggttggtagtggaaaaggagcatccccagttgggcgcaggtaccgtacttcttctgcgggcagggttctatgctgtcagctttctgtctgtggcagttgggtccacagtctattaccagggtaagtgtctcacgtggaagggaccacggcggcaattgcctgcggttgttcccgcacccctccctcctccatgcggttcaagtgcacatctccttccgccagttccaggcggctga

SEQ ID NO: 11 - DNA, 201 bp, synthetic construct
  source 1..201: mol_type = other DNA
gcaggtaccgtacttcttctgcgggcagggttctatgctgtcagctttctgtctgtggcagttgggtccacagtctattaccagggtaagtgtctcacgtggaagggaccacggcggcaattgcctgcggttgttcccgcacccctccctcctccatgcggttcaagtgcacatctccttccgccagttccaggcggctga

SEQ ID NO: 12 - DNA, 246 bp, synthetic construct
  source 1..246: mol_type = other DNA
aatgggactaggttggtagtggaaaaggagcatccccagttgggcgcaggtaccgtacttcttctgcgggcagggttctatgctgtcagctttctgtctgtggcagttgggtccacagtctattaccagggtaagtgtctcacgtggaagggaccacggcggcaattgcctgcggttgttcccgcacccctccctcctccatgcggttcaagtgcacatctccttccgccagttccaggcggctga

SEQ ID NO: 13 - DNA, 768 bp, synthetic construct
  source 1..768: mol_type = other DNA
cagtcaaaagcacaagttctgcagagtgtagcaggccagacacttacggttaggtgtcaatatccgcctaccggcagtctgtatgagaaaaagggctggtgtaaagaagcgtccgcgttggtttgcattcggctcgtaacgagctccaagcctcggactatggcatggacatcacggttcactatatgggacgaccctgatgcaggattttttactgtcaccatgacggaccttcgggaggaggattccggccactattggtgtagaatctatagaccgtcagacaattctgtcagcaagagcgtgcgcttctaccttgtggtctctccggcttccgcctccacccaaacatcttggactccgagggatctcgtgtcatcacaaacccagacacagagttgtgtgccgcccacggcgggagcaagacaggctccggagagcccatctacaattccggtccctagccaaccacagaactctaccttgaggcccggacccgctgcacccatcgctttggttccagtgttttgcggactccttgttgccaagtcacttgtcctttctgctctcctggtatggtggggcgacatttggtggaaaacgatgatggagcttcgatccttggacacacagaaggcgacatgtcatctccaacaggtgacagacctgccatggactagtgtgtcaagtcccgtcgagcgcgaaatcctttatcataccgtggcccgaaccaaaataagcgacgatgacgatgagcacactctgtga

SEQ ID NO: 14 - DNA, 201 bp, synthetic construct; identical to SEQ ID NO: 11
  source 1..201: mol_type = other DNA

SEQ ID NO: 15 - DNA, 312 bp, synthetic construct
  source 1..312: mol_type = other DNA
gtccctagccaaccacagaactctaccttgaggcccggacccgctgcacccatcgctttggttccagtgttttgcggactccttgttgccaagtcacttgtcctttctgctctcctggtatggtggggcgacatttggtggaaaacgatgatggagcttcgatccttggacacacagaaggcgacatgtcatctccaacaggtgacagacctgccatggactagtgtgtcaagtcccgtcgagcgcgaaatcctttatcataccgtggcccgaaccaaaataagcgacgatgacgatgagcacactctgtga

SEQ ID NO: 16 - DNA, 852 bp, synthetic construct
  source 1..852: mol_type = other DNA
cagcaacagacgctgcccaaaccatttatttgggctgaacctcactttatggtccccaaggaaaaacaagttacaatctgttgccaggggaattacggagctgtggagtaccagctgcatttcgagggatctctttttgcggttgataggcccaagccgcccgagcggatcaacaaggtgaagttttacataccagatatgaactccaggatggcgggacagtactcctgtatctatcgcgtgggcgagctgtggagtgagccttccaatctgcttgatcttgtcgtcaccgagatgtacgatacacctacgctgagcgttcatcccgggccggaagttattagtggcgaaaaggtcactttctattgccgccttgacacggctacgtcaatgttcctcttgctgaaagaaggaagatcttcccatgtccaacgaggatacggaaaagtccaagcggaatttcccctgggaccagtaactaccgctcataggggaacataccgatgcttcggcagctacaacaaccacgcttggagttttccgtctgagcctgtaaaattgctcgttaccggagacattgagaacacgagccttgcccctgaagatccgacgttcccagcagatacatgggggacttatctgttgactacggagacaggacttcaaaaggaccatgcgttgtgggatcatacagctcagaacttgctccgcatgggcctggcctttcttgtactcgttgcactcgtttggttccttgttgaggattggctctctagaaagagaactagagaacgggcctccagggcatccacgtgggaaggccgcagacgactcaatacccagaccctgtga

SEQ ID NO: 17 - DNA, 141 bp, synthetic construct
  source 1..141: mol_type = other DNA
atgggcctggcctttcttgtactcgttgcactcgtttggttccttgttgaggattggctctctagaaagagaactagagaacgggcctccagggcatccacgtgggaaggccgcagacgactcaatacccagaccctgtga

SEQ ID NO: 18 - DNA, 195 bp, synthetic construct
  source 1..195: mol_type = other DNA
ggacttcaaaaggaccatgcgttgtgggatcatacagctcagaacttgctccgcatgggcctggcctttcttgtactcgttgcactcgtttggttccttgttgaggattggctctctagaaagagaactagagaacgggcctccagggcatccacgtgggaaggccgcagacgactcaatacccagaccctgtga

SEQ ID NO: 19 - DNA, 291 bp, synthetic construct
  source 1..291: mol_type = other DNA
tgctacagcgagaccctgcccatccaggtggagcagaacttcctgagcaacctgttcgtggccagctggatcaccgtgatgatcatcttcaggatcggcatggccgtggccatcttctgctgcttcttcttccccagcgccaacgagaggtgcaagagcaaggtggtgccctgcaggcagaagcagtggaggaccagcttcgacagcaagaagctggacctgaactacaaccacttcgagagcatggagtggagccacaggagcaggaggggcaggatctggggcatgtga

SEQ ID NO: 20 - protein, 363 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAAGSLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 21 - protein, 333 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAAGSGLAVSTISSFFPPGYQVSFCLVMVLLFAVDTGLYFSVKTNIRSSTRDWKDHKFKWRKDPQDK

SEQ ID NO: 22 - protein, 363 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAATTTPAPRPPTPAPTIASQPLSLRPEACRPAAGGAVHTRGLDFACDGYQVSFCLVMVLLFAVDTGLYFSVKTNIRSSTRDWKDHKFKWRKDPQDK

SEQ ID NO: 23 - protein, 363 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAATTTPAPRPPTPAPTIASQPLSLRPEASRPAAGGAVHTRGLDFASDGYQVSFCLVMVLLFAVDTGLYFSVKTNIRSSTRDWKDHKFKWRKDPQDK

SEQ ID NO: 24 - protein, 340 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAASGQVLLESNIKVLPTWSTPVQPGYQVSFCLVMVLLFAVDTGLYFSVKTNIRSSTRDWKDHKFKWRKDPQDK

SEQ ID NO: 25 - protein, 600 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAAYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAGYQVSFCLVMVLLFAVDTGLYFSVKTNIRSSTRDWKDHKFKWRKDPQDK

SEQ ID NO: 26 - protein, 333 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGAAAGSGLAVSTISSFFPPGYQVSFCLVMVLLFAVDTGLYFSVKTNIRSSTRDWKDHKFKWRKDPQDK

SEQ ID NO: 27 - protein, 485 aa, synthetic construct
MNKQRGTFSEVSLAQDPKRQQRKPKGNKSSISGTEQEIFQVELNLQNPSLNHQGIDKIYDCQGLLPPPEKLTAEVLGIICIVLMATVLKTIVLIPFLEQNNFSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKLSGGGGAAAGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 28 - protein, 347 aa, synthetic construct
MNKQRGTFSEVSLAQDPKRQQRKPKGNKSSISGTEQEIFQVELNLQNPSLNHQGIDKIYDCQGLLPPPEKLTAEVLGIICIVLMATVLKTIVLSGGGGAAAGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 29 - protein, 470 aa, synthetic construct
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSGGGGAAAGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 30 - protein, 326 aa, synthetic construct
MGWIRGRRSRHSWEMSEFHNYNLDLKKSDFSTRWQKQRCPVVKSKCRENASPFFFCCFIAVAMGIRFIIMVTSGGGGAAAGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 31 - protein, 454 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGAAAGSLWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 32 - protein, 337 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGAAAGSAGTVLLLRAGFYAVSFLSVAVGSTVYYQGKCLTWKGPRRQLPAVVPAPLPPPCGSSAHLLPPVPGG

SEQ ID NO: 33 - protein, 526 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGAAAGSQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIALVPVFCGLLVAKSLVLSALLVWWGDIWWKTMMELRSLDTQKATCHLQQVTDLPWTSVSSPVEREILYHTVARTKISDDDDEHTL

SEQ ID NO: 34 - protein, 355 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGAAAGSLVPVFCGLLVAKSLVLSALLVWWGDIWWKTMMELRSLDTQKATCHLQQVTDLPWTSVSSPVEREILYHTVARTKISDDDDEHTL

SEQ ID NO: 35 - protein, 554 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGAAAGSQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 36 - protein, 317 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGAAAGSMGLAFLVLVALVWFLVEDWLSRKRTRERASRASTWEGRRRLNTQTL

SEQ ID NO: 37 - protein, 366 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGAAAGCYSETLPIQVEQNFLSNLFVASWITVMIIFRIGMAVAIFCCFFFPSANERCKSKVVPCRQKQWRTSFDSKKLDLNYNHFESMEWSHRSRRGRIWGM

SEQ ID NO: 38 - protein, 351 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGAAAGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 39 - protein, 373 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGAAAGVPSQPQNSTLRPGPAAPIALVPVFCGLLVAKSLVLSALLVWWGDIWWKTMMELRSLDTQKATCHLQQVTDLPWTSVSSPVEREILYHTVARTKISDDDDEHTL

SEQ ID NO: 40 - protein, 334 aa, synthetic construct
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGAAAGGLQKDHALWDHTAQNLLRMGLAFLVLVALVWFLVEDWLSRKRTRERASRASTWEGRRRLNTQTL

SEQ ID NO: 41 - protein, 35 aa, synthetic construct
YEDMRGILYAAPQLRSIRGQPGPNHEEDADSYENM

SEQ ID NO: 42 - protein, 12 aa, synthetic construct
GGFGSSYWYFDV

SEQ ID NO: 43 - protein, 9 aa, synthetic construct
QQHYITPLT

SEQ ID NO: 44 - protein, 118 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSS

SEQ ID NO: 45 - protein, 240 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 46 - protein, 11 aa, synthetic construct
WGGDGFYAMDV

SEQ ID NO: 47 - protein, 120 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 48 - protein, 108 aa, synthetic construct
LVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 49 - protein, 9 aa, synthetic construct
QQHYTTPPT

SEQ ID NO: 50 - protein, 130 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVE

SEQ ID NO: 51 - protein, 107 aa, synthetic construct [sequence omitted]

SEQ ID NO: 52 - protein, 255 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESSGGGGSGGGGSGGGGSLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 53 - protein, 244 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 54 - protein, 119 aa, synthetic construct
QVQLQESGGGLVQAGGSLRLSCAAPRSIFSINAMGWYRQAPGKQRELVAAITSGGSPTYADSVKGRFTISRDNAKNTVYLQMNSLKAEDTAVYYCATGPYGLDNALDAWGQGTQVTVSS

SEQ ID NO: 55 - protein, 122 aa, synthetic construct
QVQLQESGGGLVQTGGSLRLACTASGFTFDDYAIAWFRQAPGKEREFVAAISWSGGTTHYADSVKGRFTISRDNAKNTLYLQMSSLKPEDTAVYFCAKSLRSSPSSRWFGSRGQGTQVTVSS

SEQ ID NO: 56 - protein, 14 aa, synthetic construct
ATACADTTQYAYDY

SEQ ID NO: 57 - protein, 14 aa, synthetic construct
ATACADTTLYEYDY

SEQ ID NO: 58 - protein, 14 aa, synthetic construct
ATACVDTTQYEYDY

SEQ ID NO: 59 - protein, 14 aa, synthetic construct
ATACADATQHEYDY

SEQ ID NO: 60 - protein, 14 aa, synthetic construct
ATACADTTQYDYDY

SEQ ID NO: 61 - protein, 14 aa, synthetic construct
ATACADTTQYEYDY

SEQ ID NO: 62 - protein, 14 aa, synthetic construct
ATACADTTHYEYDY

SEQ ID NO: 63 - protein, 14 aa, synthetic construct
ATACVITTLYEYDY

SEQ ID NO: 64 - protein, 14 aa, synthetic construct
ATACAETTLYEYDY

SEQ ID NO: 65 - protein, 14 aa, synthetic construct
ATACADTTQHEYDY

SEQ ID NO: 66 - protein, 14 aa, synthetic construct
ATACVDTTHYEYDY

SEQ ID NO: 67 - protein, 14 aa, synthetic construct
ATACASTTLYEYDY

SEQ ID NO: 68 - protein, 14 aa, synthetic construct
ATACVVTTLYEYDY

SEQ ID NO: 69 - protein, 13 aa, synthetic construct
ATACGGATGPYDY

SEQ ID NO: 70 - protein, 13 aa, synthetic construct
ATACAGAIGPYDY

SEQ ID NO: 71 - protein, 13 aa, synthetic construct
ATACVVVGDQNDY

SEQ ID NO: 72 - protein, 13 aa, synthetic construct
ATACVVVGDRNDY

SEQ ID NO: 73 - protein, 14 aa, synthetic construct
ATDCAGGTSTPYDY

SEQ ID NO: 74 - protein, 14 aa, synthetic construct
ATDCAGGTATPYDY

SEQ ID NO: 75 - protein, 14 aa, synthetic construct
ATACVVADRNEYDY

SEQ ID NO: 76 - protein, 14 aa, synthetic construct
ATSCVVVTKNEYDY

SEQ ID NO: 77 - protein, 13 aa, synthetic construct
ATACSGLTHEYDY

SEQ ID NO: 78 - protein, 13 aa, synthetic construct
ATTCSGLTHEYDY

SEQ ID NO: 79 - protein, 15 aa, synthetic construct
ATACANWSSLGPYDY

SEQ ID NO: 80 - protein, 15 aa, synthetic construct
ATACANWSTLGPYDY

SEQ ID NO: 81 - protein, 14 aa, synthetic construct
ATACSDPRVYEYDY

SEQ ID NO: 82 - protein, 14 aa, synthetic construct
ATTCASPEKYEYDY

SEQ ID NO: 83 - protein, 15 aa, synthetic construct
ATHCGGTSWGTSYDY

SEQ ID NO: 84 - protein, 15 aa, synthetic construct
ATHCGGSSWSNEYDY

SEQ ID NO: 85 - protein, 9 aa, synthetic construct
YARYSGRTY

SEQ ID NO: 86 - protein, 17 aa, synthetic construct
ASSAWPAGPKHQVEYDY

SEQ ID NO: 87 - protein, 13 aa, synthetic construct
ATACGSLVGMYDY

SEQ ID NO: 88 - protein, 13 aa, synthetic construct
ATACGSAVHEYDY

SEQ ID NO: 89 - protein, 14 aa, synthetic construct
ATDCVGFGSNWFDY

SEQ ID NO: 90 - protein, 14 aa, synthetic construct
ATACASPVIYEYDY

SEQ ID NO: 91 - protein, 14 aa, synthetic construct
ATDCAGGVGHEYDY

SEQ ID NO: 92 - protein, 15 aa, synthetic construct
ATDCSLHGSDYPYDY

SEQ ID NO: 93 - protein, 17 aa, synthetic construct
AVRIYSGSFDNTLAYDY

SEQ ID NO: 94 - protein, 8 aa, synthetic construct
GFPLAYYA

SEQ ID NO: 95 - protein, 8 aa, synthetic construct
GFSLDYYA

SEQ ID NO: 96 - protein, 8 aa, synthetic construct
GFPLDYYA

SEQ ID NO: 97 - protein, 8 aa, synthetic construct
GFTLDYYA

SEQ ID NO: 98 - protein, 8 aa, synthetic construct
GFSLNYYA

SEQ ID NO: 99 - protein, 8 aa, synthetic construct
GFTLAYYA

SEQ ID NO: 100 - protein, 8 aa, synthetic construct
GFTLGYYA

SEQ ID NO: 101 - protein, 8 aa, synthetic construct
GFPLNYYA

SEQ ID NO: 102 - protein, 8 aa, synthetic construct
GFPLHYYA

SEQ ID NO: 103 - protein, 8 aa, synthetic construct
GFSLGYYA

SEQ ID NO: 104 - protein, 8 aa, synthetic construct
GFPLGYYA

SEQ ID NO: 105 - protein, 8 aa, synthetic construct
GFPLEYYA

SEQ ID NO: 106 - protein, 8 aa, synthetic construct
GSDFRADA

SEQ ID NO: 107 - protein, 8 aa, synthetic construct
GRTFSSYG

SEQ ID NO: 108 - protein, 8 aa, synthetic construct
GFSLAYYA

SEQ ID NO: 109 - protein, 8 aa, synthetic construct
GLTFRSVG

SEQ ID NO: 110 - protein, 8 aa, synthetic construct
ISNSDGST

SEQ ID NO: 111 - protein, 8 aa, synthetic construct
ISASDGST

SEQ ID NO: 112 - protein, 8 aa, synthetic construct
ISSSDGST

SEQ ID NO: 113 - protein, 8 aa, synthetic construct
ISSSDGNT

SEQ ID NO: 114 - protein, 8 aa, synthetic construct
ISSADGST

SEQ ID NO: 115 - protein, 8 aa, synthetic construct
ISSSGGST

SEQ ID NO: 116 - protein, 8 aa, synthetic construct
ISSGDGST

SEQ ID NO: 117 - protein, 8 aa, synthetic construct
ISAGDGNT

SEQ ID NO: 118 - protein, 8 aa, synthetic construct
ISSSDDST

SEQ ID NO: 119 - protein, 8 aa, synthetic construct
ISSNDGST

SEQ ID NO: 120 - protein, 8 aa, synthetic construct
ISSPDGST

SEQ ID NO: 121 - protein, 8 aa, synthetic construct
ISSRTGGT

SEQ ID NO: 122 - protein, 9 aa, synthetic construct
ISAGDGSST

SEQ ID NO: 123 - protein, 13 aa, synthetic construct
ISSSDGSSSDGNT

SEQ ID NO: 124 - protein, 8 aa, synthetic construct
ISSGDGNT

SEQ ID NO: 125 - protein, 8 aa, synthetic construct
ISSGDGKT

SEQ ID NO: 126 - protein, 8 aa, synthetic construct
ISSSDGGT

SEQ ID NO: 127 - protein, 8 aa, synthetic construct
ISSRTGST

SEQ ID NO: 128 - protein, 8 aa, synthetic construct
ISSRTGNT

SEQ ID NO: 129 - protein, 10 aa, synthetic construct
ISSSDGHSST

SEQ ID NO: 130 - protein, 9 aa, synthetic construct
ISSSSDGNT

SEQ ID NO: 131 - protein, 8 aa, synthetic construct
ISASNGNT

SEQ ID NO: 132 - protein, 9 aa, synthetic construct
ISSGSDGNT

SEQ ID NO: 133 - protein, 8 aa, synthetic construct
ISASDGNT

SEQ ID NO: 134 - protein, 7 aa, synthetic construct
IDSITSI

SEQ ID NO: 135 - protein, 16 aa, synthetic construct
ISWSGGSTIAASVGST

SEQ ID NO: 136 - protein, 11 aa, synthetic construct
ISSSDGSDGNT

SEQ ID NO: 137 - protein, 8 aa, synthetic construct
ASPSGVIT

SEQ ID NO: 138 - protein, 121 aa, synthetic construct
QVQLQESGGGLVHSGGSLRLSCAASGFPLAYYAIGWFRQAPGKEREGVSCISSSDGNTYYADAVKGRFTISRDNAKNTVYLQMNSLKPEDTAVYYCATACADTTQHEYDYWGQGTQVTVSS

SEQ ID NO: 139 - protein, 121 aa, synthetic construct
QVQLQESGGGLVHSGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 140 - protein, 120 aa, synthetic construct
QVQLQESGGGLVHSGGSLRLSCAASGFTLDYYAIGWFRRAPGKEREGVSCISSGDGKTYYADSVKGRFTISRDNAKNTVYLQMNSLKPEDTAVYYCATACAGAIGPYDYWGQGTQVTVSS

SEQ ID NO: 141 - protein, 121 aa, synthetic construct
QVQLQESGGGLVPPGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 142 - protein, 123 aa, synthetic construct
QVQLQESGGGLVQAGGSLRLSCAASGFSLGYYAIGWFRQAPGKEREGVSCISSSDGHSSTYYADSVKGRFTISRDNAKNTVYLQMNNLKPEDTAVYYCATDCAGGTATPYDYWGQGTQVTVSS

SEQ ID NO: 143 - protein, 124 aa, synthetic construct
QVQLQESGGGLVQAGGSLRLSCAASGRTFSSYGMGWFRQAPGKEREFVAAISWSGGSTYYADSVKGRFTISRDNAKNTVYLQMNSLKPEDTAVYYCASSAWPAGPKHQVEYDYWGQGTQVTVSS

SEQ ID NO: 144 - protein, 120 aa, synthetic construct
QVQLQESGGGLVQAGGSLRLSCTASGFSLDYYAIGWFRQAPGKEREGVACISSRTGSTYYADSVKGRFTISRDNAKNTVALQMNSLKPEDTAVYYCATACVVVGDQNDYWGQGTQVTVSS

SEQ ID NO: 145 - protein, 121 aa, synthetic construct
QVQLQESGGGLVQDGGSLRLSCAASGFPLAYYAIGWFRQAPGKEREGVSCISASDGSTYYADSVKGRFTISRDNAKNTVYLQMNSLKPEDTAVYYCATACAETTLYEYDYWGQGTQVTVSS

SEQ ID NO: 146 - protein, 122 aa, synthetic construct
QVQLQESGGGLVQPGESLRLSCAASGFPLAYYAIGWFRQAPGKEREGVSCISSSDGSTYYADSVKGRFTISRDNAKNTVYLQMNSLKPEDTAVYYCATACANWSTLGPYDYWGQGTQVTVSS

SEQ ID NO: 147 - protein, 121 aa, synthetic construct
QVQLQESGGGLVQPGESLRLSCAASGFTLAYYAIGWFRQAPGKEREGVSCISSSDGNTYYADSVKGRFTISRDNAKNTVYLQMNRLKPEDTAVYYCATACADTTQYEYDYWGQGTQVTVSS

SEQ ID NO: 148 - protein, 114 aa, synthetic construct
QVQLQESGGGLVQPGGSLKLSCAASGSDFRADAMGWYRQAPGKEREPVAIDSITSIYYVDSVEGRFTISRDNTKNTVYLQMTSLKPEDTAVYYCYARYSGRTYWGRGTQVTVSS

SEQ ID NO: 149 - protein, 121 aa, synthetic construct
QVQLQESGGGLVQPGGSLRLSCAASGFPLAYYAIGWFRQAPGKEREGVSCISASDGSTYYADSVKGRFTISRDNAKNTVYLQMNSLRPEDTAVYYCATACADTTLYEYDYWGQGTQVTVSS

SEQ ID NO: 150 - protein, 121 aa, synthetic construct
QVQLQESGGGLVQPGGSLRLSCAASGFPLAYYAIGWFRQAPGKEREGVSCISSSDGNTYYADAVKGRFAISRDNAKNTVYLQMNSLKPEDTAVYYCATACSDPRVYEYDYWGQGTQVTVSS

SEQ ID NO: 151 - protein, 121 aa, synthetic construct
QVQLQESGGGLVQPGGSLRLSCAASGFPLAYYAIGWFRQAPGKEREGVSCISSSDGNTYYADAVKGRFTISRDNAKNTVYLQMNSLKPEDTAVYYCATACADTTQHEYDYWGQGTQVTVSS

SEQ ID NO: 152 - protein, 121 aa, synthetic construct
QVQLQESGGGLVQPGGSLRLSCAASGFPLAYYAIGWFRQAPGKEREGVSCISSSDGNTYYADAVKGRFTISRDNAKNTVYLQMNSLKPEDTAVYYCATACVDTTHYEYDYWGQGTQVTVSS

SEQ ID NO: 153 - protein, 121 aa, synthetic construct
QVQLQESGGGLVQPGGSLRLSCAASGFPLAYYAIGWFRQAPGKEREGVSCISSSDGNTYYADSVKGRFTISRDNAKNTVYLQMNSLKPEDTAVYYCATACADATQHEYDYWGQGTQVTVSS

SEQ ID NO: 154 - protein, 121 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLGPEDTAVYYCATACADTTQYDYDYWGQGTQVTVSS

SEQ ID NO: 155 - protein, 121 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 156 - protein, 120 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 157 - protein, 121 aa, synthetic construct
QVQLQESGGGLVQPGGSLRLSCAASGFPLAYYAIGWFRRAPGKEREGVSCISSSDGNTYYADAVKGRFTISRDNAKNTVYLQMNSLKPEDTAVYYCATACADTTQHEYDYWGQGTQVTVSS

SEQ ID NO: 158 - protein, 122 aa, synthetic construct
QVQLQESGGGLVQPGGSLRLSCAASGFPLDYYAIGWFRQAPGKEREGVSCISAGDGSSTYYADSVKGRFTISRDNAKNTVYLQMNSLKPEDTAVYYCATACASTTLYEYDYWGQGTQVTVSS

SEQ ID NO: 159 - protein, 121 aa, synthetic construct
QVQLQESGGGLVQPGGSLRLSCAASGFPLDYYAIGWFRQAPGKEREGVSCISSADGSTYYADSVKGRFTISRDNAKNAVYLQMNSLGPEDTAVYYCATACADTTQYDYDYWGQGTQVTVSS

SEQ ID NO: 160 - protein, 121 aa, synthetic construct
QVQLQESGGGLVQPGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 161 - protein, 121 aa, synthetic construct
QVQLQESGGGLVQPGGSLRLSCAASGFPLDYYAIGWFRQAPGKEREGVSCISSADGSTYYADSVKGRFTISRDNAKNTVYLQMNSLKPEDTAVYYCATACVDTTQYEYDYWGQGTQVTVSS

SEQ ID NO: 162 - protein, 121 aa, synthetic construct
QVQLQESGGGLVQPGGSLRLSCAASGFPLDYYAIGWFRQAPGKEREGVSCISSPDGSTYYADSVKGRFTISRDNAKNTVYLQMNSLKPEDTAVYYCATACVDTTQYEYDYWGQGTQVTVSS

SEQ ID NO: 163 - protein, 125 aa, synthetic construct
QVQLQESGGGLVQPGGSLRLSCAASGFPLDYYAIGWFRQAPGKEREGVSCISSSDGSDGNTYYADSVKGRFTISRDNAKNTVYLQMNSLKPEDTAVYYCATDCSLHGSDYPYDYWGQGTQVTVSS

SEQ ID NO: 164 - protein, 121 aa, synthetic construct
QVQLQESGGGLVQPGGSLRLSCAASGFPLDYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 165 - protein, 121 aa, synthetic construct
QVQLQESGGGLVQPGGSLRLSCAASGFPLEYYAIGWFRQAPGKEREGVSCISSSDGSTYYADSVKGRFTISRDNAKNTVYLQMNSLKPEDTAVYYCATACSDPRVYEYDYWGQGTQVTVSS

SEQ ID NO: 166 - protein, 121 aa, synthetic construct
QVQLQESGGGLVQPGGSLRLSCAASGFPLGYYAIGWFRQAPGKEREGVSCISSSDDSTYYADSVKGRFTISRDNDKNTVYLQMNSLKPEDTAVYYCATDCAGGTSTPYDYWGQGTQVTVSS

SEQ ID NO: 167 - protein, 121 aa, synthetic construct
QVQLQESGGGLVQPGGSLRLSCAASGFPLHYYAIGWFRQAPGKEREGVSCISSGDGSTYYADSVKGRFTISRDNAKNTVYLQMNSLKPEDTAVYYCATSCVVVTKNEYDYWGQGTQVTVSS

SEQ ID NO: 168 - protein, 120 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 169 - protein, 121 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLKPEDTAVYYCATACVVADRNEYDYWGQGTQVTVSS

SEQ ID NO: 170 - protein, 121 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLRPEDTAVYYCATACVVADRNEYDYWGQGTQVTVSS

SEQ ID NO: 171 - protein, 121 aa, synthetic construct
QVQLQESGGGLVQPGGSLRLSCAASGFPLNYYAIGWFRQAPGKEREGVSCISASDGNTYYADSVKGRFTISRDNAKNTVYLQMNSLKPEDTAVYYCATTCASPEKYEYDYWGQGTQVTVSS

SEQ ID NO: 172 - protein, 120 aa, synthetic construct
QVQLQESGGGLVQPGGSLRLSCAASGFPLNYYAIGWFRQAPGKEREGVSCISSSDGSTYYADSVKGRFIISRDNAKNTVYLQMNSLKPEDTAVYYCATACGGATGPYDYWGQGTQVTVSS

SEQ ID NO: 173 - protein, 120 aa, synthetic construct
QVQLQESGGGLVQPGGSLRLSCAASGFPLNYYAIGWFRQAPGKEREGVSCISSSDGSTYYADSVKGRFTISRDNAKNTVYLQMNSLKPEDTAVYYCATACGSAVHEYDYWGQGTQVTVSS

SEQ ID NO: 174 - protein, 121 aa, synthetic construct
QVQLQESGGGLVQPGGSLRLSCAASGFSLAYYAIGWFRQAPGKEREGVSCIAASVGSTYYADSVKGRFTISRDDAKNTVYLQMNSLKPEDTAVYYCATDCAGGVGHEYDYWGQGTQVTVSS

SEQ ID NO: 175 - protein, 120 aa, synthetic construct
QVQLQESGGGLVQPGGSLRLSCAASGFSLDYYAIGWFRQAPGKEREGVSCISSSDGSTYYADSVKGRFTISRDNAKNAVYLQMNSLKPEDTAVYYCATACGGATGPYDYWGQGTQVTVSS

SEQ ID NO: 176 - protein, 121 aa, synthetic construct
QVQLQESGGGLVQPGGSLRLSCAASGFSLDYYAIGWFRQAPGKEREGVSCISSSDGSTYYADSVKGRFTISRDNAKNAVYLQMNSLKPEDTAVYYCATACVDTTQYEYDYWGQGTQVTVSS

SEQ ID NO: 177 - protein, 121 aa, synthetic construct
QVQLQESGGGLVQPGGSLRLSCAASGFSLDYYAIGWFRQAPGKEREGVSCISSSDGSTYYADSVKGRFTISRDNAKNTVYLQMNSLKPEDTAVYYCATDCAGGTSTPYDYWGQGTQVTVSS

SEQ ID NO: 178 - protein, 121 aa, synthetic construct
QVQLQESGGGLVQPGGSLRLSCAASGFSLNYYAIGWFRQAPGKEREGVSCISAGDGNTYYADSVKGRFTISRDNAANTVSLQMDSLKPEDTAVYYCATACVITTLYEYDYWGQGTQVTVSS

SEQ ID NO: 179 - protein, 121 aa, synthetic construct
QVQLQESGGGLVQPGGSLRLSCAASGFTLAYYAIGWFRQAPGKEREGVSCISSSDGSTYYADSVKGRFTISRDNAKNTVYLQMNSLKPEDTAVYYCATACADTTQHEYDYWGQGTQVTVSS

SEQ ID NO: 180 - protein, 121 aa, synthetic construct
QVQLQESGGGLVQPGGSLRLSCAASGFTLAYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 181 - protein, 120 aa, synthetic construct
QVQLQESGGGLVQPGGSLRLSCAASGFTLDYYAIGWFRQAPGKEREGVACISSSDGSTYYADSVKGRFTISRDNAKNTVYLQMNSLKPEDTAVYYCATACGGATGPYDYWGQGTQVTVSS

SEQ ID NO: 182 - protein, 120 aa, synthetic construct
QVQLQESGGGLVQPGGSLRLSCAASGFTLDYYAIGWFRQAPGKEREGVACISSSDGSTYYADSVKGRFTISRDNAKNTVYLQMNSLKPQDTAVYYCATACGSLVGMYDYWGQGTQVTVSP

SEQ ID NO: 183 - protein, 121 aa, synthetic construct
QVQLQESGGGLVQPGGSLRLSCAASGFTLDYYAIGWFRQAPGKEREGVSCISASDGNTYYADSVKGRFTISRDNAKNTVYLQMNSLKPEDTAVYYCATTCASPEKYEYDYWGQGTQVTVSS

SEQ ID NO: 184 - protein, 120 aa, synthetic construct
QVQLQESGGGLVQPGGSLRLSCAASGFTLDYYAIGWFRQAPGKEREGVSCISASNGNTYYADSVKGRFTISRDSAKNTVYLQMNSLKPEDTAVYYCATTCSGLTHEYDYWGQGTQVTVSS

SEQ ID NO: 185 - protein, 120 aa, synthetic construct
QVQLQESGGGLVQPGGSLRLSCAASGFTLDYYAIGWFRQAPGKEREGVSCISSGDGNTYYADSVKGRFTISRDNAKNTVYLQMNSLKPEDTAVYYCATACGGATGPYDYWGQGTQVTVSS

SEQ ID NO: 186 - protein, 122 aa, synthetic construct
QVQLQESGGGLVQPGGSLRLSCAASGFTLDYYAIGWFRQAPGKEREGVSCISSGDGSTYYADSVKGRFTISRDNAKNTVYLQMNSLKPEDTAVYYCATHCGGSSWSNEYDYWGQGTQVTVSS

SEQ ID NO: 187 - protein, 121 aa, synthetic construct
QVQLQESGGGLVQPGGSLRLSCAASGFTLDYYAIGWFRQAPGKEREGVSCISSNDGSTYYADSVKGRFTISRDNAKNTVYLQMNSLKPEDTAVYYCATACADTTQHEYDYWGQGTQVTVSS

SEQ ID NO: 188 - protein, 120 aa, synthetic construct
QVQLQESGGGLVQPGGSLRLSCAASGFTLDYYAIGWFRQAPGKEREGVSCISSSDGGTYYADSVKGRFTISRDNAKNTVYLQMNSLKPEDTAVYYCATACGGATGPYDYWGQGTQVTVSS

SEQ ID NO: 189 - protein, 126 aa, synthetic construct
QVQLQESGGGLVQPGGSLRLSCAASGFTLDYYAIGWFRQAPGKEREGVSCISSSDGSSSDGNTYYADSVKGRFTISRDNAKNTVYLQMNNLKPEDTAVYYCATACVVTTLYEYDYWGQGTQVTVSS

SEQ ID NO: 190 - protein, 121 aa, synthetic construct
QVQLQESGGGLVQPGGSLRLSCAASGFTLDYYAIGWFRQAPGKEREGVSCISSSDGSTYYADSVKGRFTISRDNAKNTVYLQMNSLKPEDTAVYYCATACADTTQYEYDYWGQGTQVTVSP

SEQ ID NO: 191 - protein, 120 aa, synthetic construct
QVQLQESGGGLVQPGGSLRLSCAASGFTLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 192 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNMLKPEDTAVYYCATACADTTQYEYDYWGQGTQVTVSS

SEQ ID NO: 193 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNSLKPEDTAVYYCATACASPVIYEYDYWGQGTQVTVSS

SEQ ID NO: 194 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNSLKPEDTAVYYCATDCAGGTSTPYDYWGQGTQVTVSS

SEQ ID NO: 195 - protein, 121 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 196 - protein, 122 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTAVYYCATACANWSSLGPYDYWGQGTQVTVSS

SEQ ID NO: 197 - protein, 120 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTGVYYCATACGGATGPYDYWGQGTQVTVSS

SEQ ID NO: 198 - protein, 121 aa, synthetic construct
QVQLQESGGGLVQPGGSLRLSCEGSGFSLDYYAIGWFRQAPGKEREGVSCISSGDGNTYYADSVKGRFTISRDNAKNTVYLQMNSLKPEDTAVYYCATDCVGFGSNWFDYWGQGTQVTVSS

SEQ ID NO: 199 - protein, 120 aa, synthetic construct
QVQLQESGGGLVQPGGSLRLSCTASGFSLDYYAIGWFRQAPGKEREGVACISSRTGSTYYADSVKGRFTISRDNAKNTVALQMNSLKPEDTAVYYCATACVVVGDQNDYWGQGTQVTVSS

SEQ ID NO: 200 - protein, 120 aa, synthetic construct
QVQLQESGGGLVQPGGSLRLSCTASGFSLDYYAIGWFRQAPGKEREGVSCISSRTGGTYYADSVKGRFTISRDDAKNTVYLQMNSLKPEDTAVYYCATACVVVGDRNDYWGQGTQVTVSS

SEQ ID NO: 201 - protein, 121 aa, synthetic construct
QVQLQESGGGLVQPGGSLRLSCTASGFSLDYYAIGWFRQAPGKEREGVSCISSRTGGTYYADSVKGRFTISRDNAKNTVYLQMNSLKPEDTAVYYCATACVDTTQYEYDYWGQGTQVTVSS

SEQ ID NO: 202 - protein, 120 aa, synthetic construct
QVQLQESGGGLVQPGGSLRLSCTASGFSLDYYAIGWFRQAPGKEREGVSCISSRTGGTYYADSVKGRFTISRDNAKNTVYLQMNSLKPEDTAVYYCATACVVVGDQNDYWGQGTQVTVSS

SEQ ID NO: 203 - protein, 120 aa, synthetic construct
QVQLQESGGGLVQPGGSLRLSCTASGFSLDYYAIGWFRQAPGKEREGVSCISSRTGNTYYADSVKGRFTISRDDAKNMVYLQMNSLKPEDTAVYYCATACVVVGDQNDYWGQGTQVTVSS

SEQ ID NO: 204 - protein, 120 aa, synthetic construct
QVQLQESGGGLVQPGGSLRLSCTASGFSLDYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 205 - protein, 120 aa, synthetic construct
QVQLQESGGGLVQPGGSLRLSCTASGFSLGYYAIGWFRQALGKEREGVSCISSRTGSTYYADSVKGRFTVSRDDAKNTVYLQMNSLKPEDTAVYYCATACVVVGDQNDYWGQGTQVTVSS

SEQ ID NO: 206 - protein, 120 aa, synthetic construct
QVQLQESGGGLVQPGGSLRLSCTASGFSLGYYAIGWFRQAPGKEREGVSCISSRTGSTYYADSVKGRFAISRDDAKNTVYLQMNSLKPEDTAVYYCATACVVVGDQNDYWGQGTQVTVSS

SEQ ID NO: 207 - protein, 120 aa, synthetic construct
QVQLQESGGGLVQPGGSLRLSCTASGFSLGYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 208 - protein, 120 aa, synthetic construct
QVQLQESGGGLVQPGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 209 - protein, 120 aa, synthetic construct
QVQLQESGGGLVQPGGSLRLSCVASGFPLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 210 - protein, 121 aa, synthetic construct
QVQLQESGGGLVQPGGSLRLSCVASGFSLDYYAIGWFRQAPGKEREGVSCISNSDGSTYYADSVKGRFTISRDNAKNTVYLQMNSLKPEDTAVYYCATACADTTQYAYDYWGQGTQVTVSS

SEQ ID NO: 211 - protein, 121 aa, synthetic construct
QVQLQESGGGLVQPGGSLRLSCVASGFTLDYYAIGWFRQAPGKEREGVSCISSGSDGNTYYADSVKGRFTISRDNAKNTVYLQMNSLKPEDTAVYYCATACSGLTHEYDYWGQGTQVTVSS

SEQ ID NO: 212 - protein, 121 aa, synthetic construct
QVQLQESGGGLVQPGGSLRLSCVASGFTLDYYAIGWFRQAPGKEREGVSCISSSDDSTYYADSVKGRFTISRDNAKNTVYLQMNSLKPEDTAVYYCATACADTTQYEYDYWGQGTQVTVSS

SEQ ID NO: 213 - protein, 121 aa, synthetic construct
QVQLQESGGGLVQPGGSLRLSCVASGFTLDYYAIGWFRQAPGKEREGVSCISSSSDGNTYYADSVKGRFTISRDNAKNTVYLQMNSLKPEDTAVYYCATTCSGLTHEYDYWGQGTQVTVSS

SEQ ID NO: 214 - protein, 121 aa, synthetic construct
QVQLQESGGGLVQPGGSLRLSCVASGFTLGYYAIGWFRQAPGKEREGVSCISSSDGSTYYADSVKGRFTISRDNAKNTVYLQMNSLKPEDTAVYYCATACADTTQYDYDYWGQGTQVTVSS

SEQ ID NO: 215 - protein, 120 aa, synthetic construct
QVQLQESGGGLVQPGGSLRLSCVGSGFTLDYYAIGWFRQAPGKEREGVSCISSNDGSTYYADSVKGRFTISRDNAKNTVYLQMNSLKPEDTAVYYCATACGGATGPYDYWGQGTQVTVSS

SEQ ID NO: 216 - protein, 121 aa, synthetic construct
QVQLQESGGGLVQSGGSLRLSCAASGFPLAYYAIGWFRQAPGKEREGVSCISASDGSTYYADSVKGRFTISRDNAKNTVYLQMNSLKPEDTAVYYCATACAETTLYEYDYWGQGTQVTVSS

SEQ ID NO: 217 - protein, 120 aa, synthetic construct
QVQLQESGGGLVQTGGSLRLSCAASGFTLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 218 - protein, 121 aa, synthetic construct
QVQLQESGGGMVQAGESLRLSCAASGFPLAYYAIGWFRQAPGKEREGVSCISSSDGNTYYADSVKGRFTISRDNAKNTVYLQMNSLKPEDTAVYYCATACADATQHEYDYWGQGTQVTVSS

SEQ ID NO: 219 - protein, 121 aa, synthetic construct
QVQLQESGGGSVQPGESLRLSCAASGFPLDYYAIGWFRQAPGKEREGVSCISASDGSTYYADSVKGRFTISRDNAKNTVYLQMNSLKPEDTAVYYCATACADTTLYEYDYWGQGTQVTVSS

SEQ ID NO: 220 - protein, 121 aa, synthetic construct
QVQLQESGGGSVQPGGSLRLSCAASGFTLDYYAIGWFRQAPGKEREGVSCISSGDGSTYYADSVKGRFTISRDNAKNTVYLQMNSLKPEDTAVYYCATACADTTHYEYDYWGQGTQVTVSS

SEQ ID NO: 221 - protein, 120 aa, synthetic construct
QVQLQESGGGSVQSGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 222 - protein, 121 aa, synthetic construct
QVQLQESGGGSVRPGGSLRLSCAASGFPLAYYAIGWFRQAPGKEREGVSCISSSDGNTYYADAVKGRFTISRDNAKNAVYLQMNSLKPEDTAVYYCATACADTTQHEYDYWGQGTQVTVSS

SEQ ID NO: 223 - protein, 121 aa, synthetic construct
QVQLQESGGGVAQPGGSLRLSCAASGFPLDYYAIGWFRQAPGKEREGVSCISASDGSTYYADSVKGRFTISRDNAKNTVYLQMNSLKPEDTAVYYCATACADTTLYEYDYWGQGTQVTVSS

SEQ ID NO: 224 - protein, 114 aa, synthetic construct
QVQLQESGGGVVQAGGSLKLSCAASGSDFRADAMGWYRQAPGKEREPVAIDSITSIYYVDSVEGRFTISRDNTKNTVYLQMTSLKPEDTAVYYCYARYSGRTYWGRGTQVTVSS

SEQ ID NO: 225 - protein, 122 aa, synthetic construct
QVQLQESGGGVVQPGGSLRLSCAASGFSLDYYAIGWFRQAPGKEREGVSCISSGDGSTYYADSVKGRFTISRDNAKNTVYLQMNSLKPEDTAVYYCATHCGGTSWGTSYDYWGQGTQVTVSS

SEQ ID NO: 226 - protein, 124 aa, synthetic construct
QVQLQESGGGVVQPGGSLRLSCAASGLTFRSVGMGWFRRAPGKEREFVATASPSGVITYYADSVKGRFTISRDNAKNTVYLEMNSLKPEDTAVYYCAVRIYSGSFDNTLAYDYWGQGTQVTVSS

SEQ ID NO: 227 - protein, 120 aa, synthetic construct
QVQLQESGGGVVQPGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 228 - protein, 120 aa, synthetic construct
QVQLQESGGGVVQSGGSLRLSCTASGFSLDYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 229 - protein, 115 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSS

SEQ ID NO: 230 - protein, 112 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 231 - protein, 242 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 232 - protein, 244 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 233 - protein, 244 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 234 - protein, 17 aa, synthetic construct
MWLQSLLLLGTVACSIS

SEQ ID NO: 235 - protein, 116 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTV

SEQ ID NO: 236 - protein, 107 aa, synthetic construct
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 237 - protein, 257 aa, synthetic construct
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 238 - protein, 191 aa, synthetic construct
MEFGLSWLFLVAILKGVQCGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 239 - protein, 247 aa, synthetic construct
QVQLVQSGAEVKKPGASVKVSCKASGYTFTNYGMNWVRQAPGQGLEWMGWINTYTGEPTYADAFKGRVTMTTDTSTSTAYMELRSLRSDDTAVYYCARDYGDYGMDYWGQGTTVTVSSGSTSGSGKPGSSEGSTKGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 240 - protein, 247 aa, synthetic construct
QVQLQESGPGLVKPSQTLSLTCTVSGGSISSGYNWHWIRQPPGKGLEWIGYIHYTGSTNYNPALRSRVTISVDTSKNQFSLKLSSVTAADTAVYYCARIYNGNSFPYWGQGTTVTVSSGGGGSGGGGSGGGGSDIVMTQSPDSLAYSLGERATINCKSSQSLFNSGNQKNYLTWYQQKPGQPPKLLIYWASTRESGVPDRFSGSGSGTDIFITISSLQAEDVAVYYCQNAYSFPYTFGGGTKLEIKR

SEQ ID NO: 241 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 242 - protein, 108 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 243 - protein, 46 aa, synthetic construct
LYCRRLKIQVRKAAITSYEKSDGVYTGLSTRNQETYETLKHEKPPQ

SEQ ID NO: 244 - protein, 45 aa, synthetic construct
LYCRLKIQVRKAAITSYEKSDGVYTGLSTRNQETYETLKHEKPPQ

SEQ ID NO: 245 - protein, 42 aa, synthetic construct
RLKIQVRKAAITSYEKSDGVYTGLSTRNQETYETLKHEKPPQ

SEQ ID NO: 246 - protein, 43 aa, synthetic construct
RRLKIQVRKAAITSYEKSDGVYTGLSTRNQETYETLKHEKPPQ

SEQ ID NO: 247 - protein, 62 aa, synthetic construct
KKVAKKPTNKAPHPKQEPQEINFPDDLPGSNTAAPVQETLHGCQPVTQEDGKESRISVQERQ

SEQ ID NO: 248 - protein, 219 aa, synthetic construct
QRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 249 - protein, 171 aa, synthetic construct
PLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 250 - protein, 35 aa, synthetic construct
MHEEEIYTSLQWDSPAPDTYQKCLSSNKCSGACCL

SEQ ID NO: 251 - DNA, 1092 bp, synthetic construct
  source 1..1092: mol_type = other DNA
atgtggctgcagtctctgctgctgctgggaacagtggcctgtagcatctctcaagtgcagctgcagcagagcggcagcgagctgaaaaagcccggagccagcgtgaaagtgtcctgcaaggcttctggctacacattcaccaattacggcatgaactgggtcaagcaggcccctggacagggcctgaagtggatgggctggatcaacacctacaccggcgaacctacatacacagatgacttcaagggcagattcgccttcagcctggacaccagcgtgtccaccgcttatctgcagatcagcagcctgaaggccgacgataccgccgtgtacttttgtgcccggggcggatttggctctagctactggtacttcgacgtgtggggccagggcagcctggtgaccgtgtctagcggaggcggaggatcaggtggcggtggatctggcggtggtggctctgacatccagctgacacagagcccatctagcctgagcgctagcgtgggcgacagagtgtctattacctgtaaagcttctcaggacgtgtccatcgccgtcgcctggtatcagcagaagcccggcaaggcccctaagctgctgatctacagcgcctcctacagatacaccggcgtgcccgatagattcagcggaagcggcagcggaacagattttaccctgacaatcagcagcctgcagcctgaggacttcgccgtgtactactgccagcaacactacatcacccctctgaccttcggcgccggcaccaaggtggaaatcaagcggtcaggcggcggaggagcggccgcaggcagccttcggcctgttcaagcacaagcgcagagtgactgctcttgtagcacggtttcacctggcgtattggccggtattgtaatgggggaccttgtactcacggttctcatagctcttgctgtctattttctcggacgactggtcccacggggacgaggggcagcagaagctgctacacgaaaacagaggattacagagacggagagtccctaccaagaactccaggggcagagaagtgatgtctattctgaccttaacacacaaagaccatactataaatga

SEQ ID NO: 252 - DNA, 51 bp, synthetic construct
  source 1..51: mol_type = other DNA
atgtggctgcagtctctgctgctgctgggaacagtggcctgtagcatctct

SEQ ID NO: 253 - DNA, 732 bp, synthetic construct
  source 1..732: mol_type = other DNA
caagtgcagctgcagcagagcggcagcgagctgaaaaagcccggagccagcgtgaaagtgtcctgcaaggcttctggctacacattcaccaattacggcatgaactgggtcaagcaggcccctggacagggcctgaagtggatgggctggatcaacacctacaccggcgaacctacatacacagatgacttcaagggcagattcgccttcagcctggacaccagcgtgtccaccgcttatctgcagatcagcagcctgaaggccgacgataccgccgtgtacttttgtgcccggggcggatttggctctagctactggtacttcgacgtgtggggccagggcagcctggtgaccgtgtctagcggaggcggaggatcaggtggcggtggatctggcggtggtggctctgacatccagctgacacagagcccatctagcctgagcgctagcgtgggcgacagagtgtctattacctgtaaagcttctcaggacgtgtccatcgccgtcgcctggtatcagcagaagcccggcaaggcccctaagctgctgatctacagcgcctcctacagatacaccggcgtgcccgatagattcagcggaagcggcagcggaacagattttaccctgacaatcagcagcctgcagcctgaggacttcgccgtgtactactgccagcaacactacatcacccctctgaccttcggcgccggcaccaaggtggaaatcaagcgg

SEQ ID NO: 254 - DNA, 30 bp, synthetic construct
  source 1..30: mol_type = other DNA
tcaggcggcggaggagcggccgcaggcagc

SEQ ID NO: 255 - DNA, 1002 bp, synthetic construct
  source 1..1002: mol_type = other DNA
atgtggctgcagtctctgctgctgctgggaacagtggcctgtagcatctctcaagtgcagctgcagcagagcggcagcgagctgaaaaagcccggagccagcgtgaaagtgtcctgcaaggcttctggctacacattcaccaattacggcatgaactgggtcaagcaggcccctggacagggcctgaagtggatgggctggatcaacacctacaccggcgaacctacatacacagatgacttcaagggcagattcgccttcagcctggacaccagcgtgtccaccgcttatctgcagatcagcagcctgaaggccgacgataccgccgtgtacttttgtgcccggggcggatttggctctagctactggtacttcgacgtgtggggccagggcagcctggtgaccgtgtctagcggaggcggaggatcaggtggcggtggatctggcggtggtggctctgacatccagctgacacagagcccatctagcctgagcgctagcgtgggcgacagagtgtctattacctgtaaagcttctcaggacgtgtccatcgccgtcgcctggtatcagcagaagcccggcaaggcccctaagctgctgatctacagcgcctcctacagatacaccggcgtgcccgatagattcagcggaagcggcagcggaacagattttaccctgacaatcagcagcctgcagcctgaggacttcgccgtgtactactgccagcaacactacatcacccctctgaccttcggcgccggcaccaaggtggaaatcaagcggtcaggcggcggaggagcggccgcaggaagtggtctggccgtaagtaccatatccagcttttttccgccaggatatcaggtttccttttgtttggtcatggtacttctctttgcggtagacactggtctctattttagtgtcaaaactaatatacgctcctccacgagggattggaaggaccataagttcaaatggaggaaggacccgcaggacaaatga

SEQ ID NO: 256 - DNA, 30 bp, synthetic construct
  source 1..30: mol_type = other DNA
tcaggcggcggaggagcggccgcaggaagt

SEQ ID NO: 257 - DNA, 1092 bp, synthetic construct
  source 1..1092: mol_type = other DNA
atgtggctgcagtctctgctgctgctgggaacagtggcctgtagcatctctcaagtgcagctgcagcagagcggcagcgagctgaaaaagcccggagccagcgtgaaagtgtcctgcaaggcttctggctacacattcaccaattacggcatgaactgggtcaagcaggcccctggacagggcctgaagtggatgggctggatcaacacctacaccggcgaacctacatacacagatgacttcaagggcagattcgccttcagcctggacaccagcgtgtccaccgcttatctgcagatcagcagcctgaaggccgacgataccgccgtgtacttttgtgcccggggcggatttggctctagctactggtacttcgacgtgtggggccagggcagcctggtgaccgtgtctagcggaggcggaggatcaggtggcggtggatctggcggtggtggctctgacatccagctgacacagagcccatctagcctgagcgctagcgtgggcgacagagtgtctattacctgtaaagcttctcaggacgtgtccatcgccgtcgcctggtatcagcagaagcccggcaaggcccctaagctgctgatctacagcgcctcctacagatacaccggcgtgcccgatagattcagcggaagcggcagcggaacagattttaccctgacaatcagcagcctgcagcctgaggacttcgccgtgtactactgccagcaacactacatcacccctctgaccttcggcgccggcaccaaggtggaaatcaagcggtcaggcggcggaggagcggccgcaactactactccagctccaaggcctcccacgccagctcccactattgcttctcaaccgttgtcactgcgaccagaggcctgtagacctgcagctggaggcgctgttcacacaaggggtctcgattttgcgtgtgacggatatcaggtttccttttgtttggtcatggtacttctctttgcggtagacactggtctctattttagtgtcaaaactaatatacgctcctccacgagggattggaaggaccataagttcaaatggaggaaggacccgcaggacaaatga

SEQ ID NO: 258 - DNA, 24 bp, synthetic construct
  source 1..24: mol_type = other DNA
tcaggcggcggaggagcggccgca

SEQ ID NO: 259 - DNA, 150 bp, synthetic construct
  source 1..150: mol_type = other DNA
ggatatcaggtttccttttgtttggtcatggtacttctctttgcggtagacactggtctctattttagtgtcaaaactaatatacgctcctccacgagggattggaaggaccataagttcaaatggaggaaggacccgcaggacaaatga

SEQ ID NO: 260 - DNA, 1092 bp, synthetic construct
  source 1..1092: mol_type = other DNA
atgtggctgcagtctctgctgctgctgggaacagtggcctgtagcatctctcaagtgcagctgcagcagagcggcagcgagctgaaaaagcccggagccagcgtgaaagtgtcctgcaaggcttctggctacacattcaccaattacggcatgaactgggtcaagcaggcccctggacagggcctgaagtggatgggctggatcaacacctacaccggcgaacctacatacacagatgacttcaagggcagattcgccttcagcctggacaccagcgtgtccaccgcttatctgcagatcagcagcctgaaggccgacgataccgccgtgtacttttgtgcccggggcggatttggctctagctactggtacttcgacgtgtggggccagggcagcctggtgaccgtgtctagcggaggcggaggatcaggtggcggtggatctggcggtggtggctctgacatccagctgacacagagcccatctagcctgagcgctagcgtgggcgacagagtgtctattacctgtaaagcttctcaggacgtgtccatcgccgtcgcctggtatcagcagaagcccggcaaggcccctaagctgctgatctacagcgcctcctacagatacaccggcgtgcccgatagattcagcggaagcggcagcggaacagattttaccctgacaatcagcagcctgcagcctgaggacttcgccgtgtactactgccagcaacactacatcacccctctgaccttcggcgccggcaccaaggtggaaatcaagcggtcaggcggcggaggagcggccgcaactactactccagctccaaggcctcccacgccagctcccactattgcttctcaaccgttgtcactgcgaccagaggccagcagacctgcagctggaggcgctgttcacacaaggggtctcgattttgcgagcgacggatatcaggtttccttttgtttggtcatggtacttctctttgcggtagacactggtctctattttagtgtcaaaactaatatacgctcctccacgagggattggaaggaccataagttcaaatggaggaaggacccgcaggacaaatga

SEQ ID NO: 261 - DNA, 135 bp, synthetic construct
  source 1..135: mol_type = other DNA
actactactccagctccaaggcctcccacgccagctcccactattgcttctcaaccgttgtcactgcgaccagaggccagcagacctgcagctggaggcgctgttcacacaaggggtctcgattttgcgagcgac

SEQ ID NO: 262 - DNA, 1023 bp, synthetic construct
  source 1..1023: mol_type = other DNA
atgtggctgcagtctctgctgctgctgggaacagtggcctgtagcatctctcaagtgcagctgcagcagagcggcagcgagctgaaaaagcccggagccagcgtgaaagtgtcctgcaaggcttctggctacacattcaccaattacggcatgaactgggtcaagcaggcccctggacagggcctgaagtggatgggctggatcaacacctacaccggcgaacctacatacacagatgacttcaagggcagattcgccttcagcctggacaccagcgtgtccaccgcttatctgcagatcagcagcctgaaggccgacgataccgccgtgtacttttgtgcccggggcggatttggctctagctactggtacttcgacgtgtggggccagggcagcctggtgaccgtgtctagcggaggcggaggatcaggtggcggtggatctggcggtggtggctctgacatccagctgacacagagcccatctagcctgagcgctagcgtgggcgacagagtgtctattacctgtaaagcttctcaggacgtgtccatcgccgtcgcctggtatcagcagaagcccggcaaggcccctaagctgctgatctacagcgcctcctacagatacaccggcgtgcccgatagattcagcggaagcggcagcggaacagattttaccctgacaatcagcagcctgcagcctgaggacttcgccgtgtactactgccagcaacactacatcacccctctgaccttcggcgccggcaccaaggtggaaatcaagcggtcaggcggcggaggagcggccgcatctggtcaagttctgctggagtcaaacattaaagtgctgcctacttggagcactcctgttcagcctggatatcaggtttccttttgtttggtcatggtacttctctttgcggtagacactggtctctattttagtgtcaaaactaatatacgctcctccacgagggattggaaggaccataagttcaaatggaggaaggacccgcaggacaaatga

SEQ ID NO: 263 - DNA, 66 bp, synthetic construct
  source 1..66: mol_type = other DNA
tctggtcaagttctgctggagtcaaacattaaagtgctgcctacttggagcactcctgttcagcct

SEQ ID NO: 264 - DNA, 1803 bp, synthetic construct
  source 1..1803: mol_type = other DNA
atgtggctgcagtctctgctgctgctgggaacagtggcctgtagcatctctcaagtgcagctgcagcagagcggcagcgagctgaaaaagcccggagccagcgtgaaagtgtcctgcaaggcttctggctacacattcaccaattacggcatgaactgggtcaagcaggcccctggacagggcctgaagtggatgggctggatcaacacctacaccggcgaacctacatacacagatgacttcaagggcagattcgccttcagcctggacaccagcgtgtccaccgcttatctgcagatcagcagcctgaaggccgacgataccgccgtgtacttttgtgcccggggcggatttggctctagctactggtacttcgacgtgtggggccagggcagcctggtgaccgtgtctagcggaggcggaggatcaggtggcggtggatctggcggtggtggctctgacatccagctgacacagagcccatctagcctgagcgctagcgtgggcgacagagtgtctattacctgtaaagcttctcaggacgtgtccatcgccgtcgcctggtatcagcagaagcccggcaaggcccctaagctgctgatctacagcgcctcctacagatacaccggcgtgcccgatagattcagcggaagcggcagcggaacagattttaccctgacaatcagcagcctgcagcctgaggacttcgccgtgtactactgccagcaacactacatcacccctctgaccttcggcgccggcaccaaggtggaaatcaagcggtcaggcggcggaggagcggccgcatatccacctgtcatagttgaaatgaattccagtgttgaggctatcgagggcagtcacgtatcactcctgtgtggtgcagattccaatccaccacccctccttacatggatgcgggatggaactgttctgagagaagcggtggcggaaagtttgctccttgaattggaggaggttactcccgccgaggacggcgtttatgcctgcctggccgagaatgcgtacggacaagacaatcgaacggtcggtttgagcgtgatgtacgcgccttggaaacctacggttaacggcactatggttgcggtagaaggggaaacggtatccatactctgtagtacacaatcaaatcctgatcccatcctcacgatctttaaagagaaacaaatcctttccacagtcatttatgagtctgagcttcagctcgaactgccagcagtctcccctgaggatgatggagaatattggtgcgttgccgaaaaccagtatggccagagagctacagcgttcaatctcagcgtagaatttgctccagttctcttgctggagagtcactgtgcggcggcacgggatactgtccagtgtctttgtgtagtgaaaagcaatcctgagccttctgtagcttttgagttgccttcacgcaacgtgacggtaaatgagagcgaacgcgagttcgtgtatagtgagagaagcggattggtgctgacttcaatcctcacgcttcggggccaggcgcaggcgccacctcgcgtgatttgcactgctcggaacctttacggcgcaaaatccttggagctgccgtttcagggagcccatcggcttatgtgggctaagattggtcctgtgggggctggatatcaggtttccttttgtttggtcatggtacttctctttgcggtagacactggtctctattttagtgtcaaaactaatatacgctcctccacgagggattggaaggaccataagttcaaatggaggaaggacccgcaggacaaatga

SEQ ID NO: 265 - DNA, 1002 bp, synthetic construct
  source 1..1002: mol_type = other DNA
atgtggctgcagtctctgctgctgctgggaacagtggcctgtagcatctctgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttcaggcggcggaggagcggccgcaggaagtggtctggccgtaagtaccatatccagcttttttccgccaggatatcaggtttccttttgtttggtcatggtacttctctttgcggtagacactggtctctattttagtgtcaaaactaatatacgctcctccacgagggattggaaggaccataagttcaaatggaggaaggacccgcaggacaaatga

SEQ ID NO: 266 - DNA, 732 bp, synthetic construct
  source 1..732: mol_type = other DNA
gacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttct

SEQ ID NO: 267 - DNA, 1458 bp, synthetic construct
  source 1..1458: mol_type = other DNA
atgaataagcagcgagggaccttttcagaagtctcactcgctcaggatcctaaaagacaacaaagaaagcctaagggaaataagtccagcatatcaggtacggaacaggaaatttttcaagtcgaactgaatttgcagaaccctagcctgaatcaccaaggtatcgacaagatctatgattgtcaagggctcctgccaccgcctgaaaagcttacggcggaggtgctgggcattatttgtatagtcctgatggcaactgtacttaaaactattgtacttatcccgtttctggaacaaaacaatttttctccgaacactcggacacaaaaggcccgacattgtggtcactgtccagaagagtggataacttactctaatagctgttactatatcggaaaagagaggagaacgtgggaagaaagcttgctcgcatgcacttccaaaaactcttcactcttgtccattgataacgaggaggagatgaaatttctggcctcaatcctgccatcatcttggataggcgtattccgcaactcaagtcatcacccttgggtaactataaatggtttggcgttcaagcacaagattaaagactctgataatgccgagttgaactgcgctgttcttcaggtgaaccgcctcaaatctgcccagtgcggaagttctatgatatatcactgcaaacataaactgtcaggcggcggaggagcggccgcaggaagtgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttga

SEQ ID NO: 268 - DNA, 735 bp, synthetic construct
  source 1..735: mol_type = other DNA
gacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttga

SEQ ID NO: 269 - DNA, 1044 bp, synthetic construct
  source 1..1044: mol_type = other DNA
atgaataagcagcgagggaccttttcagaagtctcactcgctcaggatcctaaaagacaacaaagaaagcctaagggaaataagtccagcatatcaggtacggaacaggaaatttttcaagtcgaactgaatttgcagaaccctagcctgaatcaccaaggtatcgacaagatctatgattgtcaagggctcctgccaccgcctgaaaagcttacggcggaggtgctgggcattatttgtatagtcctgatggcaactgtacttaaaactattgtactttcaggcggcggaggagcggccgcaggaagtgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttga

SEQ ID NO: 270 - DNA, 279 bp, synthetic construct
  source 1..279: mol_type = other DNA
atgaataagcagcgagggaccttttcagaagtctcactcgctcaggatcctaaaagacaacaaagaaagcctaagggaaataagtccagcatatcaggtacggaacaggaaatttttcaagtcgaactgaatttgcagaaccctagcctgaatcaccaaggtatcgacaagatctatgattgtcaagggctcctgccaccgcctgaaaagcttacggcggaggtgctgggcattatttgtatagtcctgatggcaactgtacttaaaactattgtactt

SEQ ID NO: 271 - DNA, 1413 bp, synthetic construct
  source 1..1413: mol_type = other DNA
atggggtggatacgaggcaggaggtctcggcacagctgggagatgtcagagtttcacaactacaacctcgacctgaaaaaatccgacttctctacccgatggcaaaagcagcgatgtccggtagtgaagtcaaaatgtcgggaaaacgcatctccgttttttttttgctgcttcatagccgtcgcgatgggcataagattcatcattatggtgactatttggtctgccgtgtttcttaatagcttgttcaaccaagaagtgcaaataccccttaccgagagttactgtggtccgtgtcctaagaactggatatgttacaagaacaactgctaccaattcttcgatgaaagtaaaaactggtatgagtcacaggcaagctgcatgagccaaaacgctagtctcctcaaagtctacagtaaagaggatcaggacctgctgaaacttgtgaaaagttatcactggatgggtttggtacacattccaactaacggttcctggcaatgggaagatggatcaatccttagccccaatctcctcactattatagaaatgcaaaagggtgactgtgctctgtacgcgtccagctttaaggggtatatcgagaattgtagcacacccaatacttacatctgtatgcaacgaaccgtgtcaggcggcggaggagcggccgcaggaagtgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttga

SEQ ID NO: 272 - DNA, 648 bp, synthetic construct
  source 1..648: mol_type = other DNA
atggggtggatacgaggcaggaggtctcggcacagctgggagatgtcagagtttcacaactacaacctcgacctgaaaaaatccgacttctctacccgatggcaaaagcagcgatgtccggtagtgaagtcaaaatgtcgggaaaacgcatctccgttttttttttgctgcttcatagccgtcgcgatgggcataagattcatcattatggtgactatttggtctgccgtgtttcttaatagcttgttcaaccaagaagtgcaaataccccttaccgagagttactgtggtccgtgtcctaagaactggatatgttacaagaacaactgctaccaattcttcgatgaaagtaaaaactggtatgagtcacaggcaagctgcatgagccaaaacgctagtctcctcaaagtctacagtaaagaggatcaggacctgctgaaacttgtgaaaagttatcactggatgggtttggtacacattccaactaacggttcctggcaatgggaagatggatcaatccttagccccaatctcctcactattatagaaatgcaaaagggtgactgtgctctgtacgcgtccagctttaaggggtatatcgagaattgtagcacacccaatacttacatctgtatgcaacgaaccgtg

SEQ ID NO: 273 - DNA, 1365 bp, synthetic construct
  source 1..1365: mol_type = other DNA
atgtggctgcagtctctgctgctgctgggaacagtggcctgtagcatctctgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttcaggcggcggaggagcggccgcaggaagtctgtgggttagccagccacccgagattcgaaccctcgaaggcagtagtgctttcttgccgtgtagcttcaatgcgtcccaagggcgactggcaatcggttcagtcacatggtttcgcgacgaagttgtacctggcaaggaggtcaggaatgggacaccggaatttcgcggccgactggccccgttggcatcttcccgatttcttcatgatcaccaggcagagcttcacattcgcgacgtacgaggacacgacgcaagcatctatgtatgtagagttgaagttttgggacttggagtaggcacagggaatgggactaggttggtagtggaaaaggagcatccccagttgggcgcaggtaccgtacttcttctgcgggcagggttctatgctgtcagctttctgtctgtggcagttgggtccacagtctattaccagggtaagtgtctcacgtggaagggaccacggcggcaattgcctgcggttgttcccgcacccctccctcctccatgcggttcaagtgcacatctccttccgccagttccaggcggctga

SEQ ID NO: 274 - DNA, 1014 bp, synthetic construct
  source 1..1014: mol_type = other DNA
atgtggctgcagtctctgctgctgctgggaacagtggcctgtagcatctctgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttcaggcggcggaggagcggccgcaggaagtgcaggtaccgtacttcttctgcgggcagggttctatgctgtcagctttctgtctgtggcagttgggtccacagtctattaccagggtaagtgtctcacgtggaagggaccacggcggcaattgcctgcggttgttcccgcacccctccctcctccatgcggttcaagtgcacatctccttccgccagttccaggcggctga

SEQ ID NO: 275 - DNA, 1581 bp, synthetic construct
  source 1..1581: mol_type = other DNA
atgtggctgcagtctctgctgctgctgggaacagtggcctgtagcatctctgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttcaggcggcggaggagcggccgcaggaagtcagtcaaaagcacaagttctgcagagtgtagcaggccagacacttacggttaggtgtcaatatccgcctaccggcagtctgtatgagaaaaagggctggtgtaaagaagcgtccgcgttggtttgcattcggctcgtaacgagctccaagcctcggactatggcatggacatcacggttcactatatgggacgaccctgatgcaggattttttactgtcaccatgacggaccttcgggaggaggattccggccactattggtgtagaatctatagaccgtcagacaattctgtcagcaagagcgtgcgcttctaccttgtggtctctccggcttccgcctccacccaaacatcttggactccgagggatctcgtgtcatcacaaacccagacacagagttgtgtgccgcccacggcgggagcaagacaggctccggagagcccatctacaattccggtccctagccaaccacagaactctaccttgaggcccggacccgctgcacccatcgctttggttccagtgttttgcggactccttgttgccaagtcacttgtcctttctgctctcctggtatggtggggcgacatttggtggaaaacgatgatggagcttcgatccttggacacacagaaggcgacatgtcatctccaacaggtgacagacctgccatggactagtgtgtcaagtcccgtcgagcgcgaaatcctttatcataccgtggcccgaaccaaaataagcgacgatgacgatgagcacactctgtga

SEQ ID NO: 276 - DNA, 1068 bp, synthetic construct
  source 1..1068: mol_type = other DNA
atgtggctgcagtctctgctgctgctgggaacagtggcctgtagcatctctgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttcaggcggcggaggagcggccgcaggaagtttggttccagtgttttgcggactccttgttgccaagtcacttgtcctttctgctctcctggtatggtggggcgacatttggtggaaaacgatgatggagcttcgatccttggacacacagaaggcgacatgtcatctccaacaggtgacagacctgccatggactagtgtgtcaagtcccgtcgagcgcgaaatcctttatcataccgtggcccgaaccaaaataagcgacgatgacgatgagcacactctgtga

SEQ ID NO: 277 - DNA, 255 bp, synthetic construct
  source 1..255: mol_type = other DNA
ttggttccagtgttttgcggactccttgttgccaagtcacttgtcctttctgctctcctggtatggtggggcgacatttggtggaaaacgatgatggagcttcgatccttggacacacagaaggcgacatgtcatctccaacaggtgacagacctgccatggactagtgtgtcaagtcccgtcgagcgcgaaatcctttatcataccgtggcccgaaccaaaataagcgacgatgacgatgagcacactctgtga

SEQ ID NO: 278 - DNA, 1665 bp, synthetic construct
  source 1..1665: mol_type = other DNA
atgtggctgcagtctctgctgctgctgggaacagtggcctgtagcatctctgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttcaggcggcggaggagcggccgcaggaagtcagcaacagacgctgcccaaaccatttatttgggctgaacctcactttatggtccccaaggaaaaacaagttacaatctgttgccaggggaattacggagctgtggagtaccagctgcatttcgagggatctctttttgcggttgataggcccaagccgcccgagcggatcaacaaggtgaagttttacataccagatatgaactccaggatggcgggacagtactcctgtatctatcgcgtgggcgagctgtggagtgagccttccaatctgcttgatcttgtcgtcaccgagatgtacgatacacctacgctgagcgttcatcccgggccggaagttattagtggcgaaaaggtcactttctattgccgccttgacacggctacgtcaatgttcctcttgctgaaagaaggaagatcttcccatgtccaacgaggatacggaaaagtccaagcggaatttcccctgggaccagtaactaccgctcataggggaacataccgatgcttcggcagctacaacaaccacgcttggagttttccgtctgagcctgtaaaattgctcgttaccggagacattgagaacacgagccttgcccctgaagatccgacgttcccagcagatacatgggggacttatctgttgactacggagacaggacttcaaaaggaccatgcgttgtgggatcatacagctcagaacttgctccgcatgggcctggcctttcttgtactcgttgcactcgtttggttccttgttgaggattggctctctagaaagagaactagagaacgggcctccagggcatccacgtgggaaggccgcagacgactcaatacccagaccctgtga

SEQ ID NO: 279 - DNA, 954 bp, synthetic construct
  source 1..954: mol_type = other DNA
atgtggctgcagtctctgctgctgctgggaacagtggcctgtagcatctctgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttcaggcggcggaggagcggccgcaggaagtatgggcctggcctttcttgtactcgttgcactcgtttggttccttgttgaggattggctctctagaaagagaactagagaacgggcctccagggcatccacgtgggaaggccgcagacgactcaatacccagaccctgtga

SEQ ID NO: 280 - DNA, 1101 bp, synthetic construct
  source 1..1101: mol_type = other DNA
atgtggctgcagtctctgctgctgctgggaacagtggcctgtagcatctctgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttcaggcggcggaggagcggccgcaggatgctacagcgagaccctgcccatccaggtggagcagaacttcctgagcaacctgttcgtggccagctggatcaccgtgatgatcatcttcaggatcggcatggccgtggccatcttctgctgcttcttcttccccagcgccaacgagaggtgcaagagcaaggtggtgccctgcaggcagaagcagtggaggaccagcttcgacagcaagaagctggacctgaactacaaccacttcgagagcatggagtggagccacaggagcaggaggggcaggatctggggcatgtga

SEQ ID NO: 281 - DNA, 27 bp, synthetic construct
  source 1..27: mol_type = other DNA
tcaggcggcggaggagcggccgcagga

SEQ ID NO: 282 - DNA, 1056 bp, synthetic construct
  source 1..1056: mol_type = other DNA
atgtggctgcagtctctgctgctgctgggaacagtggcctgtagcatctctgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttcaggcggcggaggagcggccgcaggaaatgggactaggttggtagtggaaaaggagcatccccagttgggcgcaggtaccgtacttcttctgcgggcagggttctatgctgtcagctttctgtctgtggcagttgggtccacagtctattaccagggtaagtgtctcacgtggaagggaccacggcggcaattgcctgcggttgttcccgcacccctccctcctccatgcggttcaagtgcacatctccttccgccagttccaggcggctga

SEQ ID NO: 283 - DNA, 1122 bp, synthetic construct
  source 1..1122: mol_type = other DNA
atgtggctgcagtctctgctgctgctgggaacagtggcctgtagcatctctgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttcaggcggcggaggagcggccgcaggagtccctagccaaccacagaactctaccttgaggcccggacccgctgcacccatcgctttggttccagtgttttgcggactccttgttgccaagtcacttgtcctttctgctctcctggtatggtggggcgacatttggtggaaaacgatgatggagcttcgatccttggacacacagaaggcgacatgtcatctccaacaggtgacagacctgccatggactagtgtgtcaagtcccgtcgagcgcgaaatcctttatcataccgtggcccgaaccaaaataagcgacgatgacgatgagcacactctgtga

SEQ ID NO: 284 - DNA, 1005 bp, synthetic construct
  source 1..1005: mol_type = other DNA
atgtggctgcagtctctgctgctgctgggaacagtggcctgtagcatctctgacatccagatgacacagagccctagcagcctgtctgcctctgtgggcgatagagtgaccatcacctgtagagccagccaggatgtgaataccgccgtggcctggtatcagcagaagcctggaaaagcccctaagctgctgatctacagcgccagctttctgtacagcggcgtgccaagcagattcagcggcagcagatctggcaccgacttcaccctgaccatctctagcctgcagcctgaggacttcgccacctactactgccagcagcactacaccacacctccaacctttggccagggcaccaaggtggaaatcaagagaacaggcagcaccagcggctctggaaagcctggatctggcgaaggatctgaggtgcagctggttgaatctggcggaggacttgttcagcctggcggctctctgagactgtcttgtgccgccagcggcttcaacatcaaggacacctacatccactgggtccgacaggcccctggaaagggacttgaatgggtcgccagaatctaccccaccaacggctacaccagatacgccgatagcgtgaagggcagattcaccatcagcgccgacaccagcaagaacaccgcctacctgcagatgaacagcctgagagccgaggacaccgccgtgtactactgttctagatggggaggcgacggcttctacgccatggattactggggacagggcaccctggtcacagtttcttcttcaggcggcggaggagcggccgcaggaggacttcaaaaggaccatgcgttgtgggatcatacagctcagaacttgctccgcatgggcctggcctttcttgtactcgttgcactcgtttggttccttgttgaggattggctctctagaaagagaactagagaacgggcctccagggcatccacgtgggaaggccgcagacgactcaatacccagaccctgtga

SEQ ID NO: 285 - RNA, 13 nt, synthetic construct
  source 1..13: mol_type = other RNA
wwwtatttatttw

SEQ ID NO: 286 - RNA, 50 nt, synthetic construct
  source 1..50: mol_type = other RNA
aaaaaaaaaaaaaaaaaaaaaaaaaaaaaaaaaaaaaaaaaaaaaaaaaa

SEQ ID NO: 287 - RNA, 90 nt, synthetic construct
  source 1..90: mol_type = other RNA
  misc_difference 1..90: note = This sequence may encompass 60, 70, 80, or 90 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 288 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 289 - RNA, 110 nt, synthetic construct
  source 1..110: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 290 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 291 - RNA, 130 nt, synthetic construct
  source 1..130: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 292 - RNA, 140 nt, synthetic construct
  source 1..140: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 293 - RNA, 200 nt, synthetic construct
  source 1..200: mol_type = other RNA
  misc_difference 1..200: note = This sequence may encompass 150, 160, 170, 180, 190 or 200 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 294 - protein, 9 aa, synthetic construct
RGYDWYFDV

SEQ ID NO: 295 - RNA, 200 nt, synthetic construct; identical to SEQ ID NO: 293
  source 1..200: mol_type = other RNA

(19) United States
(12) Patent Application Publication
(10) Pub. No.: US 2025/0249099 A1
(43) Pub. Date: Aug. 7, 2025

(54) COMPOSITIONS FOR CELL-SPECIFIC EXPRESSION AND USES THEREOF

(71) Applicant: Myeloid Therapeutics, Inc., Cambridge, MA (US)

(72) Inventors: Daniel GETTS, Medfield, MA (US); Yuxiao Wang, Belmont, MA (US); Neha Diwanji, Waltham, MA (US)

(21) Appl. No.: 19/067,329

(22) Filed: Feb. 28, 2025

Related U.S. Application Data
(63) Continuation of application No. PCT/US2023/031925, filed on Sep. 2, 2023.
(60) Provisional application No. 63/403,449, filed on Sep. 2, 2022.
(60) Provisional application No. 63/403,454, filed on Sep. 2, 2022.
(60) Provisional application No. 63/403,455, filed on Sep. 2, 2022.

(51) Int. Cl. A61K 40/15; A61K 40/31; A61K 40/42; C07K 14/725; C07K 16/32; C12N 15/88

(52) U.S. Cl. CPC A61K 40/15; A61K 40/31; A61K 40/4205; C07K 14/7051; C07K 16/32; C12N 15/88; C07K 2317/622

(57) ABSTRACT

Compositions and methods for making and using engineered NK cells, T cells and B cells that express a chimeric antigen receptor.

### CROSS REFERENCE

This application is a continuation of International Application No. PCT/US2023/031925, filed Sep. 2, 2023, which claims priority to the provisional application U.S. 63/403,449, filed on Sep. 2, 2022; U.S. 63/403,454, filed on Sep. 2, 2022; and U.S. 63/403,455, filed on Sep. 2, 2022, each of which is incorporated herein by reference in its entirety.

### SEQUENCE LISTING

This application contains a Sequence Listing which has been submitted electronically in XML format and is hereby incorporated by reference is its entirety. Said XML copy, created on Sep. 29, 2023, is named 59073-732_601_SL.xml and is 316,824 bytes in size.

### BACKGROUND

Cellular immunotherapy is a promising new technology for fighting difficult to treat diseases, such as cancer, and persistent infections and also certain diseases that are refractory to other forms of treatment. A major breakthrough has come across with the discovery of CAR-T cell and their potential use in immunotherapy. CAR-T cells are T lymphocytes expressing a chimeric antigen receptor which helps target the T cell to specific diseased cells such as cancer cells, and can induce cytotoxic responses intended to kill the target cancer cell or immunosuppression and/or tolerance depending on the intracellular domain employed and co-expressed immunosuppressive cytokines. However, several limitations along the way has slowed the progress on CAR-T cells and dampened its promise in clinical trials.

Revolutionary advancements in nucleic acid technology have encouraged the idea that recombinant polynucleic acid molecules such as CARs can be delivered locally or systemically in an organism in need thereof and induce an effect in the system caused by suitable expression of the sequence encoded by the recombinant polynucleic acid addressing a therapeutic need, and thereby bypassing the need for expensive and time-consuming generation of cells for administering. One overarching issue with such therapy, however, is directing expression of the recombinant polynucleic acid molecule, such as a CAR construct, in a specific cell or tissue type, for it to be most effective in bringing about the therapeutic effect and avoiding non-specific or harmful effects generated as a result of inadvertent expression of the polynucleic acid that has been delivered systemically or locally in a cell that was not the desired or intended cell for the purpose.

Additionally, understanding the limitations of CAR-T cells is the key to leveraging the technology and continue innovations towards better immunotherapy models. Specifically, in T cell malignancies, CAR-T cells appear to have faced a major problem. CAR-T cells and malignant T cells share surface antigen in most T cell lymphomas (TCL), therefore, CAR-T cells are subject to cytotoxicity in the same way as cancer cells. In some instances, the CAR-T products may be contaminated by malignant T cells. Additionally, T cell aplasia is a potential problem due to prolonged persistence of the CAR-T cells. Other limitations include the poor ability for CAR-T cells to penetrate into solid tumors and the potent tumor microenvironment which acts to downregulate their anti-tumor potential. CAR-T cell function is also negatively influenced by the immunosuppressive tumor microenvironment (TME) that leads to endogenous T cell inactivation and exhaustion.

Cells from the innate immune defense repertoire are being recently tapped for their therapeutic potential. Among the early responders of immune defense system, NK cells, myeloid cells and certain lymphoid lineage cells are potent cytotoxic cells, and exhibit target-specific fast and efficient clearance of infective agents, pollutants, infected cells, dead or dying cells, and cells that undergo aberrant physiological changes. Particularly, natural killer (NK) cells, a type of granulocytes, play a vital role in innate immune response. These cells are responsible for the discrimination of target cells from healthy cells and are instrumental in cytolysis without triggering tissue damage. NK cells can lyse cells that exhibit surface markers associated with oncogenic transformation. In addition, NK cells are short-lived and therefore pose no long lasting issues encountered in other modes of cell therapy, for example, T cell therapy. Therefore, NK cells can be utilized as an excellent candidate in the development of in anticancer cell therapy. Similarly B cells and a variety of T cells may likewise be considered.

Using such specific cell types that are residing in vivo as therapeutic vehicles, and targeting individual cell types to express a therapeutic polynucleic acid when administered to a subject, can be a huge challenge for future drug development.

### SUMMARY

The present disclosure relates to targeting innate immune cells, particularly NK cells as the next frontier in immuno-oncology. The present disclosure also relates to targeting T cells and B cells for the purpose of immuno-oncology. The disclosure relates to methods and compositions comprising polynucleic acids encoding one or more polypeptides, the compositions comprising polynucleic acids are formulated for delivery to a subject in need thereof in an aqueous solution via systemic routes, or local routes or topical delivery, such that when exposed to a variety of cells in vivo, the polynucleic acid is expressed in specific cells and not express in all cells in vivo. Therefore, chimeric fusion proteins as disclosed herein are encoded by one or more polynucleic acid of the design disclosed herein, such that when a liquid formulation of a composition comprising the polynucleic acids is administered to a subject, the polynucleic acid, even if taken up by multitude of cells in vivo, will express the encoded polypeptide in a certain cell type in vivo as is designed. The polypeptide encoded by the polynucleic acid may not express, may be degraded or may not be functional in other cell types that are different from the certain cell type as allowed by the design of the polynucleic acids.

In some embodiments, the certain cell type for expression of the polypeptide is an NK cell. In some embodiments, the certain cell type for expression of the polypeptide is a T cell. In some embodiments, the certain cell type for expression of the polypeptide is a B cell.

In one aspect, NK cells are engineered to potentiate an immune function. In one embodiment, the NK cells are human.

In one aspect, NK cells are engineered to express a recombinant protein, encoded by a recombinant polynucleic acid disclosed herein. In an embodiment of the invention, NK cells are engineered in vivo.

In one aspect, provided herein is a composition comprising a recombinant polynucleic acid comprising a sequence encoding a chimeric fusion protein (CFP), the CFP comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by natural killer (NK) cells; and wherein after administration of the composition to a human subject the CFP is expressed on cell surfaces of NK cells of the human subject.

In some embodiments, the recombinant polynucleic acid is encapsulated by a nanoparticle delivery vehicle.

In some embodiments, the transmembrane domain is a transmembrane domain from a cell surface receptor selected from a group consisting of CD39, CD56, CD57, CD94, CD159a, CD159c, CD314, CD335, CD336, CD337, DAP12, DAP10, NKG2C, NKG2D, NKG2E, Ly49D, Ly49D, NKp46, NKp30 and NKp44.

In some embodiments, the transmembrane domain is a transmembrane domain from CD94, CD159a, CD159c, CD314, CD335, CD336, CD337, DAP12, or DAP10.

In some embodiments, the extracellular domain is an extracellular domain from CD39, CD56, CD57, CD94, CD159a, CD159c, CD314, CD335, CD336, CD337, DAP12, DAP10, NKG2C, NKG2D, NKG2E, Ly49D, Ly49D, NKp46, NKp30 or NKp44.

In some embodiments, the extracellular domain is an extracellular domain from CD94, CD159a, CD159c, CD314, CD335, CD336, CD337, DAP12 or DAP10.

In some embodiments, the extracellular domain further a hinge domain from CD8, wherein the hinge domain is operatively linked to the transmembrane domain.

In some embodiments, the CFP is preferentially or specifically expressed in NK cells of the human subject.

In some embodiments, the antigen binding domain comprises a Fab fragment, an scFv domain or an sdAb domain.

In some embodiments, the CFP further comprises an intracellular domain.

In some embodiments, the intracellular domain comprises an intracellular signaling domain from Fc receptor γ subunit, FcαR, FcεR, CD40, CD3ζ, DAP10, DAP12, 2B4, NTB-A, CRACC, 41BB, OX40, CRTAM.

In some embodiments, the intracellular domains further comprises a phosphoinositide 3-kinase (PI3K) recruitment domain.

In some embodiments, the PI3K recruitment domain comprises a sequence with at least 90% sequence identity to YEDMRGILYAAPQLRSIRGQPGPNHEEDADSYENM (SEQ ID NO: 41).

In some embodiments, the intracellular domain comprises an intracellular domain from CD39, CD56, CD57, CD94, CD159a, CD159c, CD314, CD335, CD336, CD337, DAP12, DAP10, NKG2C, NKG2D, NKG2E, Ly49D, Ly49D, NKp46, NKp30 or NKp44.

In some embodiments, the intracellular domain comprises an intracellular domain from CD94, CD159a, CD159c, CD314, CD335, CD336, CD337, DAP12, or DAP10.

Provided herein is a recombinant polynucleic acid composition comprising a recombinant polynucleic acid sequence encoding a chimeric fusion protein (CFP) comprising a transmembrane domain that specifically integrates within a membrane protein complex of a B cell, wherein the B cell is characterized as naturally expressing the membrane protein complex; wherein, when the recombinant polynucleic acid composition comprising the recombinant polynucleic acid sequence is contacted to any cell of a heterogenous cell population, at least greater than 50% of cells within the heterogenous cell population that are characterized as naturally expressing the membrane protein complex, e.g., B cells, expresses the CFP, and a cell within the heterogenous cell population that lacks the membrane protein complex, e.g., non-B cells, fails to express the CFP. In some embodiments, the naturally expressing the membrane protein complex of a B cell may be a CD19 or CD20 TM domain and intracellular domain. In some embodiments, it comprises an extracellular domain that comprises a sequence from the CD19, and an scFv that binds to a cancer antigen.

In some embodiments, the recombinant polynucleic acid composition is expressed in at least greater than 60%, 70%, 80% or 90% B cells within the heterogenous cell population. In some embodiments, the CFR is expressed in at least 50% of B cells in a heterogenous population PBMC, e.g. obtained from peripheral blood drawn at 1, 2, or 3 days after introduction of the polynucleic acid in the system of the subject. In some embodiments, the recombinant polynucleic acid composition less than 10% cells within the heterogenous cell population that lacks a CD20 or CD19 express the CFP. In some embodiments, the CFP is expressed in less than 10% T cells in a population of cells in a biological sample from the subject at 1, 2, or 3 days after administration of a composition comprising the recombinant polynucleic acid. In some embodiments, the CFP is expressed in less than 10% of myeloid cells in a biological sample from the subject at 1, 2, or 3 days after administration of a composition comprising the recombinant polynucleic acid. In some embodiments, the CFP is expressed in less than 10% of epithelial cells in a biological sample from the subject at 1, 2, or 3 days after administration of a composition comprising the recombinant polynucleic acid. In some embodiments, the recombinant polynucleic acid is expressed at greater than 50% of B cells, e.g., greater than 60%, 70%, 80% or 90% of B cells within the heterogenous cell population tested ex vivo. In some embodiments, the recombinant polynucleic acid is expressed at less than 10% of B cells, e.g., less than 9%, 8%, 7%, 6%, 5%, 4%, 3%, 2% 1% or less in cells other than B cells, e.g. epithelial cells or myeloid cells within the heterogenous cell population tested ex vivo.

In some embodiments, the recombinant polynucleic acid is an mRNA.

In some embodiments, the nanoparticle delivery vehicle comprises a lipid nanoparticle. In some embodiments, the lipid nanoparticle comprises a polar lipid. In some embodiments, the lipid nanoparticle comprises a non-polar lipid. In some embodiments, the lipid nanoparticle is from 100 to 300 nm in diameter.

In some embodiments, the lipid nanoparticle comprises (6Z,9Z,28Z,31Z)-heptatriaconta-6,9,28,31-tetraen-19-yl 4-(dimethylamino) butanoate (DLin-MC3-DMA; MC3).

In some embodiments, the lipid nanoparticle comprises (a) a nucleic acid; (b) a cationic lipid; (c) a non-cationic lipid; and (d) a conjugated lipid that inhibits aggregation of particles.

In some embodiments, the nucleic acid comprises a charged polyanioinc nucleic acid.

In one aspect, provided herein is a pharmaceutical composition comprising the composition of one of the embodiments described above and a pharmaceutically acceptable excipient. In some embodiments, the pharmaceutical composition comprises an effective amount of the composition of one of the embodiments described above to inhibit growth of a cancer when administered to a human subject with the cancer.

In one aspect, provided herein is a method of treating cancer in a subject in need thereof comprising administering the pharmaceutical composition of described above in the preceding paragraph to the human subject.

In one aspect, provided herein is a method of introducing the composition of claim 1 into an NK cell comprising electroporating the NK cell in the presence of the recombinant polynucleic acid comprising the sequence encoding the CFP, wherein the recombinant polynucleic acid is configured for expression of the recombinant polynucleic acid in the NK cell of a human subject.

In some embodiments, the antigen binding domain binds to an antigen selected from the group consisting of CD5, HER2, GPC3 and TROP2. In some embodiments, the extracellular domain is an extracellular domain from a protein that multimerizes with a cell surface receptor expressed by NK cells. In some embodiments, the intracellular domain is an intracellular domain from a protein that multimerizes with a cell surface receptor expressed by NK cells. In some embodiments, the transmembrane domain is a transmembrane domain from a protein that is not expressed or not substantially expressed by non-NK cells. In some embodiments, the extracellular domain is an extracellular domain from a protein that is not expressed or not substantially expressed by non-NK cells. In some embodiments, the intracellular domain is an intracellular domain from a protein that is not expressed or not substantially expressed by non-NK cells. In some embodiments, the transmembrane domain is a transmembrane domain from a protein that is not expressed or not substantially expressed by T cells, B cells or myeloid cells. In some embodiments, the extracellular domain is an extracellular domain from a protein that is not expressed or not substantially expressed by T cells, B cells or myeloid cells. In some embodiments, the intracellular domain is an intracellular domain from a protein that is not expressed or not substantially expressed by T cells, B cells or myeloid cells.

In one aspect, provided herein is a composition comprising a recombinant polynucleic acid comprising a sequence encoding a chimeric antigen receptor (CAR) protein, the CAR comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by T cells; and wherein after administration of the composition to a human subject the CFP is expressed on cell surfaces of T cells of the human subject.

In some embodiments, the recombinant polynucleic acid is encapsulated by a nanoparticle delivery vehicle. In one aspect, provided herein is a composition comprising a recombinant polynucleic acid comprising a sequence encoding a chimeric fusion protein (CFP), the CFP comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by T cells; and wherein after administration of the composition to a human subject the CFP is expressed on cell surfaces of T cells of the human subject.

In some embodiments, the recombinant polynucleic acid is encapsulated by a nanoparticle delivery vehicle.

In some embodiments, the transmembrane domain is a transmembrane domain from a cell surface receptor selected from a group consisting of CD3, CD4, CD5, CD7, CD8, CD28, CD48, CD3ε, CD3δ, CD3γ, CD3ζ, TCRα chain, TCRβ chain, TCRγ chain and TCRδ chain. In some embodiments, the transmembrane domain is a transmembrane domain from a cell surface receptor selected from a group consisting of CD3, CD4, CD5, CD7, CD8, CD28 and CD48.

In some embodiments, the extracellular domain is an extracellular domain from CD3, CD4, CD5, CD7, CD8, CD28, CD48, CD3ε, CD3δ, CD3γ, CD3ζ, TCRα chain, TCRβ chain, TCRγ chain and TCRδ chain. In some embodiments, the extracellular domain is an extracellular domain from CD3, CD4, CD5, CD7, CD8, CD28 or CD48.

In some embodiments, the extracellular domain comprises a hinge domain from CD8, wherein the hinge domain is operatively linked to the transmembrane domain. In some embodiments, the CFP is preferentially or specifically expressed in T cells of the human subject. In some embodiments, the antigen binding domain comprises a Fab fragment, an scFv domain or an sdAb domain. In some embodiments, the CFP further comprises an intracellular domain. In some embodiments, the intracellular domain comprises an intracellular signaling domain from FcγR, FcαR, FcεR, CD40 or CD3ζ.

In some embodiments, the intracellular domain further comprises a phosphoinositide 3-kinase (PI3K) recruitment domain. In some embodiments, the intracellular domain comprises an intracellular domain from CD3, CD4, CD5, CD7, CD8, CD28, CD48, CD3ε, CD3δ, CD3γ or CD3ζ. In some embodiments, the intracellular domain comprises an intracellular domain from CD3, CD4, CD5, CD7, CD8, CD28 or CD48.

In some embodiments, the recombinant polynucleic acid is an mRNA.

In some embodiments, the nanoparticle delivery vehicle comprises a lipid nanoparticle. In some embodiments, the lipid nanoparticle comprises a polar lipid. In some embodiments, the lipid nanoparticle comprises a non-polar lipid. In some embodiments, the lipid nanoparticle is from 100 to 300 nm in diameter. In some embodiments, the lipid nanoparticle comprises (6Z,9Z,28Z,31Z)-heptatriaconta-6,9,28,31-tetraen-19-yl 4-(dimethylamino) butanoate (DLin-MC3-DMA; MC3). In some embodiments, the lipid nanoparticle comprises (a) a nucleic acid; (b) a cationic lipid; (c) a non-cationic lipid; and (d) a conjugated lipid that inhibits aggregation of particles. In some embodiments, the nucleic acid comprises a charged polyanioinc nucleic acid.

In one aspect, provided herein is a pharmaceutical composition comprising the composition comprising a recombinant polynucleic acid comprising a sequence encoding a chimeric fusion protein (CFP), the CFP comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by T cells; and a pharmaceutically acceptable excipient. In one embodiment, the pharmaceutical composition comprises an effective amount of the composition described above, to inhibit growth of a cancer when administered to a human subject with the cancer.

In one aspect, provided herein is a method of treating cancer in a subject in need thereof comprising administering the pharmaceutical composition comprising a recombinant polynucleic acid comprising a sequence encoding a chimeric fusion protein (CFP), and a pharmaceutically acceptable excipient to the subject; wherein the CFP comprising an extracellular domain comprising an antigen binding domain, and a transmembrane domain operatively linked to the extracellular domain; wherein the transmembrane domain is a transmembrane domain from a protein that multimerizes with a cell surface receptor expressed by T cells.

In one aspect, provided herein is a method of introducing the composition described above into a T cell, comprising, electroporating the T cell in the presence of the recombinant polynucleic acid comprising the sequence encoding the CFP, wherein the recombinant polynucleic acid is configured for expression of the recombinant polynucleic acid in T cells of a human subject.

In some embodiments, the antigen binding domain binds to an antigen selected from the group consisting of CD5, HER2, GPC3 and TROP2.

In some embodiments, the extracellular domain is an extracellular domain from a protein that multimerizes with a cell surface receptor expressed by T cells. In some embodiments, the intracellular domain is an intracellular domain from a protein that multimerizes with a cell surface receptor expressed by T cells. In some embodiments, the transmembrane domain is a transmembrane domain from a protein that is not expressed or not substantially expressed by non-T cells. In some embodiments, the extracellular domain is an extracellular domain from a protein that is not expressed or not substantially expressed by non-T cells.

In some embodiments, the intracellular domain is an intracellular domain from a protein that is not expressed or not substantially expressed by non-T cells. In some embodiments, the transmembrane domain is a transmembrane domain from a protein that is not expressed or not substantially expressed by NK cells, B cells or myeloid cells. In some embodiments, the extracellular domain is an extracellular domain from a protein that is not expressed or not substantially expressed by NK cells, B cells or myeloid cells. In some embodiments, the intracellular domain is an intracellular domain from a protein that is not expressed or not substantially expressed by NK cells, B cells or myeloid cells.

Provided herein is a recombinant polynucleic acid composition comprising a recombinant polynucleic acid sequence encoding a chimeric fusion protein (CFP) comprising a transmembrane domain that specifically integrates within a membrane protein complex of a cell, wherein the cell is characterized as naturally expressing the membrane protein complex; wherein, when the recombinant polynucleic acid composition comprising the recombinant polynucleic acid sequence is contacted to any cell of a heterogenous cell population, at least greater than 50% of cells within the heterogenous cell population characterized as naturally expressing the membrane protein complex expresses the CFP, and a cell within the heterogenous cell population that lacks the membrane protein complex fails to express the CFP; and wherein the cell that is characterized as naturally expressing the membrane protein complex is a NK cell, a B cell or a T cell.

In some embodiments, the recombinant polynucleic acid composition is expressed in at least greater than 60%, 70%, 80% or 90% of cells within the heterogenous cell population characterized as naturally expressing the membrane protein complex expresses the CFP.

In some embodiments, the recombinant polynucleic acid composition less than 10% cells within the heterogenous cell population that lacks the membrane protein complex express the CFP.

In some embodiments, the recombinant polynucleic acid composition comprises one or more recombinant polynucleic acid molecules comprising more than one recombinant polynucleic acid sequences, each recombinant polynucleic acid sequence of the more than one recombinant polynucleic acid sequences comprises a unique sequence encoding a transmembrane domain.

In some embodiments, the recombinant polynucleic acid composition comprises a polypeptide encoded by each recombinant polynucleic acid sequence is expressed in a specific cell type.

In some embodiments, the recombinant polynucleic acid composition each recombinant polynucleic acid sequence is expressed in a cell type distinct from a different sequence.

In some embodiments, the recombinant polynucleic acid composition the transmembrane domain is operably linked with an extracellular domain, wherein the extracellular domain comprises an antigen binding domain. In some embodiments, the recombinant polynucleic acid composition the antigen binding domain binds to a cell surface antigen on a target cell. In some embodiments, the recombinant polynucleic acid composition the target cell is a cancer cell. In some embodiments, the recombinant polynucleic acid composition the target cell is an infected cell. In some embodiments, the recombinant polynucleic acid composition the target cell is an autoimmune cell.

In some embodiments, the recombinant polynucleic acid composition the recombinant polynucleic acid further comprises a nucleic acid delivery vehicle. In some embodiments, the recombinant polynucleic acid composition the recombinant polynucleic acid composition comprises a lipid. In some embodiments, the recombinant polynucleic acid composition the recombinant polynucleic acid composition comprises a lipid nanoparticle (LNP). In some embodiments, the recombinant polynucleic acid composition the recombinant polynucleic acid composition further comprises a nucleic acid delivery vehicle comprising a cationic lipid, a non-cationic lipid, a neutral lipid, a cholesterol or a polyethylenel glycol (PEG)-lipid. In some embodiments, the recombinant polynucleic acid composition comprises a polymeric nucleic acid delivery vehicle. Provided herein is a pharmaceutical composition comprising any one or more of the recombinant polynucleic acid composition described herein and a pharmaceutically acceptable excipient. In some embodiments, the pharmaceutical composition is formulated for an in vivo delivery. In one aspect, provided herein is a composition comprising a recombinant polynucleic acid comprising a sequence encoding a chimeric fusion protein (CFP), the CFP comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by T cells; and wherein after administration of the composition to a human subject the CFP is expressed on cell surfaces of T cells of the human subject.

In some embodiments, the recombinant polynucleic acid is encapsulated by a nanoparticle delivery vehicle.

In some embodiments, the transmembrane domain is a transmembrane domain from a cell surface receptor selected from a group consisting of CD3, CD4, CD5, CD7, CD8, CD28, CD48, CD3δ, CD3δ, CD3γ, CD3ζ, TCRα chain, TCRβ chain, TCRγ chain and TCRδ chain. In some embodiments, the transmembrane domain is a transmembrane domain from a cell surface receptor selected from a group consisting of CD3, CD4, CD5, CD7, CD8, CD28 and CD48. In some embodiments, in the extracellular domain is an extracellular domain from CD3, CD4, CD5, CD7, CD8, CD28, CD48, CD3δ, CD3δ, CD3γ, CD3ζ, TCRα chain, TCRβ chain, TCRγ chain and TCRδ chain. In some embodiments, the extracellular domain is an extracellular domain from CD3, CD4, CD5, CD7, CD8, CD28 or CD48. In some embodiments, the extracellular domain comprises a hinge domain from CD8, wherein the hinge domain is operatively linked to the transmembrane domain. In some embodiments, the CFP is preferentially or specifically expressed in T cells of the human subject. In some embodiments, the antigen binding domain comprises a Fab fragment, an scFv domain or an sdAb domain. In some embodiments, the CFP further comprises an intracellular domain. In some embodiments, the intracellular domain comprises an intracellular signaling domain from FcγR, FcαR, FcεR, CD40 or CD3ζ. In some embodiments, the intracellular domain further comprises a phosphoinositide 3-kinase (PI3K) recruitment domain. In some embodiments, the PI3K recruitment domain comprises a sequence with at least 90% sequence identity to the sequence YEDMRGILYAAPQLRSIRGQPGPNHEEDADSYENM (SEQ ID NO: 41). In some embodiments, the intracellular domain comprises an intracellular domain from CD3, CD4, CD5, CD7, CD8, CD28, CD48, CD3δ, CD3δ, CD3γ or CD3ζ. In some embodiments, the intracellular domain comprises an intracellular domain from CD3, CD4, CD5, CD7, CD8, CD28 or CD48. In some embodiments, the recombinant polynucleic acid is an mRNA. In some embodiments, the nanoparticle delivery vehicle comprises a lipid nanoparticle. In some embodiments, the lipid nanoparticle comprises a polar lipid. In some embodiments, the lipid nanoparticle comprises a non-polar lipid. In some embodiments, the lipid nanoparticle is from 100 to 300 nm in diameter. In some embodiments, the lipid nanoparticle comprises (6Z,9Z,28Z,31Z)-heptatriaconta-6,9,28,31-tetraen-19-yl 4-(dimethylamino) butanoate (DLin-MC3-DMA; MC3). In some embodiments, the lipid nanoparticle comprises (a) a nucleic acid; (b) a cationic lipid; (c) a non-cationic lipid; and (d) a conjugated lipid that inhibits aggregation of particles. In some embodiments, the nucleic acid comprises a charged polyanioinc nucleic acid.

Provided herein is a pharmaceutical composition comprising the composition comprising the CFP comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by T cells; and a pharmaceutically acceptable excipient.

In some embodiments, the pharmaceutical composition comprises an effective amount of the composition described herein to inhibit growth of a cancer when administered to a human subject with the cancer.

Provided herein is a method of treating cancer in a subject in need thereof comprising administering the pharmaceutical composition described herein to the subject. In some embodiments the method comprises introducing the composition into a T cell comprising electroporating the T cell in the presence of the recombinant polynucleic acid comprising the sequence encoding the CFP, wherein the recombinant polynucleic acid is configured for expression of the recombinant polynucleic acid in T cells of a human subject. In some embodiments, the antigen binding domain binds to an antigen selected from the group consisting of CD5, HER2, GPC3 and TROP2. In some embodiments, the extracellular domain is an extracellular domain from a protein that multimerizes with a cell surface receptor expressed by T cells. In some embodiments, the intracellular domain is an intracellular domain from a protein that multimerizes with a cell surface receptor expressed by T cells. In some embodiments, the transmembrane domain is a transmembrane domain from a protein that is not expressed or not substantially expressed by non-T cells. In some embodiments, the extracellular domain is an extracellular domain from a protein that is not expressed or not substantially expressed by non-T cells. In some embodiments, the intracellular domain is an intracellular domain from a protein that is not expressed or not substantially expressed by non-T cells. In some embodiments, the transmembrane domain is a transmembrane domain from a protein that is not expressed or not substantially expressed by NK cells, B cells or myeloid cells. In some embodiments, the extracellular domain is an extracellular domain from a protein that is not expressed or not substantially expressed by NK cells, B cells or myeloid cells. In some embodiments, the intracellular domain is an intracellular domain from a protein that is not expressed or not substantially expressed by NK cells, B cells or myeloid cells.

Provided herein is a recombinant polynucleic acid composition comprising a recombinant polynucleic acid sequence encoding a chimeric fusion protein (CFP) comprising a transmembrane domain that specifically integrates within a membrane protein complex of a B cell, wherein the B cell is characterized as naturally expressing the membrane protein complex; wherein, when the recombinant polynucleic acid composition comprising the recombinant polynucleic acid sequence is contacted to any cell of a heterogenous cell population, at least greater than 50% of cells within the heterogenous cell population that are characterized as naturally expressing the membrane protein complex, e.g., B cells, expresses the CFP, and a cell within the heterogenous cell population that lacks the membrane protein complex, e.g., non-B cells, fails to express the CFP. In some embodiments, the naturally expressing the membrane protein complex of a B cell may be a CD19 or CD20 TM domain and intracellular domain. In some embodiments, it comprises an extracellular domain that comprises a sequence from the CD19, and an scFv that binds to a cancer antigen.

In some embodiments, the recombinant polynucleic acid composition is expressed in at least greater than 60%, 70%, 80% or 90% B cells within the heterogenous cell population. In some embodiments, the CFR is expressed in at least 50% of B cells in a heterogenous population PBMC, e.g. obtained from peripheral blood drawn at 1, 2, or 3 days after introduction of the polynucleic acid in the system of the subject. In some embodiments, the recombinant polynucleic acid composition less than 10% cells within the heterogenous cell population that lacks a CD20 or CD19 express the CFP. In some embodiments, the CFP is expressed in less than 10% T cells in a population of cells in a biological sample from the subject at 1, 2, or 3 days after administration of a composition comprising the recombinant polynucleic acid. In some embodiments, the CFP is expressed in less than 10% of myeloid cells in a biological sample from the subject at 1, 2, or 3 days after administration of a composition comprising the recombinant polynucleic acid. In some embodiments, the CFP is expressed in less than 10% of epithelial cells in a biological sample from the subject at 1, 2, or 3 days after administration of a composition comprising the recombinant polynucleic acid. In some embodiments, the recombinant polynucleic acid is expressed at greater than 50% of B cells, e.g., greater than 60%, 70%, 80% or 90% of B cells within the heterogenous cell population tested ex vivo. In some embodiments, the recombinant polynucleic acid is expressed at less than 10% of B cells, e.g., less than 9%, 8%, 7%, 6%, 5%, 4%, 3%, 2% 1% or less in cells other than B cells, e.g. epithelial cells or myeloid cells within the heterogenous cell population tested ex vivo.

In one aspect, provided herein is a composition comprising a nucleic acid encoding a sequence having at least 80% sequence identity to any one of sequences of SEQ ID NOs: 20-40. In some embodiments, the composition comprises a nucleic acid encoding a sequence having at least 90% sequence identity to any one of sequences of SEQ ID NOs: 20-40. In some embodiments, the composition comprises a nucleic acid encoding a sequence having at least 95% sequence identity to any one of sequences of SEQ ID NOs: 20-40.

In one aspect, provided herein is a composition comprising a nucleic acid encoding a sequence having at 80% identical to any one the sequences of SEQ ID NO: 1-19; further comprising a lipid molecule.

In some embodiments, the composition comprises a nucleic acid encoding a sequence having a polynucleic acid molecule having a sequence that is at least 90% identical to any one the sequences of SEQ ID NO: 1-19. In some embodiments, the composition comprises a nucleic acid encoding a sequence having composition comprises a polynucleic acid comprising a sequence having at least 80% sequence identity to any one of sequences of SEQ ID NOs: 1-19, further comprising a sequence encoding an anti-TROP2 binding domain. In some embodiments, the anti-TROP2 binding domain comprises a HC CDR3 sequence, GGFGSSYWYFDV (SEQ ID NO: 42) and a LC CDR3 sequence, QQHYITPLT (SEQ ID NO: 43).

Provided herein is a composition comprising a polynucleic acid comprising a sequence having at least 80% sequence identity to any one of sequences of SEQ ID NOs: 1-19, further comprising a sequence encoding an anti-GPC3 binding domain. Provided herein is a composition comprising a polynucleic acid comprising a sequence having at least 80% sequence identity to any one of sequences of SEQ ID NOs: 1-19, further comprising a sequence encoding an anti-HER2 binding domain. Provided herein is a composition comprising a polynucleic acid comprising a sequence having at least 80% sequence identity to any one of sequences of SEQ ID NOs: 1-19, further comprising a sequence encoding an anti-CD5 binding domain.

### BRIEF DESCRIPTION OF FIGURES

FIG. 1 shows schematic diagram of an exemplary screening assay set up for identifying immune receptors that exhibits dependency on coreceptors that are endogenous to NK cells for expression. This assay is designed to screen through immune receptors to determine whether their expression are dependent on the presence of a ITAM co-receptor as described elsewhere (e.g. Table 3) and then build chimeric fusion protein (CFP) for in vivo delivery. The screening and design of such CFPs are intended for preparing CFP mRNA constructs that can be delivered directly in vivo using a delivery vehicle (e.g., a suitable nanoparticle) and that can, by design, express in the intended cell (e.g. NK cell) in vivo. Such CFP constructs can be prepared for an “off the shelf” product.

The schematic shows exemplary immune receptors, marked in the figure as immune receptor A, immune receptor B and immune receptor C, that are known by literature survey or bioinformatics to potentially pair with coreceptors, such as ITAM-domain containing coreceptors described here, are tested in HEK 293 cells. For each pair of receptor and coreceptor tested, HEK 293 cells are divided into two groups (i) control group (top right of the diagram, that is electroporated with the immune receptor construct but not with the coreceptor construct (vehicle), (ii) experimental group in which the immune receptor and the coreceptor are both electroporated. Each coreceptor construct can comprise a fluorescent tag, e.g., GFP as shown. Expression of both the immune receptor and the coreceptor are tested. The immune receptor that does not express in (i) and expressed in (ii) is selected as an NK cell specific receptor, and is further developed into a CFP by methods described herein.

FIG. 2A (Top) shows an exemplary myeloid cell-specific chimeric fusion protein (CFP) receptor design and expression in monocytes. Expression determined by flow cytometry. The CFP comprises TM domain of CD89 that oligomerize with CD89 receptor complex and integrates in the cell membrane of NK cells. FIG. 2A (Bottom) shows an exemplary myeloid and NK cell-specific CFP receptor design and expression in NK cells.

FIG. 2B shows an exemplary NK cell-specific CFP receptor design and expression in NK cells.

FIG. 3 shows graphical images of new designs for receptors tested for expression in primary NK cells and functional assay scheme. The new CFPs comprise and extracellular domain, a TM domain from NKp30, NKp44, NKp46 TM, NKG2C, NKG2D or NK16 transmembrane domains with our without associated cytoplasmic and extracellular domains, and each construct comprises an extracellular antigen binding domain which may be a scFv or a SdB binder that can bind to a target antigen on a target cell. NKp30 intracellular domain can interact with associated adaptor proteins e.g., CD3z/FcεRγ. NKp46 intracellular domain also can interact with associated adaptor proteins e.g., CD3z/FcεRγ. CD16 intracellular domain can interact with associated adaptor proteins e.g., CD3z/FcεRγ. The NKp44 intracellular domain can interact with associated adaptor proteins e.g., DAP12. The NKG2C or NKG2D intracellular domain can interact with associated adaptor proteins e.g., DAP10.

FIG. 4A-4C shows construct design and data from the same experiment. In this case NK cell-specific CFP designs were as follows: N-terminal cytoplasmic domain (also designated as intracellular domain, ICD) and transmembrane (TM) domain of NKG2C or NKG2D, with or without the NKG2C or NKG2D extracellular domain respectively and with a short linker for the construct lacking the NKG2C or NKG2D extracellular domain and an scFv that can bind to the target at the C terminus. In the exemplary construct, the scFv is an anti-HER2 scFv, which binds to HER2. Expression results demonstrate poor expression of these constructs.

FIG. 5 shows graphical representations of the NKp30 CFPs from N-C terminal and demonstrates expression data for the CFP expression in NK cells detected by flow cytometry. Domain arrangements were flipped in these constructs as the scFV is in the N-terminal portion and the intracellular domain is in the C terminal ends compared to constructs described in FIGS. 4A-4C.

FIG. 6A shows graphical representations of the NKp44/46 CFPs from N-C terminal and demonstrates expression data in NK cells detected by flow cytometry.

FIG. 6B shows graphical representations of the NKp44/46 CFPs from N-C terminal and demonstrates expression data in NK cells detected by flow.

FIG. 7 (Top panel) shows the cartoon structures of the CFPs as discussed before. Bottom panel shows data demonstrating tumor cell killing activity of NK cells expressing the different constructs as shown below.

FIG. 8 (Top panel) shows cartoon structures of CFPs with CD16 structural domains including a TM domain, that is expressed in NK cells. FIG. 8 (Bottom panel) shows data demonstrating tumor cell killing activity by NK cells expressing TROP2-binding CD16 TM binders as indicated, and compared to the first generation constructs having CD8TM-CD3z ICD domain structures.

FIG. 9 shows data on time course of cell lysis by NK cells expressing the indicated CFPs.

FIG. 10 shows data on cytokine generation by NK cells expressing the indicated CFP constructs in presence of the target antigen (TROP2+ cancer cell) or unstimulated.

FIG. 11A shows graphical representations of CFP constructs having the respective extracellular, transmembrane and intracellular domains as indicated. HER2 scFv, anti-HER2 scFV antigen binding domain; ectodomain, a portion of the extracellular domain from the same protein as the TM domain having about 20 aa.

FIG. 11B shows expression data of the indicated constructs in NK cells 24h after transfection, determined by flow cytometry.

FIG. 11C shows data on target cell killing (cytotoxicity) activity of NK cells expressing the indicated CFP constructs. Target cells are HER2+ cancer cells expressing luciferase. Level of significance indicated.

FIG. 11D shows NK-kappa B activation data in NK cells expressing the indicated CFP in presence or absence CFP stimulation by the target antigen (HER2+ cancer cell).

FIG. 12A shows a diagrammatic view of CFP designs for testing impact of the hinge domain on the CFP activation upon expression in NK cells. The CFPs either have no hinge, left, or have CD4 or CD8 hinge domains, monomer or dimer formats, or siglec4 hinge as indicated.

FIG. 12B shows data demonstrating that CD4 hinge improved Fey chain dependent expression in liver cell line Huh7 cells.

FIG. 12C shows data demonstrating inclusion of CD4 hinge improved Fey chain dependent expression in Huh7 cells over time as indicated by days after transfected.

FIG. 12D shows data demonstrating inclusion of CD4 hinge improved tumor specific killing activity.

FIG. 13 shows exemplary T cell-specific CFP receptors. Left, diagrammatic view of a natural T cell receptor complex. CFP designed for T cell specific expression to be integrated in the TCR complex is shown by an arrow, comprising a scFv targeting CD19 (anti-CD19 scFv) and CD3e extracellular, transmembrane (TM) and intracellular domains. Middle panel shows CFP expression data. Expression was demonstrated in T cells only as determined by flow cytometry. On the right is shown results from cell killing assay by incubating T cells expressing the CFP and CD19+ target cells, demonstrating significant cell death by the T cells expressing the CFP.

### DETAILED DESCRIPTION

In one aspect of the disclosure, provided herein is a composition comprising a recombinant polynucleic acid comprising a sequence encoding a chimeric fusion protein for expression in an NK cell. In another aspect, provided herein is a composition for engineering NK cells to perform a therapeutic function in vivo. In one embodiment, provided herein is a composition comprising a recombinant chimeric fusion protein (CFP), the CFP comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by natural killer (NK) cells; and wherein after administration of the composition to a human subject the CFP is expressed on cell surfaces of NK cells of the human subject.

A composition comprising a recombinant polynucleic acid as described above may be a pharmaceutical composition. In some embodiments, the pharmaceutical composition is suitable for direct in vivo administration. In some embodiments, the composition is a solution comprising the recombinant polynucleic acid, suitably designed and formulated for uptake by specific cells in vivo.

A recombinant polynucleic acid, as described herein is that is constructed artificially using recombinant technology, and comprises sequences that are not found in nature.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” as used herein when referring to a measurable value such as a parameter, an amount, a temporal duration, and the like, is meant to encompass variations of +/−30% or less, +/−20% or less, +/−10% or less, +1-5% or less, or +/−1% or less of and from the specified value, insofar such variations are appropriate to perform in the present disclosure. It is to be understood that the value to which the modifier “about” or “approximately” refers is itself also specifically disclosed.

An “antigen” is a molecule capable of stimulating an immune response. Antigens recognized by T cells, whether helper T lymphocytes (T helper (TH) cells) or cytotoxic T lymphocytes (CTLs), are not recognized as intact proteins, but rather as small peptides that associate with MHC proteins (such as class I or class II MHC proteins) on the surface of cells. During the course of a naturally occurring immune response, antigens that are recognized in association with class II MHC molecules on antigen presenting cells (APCs) are acquired from outside the cell, internalized, and processed into small peptides that associate with the class II MHC molecules.

A “polypeptide” can refer to a molecule containing amino acids linked together via a peptide bond, such as a glycoprotein, a lipoprotein, a cellular protein or a membrane protein. A polypeptide may comprise one or more subunits of a protein. A polypeptide may be encoded by a recombinant polynucleic acid. In some embodiments, polypeptide may comprise more than one peptide sequence in a single amino acid chain, which may be separated by a spacer, a linker or peptide cleavage sequence. A polypeptide may be a fused polypeptide. A polypeptide may comprise one or more domains, modules or moieties.

A “receptor” can refer to a chemical structure composed of a polypeptide, which transduces a signal, such as a polypeptide that transduces an extracellular signal to a cell. A receptor can serve to transmit information in a cell, a cell formation or an organism. A receptor comprises at least one receptor unit and can contain two or more receptor units, where each receptor unit comprises a protein molecule, e.g., a glycoprotein molecule. A receptor can contain a structure that binds to a ligand and can form a complex with the ligand. Signaling information can be transmitted by a conformational change of the receptor following binding with the ligand on the surface of a cell.

The term “antibody” refers to a class of proteins that are generally known as immunoglobulins, including, but not limited to IgG1, IgG2, IgG3, and IgG4), IgA (including IgA1 and IgA2), IgD, IgE, IgM, and IgY, The term “antibody” includes, but is not limited to, full length antibodies, single-chain antibodies, single domain antibodies (sdAb) and antigen-binding fragments thereof. Antigen-binding antibody fragments include, but are not limited to, Fab, Fab′ and F(ab′)2, Fd (consisting of V_{H} and C_{H}1), single-chain variable fragment (scFv), single-chain antibodies, disulfide-linked variable fragment (dsFv) and fragments comprising a V_{L} and/or a VH domain. Antibodies can be from any animal origin. Antigen-binding antibody fragments, including single-chain antibodies, can comprise variable region(s) alone or in combination with tone or more of a hinge region, a CH1 domain, a CH2 domain, and a CH3 domain. Also included are any combinations of variable region(s) and hinge region, CH 1, CH2, and CH3 domains. Antibodies can be monoclonal, polyclonal, chimeric, humanized, and human monoclonal and polyclonal antibodies which, e.g., specifically bind an HLA-associated polypeptide or an HLA-peptide complex.

A “biological sample” can refer to any tissue, cell, fluid, or other material derived from an organism.

The term “epitope” can refer to any protein determinant, such as a sequence or structure or amino acid residues, capable of binding to an antibody or binding fragment thereof, a T cell receptor, and/or an antibody-like molecule. Epitopic determinants typically consist of chemically active surface groups of molecules such as amino acids or sugar side chains and generally have specific three dimensional structural characteristics as well as specific charge characteristics. A “T cell epitope” can refer to peptide or peptide-MHC complex recognized by a T cell receptor.

An engineered cell, such as an engineered NK cell, can refer to a cell that has at least one exogenous nucleic acid sequence in the cell, even if transiently expressed. Expressing an exogenous nucleic acid may be performed by various methods described elsewhere, and encompasses methods known in the art. The present disclosure relates to preparing and using engineered cells, for example, engineered myeloid cells, such as engineered phagocytic cells. The present disclosure relates to, inter alia, an engineered cell comprising an exogenous nucleic acid encoding, for example, a chimeric fusion protein (CFP). The cell may be engineered in vivo.

The term “immune response” includes, but is not limited to, T cell mediated, NK cell mediated and/or B cell mediated immune responses. These responses may be influenced by modulation of T cell costimulation and NK cell costimulation. Exemplary immune responses include T cell responses, e.g., cytokine production, and cellular cytotoxicity. In addition, immune responses include immune responses that are indirectly affected by NK cell activation, B cell activation and/or T cell activation, e.g., antibody production (humoral responses) and activation of cytokine responsive cells, e.g., macrophages. Immune responses include adaptive immune responses. The adaptive immune system can react to foreign molecular structures, such as antigens of an intruding organism. Unlike the innate immune system, the adaptive immune system is highly specific to a pathogen. Adaptive immunity can also provide long-lasting protection. Adaptive immune reactions include huImoral immune reactions and cell-mediated immune reactions. In humoral immune reactions, antibodies secreted by B cells into bodily fluids bind to pathogen-derived antigens leading to elimination of the pathogen through a variety of mechanisms, e.g. complement-mediated lysis. In cell-mediated immune reactions, T cells capable of destroying other cells are activated. For example, if proteins associated with a disease are present in a cell, they can be fragmented proteolytically to peptides within the cell. Specific cell proteins can then attach themselves to the antigen or a peptide formed in this manner, and transport them to the surface of the cell, where they can be presented to molecular defense mechanisms, such as T cells. Cytotoxic T cells can recognize these antigens and kill cells that harbor these antigens.

A “ligand” can refer to a molecule which is capable of binding or forming a complex with another molecule, such as a receptor. A ligand can include, but is not limited to, a protein, a glycoprotein, a carbohydrate, a lipoprotein, a hormone, a fatty acid, a phospholipid, or any component that binds to a receptor. In some embodiments, a receptor has a specific ligand. In some embodiments, a receptor may have promiscuous binding to a ligand, in which case it can bind to several ligands that share at least a similarity in structural configuration, charge distribution or any other physicochemical characteristic. A ligand may be a biomolecule. A ligand may be an abiotic material. For example, a ligand may be a negative charged particle that is a ligand for scavenger receptor MARCO. For example, a ligand may be TiO₂, which is a ligand for the scavenger receptor SRA 1.In the context of a CFP described herein, the extracellular binding domain may bind to a ligand, which is a also designated as a target of the binding domain. In some embodiments, the target is an antigen expressed on a diseased cell, such as a cancer cell, which in this case is a target cell, in the sense that the target cell expresses on its cell surface a target antigen to which the extracellular antigen binding domain of the CFP binds. Anti-(target) binding domain or anti-(target) binding extracellular domain or anti-(target) CFP are often interchangeably used with terms such as (target) binding domain or (target) binding extracellular domain or (target) CFP respectively in the disclosure. For example, HER2 expressed on cancer cells is an antigen (ligand) to which the anti-HER2 binding extracellular domain of a CFP binds; or alternatively stated as, a HER2-binding extracellular domain of a CFP binds.

The term “major histocompatibility complex (MHC)”, “MHC molecule”, or “MHC protein” refers to a protein capable of binding an antigenic peptide and present the antigenic peptide to T lymphocytes. Such antigenic peptides can represent T cell epitopes. The human MHC is also called the HLA complex. Thus, the terms “human leukocyte antigen (HLA)”, “HLA molecule” or “HLA protein” are used interchangeably with the terms “major histocompatibility complex (MHC)”, “MHC molecule”, and “MHC protein”. HLA proteins can be classified as HLA class I or HLA class II. The structures of the proteins of the two HLA classes are very similar; however, they have very different functions. Class I HLA proteins are present on the surface of almost all cells of the body, including most tumor cells. Class I HLA proteins are loaded with antigens that usually originate from endogenous proteins or from pathogens present inside cells, and are then presented to naive or cytotoxic T-lymphocytes (CTLs). HLA class II proteins are present on antigen presenting cells (APCs), including but not limited to dendritic cells, B cells, and macrophages. They mainly present peptides which are processed from external antigen sources, e.g. outside of cells, to helper T cells.

In the HLA class II system, phagocytes such as macrophages and immature dendritic cells can take up entities by phagocytosis into phagosomes—though B cells exhibit the more general endocytosis into endosomes—which fuse with lysosomes whose acidic enzymes cleave the uptaken protein into many different peptides. Autophagy is another source of HLA class II peptides. The most studied subclass II HLA genes are: HLA-DPA1, HLA-DPB1, HLA-DQA1, HLA-DQB1, HLA-DRA, and HLA-DRB1.

Presentation of peptides by HLA class II molecules to CD4+ helper T cells can lead to immune responses to foreign antigens. Once activated, CD4+ T cells can promote B cell differentiation and antibody production, as well as CD8+ T cell (CTL) responses. CD4+ T cells can also secrete cytokines and chemokines that activate and induce differentiation of other immune cells. HLA class II molecules are typically heterodimers of α- and β-chains that interact to form a peptide-binding groove that is more open than class I peptide-binding grooves.

HLA alleles are typically expressed in codominant fashion. For example, each person carries 2 alleles of each of the 3 class I genes, (HLA-A, HLA-B and HLA-C) and so can express six different types of class II HLA. In the class II HLA locus, each person inherits a pair of HLA-DP genes (DPA1 and DPB1, which encode a and 0 chains), HLA-DQ (DQA1 and DQB1, for a and 0 chains), one gene HLA-DRa (DRA1), and one or more genes HLA-DRD (DRB1 and DRB3, −4 or −5). HLA-DRB1, for example, has more than nearly 400 known alleles. That means that one heterozygous individual can inherit six or eight functioning class II HLA alleles: three or more from each parent. Thus, the HLA genes are highly polymorphic; many different alleles exist in the different individuals inside a population. Genes encoding HLA proteins have many possible variations, allowing each person's immune system to react to a wide range of foreign invaders. Some HLA genes have hundreds of identified versions (alleles), each of which is given a particular number. In some embodiments, the class I HLA alleles are HLA-A*02:01, HLA-B*14:02, HLA-A*23:01, HLA-E*01:01 (non-classical). In some embodiments, class II HLA alleles are HLA-DRB*01:01, HLA-DRB*01:02, HLA-DRB*11:01, HLA-DRB*15:01, and HLA-DRB*07:01.

The term “recombinant polynucleic acid” refers a nucleic acid prepared, expressed, created or isolated by recombinant means. A recombinant polynucleic acid can contain a nucleotide sequence that is not naturally occurring. A recombinant polynucleic acid may be synthesized in the laboratory. A recombinant polynucleic acid may be prepared by using recombinant DNA technology, for example, enzymatic modification of DNA, such as enzymatic restriction digestion, ligation, and DNA cloning. A recombinant polynucleic acid can be DNA, RNA, analogues thereof, or a combination thereof. A recombinant DNA may be transcribed ex vivo or in vitro, such as to generate a messenger RNA (mRNA). A recombinant mRNA may be isolated, purified and used to transfect a cell. A recombinant polynucleic acid may encode a protein or a polypeptide. Throughout the specification, nucleic acid sequences are described which may comprise deoxyribonucleotides (DNA), ribonucleotides (RNA), or in some embodiments, modified deoxyribonucleotides, or modified ribonucleotides. For example, a modified nucleotide may be a 5-hydroxymethylcytosine (5hmC), a 5-formylacytosine (5fC), a 7-methylguanosine, a pseudouridine, a dihydrouridine etc. One of skill in the art can determine an RNA sequence, e.g., an mRNA sequence from a given polynucleotide sequence without difficulty. Sequences may be codon optimized.

The process of introducing or incorporating a nucleic acid into a cell can be via transformation, transfection or transduction. Transformation is the process of uptake of foreign nucleic acid by a bacterial cell. This process is adapted for propagation of plasmid DNA, protein production, and other applications. Transformation introduces recombinant plasmid DNA into competent bacterial cells that take up extracellular DNA from the environment. Some bacterial species are naturally competent under certain environmental conditions, but competence is artificially induced in a laboratory setting. Transfection is the introduction of small molecules such as DNA, RNA, or antibodies into eukaryotic cells. Transfection may also refer to the introduction of bacteriophage into bacterial cells. ‘Transduction’ is mostly used to describe the introduction of recombinant viral vector particles into target cells, while ‘infection’ refers to natural infections of humans or animals with wild-type viruses.

The term “vector”, can refer to a nucleic acid molecule capable of autonomous replication in a host cell, and which allow for cloning of nucleic acid molecules. As known to those skilled in the art, a vector includes, but is not limited to, a plasmid, cosmid, phagemid, viral vectors, phage vectors, yeast vectors, mammalian vectors and the like. For example, a vector for exogenous gene transformation may be a plasmid. In certain embodiments, a vector comprises a nucleic acid sequence containing an origin of replication and other elements necessary for replication and/or maintenance of the nucleic acid sequence in a host cell. In some embodiments, a vector or a plasmid provided herein is an expression vector. Expression vectors are capable of directing the expression of genes and/or nucleic acid sequence to which they are operatively linked. In some embodiments, an expression vector or plasmid is in the form of circular double stranded DNA molecules. A vector or plasmid may or may not be integrated into the genome of a host cell. In some embodiments, nucleic acid sequences of a plasmid are not integrated in a genome or chromosome of the host cell after introduction. For example, the plasmid may comprise elements for transient expression or stable expression of the nucleic acid sequences, e.g. genes or open reading frames harbored by the plasmid, in a host cell. In some embodiments, a vector is a transient expression vector. In some embodiments, a vector is a stably expressed vector that replicates autonomously in a host cell. In some embodiments, nucleic acid sequences of a plasmid are integrated into a genome or chromosome of a host cell upon introduction into the host cell. Expression vectors that can be used in the methods as disclosed herein include, but are not limited to, plasmids, episomes, bacterial artificial chromosomes, yeast artificial chromosomes, bacteriophages or viral vectors. A vector can be a DNA or RNA vector. In some embodiments, a vector provide herein is a RNA vector that is capable of integrating into a host cell's genome upon introduction into the host cell (e.g., via reverse transcription), for example, a retroviral vector or a lentiviral vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

In some embodiments, nucleic acid may be delivered into a living system in the form of nanoparticles. Nucleic acid sequences disclosed herein may be delivered in vivo via suitable nanoparticles, e.g., liposomes, lipid nanoparticles, or polymeric nanoparticles. A lipid nanoparticle may comprise a polar lipid. In some embodiments, the lipid nanoparticle comprises a cationic lipid. In some embodiments, the lipid nanoparticle comprises a cationic lipid and a non-cationic lipid. In some embodiments, the lipid nanoparticle comprises a neutral lipid. In some embodiments, the lipid nanoparticle comprises a PEGylated lipid.

Alternatively, in some embodiments, the nucleic acid can be electroporated in a living cell ex vivo for preparation of a cellular therapy, wherein the cell is a myeloid cell.

The terms “spacer” or “linker” as used in reference to a fusion protein may refer to a peptide sequence that joins two other peptide sequences of the fusion protein. In some embodiments, a linker or spacer has no specific biological activity other than to join or to preserve some minimum distance or other spatial relationship between the proteins or RNA sequences. In some embodiments, the constituent amino acids of a spacer can be selected to influence some property of the molecule such as the folding, flexibility, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. In some embodiments, a linker is used to separate two or more polypeptides, e.g. two antigenic peptides by a distance sufficient to ensure that each antigenic peptide properly folds. Exemplary peptide linker sequences adopt a flexible extended conformation and do not exhibit a propensity for developing an ordered secondary structure. Amino acids in flexible linker protein region may include Gly, Asn and Ser, or any permutation of amino acid sequences containing Gly, Asn and Ser. Other near neutral amino acids, such as Thr and Ala, also can be used in the linker sequence.

The terms “treat,” “treated,” “treating,” “treatment,” and the like may refer to reducing, preventing, or ameliorating a disorder and/or symptoms associated therewith (e.g., a neoplasia or tumor or infectious agent or an autoimmune disease). “Treating” can refer to administration of the therapy to a subject after the onset, or suspected onset, of a disease (e.g., cancer or infection by an infectious agent or an autoimmune disease). “Treating” includes the concepts of “alleviating”, which can refer to lessening the frequency of occurrence or recurrence, or the severity, of any symptoms or other ill effects related to the disease and/or the side effects associated with therapy. The term “treating” also encompasses the concept of “managing” which refers to reducing the severity of a disease or disorder in a patient, e.g., extending the life or prolonging the survivability of a patient with the disease, or delaying its recurrence, e.g., lengthening the period of remission in a patient who had suffered from the disease. It is appreciated that, although not precluded, treating a disorder or condition does not require that the disorder, condition, or symptoms associated therewith be completely eliminated. The term “prevent”, “preventing”, “prevention” and their grammatical equivalents as used herein, can refer to avoiding or delaying the onset of symptoms associated with a disease or condition in a subject that has not developed such symptoms at the time the administering of an agent or compound commences. In certain embodiments, treating a subject or a patient as described herein comprises administering a therapeutic composition, such as a drug, a metabolite, a preventive component, a nucleic acid, a peptide, or a protein that encodes or otherwise forms a drug, a metabolite or a preventive component. In some embodiments, treating comprises administering a recombinant polynucleic acid encoding a fusion protein that is designed to be specifically expressed in an NK cell, when the recombinant polynucleic acid is administered in vivo. Treating comprises treating a disease or a condition or a syndrome, which may be a pathological disease, condition or syndrome, or a latent disease, condition or syndrome. In some cases, treating, as used herein may comprise administering a therapeutic vaccine. In some embodiments, the engineered phagocytic cell is administered to a patient or a subject. In some embodiments, a cell administered to a human subject results in reduced immunogenicity. For example, an engineered phagocytic cell may lead to no or reduced graft versus host disease (GVHD) or fratricide effect. In some embodiments, an engineered cell administered to a human subject is immunocompatible to the subject (i.e. having a matching HLA subtype that is naturally expressed in the subject). Subject specific HLA alleles or HLA genotype of a subject can be determined by any method known in the art. In exemplary embodiments, the methods include determining polymorphic gene types that can comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

A “fragment” can refer to a portion of a protein or nucleic acid. In some embodiments, a fragment retains at least 50%, 75%, or 80%, or 90%, 95%, or even 99% of the biological activity of a reference protein or nucleic acid. Unless otherwise indicated, a fragment contemplated in the descriptions herein are intended to be functionally relevant fragment of the protein or nucleic acid.

The terms “isolated,” “purified”, “biologically pure” and their grammatical equivalents may refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the present disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications can give rise to different isolated proteins, which can be separately purified.

The terms “neoplasia” or “cancer” may refer to any disease that is caused by or results in inappropriately high levels of cell division, inappropriately low levels of apoptosis, or both. Glioblastoma is one non-limiting example of a neoplasia or cancer. The terms “cancer” or “tumor” or “hyperproliferative disorder” can refer to the presence of cells possessing characteristics typical of cancer-causing cells, such as uncontrolled proliferation, immortality, metastatic potential, rapid growth and proliferation rate, and certain characteristic morphological features. Cancer cells may often be in the form of a tumor, but such cells can exist alone within an animal, or can be a non-tumorigenic cancer cell, such as a leukemia cell.

The term “vaccine” may be understood as meaning a composition for generating immunity for the prophylaxis and/or treatment of diseases (e.g., neoplasia/tumor/infectious agents/autoimmune diseases). Accordingly, vaccines can be used herein that are medicaments which comprise recombinant polynucleic acids, or cells comprising and expressing a recombinant polynucleic acid and are intended to be used in humans or animals for generating specific defense and protective substance by vaccination. A “vaccine composition” can include a pharmaceutically acceptable excipient, carrier or diluent. Aspects of the present disclosure relate to use of the technology in preparing a phagocytic cell-based vaccine.

The term “pharmaceutically acceptable” often refers to approved or approvable by a regulatory agency of the Federal or a state government or listed in the U.S. Pharmacopeia or other generally recognized pharmacopeia for use in animals, including humans. A “pharmaceutically acceptable excipient, carrier or diluent” may refer to an excipient, carrier or diluent that can be administered to a subject, together with an agent, and which does not destroy the pharmacological activity thereof and is nontoxic when administered in doses sufficient to deliver a therapeutic amount of the agent.

Nucleic acid molecules useful in the methods of the disclosure include, but may not be limited to, any nucleic acid molecule with activity or that encodes a polypeptide. Polynucleotides having substantial identity to an endogenous sequence are typically capable of hybridizing with at least one strand of a double-stranded nucleic acid molecule. “Hybridize” refers to when nucleic acid molecules pair to form a double-stranded molecule between complementary polynucleotide sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration can ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions can ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an exemplary embodiment, hybridization can occur at 30° C. in 750 mM NaCl, 75 mM trisodium citrate, and 1% SDS. In another exemplary embodiment, hybridization can occur at 37° C. in 500 mM NaCl, 50 mM trisodium citrate, 1% SDS, 35% formamide, and 100 μg/ml denatured salmon sperm DNA (ssDNA). In another exemplary embodiment, hybridization can occur at 42° C. in 250 mM NaCl, 25 mM trisodium citrate, 1% SDS, 50% formamide, and 200 μg/ml ssDNA. Useful variations on these conditions will be readily apparent to those skilled in the art. For most applications, washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, or less than about 15 mM NaCl and 1.5 mM trisodium citrate. Stringent temperature conditions for the wash steps can include a temperature of at least about 25° C., of at least about 42° C., or at least about 68° C. In exemplary embodiments, wash steps can occur at 25° C. in 30 mM NaCl, 3 mM trisodium citrate, and 0.1% SDS. In other exemplary embodiments, wash steps can occur at 42° C. in 15 mM NaCl, 1.5 mM trisodium citrate, and 0.1% SDS. In another exemplary embodiment, wash steps can occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York. [00127]“Substantially identical” may refer to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence can be at least 60%, 80% or 85%, 90%, 95%, 96%, 97%, 98%, or even 99% or more identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e-3 and e-m° indicating a closely related sequence. A “reference” is a standard of comparison. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment to a reference sequence and determination of homologous residues.

“Expressed” as used herein, e.g. for a recombinant polynucleic acid, pertaining to a cell, can refer to the plain meaning as is understood by one of minimum skill in the art, as that the encoded product is present or evident if tested in the cell that comprises the recombinant polynucleic acid. “Substantial expression” of a polynucleotide refers to a relative expression of the polynucleotide encoded product, pertaining to a degree or intensity of expression that denotes positive expression. Conversely, “not substantially expressed” indicates that the expression is not positively determined, is undetectable or is negligible. For example in a hypothetical range of expression of a GFP protein in cells in an experiment, where the cells have been transfected with a range of dose of the GFP-construct, it may be envisioned that the cells fall in the range of 0%-100% expression as determined by intensity of GFP detected by a fluorescence detector, where 0% is undetectable and 100% is the brightest possible fluorescence. Considering this example, it is possible that 2%, 5% or even 10% fluorescence from GFP may still be within the undetectable range based on the detection device or measurement criteria (e.g., gating) used, and is therefore considered not substantially expressed. On the other hand, if 10% falls within the detectable range, it may be considered substantially expressed. Similarly, 20%, 25%, 50%, 60% 75% fluorescence level, or more is considered substantially expressed in the cell. “Predominantly expressed” in a cell indicates cell-specific or selective expression of a gene or a construct. In the present context, a construct can be considered predominantly expressed e.g., in NK cells, where it is expressed in NK cells and not substantially expressed in the vast majority of other cells types, e.g., B cells, or dendritic cells, or epithelial cells or muscle cells. In another instance, “predominantly expressed” may not exclude expression in related cell types, e.g., NKT cells, or may allow, under circumstances, substantially low expression in some other cell types, as deemed acceptable by one of skill in the art. In some embodiments, the methods and compositions described here comprise polynucleic acid designs that are designed for expression in a cell type and not substantially express in another cell type. The desire or intention of the program to have a polynucleic acid express in a certain cell type may be such that the polynucleic acid expresses in a cell that can be detected reliably, at least over a period of time, for example, say for about 18 to at least about 42 hours after introduction of the polynucleic acid, at a level of expression that can be determined by commonly known methods at disposal to one of ordinary skill in the art. Along the same lines, when a polynucleic acid is not substantially or predominantly expressed in a cell type, it may mean that the translated protein or polypeptide encoded by the polypeptide, generally understood as the entire polypeptide encoded by the sequence, is not within reliably detectable range by commonly known methods at disposal to one of ordinary skill in the art. It may even be transiently expressed and outside the window that is ordinarily perceived as a reliable protein expression from an exogenous nucleic acid sequence.

The term “subject” or “patient” may refer to an organism, such as an animal (e.g., a human) which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

The term “therapeutic effect” may refer to some extent of relief of one or more of the symptoms of a disorder (e.g., a neoplasia, tumor, or infection by an infectious agent or an autoimmune disease) or its associated pathology. On one hand it may indicate a reduction of a symptom of the disease, e.g., a 10%, 20%, 30% and so on reduction in tumor mass following administration of the therapeutic composition. On another embodiment, it can relate to partial or complete remission of one or more symptoms, or amelioration of the disease. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required.

All terms are intended to be understood as they would be understood by a person skilled in the art. Unless defined otherwise, all technical and scientific terms used herein can have the same meaning as commonly understood by one of ordinary skill in the art to which the disclosure pertains. All terms are intended to be understood as they would be understood by a person skilled in the art. Unless defined otherwise, all technical and scientific terms used herein have the same meaning as commonly understood by one of ordinary skill in the art to which the disclosure pertains.

Provided herein is a recombinant polynucleic acid composition comprising a recombinant polynucleic acid sequence encoding a chimeric fusion protein (CFP) comprising a transmembrane domain that specifically integrates within a membrane protein complex of a cell, wherein the cell is characterized as naturally expressing the membrane protein complex; wherein, when the recombinant polynucleic acid composition comprising the recombinant polynucleic acid sequence is contacted to any cell of a heterogenous cell population, at least greater than 50% of cells within the heterogenous cell population characterized as naturally expressing the membrane protein complex expresses the CFP, and a cell within the heterogenous cell population that lacks the membrane protein complex fails to express the CFP; and wherein the cell that is characterized as naturally expressing the membrane protein complex is a NK cell, a B cell or a T cell.

In some embodiments, the recombinant polynucleic acid composition is expressed in at least greater than 60%, 70%, 80% or 90% of cells within the heterogenous cell population characterized as naturally expressing the membrane protein complex expresses the CFP.

In some embodiments, the recombinant polynucleic acid composition less than 10% cells within the heterogenous cell population that lacks the membrane protein complex express the CFP.

In some embodiments, the recombinant polynucleic acid composition comprises one or more recombinant polynucleic acid molecules comprising more than one recombinant polynucleic acid sequences, each recombinant polynucleic acid sequence of the more than one recombinant polynucleic acid sequences comprises a unique sequence encoding a transmembrane domain.

In some embodiments, the recombinant polynucleic acid composition comprises a polypeptide encoded by each recombinant polynucleic acid sequence is expressed in a specific cell type.

In some embodiments, the recombinant polynucleic acid composition each recombinant polynucleic acid sequence is expressed in a cell type distinct from a different sequence.

In some embodiments, the recombinant polynucleic acid composition the transmembrane domain is operably linked with an extracellular domain, wherein the extracellular domain comprises an antigen binding domain.

In some embodiments, the recombinant polynucleic acid composition the antigen binding domain binds to a cell surface antigen on a target cell.

In some embodiments, the recombinant polynucleic acid composition the target cell is a cancer cell.

In some embodiments, the recombinant polynucleic acid composition the target cell is an infected cell.

In some embodiments, the recombinant polynucleic acid composition the target cell is an autoimmune cell.

In some embodiments, the recombinant polynucleic acid composition the recombinant polynucleic acid further comprises a nucleic acid delivery vehicle.

In some embodiments, the recombinant polynucleic acid composition the recombinant polynucleic acid composition comprises a lipid.

In some embodiments, the recombinant polynucleic acid composition the recombinant polynucleic acid composition comprises a lipid nanoparticle (LNP).

In some embodiments, the recombinant polynucleic acid composition the recombinant polynucleic acid composition further comprises a nucleic acid delivery vehicle comprising a cationic lipid, a non-cationic lipid, a neutral lipid, a cholesterol or a polyethylenel glycol (PEG)-lipid.

In some embodiments, the recombinant polynucleic acid composition comprises a polymeric nucleic acid delivery vehicle.

Provided herein is a pharmaceutical composition comprising any one or more of the recombinant polynucleic acid composition described herein and a pharmaceutically acceptable excipient.

In some embodiments, the pharmaceutical composition is formulated for an in vivo delivery. NK cell specific chimeric fusion protein (CFP) designs

In one aspect, a recombinant polynucleic acid is described herein, e.g. the recombinant chimeric fusion protein (CFP), the CFP comprising: (a) an extracellular domain comprising an antigen binding domain, wherein the antigen is expressed on a target cell that is a diseased cell and that will be lysed by the engineered NK cell expressing the receptor, and (b) a transmembrane domain operatively linked to the extracellular domain; wherein the transmembrane domain is a transmembrane domain from a protein that multimerizes with a cell surface receptor expressed by natural killer (NK) cells. This design of the recombinant polynucleic acid ensures targeting an NK cell for selective destruction of a diseased cell, e.g., a cancer cell, and that the recombinant polynucleic acid will selectively express in an NK cell owing to the transmembrane domain which is properly expressed or is functional upon multimerization with other cell surface receptors that are endogenously naturally express in an NK cell. A large number of naturally occurring NK cell receptors are described, and exemplary recombinant polynucleic acids having specific extracellular binding domains are exemplified herein for describing the invention, and the design of the recombinant polynucleic acid is contemplated to comprise any number of combinations of possible domains as deemed possible by on of skill in the art.

In some embodiments, the recombinant polynucleic acid, e.g. the recombinant chimeric fusion protein (CFP), the CFP comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by natural killer (NK) cells is RNA. In some embodiments, the recombinant polynucleic acid is messenger RNA (mRNA). In some embodiments, the recombinant polynucleic acid, e.g. the recombinant chimeric fusion protein (CFP), the CFP comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by natural killer (NK) cells is DNA.

While a recombinant chimeric fusion protein (CFP) encoding a transmembrane protein or chimeric receptor is described in detail in the disclosure, any recombinant protein is contemplated herein that can be expressed specifically in a NK cell, i.e., the expression construct is designed specifically for (i) preferential and uptake by a NK cell; (ii) is expressed specifically in a NK cell, and does not detectably express on a cell that is not a NK cell, or, (iii) is functional when expressed in a NK cell not functional when expressed in a cell other than a NK cell.

Provided herein are compositions and methods for generating engineered NK cells for application in immunotherapy. In one aspect, the engineered NK cells have an augmented immune function. In some embodiments, the engineered NK cells described herein are for application in cancer immunotherapy. The natural killer cell was discovered in the mid-1970s based on its ability to lyse certain tumor cells without prior sensitization of the host. NK cells have traditionally been classified as group 1 innate lymphoid cells and develop from hematopoietic stem cells (HSCs) while maturing outside the bone marrow compartment. These cells are often characterized as large granular lymphocytes. Their derivation from either lymphoid or myeloid lineages was debated early in their discovery. Further research showed that NK cells can be derived from common lymphoid progenitors (CLPs). Typically, NK cells are involved in our defense against certain virus-infected and malignant cells. These cells can swiftly kill adjacent target cells. However, NK cells are subject to environmental influence, e.g. suppression or deactivation signals, e.g., in a tumor microenvironment, such that the target cell me overcome or resist NK cell attack. Hence, engineered NK cells can be designed that are less susceptible to environmental influence or otherwise possess augmented activities that help eliminate the target and/or induce or alert the immune system against the target cell. In some embodiments, NK cell are engineered to enhance activating signals and proliferation. In some embodiments, engineered NK cells suppress inhibitory signals. In some embodiments, NK cells are engineered to promote their homing to tumors. In some embodiments, NK cells are engineered to specifically target cells that express a surface antigen and lyse the target cell.

In one aspect, provided herein is a composition comprising a recombinant polynucleic acid comprising a sequence encoding a chimeric fusion protein (CFP), the CFP comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by natural killer (NK) cells; and wherein after administration of the composition to a human subject the CFP is expressed on cell surfaces of NK cells of the human subject.

In some embodiments, any recombinant polynucleic acid encoding an intracellular or transmembrane protein can be designed to express in an NK cell following any one of the embodiments described herein. In some embodiments, the disclosure is not limited to compositions, methods of making and use of a certain recombinant protein for expression in NK cells, rather, any recombinant protein that can be designed for expression in an NK cell.

In some embodiments, the recombinant polynucleic acid e.g., the recombinant polynucleic acid comprising a sequence encoding a CFP, is designed for specific expression in an NK cell, e.g., an NK cell of a human subject, and is not expressed in a B cell, a T cell, a dendritic cell, an epithelial cell, an endothelial cell, a neuronal cell, a cardiac smooth muscle cell, an alveolar cell or any other lineage cell. In some embodiments, the recombinant polynucleic acid is designed specifically for expression in an NK cell, if the polynucleic acid is administered in a delivery vehicle, e.g., a nanoparticle, by injecting systemically or locally into a subject for expression in vivo.

In one embodiment, the recombinant polynucleic acid is designed to comprise at least one exclusive domain that is structurally or functionally governs, or directs the expression of the encoded protein or polypeptide predominantly or exclusively in an NK cell or negatively regulates the expression or functionality of the encoded protein or polypeptide in cells other than NK cells.

In some embodiments, a target cell is, for example, a cancer cell. In some embodiments, a target cell is a virus infected cell. Alternatively, a target can be an immunogen, a pathogen or an infectious agent or an infected cell. In some embodiments, a target cell can be a stressed cell, or an apoptosing cell.

The cytolytic function of NK cells is tightly controlled by activating and inhibitory receptors expressed on the cell surface. There are two major classes of NK receptors (NKR). The first is represented by C-type lectn NKG2D receptor that binds to a family of MHC-like molecules expressed on healthy cells only after periods of cellular stress which inclide the human cytomegalo virus UL-16 binding proteins (ULBP) and MHC4 related chain (MIC) proteins. The second class of receptors includes the natural cytotoxicity receptors (NCR) NKp30, NKp44, and NKp46, which can bind to membrane-associated heparan sulfate glycosarninoglycans, viral hemagglutinin and β-1,3-glucan. Ligands that interact with a variety of NK-cell receptors include the human leukocyte antigen (HLA) molecules. NK-cell function can be regulated by HLA class I molecules. HLA class I molecules are ligands for NK cell receptors called KIR receptors (killer cell immunoglobulin like receptors (KIR). NK-cell can also crosstalk with immune cells expressing HLA class II molecules. In some embodiments, NK cells are activated by receptor KIR-S activation, which can bind to an HLA, e.g., HLA-C. In some embodiments, NK cells are activated by receptor CD94-NKG2C activation upon binding to an HLA, e.g., HLA-E. In some embodiments, NK cells are activated by receptor CD94-NKG2E activation upon binding to an HLA, e.g., HLA-E. In some embodiments, NK cells are activated by receptor NKp46 activation upon binding to viral hemagglutinins. In some embodiments, NK cells are activated by receptor NKp44 activation upon binding to viral hemagglutinins. In some embodiments, NK cells are activated by receptor NKp30 activation upon binding to pp65. In some embodiments, NK cells are activated by receptor NKG2D activation upon binding to a ligand e.g., MICA, MICB, or ULBP. In some embodiments, NK cells are activated by receptor CD244, which is activated upon binding to CD48 on a target cell. In some embodiments, NK cells are activated by receptor integrins. In some embodiments, the α2β1 integrin receptor on an NK cell is activated upon binding VCAM-1 (CD106). In some embodiments, NK cells are activated by activation of β2 integrins expressed on the NK cell, that bind to ICAM-1 (CD54) on a target cell. In some embodiments, NK cells are activated by CD11α-CD18 activation on the NK cell upon binding ICAM-2 on a target cell. In some embodiments, NK cells are activated by activation of CD11b-CD18 upon binding CD23 on a target cell. In some embodiments NK cells are activated by activation of CD11c-CD18 upon binding iC3b on a target cell. In some embodiments, NK cells are activated by activation of CD96 upon binding Ned5 on a target cell. In some embodiments NK cells are activated by activation of CD11c-CD18 upon binding iC3b on a target cell. In some embodiments NK cells are activated by activation of CD100 upon binding CD72 on a target cell. These constitute a non-exhaustive list of receptors that naturally occur on a NK cell. NK cell upon engineering nay exhibit a higher functioning or activation of any of the receptors discussed above. Additionally, the instant disclosure is directed to design of chimeric receptors that comprise at least a portion of any one of these receptors that naturally occur in a NK cell. In some embodiments the CFP comprises an extracellular domain or portion thereof, a hinge or a transmembrane domain, or a signaling domain from an NK cell activate receptor, as described in this paragraph.

Expression of CD80, CD86 or NKG2D can activate NKRs and trigger NK cytotoxicity. In one embodiment, for example, a costimulatory molecule, such as NKG2D is included in the CFP design on the extracellular domain in addition to the antigen binding domain, e.g., via a short linker (similar to a BiME or TRiME domain) for activating an NKR on the same cell.

In some embodiments, the transmembrane domain is a transmembrane domain from a cell surface receptor selected from a group consisting of CD39, CD56, CD57, CD94, CD159a (NKG2A), CD159c (NKG2C), CD314 (NKG2D), CD335 (NKp46), CD336 (NKp44), CD337 (NKp30), DAP12, DAP10, NKG2C, NKG2D, NKG2E, Ly49D, Ly49D, NKp46, NKp30 and NKp44. For example, CD39 is an integral membrane protein that expresses in NK cells, that phosphohydrolyzes ATP, and less efficiently ADP, in a Ca²⁺—and Mg²⁺-dependent fashion into AMP. Human CD39 is a putative 510-amino acid protein with two transmembrane regions. Structurally, it is characterized by two transmembrane domains, a small cytoplasmic domain comprising the NH₂—and COOH-terminal segments, and a large extracellular hydrophobic domain consisting of five highly conserved domains, known as apyrase conserved regions (ACR) 1-5, which are pivotal for the catabolic activity of the enzyme. Expression of CD39 is upregulated in tumor cells and infections. For example CD159 (NKG2) is a receptor specific to NK cells. CD159 (NKG2) has 7 known subtypes, A, B, C, D, E and F. The NKG2 receptors can dimerize with other receptors, e.g. CD94 and induce activating or inhibitory function for the cell.

In some embodiments, the transmembrane domain is a transmembrane domain from CD94, CD159a, CD159c, CD314, CD335, CD336, CD337, DAP12, or DAP10. In some embodiments, the extracellular domain is an extracellular domain from CD39, CD56, CD57, CD94, CD159a, CD159c, CD314, CD335, CD336, CD337, DAP12, DAP10, NKG2C, NKG2D, NKG2E, Ly49D, Ly49D, NKp46, NKp30 or NKp44.

In some embodiments, the antigen binding domain comprises a Fab fragment, an scFv domain or an sdAb domain. In some embodiments, the extracellular domain is an extracellular domain from CD94, CD159a, CD159c, CD314, CD335, CD336, CD337, DAP12 or DAP10.

In some embodiments, the extracellular domain further a hinge domain from CD8, wherein the hinge domain is operatively linked to the transmembrane domain.

In some embodiments, the antigen binding domain comprises a sequence of an antigen binding domain provided herein, such as a sequence of an antigen binding domain in a Table 1 provided herein.

In some embodiments, the target protein is CD5. In some embodiments, the antigen binding domain comprises an anti-CD5 antibody or binding fragment thereof, e.g., an scFv that comprises a heavy chain complementarity determining region 3 (HC CDR3) that is the HC CDR3 RGYDWYFDV (SEQ ID NO: 294). In some embodiments, the extracellular domain comprising an anti-CD5 antibody or binding fragment thereof comprises an Anti-CD5 heavy chain variable domain having a sequence that is at least 80%, 90%, 91%, 92%, 93%, 94%, 95%, 96%, 97%, 98%, 99% or 100% identical to

(SEQ ID NO: 44)
EIQLVQSGGGLVKPGGSVRISCAASGYTFTNYGMNWVRQAPGKGLEWMG
WINTHTGEPTYADSFKGRFTFSLDDSKNTAYLQINSLRAEDTAVYFCTR
RGYDWYFDVWGQGTTVTVSS.

In some embodiments, the extracellular domain comprising an anti-CD5 antibody or binding fragment thereof comprises an anti-CD5 scFv having a sequence, that is at least 80%, 90%, 91%, 92%, 93%, 94%, 95%, 96%, 97%, 98%, 99% or 100% identical to

(SEQ ID NO: 45)
EIQLVQSGGGLVKPGGSVRISCAASGYTFTNYGMNWVRQAPGKGLEWMG
WINTHTGEPTYADSFKGRFTFSLDDSKNTAYLQINSLRAEDTAVYFCTR
RGYDWYFDVWGQGTTVTVSSGGGGSGGGGSGGGGSDIQMTQSPSSLSAS
VGDRVTITCRASQDINSYLSWFQQKPGKAPKTLIYRANRLESGVPSRFS
GSGSGTDYTLTISSLQYEDFGIYYCQQYDESPWTFGGGTKLEIK,

where the CDR regions are underlined.

In one embodiment, the chimeric fusion protein comprises an extracellular domain having a CD5 binding domain, according to the paragraphs above, and comprises a domain that allows NK cell specific expression of the CFP. For example, the CFP having a CD5 binding domain according to the paragraphs above comprises a domain that exhibits a dependency on an ITAM motif containing co-receptor for specific expression in an NK cell, and can express predominantly or only in an NK cell, and not express substantively in non-NK cells. In some embodiments, for example, a CD5 binding CFP is designed that comprises a domain from a NKG2D immune receptor, wherein the domain from a NKG2D immune receptor can dimerize with an ITAM motif containing receptor, DAP10 or a DAP12. In another exemplary embodiment, the CD5 binding CFP comprises a domain from a NKG2C or an NKG2E immune receptor, that can heterodimerize with CD94, which bind to DAP12. In yet another exemplary embodiment, the CD5 binding CFP comprises a domain from a Ly49D or a Ly49H immune receptor, that can associate with or bind to the ITAM containing co-receptor DAP12. In some embodiments, the CD5 binding CFP comprises a domain from a KIR receptor, that can associate with or bind to the ITAM containing co-receptor DAP12. In some embodiments, the CD5 binding CFP comprises a domain from an NKp46 immune receptor, or an NKp44 receptor or an NKp30 receptor that can associate with CD3zeta or Fcgamma chain. The construct designs described herein are then tested for NK cell specific expression and or functionality. In some embodiments, any one or more of the chimeric fusion protein designs described above exhibit an NK cell-specific expression. In some embodiments, the chimeric fusion protein is tested for NK cell specific function. In some embodiments, the NK cell-specific chimeric fusion protein is used for therapeutic application.

In some embodiments, the extracellular domain comprising an anti-HER2 antibody or binding fragment thereof comprises an anti-HER2 binding domain, having an HC CDR3 sequence WGGDGFYAMDV (SEQ ID NO: 46).

In some embodiments, the extracellular domain comprising an anti-HER2 antibody or binding fragment thereof comprises an anti-HER2 heavy chain variable domain having a sequence, that is at least 80%, 90%, 91%, 92%, 93%, 94%, 95%, 96%, 97%, 98%, 99% or 100% identical to

(SEQ ID NO: 47)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDVWGQGTLVTVSS.

In some embodiments, the extracellular domain comprising an anti-HER2 antibody or binding fragment thereof comprises an anti-HER2 heavy chain variable domain having a sequence, that is at least 80%, 90%, 91%, 92%, 93%, 94%, 95%, 96%, 97%, 98%, 99% or 100% identical to

(SEQ ID NO: 48)
LVQPGGSLRLSCAASGFNIKDTYIHWVRQAPGKGLEWVARIYPTNGYTRY
ADSVKGRFTISADTSKNTAYLQMNSLRAEDTAVYYCSRWGGDGFYAMDVW
GQGTLVTV.

In some embodiments, the extracellular domain compr sh9g1 Ati HER2 antibody or binding fragment comprises a LC CDR3 QQHYTTPPT (SEQ ID NO: 49). In some embodiments, the extracellular domain comprising an anti HER2 antibody or binding fragment comprises an anti-HER2 light chain variable domain having a sequence that is at least 80%, 90%, 91%, 92%, 93%, 94%, 95%, 96%, 97%, 98%, 99% or 100% identical to

(SEQ ID NO: 50)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTGSTSGSGKPGSGEGSEVQLVE.

In some embodiments, the extracellular domain comprising an anti HER2 antibody or binding fragment comprises an anti-HER2 light chain variable domain having a sequence that is at least 80%, 90%, 91%, 92%, 93%, 94%, 95%, 96%, 97%, 98%, 99% or 100% identical to

(SEQ ID NO: 51)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIK.

In some embodiments, the extracellular domain comprising an anti-HER2 antibody or GP-26,DNA binding fragment thereof comprises an anti-HER2 scFv having a sequence, that is at least 80%, 90%, 91%, 92%, 93%, 94%, 95%, 96%, 97%, 98%, 99% or 100% identical to

(SEQ ID NO: 52)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTGSTSGSGKPGSGEGSEVQLVESSGGGGSGGGGSGGGGSLVQ
PGGSLRLSCAASGFNIKDTYIHWVRQAPGKGLEWVARIYPTNGYTRYADS
VKGRFTISADTSKNTAYLQMNSLRAEDTAVYYCSRWGGDGFYAMDVWGQG
TLVTV.

In some embodiments, the extracellular domain comprising an anti-HER2 antibody or binding fragment thereof comprises an anti-HER2 scFv having a sequence that is at least 80%, 90%, 91%, 92%, 93%, 94%, 95%, 96%, 97%, 98%, 99% or 100% identical to

(SEQ ID NO: 53)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTGSTSGSGKPGSGEGSEVQLVESGGGLVQPGGSLRLSCAASG
FNIKDTYIHWVRQAPGKGLEWVARIYPTNGYTRYADSVKGRFTISADTSK
NTAYLQMNSLRAEDTAVYYCSRWGGDGFYAMDVWGQGTLVTVSS.

In some embodiments, the extracellular domain comprising an anti-HER2 antibody or binding fragment thereof comprises an anti-HER2 scFv having a 70-100% sequence identity to a sequence, that is at least 80%, 90%, 91%, 92%, 93%, 94%, 95%, 96%, 97%, 98%, 99% or 100% identical to

(SEQ ID NO: 53)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTGSTSGSGKPGSGEGSEVQLVESGGGLVQPGGSLRLSCAASG
FNIKDTYIHWVRQAPGKGLEWVARIYPTNGYTRYADSVKGRFTISADTSK
NTAYLQMNSLRAEDTAVYYCSRWGGDGFYAMDVWGQGTLVTVSS.

In one embodiment, the chimeric fusion protein comprises an extracellular domain having a HER2 binding domain, according to the paragraphs above, and comprises a domain that allows NK cell specific expression of the CFP. For example, the CFP having a HER2 binding domain according to the paragraphs above comprises a domain that exhibits a dependency on an ITAM motif containing co-receptor for specific expression in an NK cell, and can express predominantly or only in an NK cell, and not express substantively in non-NK cells. In some embodiments, for example, a HER2 binding CFP is designed that comprises a domain from a NKG2D immune receptor, wherein the domain from a NKG2D immune receptor can dimerize with an ITAM motif containing receptor, DAP10 or a DAP12. In another exemplary embodiment, the HER2 binding CFP comprises a domain from a NKG2C or an NKG2E immune receptor, that can heterodimerize with CD94, which bind to DAP12. In yet another exemplary embodiment, the HER2 binding CFP comprises a domain from a Ly49D or a Ly49H immune receptor, that can associate with or bind to the ITAM containing co-receptor DAP12. In some embodiments, the HER2 binding CFP comprises a domain from a KIR receptor, that can associate with or bind to the ITAM containing co-receptor DAP12. In some embodiments, the HER2 binding CFP comprises a domain from an NKp46 immune receptor, or an NKp44 receptor or an NKp30 receptor that can associate with CD3zeta or Fcgamma chain. In some embodiments, any one or more of the chimeric fusion protein designs described above exhibit an NK cell-specific expression. In some embodiments, the chimeric fusion protein is tested for NK cell specific expression. In some embodiments, the NK cell-specific chimeric fusion protein is used for therapeutic application.

In some embodiments, the target protein is CD70. In some embodiments, the antigen binding domain comprises an anti-CD70 antibody or binding fragment thereof, wherein the antigen binding domain comprises a heavy chain variable domain (VH) comprising a heavy chain complementarity determining region 3 (HC CDR3) that is the HC CDR3 of any one of the VH sequences selected from the group consisting of QVQLQESGGGLVQAGGSLRLSCAAPRSIFSINAMGWYRQAPGKQRELVAAITSGGSPTYAD SVKGRFTISRDNAKNTVYLQMNSLKAEDTAVYYCATGPYGLDNALDAWGQGTQVTVSS (SEQ ID NO: 54) and QVQLQESGGGLVQTGGSLRLACTASGFTFDDYAIAWFRQAPGKEREFVAAISWSGGTTHY ADSVKGRFTISRDNAKNTLYLQMSSLKPEDTAVYFCAKSLRSSPSSRWFGSRGQGTQVTVS S (SEQ ID NO: 55), or a sequence that is at least 80%, 90%, 91%, 92%, 93%, 94%, 95%, 96%, 97%, 98%, 99% or 100% identical to any one of the above sequences. In some embodiments, the VH of the anti-CD70 antibody or binding fragment thereof further comprises a heavy chain complementarity determining region 1 (HC CDR1) that is the HC CDR1 of any one of the VH sequences selected from the group consisting of QVQLQESGGGLVQAGGSLRLSCAAPRSIFSINAMGWYRQAPGKQRELVAAITSGGSPTYAD SVKGRFTISRDNAKNTVYLQMNSLKAEDTAVYYCATGPYGLDNALDAWGQGTQVTVSS (SEQ ID NO: 54) and QVQLQESGGGLVQTGGSLRLACTASGFTFDDYAIAWFRQAPGKEREFVAAISWSGGTTHY ADSVKGRFTISRDNAKNTLYLQMSSLKPEDTAVYFCAKSLRSSPSSRWFGSRGQGTQVTVS S (SEQ ID NO: 55). In some embodiments, the VH of the anti-CD70 antibody or binding fragment thereof further comprises a heavy chain complementarity determining region 2 (HC CDR2) that is the HC CDR2 of any one of the VH sequences selected from the group consisting of QVQLQESGGGLVQAGGSLRLSCAAPRSIFSINAMGWYRQAPGKQRELVAAITSGGSPTYAD SVKGRFTISRDNAKNTVYLQMNSLKAEDTAVYYCATGPYGLDNALDAWGQGTQVTVSS (SEQ ID NO: 54) and QVQLQESGGGLVQTGGSLRLACTASGFTFDDYAIAWFRQAPGKEREFVAAISWSGGTTHY ADSVKGRFTISRDNAKNTLYLQMSSLKPEDTAVYFCAKSLRSSPSSRWFGSRGQGTQVTVS S (SEQ ID NO: 55). In some embodiments, the VH of the anti-CD70 antibody or binding fragment thereof comprises with 70-100% sequence identity to any one of the sequences selected from the group consisting of QVQLQESGGGLVQAGGSLRLSCAAPRSIFSINAMGWYRQAPGKQRELVAAITSGGSPTYAD SVKGRFTISRDNAKNTVYLQMNSLKAEDTAVYYCATGPYGLDNALDAWGQGTQVTVSS (SEQ ID NO: 54) and QVQLQESGGGLVQTGGSLRLACTASGFTFDDYAIAWFRQAPGKEREFVAAISWSGGTTHY ADSVKGRFTISRDNAKNTLYLQMSSLKPEDTAVYFCAKSLRSSPSSRWFGSRGQGTQVTVS S (SEQ ID NO: 55) In some embodiments, the VH is a single domain antibody domain. In some embodiments, the VH is a VHH.

In one embodiment, the chimeric fusion protein comprises an extracellular domain having a CD70 binding domain, according to the paragraphs above, and comprises a domain that allows NK cell specific expression of the CFP. For example, the CFP having a CD70 binding domain according to the paragraphs above comprises a domain that exhibits a dependency on an ITAM motif containing co-receptor for specific expression in an NK cell, and can express predominantly or only in an NK cell, and not express substantively in non-NK cells. In some embodiments, for example, a CD70 binding CFP is designed that comprises a domain from a NKG2D immune receptor, wherein the domain from a NKG2D immune receptor can dimerize with an ITAM motif containing receptor, DAP10 or a DAP12. In another exemplary embodiment, the CD70 binding CFP comprises a domain from a NKG2C or an NKG2E immune receptor, that can heterodimerize with CD94, which bind to DAP12. In yet another exemplary embodiment, the CD70 binding CFP comprises a domain from a Ly49D or a Ly49H immune receptor, that can associate with or bind to the ITAM containing co-receptor DAP12. In some embodiments, the CD70 binding CFP comprises a domain from a KIR receptor, that can associate with or bind to the ITAM containing co-receptor DAP12. In some embodiments, the CD70 binding CFP comprises a domain from an NKp46 immune receptor, or an NKp44 receptor or an NKp30 receptor that can associate with CD3zeta or Fcgamma chain. In some embodiments, any one or more of the chimeric fusion protein designs described above exhibit an NK cell-specific expression. In some embodiments, the chimeric fusion protein is tested for NK cell specific expression. In some embodiments, the NK cell-specific chimeric fusion protein is used for therapeutic application.

In some embodiments the target protein is GPC3. In some embodiments, the antigen binding domain comprises an anti-GPC3 antibody or binding fragment thereof, wherein the antigen binding domain comprises a heavy chain variable domain (VH) comprising a heavy chain complementarity determining region 3 (HC CDR3) of any one of the sequences selected from the group consisting of ATACADTTQYAYDY (SEQ ID NO: 56), ATACADTTLYEYDY (SEQ ID NO: 57), ATACVDTTQYEYDY (SEQ ID NO: 58), ATACADATQHEYDY (SEQ ID NO: 59), ATACADTTQYDYDY (SEQ ID NO: 60), ATACADTTQYEYDY (SEQ ID NO: 61), ATACADTTHYEYDY (SEQ ID NO: 62), ATACVITTLYEYDY (SEQ ID NO: 63), ATACAETTLYEYDY (SEQ ID NO: 64), ATACADTTQHEYDY (SEQ ID NO: 65), ATACVDTTHYEYDY (SEQ ID NO: 66), ATACASTTLYEYDY (SEQ ID NO: 67), ATACVVTTLYEYDY (SEQ ID NO: 68), ATACGGATGPYDY (SEQ ID NO: 69), ATACAGAIGPYDY (SEQ ID NO: 70), ATACVVVGDQNDY (SEQ ID NO: 71), ATACVVVGDRNDY (SEQ ID NO: 72), ATDCAGGTSTPYDY (SEQ ID NO: 73), ATDCAGGTATPYDY (SEQ ID NO: 74), ATACVVADRNEYDY (SEQ ID NO: 75), ATSCVVVTKNEYDY (SEQ ID NO: 76), ATACSGLTHEYDY (SEQ ID NO: 77), ATTCSGLTHEYDY (SEQ ID NO: 78), ATACANWSSLGPYDY (SEQ ID NO: 79), ATACANWSTLGPYDY (SEQ ID NO: 80), ATACSDPRVYEYDY (SEQ ID NO: 81), ATTCASPEKYEYDY (SEQ ID NO: 82), ATHCGGTSWGTSYDY (SEQ ID NO: 83), ATHCGGSSWSNEYDY (SEQ ID NO: 84), YARYSGRTY (SEQ ID NO: 85), ASSAWPAGPKHQVEYDY (SEQ ID NO: 86), ATACGSLVGMYDY (SEQ ID NO: 87), ATACGSAVHEYDY (SEQ ID NO: 88), ATDCVGFGSNWFDY (SEQ ID NO: 89), ATACASPVIYEYDY (SEQ ID NO: 90), ATDCAGGVGHEYDY (SEQ ID NO: 91), ATDCSLHGSDYPYDY (SEQ ID NO: 92) and AVRIYSGSFDNTLAYDY (SEQ ID NO: 93). In some embodiments, the VH of the anti-GPC3 antibody or binding fragment thereof further comprises a heavy chain complementarity determining region 1 (HC CDR1) of any one of the sequences selected from the group consisting of sequences: GFPLAYYA (SEQ ID NO: 94), GFSLDYYA (SEQ ID NO: 95), GFPLDYYA (SEQ ID NO: 96), GFTLDYYA (SEQ ID NO: 97), GFSLNYYA (SEQ ID NO: 98), GFTLAYYA (SEQ ID NO: 99), GFTLGYYA (SEQ ID NO: 100), GFPLNYYA (SEQ ID NO: 101), GFPLHYYA (SEQ ID NO: 102), GFSLGYYA (SEQ ID NO: 103), GFPLGYYA (SEQ ID NO: 104), GFPLEYYA (SEQ ID NO: 105), GSDFRADA (SEQ ID NO: 106), GRTFSSYG (SEQ ID NO: 107), GFSLAYYA (SEQ ID NO: 108) and GLTFRSVG (SEQ ID NO: 109). In some embodiments, the VH of the anti-GPC3 antibody or binding fragment thereof further comprises a heavy chain complementarity determining region 2 (HC CDR2) of any one of the sequences selected from the group consisting of sequences: ISNSDGST (SEQ ID NO: 110), ISASDGST (SEQ ID NO: 111), ISSSDGST (SEQ ID NO: 112), ISSSDGNT (SEQ ID NO: 113), ISSADGST (SEQ ID NO: 114), ISSSGGST (SEQ ID NO: 115), ISSGDGST (SEQ ID NO: 116), ISAGDGNT (SEQ ID NO: 117), ISSSDDST (SEQ ID NO: 118), ISSNDGST (SEQ ID NO: 119), ISSPDGST (SEQ ID NO: 120), ISSRTGGT (SEQ ID NO: 121), ISAGDGSST (SEQ ID NO: 122), ISSSDGSSSDGNT (SEQ ID NO: 123), ISSGDGNT (SEQ ID NO: 124), ISSGDGKT (SEQ ID NO: 125), ISSSDGGT (SEQ ID NO: 126), ISSRTGST (SEQ ID NO: 127), ISSRTGNT (SEQ ID NO: 128), ISSSDGHSST (SEQ ID NO: 129), ISSSSDGNT (SEQ ID NO: 130), ISASNGNT (SEQ ID NO: 131), ISSGSDGNT (SEQ ID NO: 132), ISASDGNT (SEQ ID NO: 133), IDSITSI (SEQ ID NO: 134), ISWSGGSTIAASVGST (SEQ ID NO: 135), ISSSDGSDGNT (SEQ ID NO: 136) and ASPSGVIT (SEQ ID NO: 137). In some embodiments, the VH of the anti-GPC3 antibody or binding fragment thereof comprises with 70-100% sequence identity to any one of the sequences selected from the group consisting of QVQLQESGGGLVHSGGSLRLSCAASGFPLAYYAIGWFRQAPGKEREGVSCISSSDGNTYYA DAVKGRFTISRDNAKNTVYLQMNSLKPEDTAVYYCATACADTTQHEYDYWGQGTQVTVS S (SEQ ID NO: 138), QVQLQESGGGLVHSGGSLRLSCAASGFPLDYYAIGWFRQAPGKEREGVSCISSADGSTYYA DSVKGRFTISRDNAKNTVYLQMNSLGPEDTAVYYCATACADTTQYDYDYWGQGTQVTVS S (SEQ ID NO: 139), QVQLQESGGGLVHSGGSLRLSCAASGFTLDYYAIGWFRRAPGKEREGVSCISSGDGKTYYA DSVKGRFTISRDNAKNTVYLQMNSLKPEDTAVYYCATACAGAIGPYDYWGQGTQVTVSS (SEQ ID NO: 140), QVQLQESGGGLVPPGGSLRLSCAASGFPLDYYAIGWFRQAPGKEREGVSCISSADGSTYYA DSVKGRFTISRDNAKNTVYLQMNSLGPEDTAVYYCATACADTTQYDYDYWGQGTQVTVS S (SEQ ID NO: 141), QVQLQESGGGLVQAGGSLRLSCAASGFSLGYYAIGWFRQAPGKEREGVSCISSSDGHSSTY YADSVKGRFTISRDNAKNTVYLQMNNLKPEDTAVYYCATDCAGGTATPYDYWGQGTQVT VSS (SEQ ID NO: 142), QVQLQESGGGLVQAGGSLRLSCAASGRTFSSYGMGWFRQAPGKEREFVAAISWSGGSTYY ADSVKGRFTISRDNAKNTVYLQMNSLKPEDTAVYYCASSAWPAGPKHQVEYDYWGQGTQ VTVSS (SEQ ID NO: 143), QVQLQESGGGLVQAGGSLRLSCTASGFSLDYYAIGWFRQAPGKEREGVACISSRTGSTYYA DSVKGRFTISRDNAKNTVALQMNSLKPEDTAVYYCATACVVVGDQNDYWGQGTQVTVSS (SEQ ID NO: 144), QVQLQESGGGLVQDGGSLRLSCAASGFPLAYYAIGWFRQAPGKEREGVSCISASDGSTYYA DSVKGRFTISRDNAKNTVYLQMNSLKPEDTAVYYCATACAETTLYEYDYWGQGTQVTVSS (SEQ ID NO: 145), QVQLQESGGGLVQPGESLRLSCAASGFPLAYYAIGWFRQAPGKEREGVSCISSSDGSTYYA DSVKGRFTISRDNAKNTVYLQMNSLKPEDTAVYYCATACANWSTLGPYDYWGQGTQVTV SS (SEQ ID NO: 146), QVQLQESGGGLVQPGESLRLSCAASGFTLAYYAIGWFRQAPGKEREGVSCISSSDGNTYYA DSVKGRFTISRDNAKNTVYLQMNRLKPEDTAVYYCATACADTTQYEYDYWGQGTQVTVS S (SEQ ID NO: 147), QVQLQESGGGLVQPGGSLKLSCAASGSDFRADAMGWYRQAPGKEREPVAIDSITSIYYVDS VEGRFTISRDNTKNTVYLQMTSLKPEDTAVYYCYARYSGRTYWGRGTQVTVSS (SEQ ID NO: 148), QVQLQESGGGLVQPGGSLRLSCAASGFPLAYYAIGWFRQAPGKEREGVSCISASDGSTYYA DSVKGRFTISRDNAKNTVYLQMNSLRPEDTAVYYCATACADTTLYEYDYWGQGTQVTVSS (SEQ ID NO: 149), QVQLQESGGGLVQPGGSLRLSCAASGFPLAYYAIGWFRQAPGKEREGVSCISSSDGNTYYA DAVKGRFAISRDNAKNTVYLQMNSLKPEDTAVYYCATACSDPRVYEYDYWGQGTQVTVS S (SEQ ID NO: 150), QVQLQESGGGLVQPGGSLRLSCAASGFPLAYYAIGWFRQAPGKEREGVSCISSSDGNTYYA DAVKGRFTISRDNAKNTVYLQMNSLKPEDTAVYYCATACADTTQHEYDYWGQGTQVTVS S (SEQ ID NO: 151), QVQLQESGGGLVQPGGSLRLSCAASGFPLAYYAIGWFRQAPGKEREGVSCISSSDGNTYYA DAVKGRFTISRDNAKNTVYLQMNSLKPEDTAVYYCATACVDTTHYEYDYWGQGTQVTVS S (SEQ ID NO: 152), QVQLQESGGGLVQPGGSLRLSCAASGFPLAYYAIGWFRQAPGKEREGVSCISSSDGNTYYA DSVKGRFTISRDNAKNTVYLQMNSLKPEDTAVYYCATACADATQHEYDYWGQGTQVTVS S (SEQ ID NO: 153), QVQLQESGGGLVQPGGSLRLSCAASGFPLAYYAIGWFRQAPGKEREGVSCISSSDGSTYYA DSVKGRFTISRDNAKNTVYLQMNSLGPEDTAVYYCATACADTTQYDYDYWGQGTQVTVS S (SEQ ID NO: 154), QVQLQESGGGLVQPGGSLRLSCAASGFPLAYYAIGWFRQAPGKEREGVSCISSSDGSTYYA DSVKGRFTISRDNAKNTVYLQMNSLKPEDTAVYYCATACADTTQYEYDYWGQGTQVTVS S (SEQ ID NO: 155), QVQLQESGGGLVQPGGSLRLSCAASGFPLAYYAIGWFRQAPGKEREGVSCISSSDGSTYYA DSVKGRFTISRDNAKNTVYLQMNSLKPEDTAVYYCATACGGATGPYDYWGQGTQVTVSS (SEQ ID NO: 156), QVQLQESGGGLVQPGGSLRLSCAASGFPLAYYAIGWFRRAPGKEREGVSCISSSDGNTYYA DAVKGRFTISRDNAKNTVYLQMNSLKPEDTAVYYCATACADTTQHEYDYWGQGTQVTVS S (SEQ ID NO: 157), QVQLQESGGGLVQPGGSLRLSCAASGFPLDYYAIGWFRQAPGKEREGVSCISAGDGSSTYY ADSVKGRFTISRDNAKNTVYLQMNSLKPEDTAVYYCATACASTTLYEYDYWGQGTQVTVS S (SEQ ID NO: 158), QVQLQESGGGLVQPGGSLRLSCAASGFPLDYYAIGWFRQAPGKEREGVSCISSADGSTYYA DSVKGRFTISRDNAKNAVYLQMNSLGPEDTAVYYCATACADTTQYDYDYWGQGTQVTVS S (SEQ ID NO: 159), QVQLQESGGGLVQPGGSLRLSCAASGFPLDYYAIGWFRQAPGKEREGVSCISSADGSTYYA DSVKGRFTISRDNAKNTVYLQMNSLGPEDTAVYYCATACADTTQYDYDYWGQGTQVTVS S (SEQ ID NO: 160), QVQLQESGGGLVQPGGSLRLSCAASGFPLDYYAIGWFRQAPGKEREGVSCISSADGSTYYA DSVKGRFTISRDNAKNTVYLQMNSLKPEDTAVYYCATACVDTTQYEYDYWGQGTQVTVS S (SEQ ID NO: 161), QVQLQESGGGLVQPGGSLRLSCAASGFPLDYYAIGWFRQAPGKEREGVSCISSPDGSTYYA DSVKGRFTISRDNAKNTVYLQMNSLKPEDTAVYYCATACVDTTQYEYDYWGQGTQVTVS S (SEQ ID NO: 162), QVQLQESGGGLVQPGGSLRLSCAASGFPLDYYAIGWFRQAPGKEREGVSCISSSDGSDGNT YYADSVKGRFTISRDNAKNTVYLQMNSLKPEDTAVYYCATDCSLHGSDYPYDYWGQGTQ VTVSS (SEQ ID NO: 163), QVQLQESGGGLVQPGGSLRLSCAASGFPLDYYAIGWFRQAPGKEREGVSCISSSDGSTYYA DSVKGRFTISRDNAKNTVYLQMNSLKPEDTAVYYCATACADTTQYEYDYWGQGTQVTVS S (SEQ ID NO: 164), QVQLQESGGGLVQPGGSLRLSCAASGFPLEYYAIGWFRQAPGKEREGVSCISSSDGSTYYA DSVKGRFTISRDNAKNTVYLQMNSLKPEDTAVYYCATACSDPRVYEYDYWGQGTQVTVSS (SEQ ID NO: 165), QVQLQESGGGLVQPGGSLRLSCAASGFPLGYYAIGWFRQAPGKEREGVSCISSSDDSTYYA DSVKGRFTISRDNDKNTVYLQMNSLKPEDTAVYYCATDCAGGTSTPYDYWGQGTQVTVSS (SEQ ID NO: 166), QVQLQESGGGLVQPGGSLRLSCAASGFPLHYYAIGWFRQAPGKEREGVSCISSGDGSTYYA DSVKGRFTISRDNAKNTVYLQMNSLKPEDTAVYYCATSCVVVTKNEYDYWGQGTQVTVS S (SEQ ID NO: 167), QVQLQESGGGLVQPGGSLRLSCAASGFPLHYYAIGWFRQAPGKEREGVSCISSSDGSTYYA DSVKGRFTISRDNAKNTVYLQMNSLKPEDTAVYYCATACGGATGPYDYWGQGTQVTVSS (SEQ ID NO: 168), QVQLQESGGGLVQPGGSLRLSCAASGFPLHYYAIGWFRQAPGKEREGVSCISSSDGSTYYA DSVKGRFTISRDNAKNTVYLQMNSLKPEDTAVYYCATACVVADRNEYDYWGQGTQVTVS S (SEQ ID NO: 169), QVQLQESGGGLVQPGGSLRLSCAASGFPLHYYAIGWFRQAPGKEREGVSCISSSDGSTYYA DSVKGRFTISRDNAKNTVYLQMNSLRPEDTAVYYCATACVVADRNEYDYWGQGTQVTVS S (SEQ ID NO: 170), QVQLQESGGGLVQPGGSLRLSCAASGFPLNYYAIGWFRQAPGKEREGVSCISASDGNTYYA DSVKGRFTISRDNAKNTVYLQMNSLKPEDTAVYYCATTCASPEKYEYDYWGQGTQVTVSS (SEQ ID NO: 171), QVQLQESGGGLVQPGGSLRLSCAASGFPLNYYAIGWFRQAPGKEREGVSCISSSDGSTYYA DSVKGRFIISRDNAKNTVYLQMNSLKPEDTAVYYCATACGGATGPYDYWGQGTQVTVSS (SEQ ID NO: 172), QVQLQESGGGLVQPGGSLRLSCAASGFPLNYYAIGWFRQAPGKEREGVSCISSSDGSTYYA DSVKGRFTISRDNAKNTVYLQMNSLKPEDTAVYYCATACGSAVHEYDYWGQGTQVTVSS (SEQ ID NO: 173), QVQLQESGGGLVQPGGSLRLSCAASGFSLAYYAIGWFRQAPGKEREGVSCIAASVGSTYYA DSVKGRFTISRDDAKNTVYLQMNSLKPEDTAVYYCATDCAGGVGHEYDYWGQGTQVTVS S (SEQ ID NO: 174), QVQLQESGGGLVQPGGSLRLSCAASGFSLDYYAIGWFRQAPGKEREGVSCISSSDGSTYYA DSVKGRFTISRDNAKNAVYLQMNSLKPEDTAVYYCATACGGATGPYDYWGQGTQVTVSS (SEQ ID NO: 175), QVQLQESGGGLVQPGGSLRLSCAASGFSLDYYAIGWFRQAPGKEREGVSCISSSDGSTYYA DSVKGRFTISRDNAKNAVYLQMNSLKPEDTAVYYCATACVDTTQYEYDYWGQGTQVTVS S (SEQ ID NO: 176), QVQLQESGGGLVQPGGSLRLSCAASGFSLDYYAIGWFRQAPGKEREGVSCISSSDGSTYYA DSVKGRFTISRDNAKNTVYLQMNSLKPEDTAVYYCATDCAGGTSTPYDYWGQGTQVTVSS (SEQ ID NO: 177), QVQLQESGGGLVQPGGSLRLSCAASGFSLNYYAIGWFRQAPGKEREGVSCISAGDGNTYYA DSVKGRFTISRDNAANTVSLQMDSLKPEDTAVYYCATACVITTLYEYDYWGQGTQVTVSS (SEQ ID NO: 178), QVQLQESGGGLVQPGGSLRLSCAASGFTLAYYAIGWFRQAPGKEREGVSCISSSDGSTYYA DSVKGRFTISRDNAKNTVYLQMNSLKPEDTAVYYCATACADTTQHEYDYWGQGTQVTVS S (SEQ ID NO: 179), QVQLQESGGGLVQPGGSLRLSCAASGFTLAYYAIGWFRQAPGKEREGVSCISSSDGSTYYA DSVKGRFTISRDNAKNTVYLQMNSLKPEDTAVYYCATACADTTQYEYDYWGQGTQVTVS S (SEQ ID NO: 180), QVQLQESGGGLVQPGGSLRLSCAASGFTLDYYAIGWFRQAPGKEREGVACISSSDGSTYYA DSVKGRFTISRDNAKNTVYLQMNSLKPEDTAVYYCATACGGATGPYDYWGQGTQVTVSS (SEQ ID NO: 181), QVQLQESGGGLVQPGGSLRLSCAASGFTLDYYAIGWFRQAPGKEREGVACISSSDGSTYYA DSVKGRFTISRDNAKNTVYLQMNSLKPQDTAVYYCATACGSLVGMYDYWGQGTQVTVSP (SEQ ID NO: 182), QVQLQESGGGLVQPGGSLRLSCAASGFTLDYYAIGWFRQAPGKEREGVSCISASDGNTYYA DSVKGRFTISRDNAKNTVYLQMNSLKPEDTAVYYCATTCASPEKYEYDYWGQGTQVTVSS (SEQ ID NO: 183), QVQLQESGGGLVQPGGSLRLSCAASGFTLDYYAIGWFRQAPGKEREGVSCISASNGNTYYA DSVKGRFTISRDSAKNTVYLQMNSLKPEDTAVYYCATTCSGLTHEYDYWGQGTQVTVSS (SEQ ID NO: 184), QVQLQESGGGLVQPGGSLRLSCAASGFTLDYYAIGWFRQAPGKEREGVSCISSGDGNTYYA DSVKGRFTISRDNAKNTVYLQMNSLKPEDTAVYYCATACGGATGPYDYWGQGTQVTVSS (SEQ ID NO: 185), QVQLQESGGGLVQPGGSLRLSCAASGFTLDYYAIGWFRQAPGKEREGVSCISSGDGSTYYA DSVKGRFTISRDNAKNTVYLQMNSLKPEDTAVYYCATHCGGSSWSNEYDYWGQGTQVTV SS (SEQ ID NO: 186), QVQLQESGGGLVQPGGSLRLSCAASGFTLDYYAIGWFRQAPGKEREGVSCISSNDGSTYYA DSVKGRFTISRDNAKNTVYLQMNSLKPEDTAVYYCATACADTTQHEYDYWGQGTQVTVS S (SEQ ID NO: 187), QVQLQESGGGLVQPGGSLRLSCAASGFTLDYYAIGWFRQAPGKEREGVSCISSSDGGTYYA DSVKGRFTISRDNAKNTVYLQMNSLKPEDTAVYYCATACGGATGPYDYWGQGTQVTVSS (SEQ ID NO: 188), QVQLQESGGGLVQPGGSLRLSCAASGFTLDYYAIGWFRQAPGKEREGVSCISSSDGSSSDGN TYYADSVKGRFTISRDNAKNTVYLQMNNLKPEDTAVYYCATACVVTTLYEYDYWGQGTQ VTVSS (SEQ ID NO: 189), QVQLQESGGGLVQPGGSLRLSCAASGFTLDYYAIGWFRQAPGKEREGVSCISSSDGSTYYA DSVKGRFTISRDNAKNTVYLQMNSLKPEDTAVYYCATACADTTQYEYDYWGQGTQVTVSP(SEQ ID NO: 190), QVQLQESGGGLVQPGGSLRLSCAASGFTLDYYAIGWFRQAPGKEREGVSCISSSDGSTYYA DSVKGRFTISRDNAKNTVYLQMNSLKPEDTAVYYCATACGGATGPYDYWGQGTQVTVSS (SEQ ID NO: 191), QVQLQESGGGLVQPGGSLRLSCAASGFTLDYYAIGWFRQAPGKEREGVSCISSSGGSTYYA DSVKGRFTISRDNAKNTVYLQMNMLKPEDTAVYYCATACADTTQYEYDYWGQGTQVTVS S (SEQ ID NO: 192), QVQLQESGGGLVQPGGSLRLSCAASGFTLDYYAIGWFRQAPGKEREGVSCISSSGGSTYYA DSVKGRFTISRDNAKNTVYLQMNSLKPEDTAVYYCATACASPVIYEYDYWGQGTQVTVSS (SEQ ID NO: 193), QVQLQESGGGLVQPGGSLRLSCAASGFTLDYYAIGWFRQAPGKEREGVSCISSSGGSTYYA DSVKGRFTISRDNAKNTVYLQMNSLKPEDTAVYYCATDCAGGTSTPYDYWGQGTQVTVSS (SEQ ID NO: 194), QVQLQESGGGLVQPGGSLRLSCAASGFTLGYYAIGWFRQAPGKEREGVSCISSSDGSTYYA DSVKGRFTISRDNAKNTVYLQMNSLKPEDTAVYYCATACADTTQYEYDYWGQGTQVTVS S (SEQ ID NO: 195), QVQLQESGGGLVQPGGSLRLSCAASGFTLGYYAIGWFRQAPGKEREGVSCISSSDGSTYYA DSVKGRFTISRDNAKNTVYLQMNSLKPEDTAVYYCATACANWSSLGPYDYWGQGTQVTV SS (SEQ ID NO: 196), QVQLQESGGGLVQPGGSLRLSCAASGFTLGYYAIGWFRQAPGKEREGVSCISSSDGSTYYA DSVKGRFTISRDNAKNTVYLQMNSLKPEDTGVYYCATACGGATGPYDYWGQGTQVTVSS (SEQ ID NO: 197), QVQLQESGGGLVQPGGSLRLSCEGSGFSLDYYAIGWFRQAPGKEREGVSCISSGDGNTYYA DSVKGRFTISRDNAKNTVYLQMNSLKPEDTAVYYCATDCVGFGSNWFDYWGQGTQVTVS S (SEQ ID NO: 198), QVQLQESGGGLVQPGGSLRLSCTASGFSLDYYAIGWFRQAPGKEREGVACISSRTGSTYYA DSVKGRFTISRDNAKNTVALQMNSLKPEDTAVYYCATACVVVGDQNDYWGQGTQVTVSS (SEQ ID NO: 199), QVQLQESGGGLVQPGGSLRLSCTASGFSLDYYAIGWFRQAPGKEREGVSCISSRTGGTYYA DSVKGRFTISRDDAKNTVYLQMNSLKPEDTAVYYCATACVVVGDRNDYWGQGTQVTVSS (SEQ ID NO: 200), QVQLQESGGGLVQPGGSLRLSCTASGFSLDYYAIGWFRQAPGKEREGVSCISSRTGGTYYA DSVKGRFTISRDNAKNTVYLQMNSLKPEDTAVYYCATACVDTTQYEYDYWGQGTQVTVS S (SEQ ID NO: 201), QVQLQESGGGLVQPGGSLRLSCTASGFSLDYYAIGWFRQAPGKEREGVSCISSRTGGTYYA DSVKGRFTISRDNAKNTVYLQMNSLKPEDTAVYYCATACVVVGDQNDYWGQGTQVTVSS (SEQ ID NO: 202), QVQLQESGGGLVQPGGSLRLSCTASGFSLDYYAIGWFRQAPGKEREGVSCISSRTGNTYYA DSVKGRFTISRDDAKNMVYLQMNSLKPEDTAVYYCATACVVVGDQNDYWGQGTQVTVS S (SEQ ID NO: 203), QVQLQESGGGLVQPGGSLRLSCTASGFSLDYYAIGWFRQAPGKEREGVSCISSRTGSTYYA DSVKGRFTISRDDAKNTVYLQMNSLKPEDTAVYYCATACVVVGDQNDYWGQGTQVTVSS (SEQ ID NO: 204), QVQLQESGGGLVQPGGSLRLSCTASGFSLGYYAIGWFRQALGKEREGVSCISSRTGSTYYA DSVKGRFTVSRDDAKNTVYLQMNSLKPEDTAVYYCATACVVVGDQNDYWGQGTQVTVS S (SEQ ID NO: 205), QVQLQESGGGLVQPGGSLRLSCTASGFSLGYYAIGWFRQAPGKEREGVSCISSRTGSTYYA DSVKGRFAISRDDAKNTVYLQMNSLKPEDTAVYYCATACVVVGDQNDYWGQGTQVTVSS (SEQ ID NO: 206), QVQLQESGGGLVQPGGSLRLSCTASGFSLGYYAIGWFRQAPGKEREGVSCISSRTGSTYYA DSVKGRFTISRDDAKNTVYLQMNSLKPEDTAVYYCATACVVVGDQNDYWGQGTQVTVSS (SEQ ID NO: 207), QVQLQESGGGLVQPGGSLRLSCTASGFSLGYYAIGWFRQAPGKEREGVSCISSRTGSTYYA DSVKGRFTVSRDDAKNTVYLQMNSLKPEDTAVYYCATACVVVGDQNDYWGQGTQVTVS S (SEQ ID NO: 208), QVQLQESGGGLVQPGGSLRLSCVASGFPLDYYAIGWFRQAPGKEREGVSCISSSDGSTYYA DSVKGRFTISRDNAKNTVYLQMNSLKPEDTAVYYCATACGGATGPYDYWGQGTQVTVSS (SEQ ID NO: 209), QVQLQESGGGLVQPGGSLRLSCVASGFSLDYYAIGWFRQAPGKEREGVSCISNSDGSTYYA DSVKGRFTISRDNAKNTVYLQMNSLKPEDTAVYYCATACADTTQYAYDYWGQGTQVTVS S (SEQ ID NO: 210), QVQLQESGGGLVQPGGSLRLSCVASGFTLDYYAIGWFRQAPGKEREGVSCISSGSDGNTYY ADSVKGRFTISRDNAKNTVYLQMNSLKPEDTAVYYCATACSGLTHEYDYWGQGTQVTVSS (SEQ ID NO: 211), QVQLQESGGGLVQPGGSLRLSCVASGFTLDYYAIGWFRQAPGKEREGVSCISSSDDSTYYA DSVKGRFTISRDNAKNTVYLQMNSLKPEDTAVYYCATACADTTQYEYDYWGQGTQVTVS S (SEQ ID NO: 212), QVQLQESGGGLVQPGGSLRLSCVASGFTLDYYAIGWFRQAPGKEREGVSCISSSSDGNTYY ADSVKGRFTISRDNAKNTVYLQMNSLKPEDTAVYYCATTCSGLTHEYDYWGQGTQVTVSS (SEQ ID NO: 213), QVQLQESGGGLVQPGGSLRLSCVASGFTLGYYAIGWFRQAPGKEREGVSCISSSDGSTYYA DSVKGRFTISRDNAKNTVYLQMNSLKPEDTAVYYCATACADTTQYDYDYWGQGTQVTVS S (SEQ ID NO: 214), QVQLQESGGGLVQPGGSLRLSCVGSGFTLDYYAIGWFRQAPGKEREGVSCISSNDGSTYYA DSVKGRFTISRDNAKNTVYLQMNSLKPEDTAVYYCATACGGATGPYDYWGQGTQVTVSS (SEQ ID NO: 215), QVQLQESGGGLVQSGGSLRLSCAASGFPLAYYAIGWFRQAPGKEREGVSCISASDGSTYYA DSVKGRFTISRDNAKNTVYLQMNSLKPEDTAVYYCATACAETTLYEYDYWGQGTQVTVSS (SEQ ID NO: 216), QVQLQESGGGLVQTGGSLRLSCAASGFTLDYYAIGWFRQAPGKEREGVSCISSSDGSTYYA DSVKGRFTISRDNAKNTVYLQMNSLKPEDTAVYYCATACGGATGPYDYWGQGTQVTVSS (SEQ ID NO: 217), QVQLQESGGGMVQAGESLRLSCAASGFPLAYYAIGWFRQAPGKEREGVSCISSSDGNTYYA DSVKGRFTISRDNAKNTVYLQMNSLKPEDTAVYYCATACADATQHEYDYWGQGTQVTVS S (SEQ ID NO: 218), QVQLQESGGGSVQPGESLRLSCAASGFPLDYYAIGWFRQAPGKEREGVSCISASDGSTYYA DSVKGRFTISRDNAKNTVYLQMNSLKPEDTAVYYCATACADTTLYEYDYWGQGTQVTVSS (SEQ ID NO: 219), QVQLQESGGGSVQPGGSLRLSCAASGFTLDYYAIGWFRQAPGKEREGVSCISSGDGSTYYA DSVKGRFTISRDNAKNTVYLQMNSLKPEDTAVYYCATACADTTHYEYDYWGQGTQVTVS S (SEQ ID NO: 220), QVQLQESGGGSVQSGGSLRLSCTASGFSLGYYAIGWFRQAPGKEREGVSCISSRTGSTYYAD SVKGRFTVSRDDAKNTVYLQMNSLKPEDTAVYYCATACVVVGDQNDYWGQGTQVTVSS (SEQ ID NO: 221), QVQLQESGGGSVRPGGSLRLSCAASGFPLAYYAIGWFRQAPGKEREGVSCISSSDGNTYYA DAVKGRFTISRDNAKNAVYLQMNSLKPEDTAVYYCATACADTTQHEYDYWGQGTQVTVS S (SEQ ID NO: 222), QVQLQESGGGVAQPGGSLRLSCAASGFPLDYYAIGWFRQAPGKEREGVSCISASDGSTYYA DSVKGRFTISRDNAKNTVYLQMNSLKPEDTAVYYCATACADTTLYEYDYWGQGTQVTVSS (SEQ ID NO: 223), QVQLQESGGGVVQAGGSLKLSCAASGSDFRADAMGWYRQAPGKEREPVAIDSITSIYYVDS VEGRFTISRDNTKNTVYLQMTSLKPEDTAVYYCYARYSGRTYWGRGTQVTVSS (SEQ ID NO: 224), QVQLQESGGGVVQPGGSLRLSCAASGFSLDYYAIGWFRQAPGKEREGVSCISSGDGSTYYA DSVKGRFTISRDNAKNTVYLQMNSLKPEDTAVYYCATHCGGTSWGTSYDYWGQGTQVTV SS (SEQ ID NO: 225), QVQLQESGGGVVQPGGSLRLSCAASGLTFRSVGMGWFRRAPGKEREFVATASPSGVITYYA DSVKGRFTISRDNAKNTVYLEMNSLKPEDTAVYYCAVRIYSGSFDNTLAYDYWGQGTQVT VSS (SEQ ID NO: 226), QVQLQESGGGVVQPGGSLRLSCTASGFSLGYYAIGWFRQAPGKEREGVSCISSRTGSTYYA DSVKGRFTVSRDDAKNTVYLQMNSLKPEDTAVYYCATACVVVGDQNDYWGQGTQVTVS S (SEQ ID NO: 227), and QVQLQESGGGVVQSGGSLRLSCTASGFSLDYYAIGWFRQAPGKEREGVSCISSRTGSTYYA DSVKGRFTISRDDAKNTVYLQMNSLKPEDTAVYYCATACVVVGDQNDYWGQGTQVTVSS (SEQ ID NO: 228). In some embodiments, the VH is a single domain antibody domain. In some embodiments, the VH is a VHH.

In some embodiments, the extracellular domain of a CFP comprises an anti-GPC3 variable heavy chain (VH) domain, having a 70-100% sequence identity to

(SEQ ID NO: 229)
QVQLVQSGAEVKKPGASVKVSCKASGYTFTDYEMHWVRQAPGQGLEWMGA
LDPKTGDTAYSQKFKGKATLTADKSTSTAYMELSSLTSEDTAVYYCTRFY
SYTYWGQGTLVTVSS.

In some embodiments, the extracellular domain of a CFP comprises an anti-GPC3 variable light chain (VL) domain, having a 70-100% sequence identity to

(SEQ ID NO: 230)
DVVMTQSPLSLPVTPGEPASISCRSSQSLVHSNRNTYLHWYLQKPGQSPQ
LLIYKVSNRFSGVPDRFSGSGSGTDFTLKISRVEAEDVGVYYCSQNTHVP
PTFGQGTKLEIK.

In some embodiments, the extracellular domain of a CFP comprises an anti-GPC3 scFv, comprising a sequence that has 70%-100% sequence identity to the sequence,

(SEQ ID NO: 231)
QVQLVQSGAEVKKPGASVKVSCKASGYTFTDYEMHWVRQAPGQGLEWMGA
LDPKTGDTAYSQKFKGKATLTADKSTSTAYMELSSLTSEDTAVYYCTRFY
SYTYWGQGTLVTVSSGGGGSGGGGSGGGGSDVVMTQSPLSLPVTPGEPAS
ISCRSSQSLVHSNRNTYLHWYLQKPGQSPQLLIYKVSNRFSGVPDRFSGS
GSGTDFTLKISRVEAEDVGVYYCSQNTHVPPTFGQGTKLEIK.

In one embodiment, the chimeric fusion protein comprises an extracellular domain having a GPC3 binding domain, according to the paragraphs above, and comprises a domain that allows NK cell specific expression of the CFP. For example, the CFP having a GPC3 binding domain according to the paragraphs above comprises a domain that exhibits a dependency on an ITAM motif containing co-receptor for specific expression in an NK cell, and can express predominantly or only in an NK cell, and not express substantively in non-NK cells. In some embodiments, for example, a GPC3 binding CFP is designed that comprises a domain from a NKG2D immune receptor, wherein the domain from a NKG2D immune receptor can dimerize with an ITAM motif containing receptor, DAP10 or a DAP12. In another exemplary embodiment, the GPC3 binding CFP comprises a domain from a NKG2C or an NKG2E immune receptor, that can heterodimerize with CD94, which bind to DAP12. In yet another exemplary embodiment, the GPC3 binding CFP comprises a domain from a Ly49D or a Ly49H immune receptor, that can associate with or bind to the ITAM containing co-receptor DAP12. In some embodiments, the GPC3 binding CFP comprises a domain from a KIR receptor, that can associate with or bind to the ITAM containing co-receptor DAP12. In some embodiments, the GPC3 binding CFP comprises a domain from an NKp46 immune receptor, or an NKp44 receptor or an NKp30 receptor that can associate with CD3zeta or Fcgamma chain. In some embodiments, any one or more of the chimeric fusion protein designs described above exhibit an NK cell-specific expression. In some embodiments, the chimeric fusion protein is tested for NK cell specific expression. In some embodiments, the NK cell-specific chimeric fusion protein is used for therapeutic application.

In some embodiments, the extracellular domain of a CFP comprises an anti-TROP2 binding domain that comprises a HC CDR3 sequence, GGFGSSYWYFDV (SEQ ID NO: 42). In some embodiments, the extracellular domain of a CFP comprises an anti-TROP2 binding domain that comprises a LC CDR3 sequence, QQHYITPLT (SEQ ID NO: 43).

In some embodiments, the extracellular domain of a CFP comprises an anti-TROP2 binding domain that has 70-100% sequence identity to the scFv,

(SEQ ID NO: 232)
DIQLTQSPSSLSASVGDRVSITCKASQDVSIAVAWYQQKPGKAPKLLIYS
ASYRYTGVPDRFSGSGSGTDFTLTISSLQPEDFAVYYCQQHYITPLTFGA
GTKVEIKRGGGGSGGGGSGGGGSQVQLQQSGSELKKPGASVKVSCKASGY
TFTNYGMNWVKQAPGQGLKWMGWINTYTGEPTYTDDFKGRFAFSLDTSVS
TAYLQISSLKADDTAVYFCARGGFGSSYWYFDVWGQGSLVTVSS.

In some embodiments, the extracellular domain of a CFP comprises an anti-TROP2 binding domain has 70-100% sequence identity to the scFv,

(SEQ ID NO: 233)
QVQLQQSGSELKKPGASVKVSCKASGYTFTNYGMNWVKQAPGQGLKWMGW
INTYTGEPTYTDDFKGRFAFSLDTSVSTAYLQISSLKADDTAVYFCARGG
FGSSYWYFDVWGQGSLVTVSSGGGGSGGGGSGGGGSDIQLTQSPSSLSAS
VGDRVSITCKASQDVSIAVAWYQQKPGKAPKLLIYSASYRYTGVPDRFSG
SGSGTDFTLTISSLQPEDFAVYYCQQHYITPLTFGAGTKVEIKR.

In one embodiment, the chimeric fusion protein comprises an extracellular domain having a TROP2 binding domain, according to the paragraphs above, and comprises a domain that allows NK cell specific expression of the CFP. For example, the CFP having a TROP2 binding domain according to the paragraphs above comprises a domain that exhibits a dependency on an ITAM motif containing co-receptor for specific expression in an NK cell, and can express predominantly or only in an NK cell, and not express substantively in non-NK cells. In some embodiments, for example, a TROP2 binding CFP is designed that comprises a domain from a NKG2D immune receptor, wherein the domain from a NKG2D immune receptor can dimerize with an ITAM motif containing receptor, DAP10 or a DAP12. In another exemplary embodiment, the TROP2 binding CFP comprises a domain from a NKG2C or an NKG2E immune receptor, that can heterodimerize with CD94, which bind to DAP12. In yet another exemplary embodiment, the TROP2 binding CFP comprises a domain from a Ly49D or a Ly49H immune receptor, that can associate with or bind to the ITAM containing co-receptor DAP12. In some embodiments, the TROP2 binding CFP comprises a domain from a KIR receptor, that can associate with or bind to the ITAM containing co-receptor DAP12. In some embodiments, the TROP2 binding CFP comprises a domain from an NKp46 immune receptor, or an NKp44 receptor or an NKp30 receptor that can associate with CD3zeta or Fcgamma chain. The construct designs described herein are then tested for NK cell specific expression and or functionality. In some embodiments, any one or more of the chimeric fusion protein designs described above exhibit an NK cell-specific expression. In some embodiments, the chimeric fusion protein is tested for NK cell specific function. In some embodiments, the NK cell-specific chimeric fusion protein is used for therapeutic application.

In some embodiments, the extracellular domain of a CFP comprises an Ig binding domain. In some embodiments, the extracellular domain comprises an IgA, IgD, IgE, IgG, IgM, FcRγ1, FcRγIIA, FcRγIIB, FcRγIIC, FcRγIIIA, FcRγIIIB, FcRn, TRIM21, FcRL5 binding domain. In some embodiments, the extracellular domain of a CFP comprises an FcR extracellular domain. In some embodiments, the extracellular domain of a CFP comprises an FcRα, FcRβ, FcRε or FcRγ extracellular domain. In some embodiments, the extracellular domain comprises an FcRα (FCAR) extracellular domain. In some embodiments, the extracellular domain comprises an FcRβ extracellular domain. In some embodiments, the extracellular domain comprises an FCER1A extracellular domain. In some embodiments, the extracellular domain comprises an FDGR1A, FCGR2A, FCGR2B, FCGR2C, FCGR3A, or FCGR3B extracellular domain. In some embodiments, the extracellular domain comprises an integrin domain or an integrin receptor domain. In some embodiments, the extracellular domain comprises one or more integrin α1, α2, αIIb, α3, α4, α5, α6, α7, α8, α9, α10, α11, αD, αE, αL, αM, αV, αX, β1, β2, β3, β4, β5, β6, β7, or β8 domains.

In some embodiments, the CFP further comprises an extracellular domain operatively linked to the transmembrane domain and the extracellular antigen binding domain. In some embodiments, the extracellular domain further comprises an extracellular domain of a receptor, a hinge, a spacer and/or a linker. In some embodiments, the extracellular domain comprises an extracellular portion of a phagocytic receptor. In some embodiments, the extracellular portion of the CFP is derived from the same receptor as the receptor from which the intracellular signaling domain is derived. In some embodiments, the extracellular domain comprises an extracellular domain of a scavenger receptor. In some embodiments, the extracellular domain comprises an immunoglobulin domain. In some embodiments, the immunoglobulin domain comprises an extracellular domain of an immunoglobulin or an immunoglobulin hinge region. In some embodiments, the extracellular domain comprises a phagocytic engulfment domain. In some embodiments, the extracellular domain comprises a structure capable of multimeric assembly. In some embodiments, the extracellular domain comprises a scaffold for multimerization. In some embodiments, the extracellular domain is at least 10, 20, 30, 40, 50, 60, 70, 80, 90, 100, 150, 200, 300, 300, 400, or 500 amino acids in length. In some embodiments, the extracellular domain is at most 500, 400, 300, 200, or 100 amino acids in length. In some embodiments, the extracellular antigen binding domain specifically binds to the antigen of a target cell. In some embodiments, the extracellular antigen binding domain comprises an antibody domain. In some embodiments, the extracellular antigen binding domain comprises a receptor domain, antibody domain, wherein the antibody domain comprises a functional antibody fragment, a single chain variable fragment (scFv), an Fab, a single-domain antibody (sdAb), a nanobody, a V_{H} domain, a V_{L} domain, a VNAR domain, a V_{HH} domain, a bispecific antibody, a diabody, or a functional fragment or a combination thereof. In some embodiments, the extracellular antigen binding domain comprises a ligand, an extracellular domain of a receptor or an adaptor. In some embodiments, the extracellular antigen binding domain comprises a single extracellular antigen binding domain that is specific for a single antigen. In some embodiments, the extracellular antigen binding domain comprises at least two extracellular antigen binding domains, wherein each of the at least two extracellular antigen binding domains is specific for a different antigen.

In some embodiments, the antigen is a cancer associated antigen, a lineage associated antigen, a pathogenic antigen or an autoimmune antigen. In some embodiments, the antigen comprises a viral antigen. In some embodiments, the antigen is a T lymphocyte antigen. In some embodiments, the antigen is an extracellular antigen. In some embodiments, the antigen is an intracellular antigen. In some embodiments, the antigen is selected from the group consisting of an antigen from Thymidine Kinase (TK1), Hypoxanthine-Guanine Phosphoribosyltransferase (HPRT), Receptor Tyrosine Kinase-Like Orphan Receptor 1 (RORI), Mucin-1, Mucin-16 (MUC16), MUC1, Epidermal Growth Factor Receptor VIII (EGFRvIII), Mesothelin, Human Epidermal Growth Factor Receptor 2 (HER2), EBNA-1, LEMD1, Phosphatidyl Serine, Carcinoembryonic Antigen (CEA), B-Cell Maturation Antigen (BCMA), Glypican 3 (GPC3), Follicular Stimulating Hormone receptor, Fibroblast Activation Protein (FAP), Erythropoietin-Producing Hepatocellular Carcinoma A2 (EphA2), EphB2, a Natural Killer Group 2D (NKG2D) ligand, Disialoganglioside 2 (GD2), CD2, CD3, CD4, CD5, CD7, CD8, CD19, CD20, CD22, CD24, CD30, CD33, CD3δ, CD44v6, CD45, CD56CD79b, CD97, CD117, CD123, CD133, CD138, CD171, CD179a, CD213A2, CD248, CD276, PSCA, CS-1, CLECLI, GD3, PSMA, FLT3, TAG72, EPCAM, IL-1, an integrin receptor, PRSS21, VEGFR2, PDGFRβ, SSEA-4, EGFR, NCAM, prostase, PAP, ELF2M, GM3, TEM7R, CLDN6, TSHR, GPRC5D, ALK, Dsg1, Dsg3, IGLL1 and combinations thereof. In some embodiments, the antigen is an antigen of a protein selected from the group consisting of CD2, CD3, CD4, CD5, CD7, CCR4, CD8, CD30, CD45, and CD56. In some embodiments, the antigen is an ovarian cancer antigen or a T lymphoma antigen. In some embodiments, the antigen is an antigen of an integrin receptor. In some embodiments, the antigen is an antigen of an integrin receptor or integrin selected from the group consisting of α1, α2, αIIb, α3, α4, α5, α6, α7, α8, α9, α10, α11, αD, αE, αL, αM, αV, αX, β1, β2, β3, β4, β5, β6, β7, and β8. In some embodiment, the antigen is an antigen of an integrin receptor ligand. In some embodiments, the antigen is an antigen of fibronectin, vitronectin, collagen, or laminin. In some embodiments, the antigen binding domain can bind to two or more different antigens.

In some embodiments, the antigen binding domain comprises an autoantigen or fragment thereof, such as Dsg1 or Dsg3. In some embodiments, the extracellular antigen binding domain comprises a receptor domain or an antibody domain wherein the antibody domain binds to an auto antigen, such as Dsg1 or Dsg3.

TABLE 1
Provided herein are exemplary extracellular antigen binding domain sequences.
The present disclosure encompasses any one of the sequences provide in Table 1, as well as a sequence
that is at least 80%, 90%, 91%, 92%, 93%, 94%, 95%, 96%, 97%, 98%, 99% or 100% identical to any
of the sequences disclosed. Underlines denote the CDR sequences in sequential order of CDR1, CDR2
and CDR3 for the respective heavy and light chains in accordance to the Kabat numbering system.

| CFP/ Domain | Sequence |
|---|---|
| GMCSF Signal | MWLQSLLLLGTVACSIS (SEQ ID NO: 234) |
| peptide | |
| Anti-CD5 heavy | EIQLVQSGGGLVKPGGSVRISCAASGYTFTNYGMNWVRQAPGKGL |
| chain variable | EWMGWINTHTGEPTYADSFKGRFTFSLDDSKNTAYLQINSLRAEDT |
| domain | AVYFCTRRGYDWYFDVWGQGTTVTV (SEQ ID NO: 235) |
| Anti-CD5 heavy | EIQLVQSGGGLVKPGGSVRISCAASGYTFTNYGMNWVRQAPGKGL |
| chain variable | EWMGWINTHTGEPTYADSFKGRFTFSLDDSKNTAYLQINSLRAEDT |
| domain | AVYFCTRRGYDWYFDVWGQGTTVTVSS (SEQ ID NO: 44) |
| Anti-CD5 light | DIQMTQSPSSLSASVGDRVTITCRASQDINSYLSWFQQKPGKAPKTL |
| chain variable | IYRANRLESGVPSRFSGSGSGTDYTLTISSLQYEDFGIYYCQQYDESP |
| domain | WTFGGGTKLEIK (SEQ ID NO: 236) |
| Anti-CD5 scFv | EIQLVQSGGGLVKPGGSVRISCAASGYTFTNYGMNWVRQAPGKGL |
| | EWMGWINTHTGEPTYADSFKGRFTFSLDDSKNTAYLQINSLRAEDT |
| | AVYFCTRRGYDWYFDVWGQGTTVTVSSGGGGSGGGGSGGGGSDI |
| | QMTQSPSSLSASVGDRVTITCRASQDINSYLSWFQQKPGKAPKTLIY |
| | RANRLESGVPSRFSGSGSGTDYTLTISSLQYEDFGIYYCQQYDESPW |
| | TFGGGTKLEIK (SEQ ID NO: 45) |
| Anti-CD5 scFv | MWLQSLLLLGTVACSISEIQLVQSGGGLVKPGGSVRISCAASGYTFT |
| with leader | NYGMNWVRQAPGKGLEWMGWINTHTGEPTYADSFKGRFTFSLDD |
| sequence | SKNTAYLQINSLRAEDTAVYFCTRRGYDWYFDVWGQGTTVTVSSG |
| | GGGSGGGGSGGGGSDIQMTQSPSSLSASVGDRVTITCRASQDINSY |
| | LSWFQQKPGKAPKTLIYRANRLESGVPSRFSGSGSGTDYTLTISSLQ |
| | YEDFGIYYCQQYDESPWTFGGGTKLEIK (SEQ ID NO: 237) |
| Anti-HER2 light | DIQMTQSPSSLSASVGDRVTITCRASQDVNTAVAWYQQKPGKAPK |
| chain variable | LLIYSASFLYSGVPSRFSGSRSGTDFTLTISSLQPEDFATYYCQQHYT |
| domain | TPPTFGQGTKVEIKRTGSTSGSGKPGSGEGSEVQLVE (SEQ ID NO: |
| | 50) |
| Anti-HER2 light | DIQMTQSPSSLSASVGDRVTITCRASQDVNTAVAWYQQKPGKAPK |
| chain variable | LLIYSASFLYSGVPSRFSGSRSGTDFTLTISSLQPEDFATYYCQQHYT |
| domain | TPPTFGQGTKVEIK (SEQ ID NO: 51) |
| Anti-HER2 | LVQPGGSLRLSCAASGFNIKDTYIHWVRQAPGKGLEWVARIYPTNG |
| heavy chain | YTRYADSVKGRFTISADTSKNTAYLQMNSLRAEDTAVYYCSRWGG |
| variable domain | DGFYAMDVWGQGTLVTV (SEQ ID NO: 48) |
| Anti-HER2 | EVOLVESGGGLVQPGGSLRLSCAASGFNIKDTYIHWVRQAPGKGLE |
| heavy chain | WVARIYPTNGYTRYADSVKGRFTISADTSKNTAYLQMNSLRAEDT |
| variable domain | AVYYCSRWGGDGFYAMDVWGQGTLVTVSS (SEQ ID NO: 47) |
| Anti-HER2 scFv | DIQMTQSPSSLSASVGDRVTITCRASQDVNTAVAWYQQKPGKAPK |
| | LLIYSASFLYSGVPSRFSGSRSGTDFTLTISSLQPEDFATYYCQQHYT |
| | TPPTFGQGTKVEIKRTGSTSGSGKPGSGEGSEVQLVESSGGGGSGGG |
| | GSGGGGSLVQPGGSLRLSCAASGFNIKDTYIHWVRQAPGKGLEWV |
| | ARIYPTNGYTRYADSVKGRFTISADTSKNTAYLQMNSLRAEDTAV |
| | YYCSRWGGDGFYAMDVWGQGTLVTV (SEQ ID NO: 52) |
| Anti-HER2 scFv | DIQMTQSPSSLSASVGDRVTITCRASQDVNTAVAWYQQKPGKAPK |
| | LLIYSASFLYSGVPSRFSGSRSGTDFTLTISSLQPEDFATYYCQQHYT |
| | TPPTFGQGTKVEIKRTGSTSGSGKPGSGEGSEVQLVESGGGLVQPG |
| | GSLRLSCAASGFNIKDTYIHWVRQAPGKGLEWVARIYPTNGYTRY |
| | ADSVKGRFTISADTSKNTAYLQMNSLRAEDTAVYYCSRWGGDGFY |
| | AMDVWGQGTLVTVSS (SEQ ID NO: 53) |
| Anti-HER2 scFv | DIQMTQSPSSLSASVGDRVTITCRASQDVNTAVAWYQQKPGKAPK |
| | LLIYSASFLYSGVPSRFSGSRSGTDFTLTISSLQPEDFATYYCQQHYT |
| | TPPTFGQGTKVEIKRTGSTSGSGKPGSGEGSEVQLVESGGGLVQPG |
| | GSLRLSCAASGFNIKDTYIHWVRQAPGKGLEWVARIYPTNGYTRY |
| | ADSVKGRFTISADTSKNTAYLQMNSLRAEDTAVYYCSRWGGDGFY |
| | AMDVWGQGTLVTVSS (SEQ ID NO: 53) |
| Anti-CD137 | MEFGLSWLFLVAILKGVQCGLLDLRQGMFAQLVAQNVLLIDGPLS |
| binding domain | WYSDPGLAGVSLTGGLSYKEDTKELVVAKAGVYYVFFQLELRRV |
| | VAGEGSGSVSLALHLQPLRSAAGAAALALTVDLPPASSEARNSAFG |
| | FQGRLLHLSAGQRLGVHLHTEARARHAWQLTQGATVLGLFRVTPE |
| | IPAGLPSPRSE (SEQ ID NO: 238) |
| Anti-CD70 | QVQLVQSGAEVKKPGASVKVSCKASGYTFTNYGMNWVRQAPGQ |
| binding domain | GLEWMGWINTYTGEPTYADAFKGRVTMTTDTSTSTAYMELRSLRS |
| | DDTAVYYCARDYGDYGMDYWGQGTTVTVSSGSTSGSGKPGSSEG |
| | STKGDIVMTQSPDSLAVSLGERATINCRASKSVSTSGYSFMHWYQQ |
| | KPGQPPKLLIYLASNLESGVPDRFSGSGSGTDFTLTISSLQAEDVAV |
| | YYCQHSREVPWTFGQGTKVEIK (SEQ ID NO: 239) |
| Anti- | QVQLQESGPGLVKPSQTLSLTCTVSGGSISSGYNWHWIRQPPGKGL |
| Claudin18.2 | EWIGYIHYTGSTNYNPALRSRVTISVDTSKNQFSLKLSSVTAADTAV |
| binding domain | YYCARIYNGNSFPYWGQGTTVTVSSGGGGSGGGGSGGGGSDIVMT |
| (scFv) | QSPDSLAYSLGERATINCKSSQSLFNSGNQKNYLTWYQQKPGQPPK |
| | LLIYWASTRESGVPDRFSGSGSGTDIFITISSLQAEDVAVYYCQNAY |
| | SFPYTFGGGTKLEIKR (SEQ ID NO: 240) |
| Anti-Trop-2 | DIQLTQSPSSLSASVGDRVSITCKASQDVSIAVAWYQQKPGKAPKL |
| binding domain | LIYSASYRYTGVPDRFSGSGSGTDFTLTISSLQPEDFAVYYCQQHYIT |
| (scFv) | PLTFGAGTKVEIKRGGGGSGGGGSGGGGSQVQLQQSGSELKKPGA |
| | SVKVSCKASGYTFTNYGMNWVKQAPGQGLKWMGWINTYTGEPT |
| | YTDDFKGRFAFSLDTSVSTAYLQISSLKADDTAVYFCARGGFGSSY |
| | WYFDVWGQGSLVTVSS (SEQ ID NO: 232) |
| Anti-Trop-2 | QVQLQQSGSELKKPGASVKVSCKASGYTFTNYGMNWVKQAPGQG |
| binding domain | LKWMGWINTYTGEPTYTDDFKGRFAFSLDTSVSTAYLQISSLKADD |
| (scFv) | TAVYFCARGGFGSSYWYFDVWGQGSLVTVSSGGGGSGGGGSGGG |
| | GSDIQLTQSPSSLSASVGDRVSITCKASQDVSIAVAWYQQKPGKAP |
| | KLLIYSASYRYTGVPDRFSGSGSGTDFTLTISSLQPEDFAVYYCQQH |
| | YITPLTFGAGTKVEIKR (SEQ ID NO: 233) |
| Anti-Trop-2 VH | QVQLQQSGSELKKPGASVKVSCKASGYTFTNYGMNWVKQAPGQG |
| domain | LKWMGWINTYTGEPTYTDDFKGRFAFSLDTSVSTAYLQISSLKADD |
| | TAVYFCARGGFGSSYWYFDVWGQGSLVTVSS (SEQ ID NO: 241) |
| Anti-TROP2 VL | DIQLTQSPSSLSASVGDRVSITCKASQDVSIAVAWYQQKPGKAPKL |
| domain | LIYSASYRYTGVPDRFSGSGSGTDFTLTISSLQPEDFAVYYCQQHYIT |
| | PLTFGAGTKVEIKR (SEQ ID NO: 242) |
| Anti-GPC3 | QVQLVQSGAEVKKPGASVKVSCKASGYTFTDYEMHWVRQAPGQG |
| binding domain | LEWMGALDPKTGDTAYSQKFKGKATLTADKSTSTAYMELSSLTSE |
| variable heavy | DTAVYYCTRFYSYTYWGQGTLVTVSS (SEQ ID NO: 229) |
| chain | |
| Anti-GPC3 | DVVMTQSPLSLPVTPGEPASISCRSSQSLVHSNRNTYLHWYLQKPG |
| binding domain | QSPQLLIYKVSNRFSGVPDRFSGSGSGTDFTLKISRVEAEDVGVYYC |
| variable light | SQNTHVPPTFGQGTKLEIK (SEQ ID NO: 230) |
| chain | |
| Anti-GPC3 | QVQLVQSGAEVKKPGASVKVSCKASGYTFTDYEMHWVRQAPGQG |
| binding domain | LEWMGALDPKTGDTAYSQKFKGKATLTADKSTSTAYMELSSLTSE |
| (scFv) | DTAVYYCTRFYSYTYWGQGTLVTVSSGGGGSGGGGSGGGGSDVV |
| | MTQSPLSLPVTPGEPASISCRSSQSLVHSNRNTYLHWYLQKPGQSPQ |
| | LLIYKVSNRFSGVPDRFSGSGSGTDFTLKISRVEAEDVGVYYCSQNT |
| | HVPPTFGQGTKLEIK (SEQ ID NO: 231) |

In some embodiments, the transmembrane domain of a CFP expressed in a NK cell is a transmembrane domain from a cell surface receptor selected from a group consisting of CD39, CD56, CD57, CD94, CD159a, CD159c, CD314, CD335, CD336, CD337, DAP12, DAP10, NKG2C, NKG2D, NKG2E, Ly49D, Ly49D, NKp46, NKp30 and NKp44. In some embodiments, the transmembrane domain is a transmembrane domain from CD94, CD159a, CD159c, CD314, CD335, CD336, CD337, DAP12, or DAP10. In some embodiments, the extracellular domain is an extracellular domain from CD39, CD56, CD57, CD94, CD159a, CD159c, CD314, CD335, CD336, CD337, DAP12, DAP10, NKG2C, NKG2D, NKG2E, Ly49D, Ly49D, NKp46, NKp30 or NKp44. In some embodiments, the extracellular domain is an extracellular domain from CD94, CD159a, CD159c, CD314, CD335, CD336, CD337, DAP12 or DAP10. In some embodiments, the extracellular domain further comprises a hinge domain from CD8, wherein the hinge domain is operatively linked to the transmembrane domain. In some embodiments, the CFP is preferentially or specifically expressed in NK cells of the human subject. In some embodiments, the antigen binding domain comprises a Fab fragment, an scFv domain or an sdAb domain.

In some embodiments, a recombinant polynucleic acid comprising a sequence encoding an NK cell-specific CFP comprises a nucleic acid sequence encoding a DAP12 domain. For examples, a polynucleic acid sequence encoding a DAP12 domain may comprises a sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence: CTTCGGCCTGTTCAAGCACAAGCGCAGAGTGACTGCTCTTGTAGCACGGTTTCACCTGG CGTATTGGCCGGTATTGTAATGGGGGACCTTGTACTCACGGTTCTCATAGCTCTTGCTGT CTATTTTCTCGGACGACTGGTCCCACGGGGACGAGGGGCAGCAGAAGCTGCTACACGA AAACAGAGGATTACAGAGACGGAGAGTCCCTACCAAGAACTCCAGGGGCAGAGAAGT GATGTCTATTCTGACCTTAACACACAAAGACCATACTATAAATGA. The polynucleic acid sequence may be a DNA or an RNA, e.g., mRNA. (SEQ ID NO: 1)

In some embodiments, a recombinant polynucleic acid comprising a sequence encoding an NK cell-specific CFP comprises a nucleic acid sequence encoding a CD16A domain. For examples, a polynucleic acid sequence encoding a CD16A domain may comprises a sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence: GGTCTGGCCGTAAGTACCATATCCAGCTTTTTTCCGCCAGGATATCAGGTTTCCTTTTGT TTGGTCATGGTACTTCTCTTTGCGGTAGACACTGGTCTCTATTTTAGTGTCAAAACTAAT ATACGCTCCTCCACGAGGGATTGGAAGGACCATAAGTTCAAATGGAGGAAGGACCCGC AGGACAAATGA. (SEQ ID NO: 2) The polynucleic acid sequence may be a DNA or an RNA, e.g., mRNA.

In one embodiment, a recombinant polynucleic acid comprising a sequence encoding an NK cell-specific CFP comprises a nucleic acid sequence encoding a CD8A hinge domain. In some embodiments, the sequence encoding a CD8A hinge domain may comprise a sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 3)
ACTACTACTCCAGCTCCAAGGCCTCCCACGCCAGCTCCCACTATTGCTTC
TCAACCGTTGTCACTGCGACCAGAGGCCTGTAGACCTGCAGCTGGAGGCG
CTGTTCACACAAGGGGTCTCGATTTTGCGTGTGAC.

In one embodiment, a recombinant polynucleic acid comprising a sequence encoding an NK cell-specific CFP comprises a nucleic acid sequence encoding a CD8A hinge domain and a CD16A domain, and is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 4)
ACTACTACTCCAGCTCCAAGGCCTCCCACGCCAGCTCCCACTATTGCTTC
TCAACCGTTGTCACTGCGACCAGAGGCCTGTAGACCTGCAGCTGGAGGCG
CTGTTCACACAAGGGGTCTCGATTTTGCGTGTGACGGATATCAGGTTTCC
TTTTGTTTGGTCATGGTACTTCTCTTTGCGGTAGACACTGGTCTCTATTT
TAGTGTCAAAACTAATATACGCTCCTCCACGAGGGATTGGAAGGACCATA
AGTTCAAATGGAGGAAGGACCCGCAGGACAAATGA
(wherein the underlined sequence is CD16A domain
encoding sequence).

In some embodiments, a recombinant polynucleic acid comprising a sequence encoding an NK cell-specific CFP comprises a nucleic acid sequence encoding a mutated CD8A hinge domain. In some embodiments the mutated CD8A sequence comprises CS mutated sequence (CD8ACS mut). In some embodiments, the sequence encoding a mutated CD8A (CD8ACS mut) hinge domain is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 5)
TCTGGTCAAGTTCTGCTGGAGTCAAACATTAAAGTGCTGCCTACTTGGAG
CACTCCTGTTCAGCCTGGATATCAGGTTTCCTTTTGTTTGGTCATGGTAC
TTCTCTTTGCGGTAGACACTGGTCTCTATTTTAGTGTCAAAACTAATATA
CGCTCCTCCACGAGGGATTGGAAGGACCATAAGTTCAAATGGAGGAAGGA
CCCGCAGGACAAATGA,
(wherein the underlined sequence is CD16A domain).

In some embodiments, a recombinant polynucleic acid comprising a sequence encoding an NK cell-specific CFP comprises a nucleic acid sequence encoding a Siglec 4 hinge domain. In some embodiments, the sequence encoding a Siglec 4 hinge domain is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 6)
TATCCACCTGTCATAGTTGAAATGAATTCCAGTGTTGAGGCTATCGAGGG
CAGTCACGTATCACTCCTGTGTGGTGCAGATTCCAATCCACCACCCCTCC
TTACATGGATGCGGGATGGAACTGTTCTGAGAGAAGCGGTGGCGGAAAGT
TTGCTCCTTGAATTGGAGGAGGTTACTCCCGCCGAGGACGGCGTTTATGC
CTGCCTGGCCGAGAATGCGTACGGACAAGACAATCGAACGGTCGGTTTGA
GCGTGATGTACGCGCCTTGGAAACCTACGGTTAACGGCACTATGGTTGCG
GTAGAAGGGGAAACGGTATCCATACTCTGTAGTACACAATCAAATCCTGA
TCCCATCCTCACGATCTTTAAAGAGAAACAAATCCTTTCCACAGTCATTT
ATGAGTCTGAGCTTCAGCTCGAACTGCCAGCAGTCTCCCCTGAGGATGAT
GGAGAATATTGGTGCGTTGCCGAAAACCAGTATGGCCAGAGAGCTACAGC
GTTCAATCTCAGCGTAGAATTTGCTCCAGTTCTCTTGCTGGAGAGTCACT
GTGCGGCGGCACGGGATACTGTCCAGTGTCTTTGTGTAGTGAAAAGCAAT
CCTGAGCCTTCTGTAGCTTTTGAGTTGCCTTCACGCAACGTGACGGTAAA
TGAGAGCGAACGCGAGTTCGTGTATAGTGAGAGAAGCGGATTGGTGCTGA
CTTCAATCCTCACGCTTCGGGGCCAGGCGCAGGCGCCACCTCGCGTGATT
TGCACTGCTCGGAACCTTTACGGCGCAAAATCCTTGGAGCTGCCGTTTCA
GGGAGCCCATCGGCTTATGTGGGCTAAGATTGGTCCTGTGGGGGCT.

In one embodiment, a recombinant polynucleic acid comprising a sequence encoding an NK cell-specific CFP comprises a nucleic acid sequence encoding a Siglec 4 hinge domain and a CD16A domain. The nucleic acid sequence encoding a Siglec 4 hinge domain and a CD16A domain is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 7)
TATCCACCTGTCATAGTTGAAATGAATTCCAGTGTTGAGGCTATCGAGGG
CAGTCACGTATCACTCCTGTGTGGTGCAGATTCCAATCCACCACCCCTCC
TTACATGGATGCGGGATGGAACTGTTCTGAGAGAAGCGGTGGCGGAAAGT
TTGCTCCTTGAATTGGAGGAGGTTACTCCCGCCGAGGACGGCGTTTATGC
CTGCCTGGCCGAGAATGCGTACGGACAAGACAATCGAACGGTCGGTTTGA
GCGTGATGTACGCGCCTTGGAAACCTACGGTTAACGGCACTATGGTTGCG
GTAGAAGGGGAAACGGTATCCATACTCTGTAGTACACAATCAAATCCTGA
TCCCATCCTCACGATCTTTAAAGAGAAACAAATCCTTTCCACAGTCATTT
ATGAGTCTGAGCTTCAGCTCGAACTGCCAGCAGTCTCCCCTGAGGATGAT
GGAGAATATTGGTGCGTTGCCGAAAACCAGTATGGCCAGAGAGCTACAGC
GTTCAATCTCAGCGTAGAATTTGCTCCAGTTCTCTTGCTGGAGAGTCACT
GTGCGGCGGCACGGGATACTGTCCAGTGTCTTTGTGTAGTGAAAAGCAAT
CCTGAGCCTTCTGTAGCTTTTGAGTTGCCTTCACGCAACGTGACGGTAAA
TGAGAGCGAACGCGAGTTCGTGTATAGTGAGAGAAGCGGATTGGTGCTGA
CTTCAATCCTCACGCTTCGGGGCCAGGCGCAGGCGCCACCTCGCGTGATT
TGCACTGCTCGGAACCTTTACGGCGCAAAATCCTTGGAGCTGCCGTTTCA
GGGAGCCCATCGGCTTATGTGGGCTAAGATTGGTCCTGTGGGGGCTGGAT
ATCAGGTTTCCTTTTGTTTGGTCATGGTACTTCTCTTTGCGGTAGACACT
GGTCTCTATTTTAGTGTCAAAACTAATATACGCTCCTCCACGAGGGATTG
GAAGGACCATAAGTTCAAATGGAGGAAGGACCCGCAGGACAAATGA,
(wherein the underlined sequence is CD16A
encoding domain).

In some embodiments, the extracellular scFv is at the N-terminal end. In some embodiments, the extracellular scFv is at the C-terminal end.

In some embodiments, the NK cell-specific CFP comprises a cytoplasmic, transmembrane and extracellular regions of NKG2C in the order mentioned. In one embodiment, a recombinant polynucleic acid comprising a sequence encoding an NK cell-specific CFP comprises a nucleic acid sequence encoding a NKG2C cytoplasmic domain, TM and extracellular domain. In some embodiments, the sequence encoding a NKG2C cytoplasmic domain, TM and extracellular domain is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence: ATGAATAAGCAGCGAGGGACCTTTTCAGAAGTCTCACTCGCTCAGGATCCTAAAAGACA ACAAAGAAAGCCTAAGGGAAATAAGTCCAGCATATCAGGTACGGAACAGGAAATTTTT CAAGTCGAACTGAATTTGCAGAACCCTAGCCTGAATCACCAAGGTATCGACAAGATCTA TGATTGTCAAGGGCTCCTGCCACCGCCTGAAAAGCTTACGGCGGAGGTGCTGGGCATTA TTTGTATAGTCCTGATGGCAACTGTACTTAAAACTATTGTACTTATCCCGTTTCTGGAAC AAAACAATTTTTCTCCGAACACTCGGACACAAAAGGCCCGACATTGTGGTCACTGTCCA GAAGAGTGGATAACTTACTCTAATAGCTGTTACTATATCGGAAAAGAGAGGAGAACGT GGGAAGAAAGCTTGCTCGCATGCACTTCCAAAAACTCTTCACTCTTGTCCATTGATAAC GAGGAGGAGATGAAATTTCTGGCCTCAATCCTGCCATCATCTTGGATAGGCGTATTCCG CAACTCAAGTCATCACCCTTGGGTAACTATAAATGGTTTGGCGTTCAAGCACAAGATTA AAGACTCTGATAATGCCGAGTTGAACTGCGCTGTTCTTCAGGTGAACCGCCTCAAATCT GCCCAGTGCGGAAGTTCTATGATATATCACTGCAAACATAAACTG. (SEQ ID NO: 8) In this sequence, the scFv may be C terminal to the TM domain and the cytoplasmic domain may be at the N terminus.

In one embodiment, a recombinant polynucleic acid comprising a sequence encoding an NK cell-specific CFP comprises a nucleic acid sequence encoding a NKG2C cytoplasmic domain, and TM domains. In some embodiments, the sequence encoding a NKG2C cytoplasmic domain, and TM domains is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 9)
ATGGGGTGGATACGAGGCAGGAGGTCTCGGCACAGCTGGGAGATGTCAGA
GTTTCACAACTACAACCTCGACCTGAAAAAATCCGACTTCTCTACCCGAT
GGCAAAAGCAGCGATGTCCGGTAGTGAAGTCAAAATGTCGGGAAAACGCA
TCTCCGTTTTTTTTTTGCTGCTTCATAGCCGTCGCGATGGGCATAAGATT
CATCATTATGGTGACT.

In some embodiments, the NK cell-specific CFP comprises a cytoplasmic, TM and extracellular regions of NKp30. In one embodiment, a recombinant polynucleic acid comprising a sequence encoding an NK cell-specific CFP comprises a nucleic acid sequence encoding a NKp30 cytoplasmic domain, TM and extracellular domain. In some embodiments, the sequence encoding a NKp30 cytoplasmic domain, TM and extracellular domain is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 10)
CTGTGGGTTAGCCAGCCACCCGAGATTCGAACCCTCGAAGGCAGTAGTGC
TTTCTTGCCGTGTAGCTTCAATGCGTCCCAAGGGCGACTGGCAATCGGTT
CAGTCACATGGTTTCGCGACGAAGTTGTACCTGGCAAGGAGGTCAGGAAT
GGGACACCGGAATTTCGCGGCCGACTGGCCCCGTTGGCATCTTCCCGATT
TCTTCATGATCACCAGGCAGAGCTTCACATTCGCGACGTACGAGGACACG
ACGCAAGCATCTATGTATGTAGAGTTGAAGTTTTGGGACTTGGAGTAGGC
ACAGGGAATGGGACTAGGTTGGTAGTGGAAAAGGAGCATCCCCAGTTGGG
CGCAGGTACCGTACTTCTTCTGCGGGCAGGGTTCTATGCTGTCAGCTTTC
TGTCTGTGGCAGTTGGGTCCACAGTCTATTACCAGGGTAAGTGTCTCACG
TGGAAGGGACCACGGCGGCAATTGCCTGCGGTTGTTCCCGCACCCCTCCC
TCCTCCATGCGGTTCAAGTGCACATCTCCTTCCGCCAGTTCCAGGCGGCT
GA.

In one embodiment, a recombinant polynucleic acid comprising a sequence encoding an NK cell-specific CFP comprises a nucleic acid sequence encoding NKp30 TM and cytoplasmic domains. In some embodiments, the sequence encoding NKp30 TM and cytoplasmic domains is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 11)
GCAGGTACCGTACTTCTTCTGCGGGCAGGGTTCTATGCTGTCAGCTTTCT
GTCTGTGGCAGTTGGGTCCACAGTCTATTACCAGGGTAAGTGTCTCACGT
GGAAGGGACCACGGCGGCAATTGCCTGCGGTTGTTCCCGCACCCCTCCCT
CCTCCATGCGGTTCAAGTGCACATCTCCTTCCGCCAGTTCCAGGCGGCTG
A.

In some embodiments, a recombinant polynucleic acid comprising a sequence encoding an NK cell-specific CFP comprises a nucleic acid sequence encoding NKp30 (short extracellular (15aa), TM, cytoplasmic), the nucleic acid sequence is at least 80%, 81%, 82%, 83%, 84%, 85%, 86%, 87%, 88%, 89%, 90%, 91%, 92%, 93%, 94%, 95%, 96%° 97%/98%, 99% or 100% identical to a sequence:

(SEQ ID NO: 12)
AATGGGACTAGGTTGGTAGTGGAAAAGGAGCATCCCCAGTTGGGCGCAGG
TACCGTACTTCTTCTGCGGGCAGGGTTCTATGCTGTCAGCTTTCTGTCTG
TGGCAGTTGGGTCCACAGTCTATTACCAGGGTAAGTGTCTCACGTGGAAG
GGACCACGGCGGCAATTGCCTGCGGTTGTTCCCGCACCCCTCCCTCCTCC
ATGCGGTTCAAGTGCACATCTCCTTCCGCCAGTTCCAGGCGGCTGA.

In some embodiments, the NK cell-specific CFP comprises a extracellular, TM and cytoplasmic regions of NKp44, in the order mentioned. In one embodiment, a recombinant polynucleic acid comprising a sequence encoding an NK cell-specific CFP comprises a nucleic acid sequence encoding a NKp44 extracellular, TM and cytoplasmic. In some embodiments, the sequence encoding aNKp44 extracellular, TM and cytoplasmic domain is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 13)
CAGTCAAAAGCACAAGTTCTGCAGAGTGTAGCAGGCCAGACACTTACGGT
TAGGTGTCAATATCCGCCTACCGGCAGTCTGTATGAGAAAAAGGGCTGGT
GTAAAGAAGCGTCCGCGTTGGTTTGCATTCGGCTCGTAACGAGCTCCAAG
CCTCGGACTATGGCATGGACATCACGGTTCACTATATGGGACGACCCTGA
TGCAGGATTTTTTACTGTCACCATGACGGACCTTCGGGAGGAGGATTCCG
GCCACTATTGGTGTAGAATCTATAGACCGTCAGACAATTCTGTCAGCAAG
AGCGTGCGCTTCTACCTTGTGGTCTCTCCGGCTTCCGCCTCCACCCAAAC
ATCTTGGACTCCGAGGGATCTCGTGTCATCACAAACCCAGACACAGAGTT
GTGTGCCGCCCACGGCGGGAGCAAGACAGGCTCCGGAGAGCCCATCTACA
ATTCCGGTCCCTAGCCAACCACAGAACTCTACCTTGAGGCCCGGACCCGC
TGCACCCATCGCTTTGGTTCCAGTGTTTTGCGGACTCCTTGTTGCCAAGT
CACTTGTCCTTTCTGCTCTCCTGGTATGGTGGGGCGACATTTGGTGGAAA
ACGATGATGGAGCTTCGATCCTTGGACACACAGAAGGCGACATGTCATCT
CCAACAGGTGACAGACCTGCCATGGACTAGTGTGTCAAGTCCCGTCGAGC
GCGAAATCCTTTATCATACCGTGGCCCGAACCAAAATAAGCGACGATGAC
GATGAGCACACTCTGTGA.

In one embodiment, a recombinant polynucleic acid comprising a sequence encoding an NK cell-specific CFP comprises a nucleic acid sequence encoding NKp44 TM and cytoplasmic domains. In some embodiments, the sequence encoding NKp44 TM and cytoplasmic domains is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 14)
GCAGGTACCGTACTTCTTCTGCGGGCAGGGTTCTATGCTGTCAGCTTTCT
GTCTGTGGCAGTTGGGTCCACAGTCTATTACCAGGGTAAGTGTCTCACGT
GGAAGGGACCACGGCGGCAATTGCCTGCGGTTGTTCCCGCACCCCTCCCT
CCTCCATGCGGTTCAAGTGCACATCTCCTTCCGCCAGTTCCAGGCGGCTG
A.

In some embodiments, the NK cell-specific CFP comprises a short (19aa) extracellular domain, TM and cytoplasmic regions of NKp44, having a sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 15)
GTCCCTAGCCAACCACAGAACTCTACCTTGAGGCCCGGACCCGCTGCACC
CATCGCTTTGGTTCCAGTGTTTTGCGGACTCCTTGTTGCCAAGTCACTTG
TCCTTTCTGCTCTCCTGGTATGGTGGGGCGACATTTGGTGGAAAACGATG
ATGGAGCTTCGATCCTTGGACACACAGAAGGCGACATGTCATCTCCAACA
GGTGACAGACCTGCCATGGACTAGTGTGTCAAGTCCCGTCGAGCGCGAAA
TCCTTTATCATACCGTGGCCCGAACCAAAATAAGCGACGATGACGATGAG
CACACTCTGTGA.

In some embodiments, the NK cell-specific CFP comprises a extracellular, TM and cytoplasmic regions of NKp46, in the order mentioned. In one embodiment, a recombinant polynucleic acid comprising a sequence encoding an NK cell-specific CFP comprises a nucleic acid sequence encoding a NKp46 extracellular, TM and cytoplasmic. In some embodiments, the sequence encoding a NKp46 extracellular, TM and cytoplasmic domain is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 16)
CAGCAACAGACGCTGCCCAAACCATTTATTTGGGCTGAACCTCACTTTAT
GGTCCCCAAGGAAAAACAAGTTACAATCTGTTGCCAGGGGAATTACGGAG
CTGTGGAGTACCAGCTGCATTTCGAGGGATCTCTTTTTGCGGTTGATAGG
CCCAAGCCGCCCGAGCGGATCAACAAGGTGAAGTTTTACATACCAGATAT
GAACTCCAGGATGGCGGGACAGTACTCCTGTATCTATCGCGTGGGCGAGC
TGTGGAGTGAGCCTTCCAATCTGCTTGATCTTGTCGTCACCGAGATGTAC
GATACACCTACGCTGAGCGTTCATCCCGGGCCGGAAGTTATTAGTGGCGA
AAAGGTCACTTTCTATTGCCGCCTTGACACGGCTACGTCAATGTTCCTCT
TGCTGAAAGAAGGAAGATCTTCCCATGTCCAACGAGGATACGGAAAAGTC
CAAGCGGAATTTCCCCTGGGACCAGTAACTACCGCTCATAGGGGAACATA
CCGATGCTTCGGCAGCTACAACAACCACGCTTGGAGTTTTCCGTCTGAGC
CTGTAAAATTGCTCGTTACCGGAGACATTGAGAACACGAGCCTTGCCCCT
GAAGATCCGACGTTCCCAGCAGATACATGGGGGACTTATCTGTTGACTAC
GGAGACAGGACTTCAAAAGGACCATGCGTTGTGGGATCATACAGCTCAGA
ACTTGCTCCGCATGGGCCTGGCCTTTCTTGTACTCGTTGCACTCGTTTGG
TTCCTTGTTGAGGATTGGCTCTCTAGAAAGAGAACTAGAGAACGGGCCTC
CAGGGCATCCACGTGGGAAGGCCGCAGACGACTCAATACCCAGACCCTGT
GA.

In one embodiment, a recombinant polynucleic acid comprising a sequence encoding an NK cell-specific CFP comprises a nucleic acid sequence encoding NKp46 TM and cytoplasmic domains. In some embodiments, the sequence encoding NKp46 TM and cytoplasmic domains is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 17)
ATGGGCCTGGCCTTTCTTGTACTCGTTGCACTCGTTTGGTTCCTTGTTGA
GGATTGGCTCTCTAGAAAGAGAACTAGAGAACGGGCCTCCAGGGCATCCA
CGTGGGAAGGCCGCAGACGACTCAATACCCAGACCCTGTGA.

In some embodiments, the NK cell-specific CFP comprises a short (18aa) extracellular domain, TM and cytoplasmic regions of NKp46, having a sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence:

(SEQ ID NO: 18)
GGACTTCAAAAGGACCATGCGTTGTGGGATCATACAGCTCAGAACTTGCT
CCGCATGGGCCTGGCCTTTCTTGTACTCGTTGCACTCGTTTGGTTCCTTG
TTGAGGATTGGCTCTCTAGAAAGAGAACTAGAGAACGGGCCTCCAGGGCA
TCCACGTGGGAAGGCCGCAGACGACTCAATACCCAGACCCTGTGA.

In some embodiments, provided herein is an experimental CFP sequence comprising a sequence from NKG2D in reverse orientation-extracellular, TM, cytoplasmic, the sequence having at least 80% sequence identity to:

(SEQ ID NO: 19)
TGCTACAGCGAGACCCTGCCCATCCAGGTGGAGCAGAACTTCCTGAGCAA
CCTGTTCGTGGCCAGCTGGATCACCGTGATGATCATCTTCAGGATCGGCA
TGGCCGTGGCCATCTTCTGCTGCTTCTTCTTCCCCAGCGCCAACGAGAGG
TGCAAGAGCAAGGTGGTGCCCTGCAGGCAGAAGCAGTGGAGGACCAGCTT
CGACAGCAAGAAGCTGGACCTGAACTACAACCACTTCGAGAGCATGGAGT
GGAGCCACAGGAGCAGGAGGGGCAGGATCTGGGGCATGTGA.

In some embodiments, the CFP further comprises an intracellular domain. In some embodiments, the intracellular domain comprises an intracellular signaling domain from FcγR, FcαR, FcεR, CD40 or CD3ζ. In some embodiments, the intracellular domains further comprises a phosphoinositide 3-kinase (PI3K) recruitment domain. In some embodiments, the PI3K recruitment domain comprises a sequence with at least 90% sequence identity to YEDMRGILYAAPQLRSIRGQPGPNHEEDADSYENM (SEQ ID NO: 41). In some embodiments, the intracellular domain comprises an intracellular domain from CD39, CD56, CD57, CD94, CD159a, CD159c, CD314, CD335, CD336, CD337, DAP12, DAP10, NKG2C, NKG2D, NKG2E, Ly49D, Ly49D, NKp46, NKp30 or NKp44. In some embodiments, the intracellular domain comprises an intracellular domain from CD94, CD159a, CD159c, CD314, CD335, CD336, CD337, DAP12, or DAP10.

TABLE 2
Exemplary intracellular domains may be considered from any one of the domains.

| Sequence | CFP/ Domain |
|---|---|
| FcRγ-chain | LYCRRLKIQVRKAAITSYEKSDGVYTGLSTRNQETYETLKHEKP |
| intracellular signaling | PQ (SEQ ID NO: 243) |
| domain | |
| FcRγ-chain | LYCRLKIQVRKAAITSYEKSDGVYTGLSTRNQETYETLKHEKPP |
| intracellular signaling | Q (SEQ ID NO: 244) |
| domain | |
| FcRγ-chain | RLKIQVRKAAITSYEKSDGVYTGLSTRNQETYETLKHEKPPQ |
| intracellular signaling | (SEQ ID NO: 245) |
| domain | |
| FcRγ-chain | RRLKIQVRKAAITSYEKSDGVYTGLSTRNQETYETLKHEKPPQ |
| intracellular signaling | (SEQ ID NO: 246) |
| domain | |
| PI3K recruitment | YEDMRGILYAAPQLRSIRGQPGPNHEEDADSYENM (SEQ ID NO: |
| domain | 41) |
| CD40 intracellular | KKVAKKPTNKAPHPKQEPQEINFPDDLPGSNTAAPVQETLHGC |
| domain | QPVTQEDGKESRISVQERQ (SEQ ID NO: 247) |
| TNFR1 intracellular | QRWKSKLYSIVCGKSTPEKEGELEGTTTKPLAPNPSFSPTPGFTP |
| domain | TLGFSPVPSSTFTSSSTYTPGDCPNFAAPRREVAPPYQGADPILA |
| | TALASDPIPNPLQKWEDSAHKPQSLDTDDPATLYAVVENVPPLR |
| | WKEFVRRLGLSDHEIDRLELQNGRCLREAQYSMLATWRRRTPR |
| | REATLELLGRVLRDMDLLGCLEDIEEALCGPAALPPAPSLLR |
| | (SEQ ID NO: 248) |
| TNFR2 intracellular | PLCLQREAKVPHLPADKARGTQGPEQQHLLITAPSSSSSSLESSA |
| domain | SALDRRAPTRNQPQAPGVEASGAGEARASTGSSDSSPGGHGTQ |
| | VNVTCIVNVCSSSDHSSQCSSQASSTMGDTDSSPSESPKDEQVPF |
| | SKEECAFRSQLETPETLLGSTEEKPLPLGVPDAGMKPS (SEQ ID |
| | NO: 249) |
| CLEC9A (1-35) | MHEEEIYTSLQWDSPAPDTYQKCLSSNKCSGACCL (SEQ ID NO: |
| intracellular signaling | 250) |
| domain | |

The inhibitory receptors expressed on a NK cell include among others, KIR-L, that can bind to HLA A, or HLA-B, or HLA-c; LAIR-i that can bind collagen; SIGLEC 3, 7, 9 that can bind to sialic acid, CD94-NKG2A that can bind to HLA-E; KLRG1, that can bind to cadherins, NKR-P1A that can bind to LLT-1.

In one embodiment, specific recombinant molecules may be designed, and generated that can inhibit or block NK cell deactivation upon engagement of the receptors described above in this paragraph to their respective ligands.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the CFP functionally incorporates into a cell membrane of a cell when the CFP is expressed in the cell. In some embodiments, upon binding of the CFP to the antigen of the target cell, the killing activity of a cell expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold,-fold, 17-fold, 18-fold, 19-fold, 20-fold, 25-fold, 30-fold, 40-fold, 50-fold, 75-fold, or 100-fold compared to a cell not expressing the CFP.

In some embodiments, the target cell expressing the antigen is a cancer cell. In some embodiments, the target cell expressing the antigen is at least 0.8 microns in diameter.

In some embodiments, a cell expressing the CFP exhibits an increase in lysis of a target cell expressing the antigen compared to a cell not expressing the CFP when tested in vitro. In some embodiments, a cell expressing the CFP exhibits at least a 1.1-fold increase in phagocytosis of a target cell expressing the antigen compared to a cell not expressing the CFP. In some embodiments, a cell expressing the CFP exhibits at least a 2-fold, 3-fold, 4-fold, 5-fold, 6-fold, 7-fold, 8-fold, 9-fold, 10-fold, 20-fold, 30-fold or 50-fold increase in phagocytosis of a target cell expressing the antigen compared to a cell not expressing the CFP. In some embodiments, a cell expressing the CFP exhibits an increase in production of a cytokine compared to a cell not expressing the CFP. In some embodiments, the cytokine is selected from the group consisting of IL-1, IL3, IL-6, IL-12, IL-13, IL-23, TNF, CCL2, CXCL9, CXCL10, CXCL11, IL-18, IL-23, IL-27, CSF, MCSF, GMCSF, IL17, IP-10, RANTES, an interferon and combinations thereof. In some embodiments, a cell expressing the CFP exhibits an increase in effector activity compared to a cell not expressing the CFP.

In some embodiments, the transmembrane domain oligomerizes with a transmembrane domain of an endogenous NK cell receptor when the CFP is expressed in a cell. In some embodiments, the transmembrane domain dimerizes with a transmembrane domain of an endogenous receptor when the CFP is expressed in a cell. In some embodiments, the transmembrane domain is derived from a protein that is different than the protein from which the intracellular signaling domain is derived. In some embodiments, the transmembrane domain is derived from a protein that is different than the protein from which the extracellular domain is derived. In some embodiments, the transmembrane domain comprises a transmembrane domain of a phagocytic receptor. In some embodiments, the transmembrane domain and the extracellular domain are derived from the same protein. In some embodiments, the transmembrane domain is derived from the same protein as the intracellular signaling domain. In some embodiments, the recombinant polynucleic acid encodes a DAP12 recruitment domain. In some embodiments, the transmembrane domain comprises a transmembrane domain that oligomerizes with DAP12.

In some embodiments, the transmembrane domain is at least 12, 13, 14, 15, 16, 17, 18, 19, 20, 21, 22, 23, 24, 25, 26, 27, 28, 29, 30, 31 or 32 amino acids in length. In some embodiments, the transmembrane domain is at most 12, 13, 14, 15, 16, 17, 18, 19, 20, 21, 22, 23, 24, 25, 26, 27, 28, 29, 30, 31 or 32 amino acids in length.

In some embodiments, the intracellular signaling domain comprises an intracellular signaling domain derived from a phagocytic receptor. In some embodiments, the intracellular signaling domain comprises an intracellular signaling domain derived from a phagocytic receptor selected from the group consisting of TNFR1, MDA5, CD40, lectin, dectin 1, CD206, scavenger receptor Al(SRA1), MARCO, CD3δ, CD163, MSR1, SCARA3, COLEC12, SCARA5, SCARBI, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fc-alpha receptor I, CR1, CD35, CD3ζ, CR3, CR4, Tim-1, Tim-4 and CD169. In some embodiments, the intracellular signaling domain comprises a PI3K recruitment domain. In some embodiments, the intracellular domain comprises a phosphatase inhibition domain. In some embodiments, the intracellular domain comprises an ARP2/3 inhibition domain. In some embodiments, the intracellular domain comprises at least one ITAM domain. In some embodiments, the intracellular domain comprises at least 1, 2, 3, 4, 5, 6, 7, 8, 9, 10, or more ITAM domains. In some embodiments, the intracellular domain comprises at least one ITAM domain select from an ITAM domain of CD3 zeta, CD3 epsilon, CD3 gamma, CD3 delta, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the at least one ITAM domain comprises a Src-family kinase phosphorylation site. In some embodiments, the at least one ITAM domain comprises a Syk recruitment domain. In some embodiments, the intracellular domain comprises an F-actin depolymerization activation domain. In some embodiments, the intracellular domain lacks enzymatic activity.

In some embodiments, the intracellular domain comprises a pro-inflammatory signaling domain. In some embodiments, the pro-inflammatory signaling domain comprises a kinase activation domain or a kinase binding domain. In some embodiments, the pro-inflammatory signaling domain comprises an IL-1 signaling cascade activation domain. In some embodiments, the pro-inflammatory signaling domain comprises an intracellular signaling domain derived from TLR3, TLR4, TLR7, TLR 9, TRIF, RIG-1, MYD88, MAL, IRAK1, MDA-5, an IFN-receptor, STING, anNLRP family member, NLRP1-14, NOD1, NOD2, Pyrin, AIM2, NLRC4, FCGR3A, FCERIG, CD40, Tank I-binding kinase (TBK), a caspase domain, a procaspase binding domain or any combination thereof.

In some embodiments, the intracellular domain comprises a signaling domain, such as an intracellular signaling domain, derived from a connexin (Cx) protein. For example, the intracellular domain can comprise a signaling domain, such as an intracellular signaling domain, derived from Cx43, Cx46, Cx37, Cx40, Cx33, Cx50, Cx59, Cx62, Cx32, Cx26, Cx31, Cx30.3, Cx31.1, Cx30, Cx25, Cx45, Cx47, Cx31.3, Cx36, Cx31.9, Cx39, Cx40.1 or Cx23. For example, the intracellular domain can comprise a signaling domain, such as an intracellular signaling domain, derived from Cx43.

In some embodiments, the intracellular domain comprises a signaling domain, such as an intracellular signaling domain, derived from a SIGLEC protein. For example, the intracellular domain can comprise a signaling domain, such as an intracellular signaling domain, derived from Siglec-1 (Sialoadhesin), Siglec-2 (CD22), Siglec-3 (CD33), Siglec-4 (MAG), Siglec-5, Siglec-6, Siglec-7, Siglec-8, Siglec-9, Siglec-10, Siglec-11, Siglec-12, Siglec-13, Siglec-14, Siglec-15, Siglec-16 or Siglec-17.

In some embodiments, the intracellular domain comprises a signaling domain, such as an intracellular signaling domain, derived from a C-type lectin protein. For example, the intracellular domain can comprise a signaling domain, such as an intracellular signaling domain, derived from a mannose receptor protein. For example, the intracellular domain can comprise a signaling domain, such as an intracellular signaling domain, derived from an asialoglycoprotein receptor protein. For example, the intracellular domain can comprise a signaling domain, such as an intracellular signaling domain, derived from macrophage galactose-type lectin (MGL), DC-SIGN (CLEC4L), Langerin (CLEC4K), Myeloid DAP12 associating lectin (MDL)-1 (CLEC5A), a DC associated C type lectin 1 (Dectin1) subfamily protein, dectin 1/CLEC7A, DNGR1/CLEC9A, Myeloid C type lectin like receptor (MICL) (CLEC12A), CLEC2 (CLECIB), CLEC12B, a DC immunoreceptor (DCIR) subfamily protein, DCIR/CLEC4A, Dectin 2/CLEC6A, Blood DC antigen 2 (BDCA2) (CLEC4C), Mincle (macrophage inducible C type lectin) (CLEC4E), a NOD-like receptor protein, NOD-like receptor MHC Class II transactivator (CIITA), IPAF, BIRC1, a RIG-I-like receptor (RLR) protein, RIG-I, MDA5, LGP2, NAIP5/Birc le, an NLRP protein, NLRP1, NLRP2, NLRP3, NLRP4, NLRP5, NLRP6, NLRP7, NLRP89, NLRP9, NLRP10, NLRP11, NLRP12, NLRP13, NLRP14, an NLR protein, NOD1 or NOD2, or any combination thereof.

In some embodiments, the intracellular domain comprises a signaling domain, such as an intracellular signaling domain, derived from a cell adhesion molecule. For example, the intracellular domain can comprise a signaling domain, such as an intracellular signaling domain, derived from an IgCAMs, a cadherin, an integrin, a C-type of lectin-like domains protein (CTLD) and/or a proteoglycan molecule. For example, the intracellular domain can comprise a signaling domain, such as an intracellular signaling domain, derived from an E-cadherin, a P-cadherin, an N-cadherin, an R-cadherin, a B-cadherin, a T-cadherin, or an M-cadherin. For example, the intracellular domain can comprise a signaling domain, such as an intracellular signaling domain, derived from a selectin, such as an E-selectin, an L-selectin or a P-selectin.

In some embodiments, the CFP does not comprise a full length intracellular signaling domain. In some embodiments, the intracellular domain is at least 5, 10, 20, 30, 40, 50, 60, 70, 80, 90, 100, 150, 200, 300, 300, 400, or 500 amino acids in length. In some embodiments, the intracellular domain is at most 10, 20, 30, 40, 50, 60, 70, 80, 90, 100, 150, 200, 300, 300, 400, or 500 amino acids in length.

In some embodiments, the recombinant polynucleic acid encodes an FcRα chain extracellular domain, an FcRα chain transmembrane domain and/or an FcRα chain intracellular domain. In some embodiments, the recombinant polynucleic acid encodes an FcRβ chain extracellular domain, an FcRβ chain transmembrane domain and/or an FcRβ chain intracellular domain. In some embodiments, the FcRα chain or the FcRβ chain forms a complex with FcRγ when expressed in a cell.

In some embodiments, the composition further comprises a pro-inflammatory polypeptide. In some embodiments, the pro-inflammatory polypeptide is a chemokine, cytokine. In some embodiments, the chemokine is selected from the group consisting of IL-1, IL3, IL5, IL-6, il8, IL-12, IL-13, IL-23, TNF, CCL2, CXCL9, CXCL10, CXCL11, IL-18, IL-23, IL-27, CSF, MCSF, GMCSF, IL 17, IP-10, RANTES, and interferon. In some embodiments, the cytokine is selected from the group consisting of IL-1, IL3, IL5, IL-6, IL-12, IL-13, IL-23, TNF, CCL2, CXCL9, CXCL10, CXCL11, IL-18, IL-23, IL-27, CSF, MCSF, GMCSF, IL17, IP-10, RANTES, and interferon.

In some embodiments, the NK cell-specific sequence comprises an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of the sequences in Table 3.

TABLE 3
Amino acid sequences of exemplary NK-specific CFP sequences

| | | SEQ |
|---|---|---|
| Name | Sequence | ID NO |
| TROP2- | MWLQSLLLLGTVACSISQVQLQQSGSELKKPGASVKVSCKASGYTFTNY | 20 |
| DAP12 | GMNWVKQAPGQGLKWMGWINTYTGEPTYTDDFKGRFAFSLDTSVSTAY | |
| | LQISSLKADDTAVYFCARGGFGSSYWYFDVWGQGSLVTVSSGGGGSGGG | |
| | GSGGGGSDIQLTQSPSSLSASVGDRVSITCKASQDVSIAVAWYQQKPGKA | |
| | PKLLIYSASYRYTGVPDRFSGSGSGTDFTLTISSLQPEDFAVYYCQQHYITP | |
| | LTFGAGTKVEIKRSGGGGAAAGSLRPVQAQAQSDCSCSTVSPGVLAGIVM | |
| | GDLVLTVLIALAVYFLGRLVPRGRGAAEAATRKQRITETESPYQELQGQR | |
| | SDVYSDLNTQRPYYK | |
| TROP2- | MWLQSLLLLGTVACSISQVQLQQSGSELKKPGASVKVSCKASGYTFTNY | 21 |
| CD16A | GMNWVKQAPGQGLKWMGWINTYTGEPTYTDDFKGRFAFSLDTSVSTAY | |
| | LQISSLKADDTAVYFCARGGFGSSYWYFDVWGQGSLVTVSSGGGGSGGG | |
| | GSGGGGSDIQLTQSPSSLSASVGDRVSITCKASQDVSIAVAWYQQKPGKA | |
| | PKLLIYSASYRYTGVPDRFSGSGSGTDFTLTISSLQPEDFAVYYCQQHYITP | |
| | LTFGAGTKVEIKRSGGGGAAAGSGLAVSTISSFFPPGYQVSFCLVMVLLFA | |
| | VDTGLYFSVKTNIRSSTRDWKDHKFKWRKDPQDK | |
| TROP2- | MWLQSLLLLGTVACSISQVQLQQSGSELKKPGASVKVSCKASGYTFTNY | 22 |
| CD8h- | GMNWVKQAPGQGLKWMGWINTYTGEPTYTDDFKGRFAFSLDTSVSTAY | |
| CD16A | LQISSLKADDTAVYFCARGGFGSSYWYFDVWGQGSLVTVSSGGGGSGGG | |
| | GSGGGGSDIQLTQSPSSLSASVGDRVSITCKASQDVSIAVAWYQQKPGKA | |
| | PKLLIYSASYRYTGVPDRFSGSGSGTDFTLTISSLQPEDFAVYYCQQHYITP | |
| | LTFGAGTKVEIKRSGGGGAAATTTPAPRPPTPAPTIASQPLSLRPEACRPAA | |
| | GGAVHTRGLDFACDGYQVSFCLVMVLLFAVDTGLYFSVKTNIRSSTRDW | |
| | KDHKFKWRKDPQDK | |
| TROP2- | MWLQSLLLLGTVACSISQVQLQQSGSELKKPGASVKVSCKASGYTFTNY | 23 |
| CD8hC | GMNWVKQAPGQGLKWMGWINTYTGEPTYTDDFKGRFAFSLDTSVSTAY | |
| Smut- | LQISSLKADDTAVYFCARGGFGSSYWYFDVWGQGSLVTVSSGGGGSGGG | |
| CD16A | GSGGGGSDIQLTQSPSSLSASVGDRVSITCKASQDVSIAVAWYQQKPGKA | |
| | PKLLIYSASYRYTGVPDRFSGSGSGTDFTLTISSLQPEDFAVYYCQQHYITP | |
| | LTFGAGTKVEIKRSGGGGAAATTTPAPRPPTPAPTIASQPLSLRPEASRPAA | |
| | GGAVHTRGLDFASDGYQVSFCLVMVLLFAVDTGLYFSVKTNIRSSTRDW | |
| | KDHKFKWRKDPQDK | |
| TROP2- | MWLQSLLLLGTVACSISQVQLQQSGSELKKPGASVKVSCKASGYTFTNY | 24 |
| CD4h- | GMNWVKQAPGQGLKWMGWINTYTGEPTYTDDFKGRFAFSLDTSVSTAY | |
| CD16A | LQISSLKADDTAVYFCARGGFGSSYWYFDVWGQGSLVTVSSGGGGSGGG | |
| | GSGGGGSDIQLTQSPSSLSASVGDRVSITCKASQDVSIAVAWYQQKPGKA | |
| | PKLLIYSASYRYTGVPDRFSGSGSGTDFTLTISSLQPEDFAVYYCQQHYITP | |
| | LTFGAGTKVEIKRSGGGGAAASGQVLLESNIKVLPTWSTPVQPGYQVSFC | |
| | LVMVLLFAVDTGLYFSVKTNIRSSTRDWKDHKFKWRKDPQDK | |
| TROP2- | MWLQSLLLLGTVACSISQVQLQQSGSELKKPGASVKVSCKASGYTFTNY | 25 |
| Siglec4h- | GMNWVKQAPGQGLKWMGWINTYTGEPTYTDDFKGRFAFSLDTSVSTAY | |
| CD16A | LQISSLKADDTAVYFCARGGFGSSYWYFDVWGQGSLVTVSSGGGGSGGG | |
| | GSGGGGSDIQLTQSPSSLSASVGDRVSITCKASQDVSIAVAWYQQKPGKA | |
| | PKLLIYSASYRYTGVPDRFSGSGSGTDFTLTISSLQPEDFAVYY | |
| | CQQHYITPLTFGAGTKVEIKRSGGGGAAAYPPVIVEMNSSVEAIEGSHVSL | |
| | LCGADSNPPPLLTWMRDGTVLREAVAESLLLELEEVTPAEDGVYACLAE | |
| | NAYGQDNRTVGLSVMYAPWKPTVNGTMVAVEGETVSILCSTQSNPDPIL | |
| | TIFKEKQILSTVIYESELQLELPAVSPEDDGEYWCVAENQYGQRATAFNLS | |
| | VEFAPVLLLESHCAAARDTVQCLCVVKSNPEPSVAFELPSRNVTVNESER | |
| | EFVYSERSGLVLTSILTLRGQAQAPPRVICTARNLYGAKSLELPFQGAHR | |
| | LMWAKIGPVGAGYQVSFCLVMVLLFAVDTGLYFSVKTNIRSSTRDWKDH | |
| | KFKWRKDPQDK | |
| HER2- | MWLQSLLLLGTVACSISDIQMTQSPSSLSASVGDRVTITCRASQDVNTAV | 26 |
| CD16A | AWYQQKPGKAPKLLIYSASFLYSGVPSRFSGSRSGTDFTLTISSLQPEDFAT | |
| | YYCQQHYTTPPTFGQGTKVEIKRTGSTSGSGKPGSGEGSEVQLVESGGGL | |
| | VQPGGSLRLSCAASGFNIKDTYIHWVRQAPGKGLEWVARIYPTNGYTRY | |
| | ADSVKGRFTISADTSKNTAYLQMNSLRAEDTAVYYCSRW | |
| | GGDGFYAMDYWGQGTLVTVSSSGGGGAAAGSGLAVSTISSFFPPGYQVS | |
| | FCLVMVLLFAVDTGLYFSVKTNIRSSTRDWKDHKFKWRKDPQDK | |
| HER2- | MNKQRGTFSEVSLAQDPKRQQRKPKGNKSSISGTEQEIFQVELNLQNPSL | 27 |
| NKG2C | NHQGIDKIYDCQGLLPPPEKLTAEVLGIICIVLMATVLKTIVLIPFLEQNNFS | |
| | PNTRTQKARHCGHCPEEWITYSNSCYYIGKERRTWEESLLACTSKNSSLLS | |
| | IDNEEEMKFLASILPSSWIGVFRNSSHHPWVTINGLAFKHKIKDSDNAELN | |
| | CAVLQVNRLKSAQCGSSMIYHCKHKLSGGGGAAAGSDIQMTQSPSSLSAS | |
| | VGDRVTITCRASQDVNTAVAWYQQKPGKAPKLLIYSASFLYSGVP | |
| | SRFSGSRSGTDFTLTISSLQPEDFATYYCQQHYTTPPTFGQGTKVEIKRTGS | |
| | TSGSGKPGSGEGSEVQLVESGGGLVQPGGSLRLSCAASGFNIKDTYIHWV | |
| | RQAPGKGLEWVARIYPTNGYTRYADSVKGRFTISADTSKNTAYLQMNSL | |
| | RAEDTAVYYCSRWGGDGFYAMDYWGQGTLVTVSS | |
| HER2- | MNKQRGTFSEVSLAQDPKRQQRKPKGNKSSISGTEQEIFQVELNLQNPSL | 28 |
| NKG2C | NHQGIDKIYDCQGLLPPPEKLTAEVLGIICIVLMATVLKTIVLSGGGGAAA | |
| TMCyto | GSDIQMTQSPSSLSASVGDRVTITCRASQDVNTAVAWYQQKPGKAPKLLI | |
| | YSASFLYSGVPSRFSGSRSGTDFTLTISSLQPEDFATYYCQQHYTTPPTFGQ | |
| | GTKVEIKRTGSTSGSGKPGSGEGSEVQLVESGGGLVQ | |
| | PGGSLRLSCAASGFNIKDTYIHWVRQAPGKGLEWVARIYPTNGYTRYADS | |
| | VKGRFTISADTSKNTAYLQMNSLRAEDTAVYYCSRWGGDGFYAMDYWG | |
| | QGTLVTVSS | |
| HER2- | MGWIRGRRSRHSWEMSEFHNYNLDLKKSDFSTRWQKQRCPVVKSKCRE | 29 |
| NKG2D | NASPFFFCCFIAVAMGIRFIIMVTIWSAVFLNSLFNQEVQIPLTESYCGPCPK | |
| | NWICYKNNCYQFFDESKNWYESQASCMSQNASLLKVYSKEDQDLLKLV | |
| | KSYHWMGLVHIPTNGSWQWEDGSILSPNLLTIIEMQKGDCALYASSFKGY | |
| | IENCSTPNTYICMQRTVSGGGGAAAGSDIQMTQSPSSLSASVGDRVTITCR | |
| | ASQDVNTAVAWYQQKPGKAPKLLIYSASFLYSGVPSRFSGSRSGTDFTLT | |
| | ISSLQPEDFATYYCQQHYTTPPTFGQGTKVEIKRTGSTSGSGKPGSGEGSE | |
| | VQLVESGGGLVQPGGSLRLSCAASGFNIKDTYIHWVRQAPGKGLEWVAR | |
| | IYPTNGYTRYADSVKGRFTISADTSKNTAYLQMNSLRAEDTAVYYCSRW | |
| | GGDGFYAMDYWGQGTLVTVSS | |
| HER2- | MGWIRGRRSRHSWEMSEFHNYNLDLKKSDFSTRWQKQRCPVVKSKCRE | 30 |
| NKG2 | NASPFFFCCFIAVAMGIRFIIMVTSGGGGAAAGSDIQMTQSPSSLSASVGDR | |
| DTMCyto | VTITCRASQDVNTAVAWYQQKPGKAPKLLIYSASFLYSGVPSRFSGSRSG | |
| | TDFTLTISSLQPEDFATYYCQQHYTTPPTFGQGTKVEIKRTGSTSGSGKPGS | |
| | GEGSEVQLVESGGGLVQPGGSLRLSCAASGFNIKDTYIHWVRQAPGKGLE | |
| | WVARIYPTNGYTRYADSVKGRFTISADTSKNTAYLQMNSLRAEDTAVY | |
| | YCSRWGGDGFYAMDYWGQGTLVTVSS | |
| HER2- | MWLQSLLLLGTVACSISDIQMTQSPSSLSASVGDRVTITCRASQDVNTAV | 31 |
| NKp30 | AWYQQKPGKAPKLLIYSASFLYSGVPSRFSGSRSGTDFTLTISSLQPEDFAT | |
| | YYCQQHYTTPPTFGQGTKVEIKRTGSTSGSGKPGSGEGSEVQLVESGGGL | |
| | VQPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYAMD | |
| | YWGQGTLVTVSSSGGGGAAAGSLWVSQPPEIRTLEGSSAFLPCSFNASQG | |
| | RLAIGSVTWFRDEVVPGKEVRNGTPEFRGRLAPLASSRFLHDHQAELHIR | |
| | DVRGHDASIYVCRVEVLGLGVGTGNGTRLVVEKEHPQLGAGTVLLLRAG | |
| | FYAVSFLSVAVGSTVYYQGKCLTWKGPRRQLPAVVPAPLPPPCGSSAHLL | |
| | PPVPGG | |
| HER2- | MWLQSLLLLGTVACSISDIQMTQSPSSLSASVGDRVTITCRASQDVNTAV | 32 |
| NKp30 | AWYQQKPGKAPKLLIYSASFLYSGVPSRFSGSRSGTDFTLTISSLQPEDFAT | |
| TMCyto | YYCQQHYTTPPTFGQGTKVEIKRTGSTSGSGKPGSGEGSEVQLVESGGGL | |
| | VQPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYAMD | |
| | YWGQGTLVTVSSSGGGGAAAGSAGTVLLLRAGFYAVSFLSVAVGSTVYY | |
| | QGKCLTWKGPRRQLPAVVPAPLPPPCGSSAHLLPPVPGG | |
| HER2- | MWLQSLLLLGTVACSISDIQMTQSPSSLSASVGDRVTITCRASQDVNTAV | 333 |
| NKp44 | AWYQQKPGKAPKLLIYSASFLYSGVPSRFSGSRSGTDFTLTISSLQPEDFAT | |
| | YYCQQHYTTPPTFGQGTKVEIKRTGSTSGSGKPGSGEGSEVQLVESGGGL | |
| | VQPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYAMD | |
| | YWGQGTLVTVSSSGGGGAAAGSQSKAQVLQSVAGQTLTVRCQYPPTGSL | |
| | YEKKGWCKEASALVCIRLVTSSKPRTMAWTSRFTIWDDPDAGFFTVTMT | |
| | DLREEDSGHYWCRIYRPSDNSVSKSVRFYLVVSPASASTQTSWTPRDLVS | |
| | SQTQTQSCVPPTAGARQAPESPSTIPVPSQPQNSTLRPGPAAPIALVPVFCG | |
| | LLVAKSLVLSALLVWWGDIWWKTMMELRSLDTQKATCHLQQVTDLPW | |
| | TSVSSPVEREILYHTVARTKISDDDDEHTL | |
| HER2- | MWLQSLLLLGTVACSISDIQMTQSPSSLSASVGDRVTITCRASQDVNTAV | 34 |
| NKp44 | AWYQQKPGKAPKLLIYSASFLYSGVPSRFSGSRSGTDFTLTISSLQPEDFAT | |
| TMCyto | YYCQQHYTTPPTFGQGTKVEIKRTGSTSGSGKPGSGEGSEVQLVESGGGL | |
| | VQPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYAMD | |
| | YWGQGTLVTVSSSGGGGAAAGSLVPVFCGLLVAKSLVLSALLVWWGDI | |
| | WWKTMMELRSLDTQKATCHLQQVTDLPWTSVSSPVEREILYHTVARTKI | |
| | SDDDDEHTL | |
| HER2- | MWLQSLLLLGTVACSISDIQMTQSPSSLSASVGDRVTITCRASQDVNTAV | 35 |
| NKp46 | AWYQQKPGKAPKLLIYSASFLYSGVPSRFSGSRSGTDFTLTISSLQPEDFAT | |
| | YYCQQHYTTPPTFGQGTKVEIKRTGSTSGSGKPGSGEGSEVQLVESGGGL | |
| | VQPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYAMD | |
| | YWGQGTLVTVSSSGGGGAAAGSQQQTLPKPFIWAEPHFMVPKEKQVTIC | |
| | CQGNYGAVEYQLHFEGSLFAVDRPKPPERINKVKFYIPDMNSRMAGQYS | |
| | CIYRVGELWSEPSNLLDLVVTEMYDTPTLSVHPGPEVISGEKVTFYCRLDT | |
| | ATSMFLLLKEGRSSHVQRGYGKVQAEFPLGPVTTAHRGTYRCFGSYNNH | |
| | AWSFPSEPVKLLVTGDIENTSLAPEDPTFPADTWGTYLLTTETGLQKDHA | |
| | LWDHTAQNLLRMGLAFLVLVALVWFLVEDWLSRKRTRERASRASTWEG | |
| | RRRLNTQTL | |
| HER2- | MWLQSLLLLGTVACSISDIQMTQSPSSLSASVGDRVTITCRASQDVNTAV | 36 |
| NKp46 | AWYQQKPGKAPKLLIYSASFLYSGVPSRFSGSRSGTDFTLTISSLQPEDFAT | |
| TMCyto | YYCQQHYTTPPTFGQGTKVEIKRTGSTSGSGKPGSGEGSEVQLVESGGGL | |
| | VQPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYAMD | |
| | YWGQGTLVTVSSSGGGGAAAGSMGLAFLVLVALVWFLVEDWLSRKRTR | |
| | ERASRASTWEGRRRLNTQTL | |
| HER2- | MWLQSLLLLGTVACSISDIQMTQSPSSLSASVGDRVTITCRASQDVNTAV | 37 |
| revNK | AWYQQKPGKAPKLLIYSASFLYSGVPSRFSGSRSGTDFTLTISSLQPEDFAT | |
| G2D | YYCQQHYTTPPTFGQGTKVEIKRTGSTSGSGKPGSGEGSEVQLVESGGGL | |
| | VQPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYAMD | |
| | YWGQGTLVTVSSSGGGGAAAGCYSETLPIQVEQNFLSNLFVASWITVMIIF | |
| | RIGMAVAIFCCFFFPSANERCKSKVVPCRQKQWRTSFDSKKLDLNYNHFE | |
| | SMEWSHRSRRGRIWGM | |
| HER2- | MWLQSLLLLGTVACSISDIQMTQSPSSLSASVGDRVTITCRASQDVNTAV | 38 |
| NKp30 | AWYQQKPGKAPKLLIYSASFLYSGVPSRFSGSRSGTDFTLTISSLQPEDFAT | |
| ectoTMCyto | YYCQQHYTTPPTFGQGTKVEIKRTGSTSGSGKPGSGEGSEVQLVESGGGL | |
| | VQPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYAMD | |
| | YWGQGTLVTVSSSGGGGAAAGNGTRLVVEKEHPQLGAGTVLLLRAGFY | |
| | AVSFLSVAVGSTVYYQGKCLTWKGPRRQLPAVVPAPLPPPCGSSAHLLPP | |
| | VPGG | |
| HER2- | MWLQSLLLLGTVACSISDIQMTQSPSSLSASVGDRVTITCRASQDVNTAV | 39 |
| NKp44 | AWYQQKPGKAPKLLIYSASFLYSGVPSRFSGSRSGTDFTLTISSLQPEDFAT | |
| ectoTMCyto | YYCQQHYTTPPTFGQGTKVEIKRTGSTSGSGKPGSGEGSEVQLVESGGGL | |
| | VQPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYAMD | |
| | YWGQGTLVTVSSSGGGGAAAGVPSQPQNSTLRPGPAAPIALVPVFCGLLV | |
| | AKSLVLSALLVWWGDIWWKTMMELRSLDTQKATCHLQQVTDLPWTSVS | |
| | SPVEREILYHTVARTKISDDDDEHTL | |
| HER2- | MWLQSLLLLGTVACSISDIQMTQSPSSLSASVGDRVTITCRASQDVNTAV | 40 |
| NKp46 | AWYQQKPGKAPKLLIYSASFLYSGVPSRFSGSRSGTDFTLTISSLQPEDFAT | |
| ectoTMCyto | YYCQQHYTTPPTFGQGTKVEIKRTGSTSGSGKPGSGEGSEVQLVESGGGL | |
| | VQPGGSLRLSCAASGFNIKDTYIHWVRQAPGKGLEWVARIYPTNGYTRY | |
| | ADSVKGRFTISADTSKNTAYLQMNSLRAEDTAVYYCSRWGGDGFYAMD | |
| | YWGQGTLVTVSSSGGGGAAAGGLQKDHALWDHTAQNLLRMGLAFLVL | |
| | VALVWFLVEDWLSRKRTRERASRASTWEGRRRLNTQTL | |

### T Cell-Specific Chimeric Fusion Protein (CFP) Designs

Provided herein is a recombinant polynucleic acid composition comprising a recombinant polynucleic acid sequence encoding a chimeric fusion protein (CFP) comprising a transmembrane domain that specifically integrates within a membrane protein complex of a T cell, wherein the T cell is characterized as naturally expressing the membrane protein complex; wherein, when the recombinant polynucleic acid composition comprising the recombinant polynucleic acid sequence is contacted to any cell of a heterogenous cell population, at least greater than 50% of cells within the heterogenous cell population that are characterized as naturally expressing the membrane protein complex, e.g., T cells, expresses the CFP, and a cell within the heterogenous cell population that lacks the membrane protein complex, e.g., non-T cells, fails to express the CFP. In some embodiments, the naturally expressing the membrane protein complex of a T cell may be the TCR complex. The TCR complex, T-cell receptor (TCR)-CD3 complex is composed of a diverse αβ TCR heterodimer noncovalently associated with the invariant CD3 dimers CD3ϵγ, CD3δ, and CD3ζζ. The TCR mediates recognition of antigenic peptides bound to MIIC molecules (pMHC), whereas the CD3 molecules transduce activation signals to the T cell, Therefore, a polypeptide designed for expression preferably in T cells is designed to have a component that may be operably linked with a member of the TCR complex. In some embodiments, the CFP comprises one or more sequences from a TCR, e g. CD3. In one embodiment, the T cell specific CFP comprises a CD3 epsilon (CD3ε) TM domain and intracellular domain. In some embodiments, it comprises an extracelhilar domain that comprises a sequence from the CD3ε, and an seFv that binds to a cancer antigen.

In some embodiments, the recombinant polynucleic acid composition is expressed in at least greater than 60%, 70%, 80% or 90% of T cells within the heterogenous cell population. In some embodiments, the CFR is expressed in at least 50% of T cells in a heterogenous population PBMC, e.g. obtained from peripheral blood drawn at 1, 2, or 3 days after introduction of the polynucleic acid in the system of the subject. In some embodiments, the recombinant polynucleic acid composition less than 10% cells within the heterogenous cell population that lacks a TCR complex express the CFP. In some embodiments, the CFP is expressed in less than 10% T cells in a population of cells in a biological sample from the subject at 1, 2, or 3 days after administration of a composition comprising the recombinant polynucleic acid. In some embodiments, the CFP is expressed in less than 10% of myeloid cells in a biological sample from the subject at 1, 2, or 3 days after administration of a composition comprising the recombinant polynucleic acid. In some embodiments, the CFP is expressed in less than 10% of epithelial cells in a biological sample from the subject at 1, 2, or 3 days after administration of a composition comprising the recombinant polynucleic acid. In some embodiments, the recombinant polynucleic acid is expressed at greater than 50% of T cells, e.g., greater than 60%, 70%, 80% or 90% of T cells within the heterogenous cell population tested ex vivo. In some embodiments, the recombinant polynucleic acid is expressed at less than 10% of T cells, e.g., less than 9%, 8%, 7%, 6%, 5%, 4%, 3%, 2% 1% or less in cells other than T cells, e.g. epithelial cells or myeloid cells within the heterogenous cell population tested ex vivo.

In some embodiments, the CFP for cell-specific expression in a T cell comprises an anti-CD19 scFv.

In some embodiments, the recombinant polynucleic acid composition comprises one or more recombinant polynucleic acid molecules comprising more than one recombinant polynucleic acid sequences, each recombinant polynucleic acid sequence of the more than one recombinant polynucleic acid sequences comprises a unique sequence encoding a transmembrane domain. In some embodiments, the recombinant polynucleic acid composition comprises a polypeptide encoded by each recombinant polynucleic acid sequence is expressed in a specific cell type. In some embodiments, the recombinant polynucleic acid composition each recombinant polynucleic acid sequence is expressed in a cell type distinct from a different sequence. In some embodiments, the recombinant polynucleic acid composition the transmembrane domain is operably linked with an extracellular domain, wherein the extracellular domain comprises an antigen binding domain.

Provided herein are designs of chimeric fusion proteins (CFP), that are T cell-specific and express in T cells, and do not substantially express in non-T cells. For example, the CFP specific for expression in T cells do not substantially express in a B cell or a myeloid cell, or an epithelial cell. For example, provided herein is a composition comprising a recombinant polynucleic acid comprising a sequence encoding a CFP, the CFP comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by T cells; and wherein after administration of the composition to a human subject the CFP is expressed on cell surfaces of T cells of the human subject. In some embodiments, the recombinant polynucleic acid is encapsulated by a nanoparticle delivery vehicle.

In some embodiments, the transmembrane domain is a transmembrane domain from a cell surface receptor selected from a group consisting of CD3, CD4, CD5, CD7, CD8, CD28, CD48, CD3ε, CD3δ, CD3γ, CD3ζ, TCRα chain, TCRβ chain, TCRγ chain and TCRδ chain. In some embodiments, the transmembrane domain is a transmembrane domain from a cell surface receptor selected from a group consisting of CD3, CD4, CD5, CD7, CD8, CD28 and CD48.

In some embodiments, the extracellular domain is an extracellular domain from CD3, CD4, CD5, CD7, CD8, CD28, CD48, CD3ε, CD3δ, CD3γ, CD3ζ, TCRα chain, TCRβ chain, TCRγ chain and TCRδ chain. In some embodiments, the extracellular domain is an extracellular domain from CD3, CD4, CD5, CD7, CD8, CD28 or CD48.

In some embodiments, the extracellular domain comprises a hinge domain from CD8, wherein the hinge domain is operatively linked to the transmembrane domain. In some embodiments, the CFP is preferentially or specifically expressed in T cells of the human subject. In some embodiments, the antigen binding domain comprises a Fab fragment, an scFv domain or an sdAb domain. In some embodiments, the CFP further comprises an intracellular domain. In some embodiments, the intracellular domain comprises an intracellular signaling domain from FcγR, FcαR, FCεR, CD40 or CD3ζ. In some embodiments, the intracellular domain further comprises a phosphoinositide 3-kinase (PI3K) recruitment domain.

In some embodiments, the intracellular domain comprises an intracellular domain from CD3, CD4, CD5, CD7, CD8, CD28, CD48, CD3ε, CD3δ, CD3γ or CD3ζ. In some embodiments, the intracellular domain comprises an intracellular domain from CD3, CD4, CD5, CD7, CD8, CD28 or CD48.

In some embodiments, the recombinant polynucleic acid is an mRNA.

In some embodiments, the nanoparticle delivery vehicle comprises a lipid nanoparticle. In some embodiments, the lipid nanoparticle comprises a polar lipid. In some embodiments, the lipid nanoparticle comprises a non-polar lipid. In some embodiments, the lipid nanoparticle is from 100 to 300 nm in diameter. In some embodiments, the lipid nanoparticle comprises (6Z,9Z,28Z,31Z)-heptatriaconta-6,9,28,31-tetraen-19-yl 4-(dimethylamino) butanoate (DLin-MC3-DMA; MC3). In some embodiments, the lipid nanoparticle comprises (a) a nucleic acid; (b) a cationic lipid; (c) a non-cationic lipid; and (d) a conjugated lipid that inhibits aggregation of particles. In some embodiments, the nucleic acid comprises a charged polyanioinc nucleic acid.

In one aspect, provided herein is a pharmaceutical composition comprising the composition comprising a recombinant polynucleic acid comprising a sequence encoding a chimeric fusion protein (CFP), the CFP comprising: (a) an extracellular domain comprising an antigen binding domain, and (b) a transmembrane domain operatively linked to the extracellular domain; wherein the transmembrane domain is a transmembrane domain from a protein that multimerizes with a cell surface receptor expressed by T cells; and a pharmaceutically acceptable excipient. In one embodiment, the pharmaceutical composition comprises an effective amount of the composition described above, to inhibit growth of a cancer when administered to a human subject with the cancer.

In one aspect, provided herein is a method of treating cancer in a subject in need thereof comprising administering the pharmaceutical composition comprising a recombinant polynucleic acid comprising a sequence encoding a chimeric fusion protein (CFP), and a pharmaceutically acceptable excipient to the subject; wherein the CFP comprising an extracellular domain comprising an antigen binding domain, and a transmembrane domain operatively linked to the extracellular domain; wherein the transmembrane domain is a transmembrane domain from a protein that multimerizes with a cell surface receptor expressed by T cells.

In one aspect, provided herein is a method of introducing the composition described above into a T cell, comprising, electroporating the T cell in the presence of the recombinant polynucleic acid comprising the sequence encoding the CFP, wherein the recombinant polynucleic acid is configured for expression of the recombinant polynucleic acid in T cells of a human subject.

In some embodiments, the antigen binding domain binds to an antigen selected from the group consisting of CD5, HER2, GPC3 and TROP2.

In some embodiments, the extracellular domain is an extracellular domain from a protein that multimerizes with a cell surface receptor expressed by T cells.

In some embodiments, the intracellular domain is an intracellular domain from a protein that multimerizes with a cell surface receptor expressed by T cells.

In some embodiments, the transmembrane domain is a transmembrane domain from a protein that is not expressed or not substantially expressed by non-T cells.

In some embodiments, the extracellular domain is an extracellular domain from a protein that is not expressed or not substantially expressed by non-T cells.

In some embodiments, the intracellular domain is an intracellular domain from a protein that is not expressed or not substantially expressed by non-T cells. In some embodiments, the transmembrane domain is a transmembrane domain from a protein that is not expressed or not substantially expressed by NK cells, B cells or myeloid cells. In some embodiments, the extracellular domain is an extracellular domain from a protein that is not expressed or not substantially expressed by NK cells, B cells or myeloid cells. In some embodiments, the intracellular domain is an intracellular domain from a protein that is not expressed or not substantially expressed by NK cells, B cells or myeloid cells.

### B Cell-Specific Chimeric Fusion Protein (CFP) Designs

Provided herein is a recombinant polynucleic acid composition comprising a recombinant polynucleic acid sequence encoding a chimeric fusion protein (CFP) comprising a transmembrane domain that specifically integrates within a membrane protein complex of a B cell, wherein the B cell is characterized as naturally expressing the membrane protein complex; wherein, when the recombinant polynucleic acid composition comprising the recombinant polynucleic acid sequence is contacted to any cell of a heterogenous cell population, at least greater than 50% of cells within the heterogenous cell population that are characterized as naturally expressing the membrane protein complex, e.g., B cells, expresses the CFP, and a cell within the heterogenous cell population that lacks the membrane protein complex, e.g., non-B cells, fails to express the CFP. In some embodiments, the naturally expressing the membrane protein complex of a B cell may be a CD19 or CD20 TM domain and intracellular domain. In some embodiments, it comprises an extracellular domain that comprises a sequence from the CD19, and an scFv that binds to a cancer antigen.

In some embodiments, the recombinant polynucleic acid composition is expressed in at least greater than 60%, 70%, 80% or 90% B cells within the heterogenous cell population. In some embodiments, the CFR is expressed in at least 50% of B cells in a heterogenous population PBMC, e.g. obtained from peripheral blood drawn at 1, 2, or 3 days after introduction of the polynucleic acid in the system of the subject. In some embodiments, the recombinant polynucleic acid composition less than 10% cells within the heterogenous cell population that lacks a CD20 or CD19 express the CFP. In some embodiments, the CFP is expressed in less than 10% T cells in a population of cells in a biological sample from the subject at 1, 2, or 3 days after administration of a composition comprising the recombinant polynucleic acid. In some embodiments, the CFP is expressed in less than 10% of myeloid cells in a biological sample from the subject at 1, 2, or 3 days after administration of a composition comprising the recombinant polynucleic acid. In some embodiments, the CFP is expressed in less than 10% of epithelial cells in a biological sample from the subject at 1, 2, or 3 days after administration of a composition comprising the recombinant polynucleic acid. In some embodiments, the recombinant polynucleic acid is expressed at greater than 50% of B cells, e.g., greater than 60%, 70%, 80% or 90% of B cells within the heterogenous cell population tested ex vivo. In some embodiments, the recombinant polynucleic acid is expressed at less than 10% of B cells, e.g., less than 9%, 8%, 7%, 6%, 5%, 4%, 3%, 2% 1% or less in cells other than B cells, e.g. epithelial cells or myeloid cells within the heterogenous cell population tested ex vivo.

The present disclosure describes immunotherapy using B cells engineered to express CARs via the delivery of a recombinant nucleic acid technology, encoding the CAR, that has a specific cancer targeting antigen domain. This recombinant nucleic acid technology can be delivered to B-cells via encapsulation within a lipid nanoparticle and electroporation into the B-cell. B cells can mediate a variety effects, such as antibody production, direction of antigen presenting cells, or direct cytotoxicity, making them an ideal candidate for immunotherapies directed towards conditions such as cancer, autoimmune diseases, fibrotic diseases, or infections. Advantageous properties of B cells that could be useful in the implementation of immunotherapies further include in vivo persistence, memory pool formation, and the potential to secrete large quantities of proteins. One challenge in the implementation of immunotherapy is the ability to recruit an effector cell, such as an immune cell, to a desired target. The present disclosure presents solutions to this challenge, in the form of engineered CAR expressing B cells.

The present disclosure involves making and using engineered B cells (e.g., CD19, or CD20 cells), which can attack and kill diseased cells directly and/or indirectly, such as cancer cells and infected cells. Engineered B cells can be prepared by incorporating nucleic acid sequences (e.g., mRNA, DNA, plasmids, viral constructs) encoding a chimeric fusion protein (CFP), that has an extracellular binding domain specific to disease associated antigens (e.g., cancer antigens), into the cells using, for example, recombinant nucleic acid technology, synthetic nucleic acids, gene editing techniques (e.g., CRISPR), transduction (e.g., using viral constructs), electroporation, lipid nanoparticles, or nucleofection. It has been found that B cells can be engineered to have a broad and diverse range of activities. For example, it has been found that B cells can be engineered to express a chimeric fusion protein (CFP) containing an antigen binding domain to have a broad and diverse range of activities. For example, it has been found that B cells can be engineered to have enhanced phagocytic activity such that upon binding of the CFP to an antigen on a target cell, the cell exhibits increased phagocytosis of the target cell. It has also been found that B cells can be engineered to promote T cell activation such that upon binding of the CFP to an antigen on a target cell, the cell promotes activation of T cells, such as T cells in the tumor microenvironment. The engineered B cells can be engineered to promote secretion of tumoricidal molecules such that upon binding of the CFP to an antigen on a target cell, the cell promotes secretion of tumoricidal molecules from nearby cells. The engineered B cells can be engineered to promote recruitment and trafficking of immune cells and molecules such that upon binding of the CFP to an antigen on a target cell, the cell promotes recruitment and trafficking of immune cells and molecules to the target cell or a tumor microenvironment.

The present disclosure is based upon the finding that engineered B cells can overcome at least some of the limitations of CAR-T cells, including, being readily recruited and present in solid tumors; B cells can avoid fratricide as they do not express the same antigens as malignant T cells; B cells have the ability to differentiate into antibody secreting cells following antigen specific activation; B cells have a natural longevity and can establish immunological memory, thereby generating long-term protective immunity; B cells from cancer patients retain the ability to proliferate; and B cells have a plethora of anti-tumor functions that can be deployed.

B cell are also found in large numbers within the tumor microenvironment, accounting for up to 25% of all cells in some tumors. B cell derived antibodies can alter the function of their antigenic targets on cancer cells, opsonize tumor cells for the presentation and cross-presentation of tumor antigens by dendritic cells, activate the complement cascade, or contribute to NK cell mediated tumor killing via antibody-dependent cell-mediated cytotoxicity.

In one aspect provided herein is a composition comprising a recombinant polynucleic acid comprising a sequence encoding a chimeric fusion protein (CFP), the CFP comprising (a) an extracellular domain comprising an antigen domain, and (b) a transmembrane domain operatively linked to the extracellular domain; wherein the transmembrane domain is a transmembrane domain from a protein that multimerizes with a cell surface receptor expressed by B cells; and wherein after administration of the composition to a human subject the CFP is expressed on cell surfaces of B cells of the human subject.

In some aspects, the recombinant polynucleic acid is encapsulated by a nanoparticle delivery vehicle.

In some aspects, the transmembrane domain is a transmembrane domain from a cell surface receptor selected from a group consisting of CD19, CD20, CD21, CD22, CD27, CD28, CD45, CD72, CD79a, CD79b and CD81.

In some aspects, the transmembrane domain is a transmembrane domain from a cell surface receptor selected from a group consisting of CD79a and CD79b.

In some aspects, wherein the extracellular domain is an extracellular domain from CD19, CD20, CD21, CD22, CD27, CD28, CD45, CD72, CD79a, CD79b or CD81.

In some aspects, the extracellular domain is an extracellular domain from CD79a or CD79b.

In some aspects, the extracellular domain comprises a hinge domain from CD8, CD28 or Siglec4, wherein the hinge domain is operatively linked to the transmembrane domain.

In some aspects, the CFP is preferentially or specifically expressed in B cells of the human subject.

In some aspects, the antigen binding domain comprises a Fab fragment, an scFv domain or an sdAb domain.

In some aspects, the CFP further comprises an intracellular domain.

In some aspects, the intracellular domain comprises an intracellular signaling domain from FcγR, FcαR, FcεR, FcR, CD40 or CD3ζ.

In some aspects, the intracellular domain comprises an intracellular signaling domain from FcγRIIB, Siglec-G, CD22, CD72, CD152, LAIR1, CD85j, PIR-B or PD-1.

In some aspects, the one or more intracellular signaling domains further comprises a phosphoinositide 3-kinase (PI3K) recruitment domain or a Spleen tyrosine kinase (SYK) recruitment domain. In some embodiments, the PI3K recruitment domain comprises a sequence of SEQ ID NO: 26.

In some aspects, the intracellular domain comprises an intracellular domain from CD19, CD20, CD21, CD22, CD27, CD28, CD45, CD72, CD79a, CD79b or CD81.

In some aspects, the intracellular domain comprises an intracellular domain from CD79a, CD79b, CD19 or CD28.

In some aspects, the recombinant polynucleic acid is an mRNA.

In some aspects, the nanoparticle delivery vehicle comprises a lipid nanoparticle.

In some aspects, the lipid nanoparticle comprises a polar lipid.

In some aspects, the lipid nanoparticle comprises a non-polar lipid.

In some aspects, the lipid nanoparticle is from 100 nm to 300 nm in diameter.

In some aspects, the lipid nanoparticle comprises (6Z,9Z,28Z,31Z)-heptatriaconta-6,9,28,31-tetraen-19-yl 4-(dimethylamino) butanoate (DLin-MC3-DMA; MC3).

In some aspects, the lipid nanoparticle comprises (a) a nucleic acid; (b) a cationic lipid; (c) a non-cationic lipid; and (d) a conjugated lipid that inhibits aggregation of particles.

In some aspects, the nucleic acid comprises a charged polyanioinc nucleic acid.

In a further aspect, provided herein is a pharmaceutical composition comprising the composition comprising the recombinant polynucleic acid, and a pharmaceutically acceptable excipient.

In some aspects, the pharmaceutical composition comprises an effective amount of the composition of claim 1 to inhibit growth of a cancer when administered to a human subject with the cancer.

In a further aspect, provided herein is a method of treating cancer in a subject in need thereof comprising administering the pharmaceutical composition comprising the recombinant polynucleic acid composition, and a pharmaceutically acceptable excipient to the subject.

In a further aspect, provided herein is a method of introducing the recombinant polynucleic acid composition into a B cell comprising electroporating the B cell in the presence of the recombinant polynucleic acid comprising the sequence encoding the CFP, wherein the recombinant polynucleic acid is configured for expression of the recombinant polynucleic acid in B cells of a human subject.

In some aspects, the antigen binding domain binds to an antigen selected from the group consisting of CD5, HER2, GPC3 and TROP2. In some aspects, the extracellular domain is an extracellular domain from a protein that multimerizes with a cell surface receptor expressed by B cells. In some aspects, the intracellular domain is an intracellular domain from a protein that multimerizes with a cell surface receptor expressed by B cells. In some aspects, the transmembrane domain is a transmembrane domain from a protein that is not expressed or not substantially expressed by non-B cells. In some aspects, the extracellular domain is an extracellular domain from a protein that is not expressed or not substantially expressed by non-B cells. In some aspects, the intracellular domain is an intracellular domain from a protein that is not expressed or not substantially expressed by non-B cells. In some aspects, wherein the transmembrane domain is a transmembrane domain from a protein that is not expressed or not substantially expressed by NK cells, T cells or myeloid cells. In some aspects, the extracellular domain is an extracellular domain from a protein that is not expressed or not substantially expressed by NK cells, T cells or myeloid cells.

In some aspects, the intracellular domain is an intracellular domain from a protein that is not expressed or not substantially expressed by NK cells, T cells or myeloid cells. In some aspects, the transmembrane domain is a transmembrane domain from a protein that forms a heterodimer with IgA or IgB. In some aspects, the extracellular domain is an extracellular domain from a protein that forms a heterodimer with IgA or IgB. In some aspects, the intracellular domain is an intracellular domain from a protein that forms a heterodimer with IgA or IgB.

Additional aspects and advantages of the present disclosure will become readily apparent to those skilled in this art from the following detailed description, wherein only illustrative aspects of the present disclosure are shown and described. As will be realized, the present disclosure is capable of other and different aspects, and its several details are capable of modifications in various obvious respects, all without departing from the disclosure. Accordingly, the drawings and description are to be regarded as illustrative in nature, and not as restrictive.

Recombinant polynucleic acid and delivery vehicle.

In one aspect, provided herein is a recombinant polynucleic acid comprising a sequence that encodes a chimeric fusion protein that is designed to be expressed predominantly in T cells, B cells or NK cells by the design of the polynucleic acid that is contacted to a heterogenous cell population comprising the T cells, the B cells or the NK cells. In one embodiment, the recombinant polynucleic acid comprising a sequence that encodes a chimeric fusion protein, which upon expression, renders the cell potent for lysing a tumor cell or other diseased cell. In some embodiments, the recombinant polynucleic acid is DNA. In some embodiments, the recombinant polynucleic acid is RNA. In some embodiments, the recombinant polynucleic acid is mRNA. In some embodiments, the recombinant polynucleic acid is an unmodified mRNA. In some embodiments, the recombinant polynucleic acid is a modified mRNA. In some embodiments, the recombinant polynucleic acid is a circRNA. In some embodiments, the recombinant polynucleic acid is a tRNA. In some embodiments, the recombinant polynucleic acid is a microRNA.

Provided herein is a composition comprising a nucleic acid comprising (i) DNA sequence encoding an mRNA or (ii) the mRNA sequence, wherein the mRNA sequence comprises (i) a 5′ UTR sequence and (ii) a 3′ UTR sequence, wherein the 5′ UTR is at least 45 nucleotides in length and a sequence encoding a target gene or protein therebetween. In some embodiments, the 5′ UTR sequence, and/or the 3′ UTR sequence can comprise a non-native sequence, that is, a sequence that is not present in an unmodified transcript. In some embodiments, the nucleic acid or nucleic acid sequence is recombinant. In some embodiments, the nucleic acid” or nucleic acid sequence is engineered. In some embodiments, the nucleic acid or nucleic acid sequence is synthetic. In some embodiments, the nucleic acid or nucleic acid sequence is in vitro transcribed. In some embodiments, the nucleic acid or nucleic acid sequence is isolated or purified.

In some embodiments, the nucleic acid, e.g., an engineered nucleic acid, an in vitro transcribed (IVT) mRNA, a synthetic or modified nucleic acid as described herein is not conjugated to or associated with a lipid nanoparticle (LNP).

In some embodiments, the nucleic acid, e.g., an engineered nucleic acid, an in vitro transcribed mRNA, a synthetic or modified nucleic acid as described herein is electroporated into a cell. In some embodiments, the nucleic acid, e.g. an IVT mRNA comprises a 3′UTR and a 5′UTR. In some embodiments, the 3′ UTR sequence is followed by a poly A sequence. In some embodiments, the poly A sequence is at least 100 nucleotides long (SEQ ID NO: 288). In some embodiments, the poly A sequence is at least about 50, 60, 70, 80, 90, 100, 110, 120, 130, 140, 150, 160, 170, 180, 190 or 200 nucleotides long. In some embodiments, the poly A sequence is greater than 200 nucleotides long (SEQ ID NO: 295). In some embodiments, within the 5′ UTR, a translation start site is at least 15 nucleotides downstream of the 5′ end of the mRNA. In some embodiments, a translation start site is at least 20 nucleotides downstream of the 5′ end of the mRNA. In some embodiments, the translation start site is at least 25 nucleotides downstream of the ribosome binding site. In some embodiments, the translation start site is at least 30 nucleotides downstream of the ribosome binding site. In some embodiments, the 5′ end of the nucleic acid comprises a methyl guanylate cap. In some embodiments, the nucleic acid comprises a single translational start site. In some embodiments, the mRNA coding sequence is 100-10,000 nucleotides long. In some embodiments, the recombinant polynucleic acid comprises a sequence encoding a homeostatic regulator of inflammation. In some embodiments, the homeostatic regulator of inflammation is a sequence in an untranslated region (UTR) of an mRNA. In some embodiments, the sequence in the UTR is a sequence that binds to an RNA binding protein. In some embodiments, translation is inhibited or prevented upon binding of the RNA binding protein to the sequence in an untranslated region (UTR). In some embodiments, the sequence in the UTR comprises a consensus sequence of WWWU(AUUUA)UUUW (SEQ ID NO: 285), wherein W is A or U. In some embodiments, the recombinant polynucleic acid is expressed on a bicistronic vector.

In some embodiments, the mRNA comprises one or more modified nucleotides for increased stability and nuclease resistance, such as is well known in the art. In some embodiments, the mRNA is modified at the termini for enhanced and/or prolonged expression in a cell, e.g., an NK cell. In some embodiments, the nucleic acid comprises one or more modified nucleotide bases, wherein a fraction of the total number of uridine bases are modified to a pseudouridine, a 1-methyl-pseudouridine or a 5-methoxyuridine. In some embodiments, less than 50% of the total number of uridine bases are modified to a pseudouridine, a 1-methyl-pseudouridine or a 5-methoxyuridine. In some embodiments the 5′ UTR is at least 20 nucleotides in length. In some embodiments the 5′ UTR is at least 30 nucleotides in length. In some embodiments the 5′ UTR is at least 60 nucleotides in length. In some embodiments the 5′ UTR is at least 100 nucleotides in length. In one embodiment, the nucleic acid is an mRNA comprising a poly A sequence that is enzymatically added. In one embodiment, the nucleic acid is an mRNA comprising a poly A sequence that is enzymatically added. In some embodiments, the nucleic acid is an mRNA comprising a poly A sequence that is encoded by the plasmid which comprises the template for generating the mRNA by in vitro transcription (IVT). The template for IVT is a linearized plasmid. Typically, the length of the poly A is controlled when encoded by the plasmid, and is less controlled when enzymatically added. An mRNA product comprising an enzymatically added poly A tail may be tailored at best to contain a narrowed range of the number of A-residues. The in vitro transcribed mRNA is thereafter purified prior. In some embodiments, the nucleic acid comprises a poly A sequence downstream of the 3′ UTR sequence. In some embodiments, the poly A sequence is at least 50 nucleotides long (SEQ ID NO: 286). In some embodiments, the poly A sequence is at least 60, 70, 80, or 90 nucleotides long (SEQ ID NO: 287). In some embodiments, the poly A sequence is at least 100 nucleotides long (SEQ ID NO: 288). In some embodiments, the poly A sequence is at least 110 nucleotides long (SEQ ID NO: 289). In some embodiments, the poly A sequence is at least 120 nucleotides long (SEQ ID NO: 290). In some embodiments, the poly A sequence is at least 130 nucleotides long (SEQ ID NO: 291). In some embodiments, the poly A sequence is at least 140 nucleotides long (SEQ ID NO: 292). In some embodiments, the poly A sequence is at least 150, 160, 170, 180, 190 or 200 nucleotides long (SEQ ID NO: 293). In some embodiments, the within the 5′ UTR, a translation start site is at least 15 nucleotides downstream of the 5′ end. In some embodiments, the translation start site is at least 20 nucleotides downstream of the 5′ end. In some embodiments, the translation start site is at least 25 nucleotides downstream of the 5′ end. In some embodiments, the translation start site is at least 30 nucleotides downstream of the 5′ end. In some embodiments, the nucleic acid comprises a single translational start site.

In some embodiments, the nucleic acid comprises a 5′ methyl guanylate cap. A proper 5′-cap structure is important in the synthesis of functional messenger RNA. In some embodiments, the mRNA design as described herein comprises a proper 5′—cap structure, wherein the 5′—cap comprises a guanosine triphosphate arranged as GpppG at the 5′ terminus of the nucleic acid. In some embodiments, the mRNA comprises a 5′ 7-methylguanosine cap, m7-GpppG. A 5′ 7-methylguanosine cap can increase mRNA translational efficiency and prevents degradation of mRNA 5′-3′ exonucleases. In some embodiments, the mRNA comprises “anti-reverse” cap analog (ARCA, m7,3′-O GpppG).

In some embodiments, the nucleic acid is isolated.

In some embodiments, the nucleic acid is purified.

In some embodiments, the nucleic acid comprises at least 1 modified nucleotide.

In some embodiments, the nucleic acid comprises at least 10% modified nucleotides.

In some embodiments, the nucleic acid comprises at least 20% modified nucleotides.

In some embodiments, the nucleic acid comprises at least 30%, 40%, or 50% modified nucleotides. In some embodiments, less than 70% of the uridine residues in the nucleic acid are modified.

In some embodiments, less than 50% of the uridine residues in the nucleic acid are modified.

In some embodiments, the modified nucleotide is a pseudouridine, 1-methyl-pseudouridine or a 5-methoxyuridine that replaces a uridine.

Additionally, in some embodiments the phosphate backbone of an mRNA described herein is modified for stability. In some embodiments, the phosphate group of a chemically modified nucleotide can be modified by replacing one or more of the oxygens with a different substituent. In some aspects, the chemically modified nucleotide can include replacement of an unmodified phosphate moiety with a modified phosphate as described herein. In some aspects, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters.

In some embodiments, a stable integration of transgenes into NK cells, B cells or T cells can be accomplished via the use of a transposase and transposable elements, in particular, mRNA-encoded transposase. In one embodiment, Long Interspersed Element-I (LI) RNAs may be contemplated for retrotransposition of the transgene and stable integration into NK cells, or a T cell or a B cell as designed. such as macrophages or phagocytic cells. Retrotransposon may be used for stable integration of a engineered nucleic acid encoding a CFP as described herein.

Also provided herein is a delivery vehicle, e.g., a vector, or a nanoparticle, comprising the recombinant polynucleic acid sequence encoding a CFP described herein. Exemplary delivery vehicles contemplated herein are described.

Viral Vectors: In some embodiments, the vector for expression of the recombinant protein is of a viral origin, namely a lentiviral vector or an adenoviral vector. In some embodiments, the nucleic acid encoding the recombinant polynucleic acid is encoded by a lentiviral vector. In some embodiments the lentiviral vector is prepared in-house and manufactured in large scale for the purpose. In some embodiments, commercially available lentiviral vectors are utilized, as is known to one of skill in the art.

In some embodiments the viral vector is an Adeno-Associated Virus (AAV) vector.

### Nanoparticle Mediated Delivery:

In some embodiments, the recombinant polynucleic acid is encapsulated in a liposome. In some embodiments, the liposome is a lipid nanoparticle. In some embodiments, the recombinant polynucleic acid is encapsulated in polymeric nanoparticles.

In some embodiments, the recombinant polynucleic acid is encapsulated in a lipid nanoparticle. In some embodiments, the lipid nanoparticle comprises (a) a nucleic acid; (b) a cationic lipid; (c) a non-cationic lipid; and (d) a conjugated lipid that inhibits aggregation of particles. In some embodiments, the nucleic acid comprises a charged polyanionic nucleic acid. A lipid nanoparticle may comprise a polar lipid. In some embodiments, the lipid nanoparticle comprises a cationic lipid. Cationic lipids have a head group with permanent positive charges. In some embodiments, the lipid nanoparticle comprises a cationic lipid and a non-cationic lipid. In some embodiments, the lipid nanoparticle comprises a neutral lipid. In some embodiments, the lipid nanoparticle comprises a PEGylated lipid. In some embodiments, the delivery vehicle encapsulates the recombinant polynucleic acid. A lipid nanoparticle for use in delivery of a nucleic acid, e.g., mRNA as in the present context comprises any one or more of the lipid components: 30601, tetrakis(8-methylnonyl) 3,3′3″,3″′-(((methylazanediyl)bis(propane-3,1 diyl))bis(azanetriyl))tetrapropionate; 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl)propyl)-2,5-dihydro-1H-irnidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-1,-ditetradecylacetanide; P3-sitosterol, (3S8SS,10R, 13R,14S,17R)-17-((2R,5R)-5-etiyl-6-methylheptan-2-yl)-10 13-dimethyl-2,3,4,7,8,9,10,11,12,13,14,15,16,17-tetradecahydro-1H-cyclopenta[alphenanthren-3-ol; BAME-O16B, bis(2-(dodecyldisulfanyl)etlwi) 3,3′-((3-methiyl-9-oxo-10-oxa-13,14-dithia-3,6-diazahexacosyl)azanediyl)dipropionate; BHEM-Cholesterol, 2-(((((3,8S9S,10R1,13R,14S,17R)-10,13-dimethyl-i7-((R)-6-methylheptan-2-yl)-2,3,4,7,8,9,10,11,12,13,14,15,16,17-tetradecahydro-1H-cvclopenta[a]phenanthren-3-yl)oxy)carbonyl)amino)-,NV-bis(2-hydroxyethyl)-N-methylethan-1-aminium bromide; C12-200, 1,1′-((24-(2-((2-(bis(2-hydroxydodecyv)amino)ethyv) (2-hydroxydodecyl)amino)ethyl) piperazin-1-yl)ethyl)azanediyl)bis(dodecan-2-ol); cKK-E12 3,6-bis(4-(bis(2-hydroxvdodecyl)amino)butyl) piperazine-2,5-dione; DC-Cholesterol, 3P-[N-(N′,N′-dimethylaminoethane)-carbamoyl]cholesterol; DLin-MC3-DMA, (6Z,97,28Z,31IZ)-heptatriaconta-6,9,28,31-tetracn-19-l 4-(dimethylamino) butanoate; DOPE, 1,2-dioleoyl-sn-glycero-3-phosphoethanolamine; DOSPA, 2,3-dioleylox-V-[2-(sperminecarboxamido)ethiyl]-iV,N-dimmethyl-1-propanaminium trifluoroacetate; DOTAP, 1,2-dioleoyl-3-trimethylanmmonium-propane; DOTMA, 1,2-di-O-octadecenyl-3-trimethylammoniurm-propane; DSPC, 1,2-distearoyl-sn-glycero-3-phosphocholine; ePC, ethylphosphatidylcholine; FTT5, hexa(octan-3-y]) 9,9′,9″0.9″′,9″″,9′″″-((((benzene-1,3,5-tricarbonyl)yris(azanediyl))tris(propane-3,1-diyl))tris(azanetriyl))hexanonanoate; LipidH(SM-102), heptadecan-9-v 8-((2-hydroxyethyl)(6-oxo-6-(undecyloxy)hexyl)amino) octanoate; OF-Deg-Lin, (((3,6-dioxopiperazine-2,5-diyl)bis(butane-4, I-diyl))bis(azanetriyl))tetrakis(ethane-2,1-diyl) (9Z,9′Z,9″Z,″Z,12Z,12′,12″712Z-tetrakis(octadeca-9,12-dienoate); PEG2000-DMG, 1,2-dimvristovl-rac-glycero-3-methoxypolyethvlene glycol-2000; TT3, N′,N³,Atris(3-(didodecvlamino)propyl)benzene-1,3,5-tricarboxamide. In some embodiments, the lipid nanoparticle comprises any one of the cationic lipid components, DOTMA (1,2-di-O-octadecenvl-3-trimethylainunonium-propane), or DOTAP (1,2-dioleoyl-3-trimethylamnmonium-propane) or DOPE (1,2-dioleovl-sn-glycero-3-phosphoethanolamine). In sonme embodiments, the ionizable lipids may be used. Ionizable lipids are protonated at low pH, which renders them positively charged, that promote membrane destabilization and endosomal escape of the nanoparticle. Exemplary nanoparticles are (2S)-2,5-bis(3-aminopropylamino)-[2-(dioctadecvlamino)acetyl]pentanamide, (DOGS), N′-[2-((1S)-I-[(3-arinopropyl)amino]-4-[di(3-aminopropyl)amino]butylcarboxamido)ethyl]-3,4-di[oleyioxy]-benzamide (MVL5), DC-Cholesterol, N4-cholestervl-spermine (GL67), 2,2-dilinolevl-4-dimethylaminoethvi-[1,3]-dioxolane (DLin-KC2-DMA) and DLin-KC2-DMA led to (6Z,97,28Z,31Z)-heptatriaconta-6,9,28,31-tetraen-19-yl 44dimethylamino) butanoate (DLin-MC3-DMA; MC3). In some embodiments, the lipid nanoparticle comprises (6Z,9Z,28Z,31Z)-heptatriaconta-6,9,28,31-tetraen-19-yl 4-(dimethylamino) butanoate (DLin-MC3-DMA; MC3. In some embodiments, any one or more of the nanoparticle components may be functionalized to attach a targeting moiety.

In some embodiments, the delivery vehicle is an exosome or an extracellular vesicle. In some embodiments, the exosome or extracellular vesicle is electroporated with the recombinant polynucleic acid. In some embodiments, the exosome or extracellular vesicle is obtained from a cell electroporated with the recombinant polynucleic acid.

Lipid nanoparticles (LNP) may comprise a polar and or a nonpolar lipid. In some embodiments cholesterol is present in the LNPs for efficient delivery. LNPs are 100-300 nm in diameter provide efficient means of mRNA delivery to various cell types, including monocytes or macrophages. In some embodiments, LNP may be used to introduce the recombinant polynucleic acids into a cell in in vitro cell culture. In some embodiments, the LNP encapsulates the nucleic acid wherein the nucleic acid is a naked DNA molecule. In some embodiments, the LNP encapsulates the nucleic acid wherein the nucleic acid is an mRNA molecule. In some embodiments, the LNP encapsulates the nucleic acid wherein the nucleic acid is inserted in a vector, such as a plasmid vector. In some embodiments, the LNP encapsulates the nucleic acid wherein the nucleic acid is a circRNA molecule.

In some embodiments, the LNP is used to deliver the nucleic acid into a subject. LNP can be used to deliver nucleic acid systemically in a subject. It can be delivered by injection. In some embodiments, the LNP comprising the nucleic acid is injected by intravenous route. In some embodiments the LNP is injected subcutaneously.

Microbubble mediated delivery: In some embodiments, microbubbles can be used for delivery of a composition comprising e.g., a nucleic acid in a subject. Perfluorocarbon-filled microbubbles are stable for circulating in the vasculature as blood pool agents, they act as carriers of these agents until the site of interest is reached. Ultrasound applied over the skin surface can then be used to burst the microbubbles at this site, causing localized release of the drug. Various other forms of microbubbles include Sonazoid Optison, gas-filled albumin microbubble, and PESDA. Optimization of the composition of the microbubble with respect to the composition of the therapeutic agent that is delivered, along with the site of delivery intended is necessary.

Delivery vehicles may include specialized biodegradable polymers, such as PLGA (poly(lactic-co-glycolic) acid and/or polyvinyl alcohol (PVA). In some embodiments, one or more compounds can be selectively incorporated in such polymeric structures to affect the NK cell function. In some embodiments, the targeting structures are multilayered, e.g., of one or more PLGA and one or more PVA layers. In some embodiments, the targeting structures are assembled in an order for a layered activity. In some embodiments, the targeted polymeric structures are organized in specific shaped components, such as labile structures that can adhere to NK cell surface and deliver one or more components such as growth factors and cytokines, such as to maintain the NK cell in a microenvironment that endows a specific polarization. In some embodiments the polymeric structures are capable of sustained release of the one or more growth factors in an in vivo environment, such as in a solid tumor.

Also provided herein is a polypeptide encoded by the recombinant polynucleic acid of a composition described herein.

Also provided herein is a cell comprising a recombinant polynucleic acid composition described herein, a vector described herein or a polypeptide described herein. In some embodiments, the cell is a phagocytic cell. In some embodiments, the cell is a stem cell derived cell. In some embodiments, the cell is an autologous cell. In some embodiments, the cell is an allogeneic cell.

Also provided herein is a pharmaceutical composition comprising a composition described herein, such as a recombinant polynucleic acid described herein, a vector described herein, a polypeptide described herein or a cell described herein; and a pharmaceutically acceptable excipient.

Methods for preparing CFPs for expression in NK cells T cells or B cells

In one aspect, provided herein are methods of making a recombinant polynucleic acid encoding a chimeric fusion protein that are preferentially and predominantly expressed in NK cells. Similarly, in another aspect, provided herein are methods of making a recombinant polynucleic acid encoding a chimeric fusion protein that are preferentially and predominantly expressed in T cells. In yet another aspect, provided herein are methods of making a recombinant polynucleic acid encoding a chimeric fusion protein that are preferentially and predominantly expressed in NK cells or T cells or B cells, depending on the design of the recombinant polynucleic acid. In one embodiment, recombinant polynucleic acid encoding the chimeric fusion protein is designed for therapeutic application. In one embodiment, the recombinant polynucleic acid encoding the CFP is designed for administering in a subject, e.g., a human subject in vivo, as a nucleic acid molecule, preferably as a nucleic acid molecule delivered via a nanoparticle, and administered systemically or locally to the subject. In one aspect, the recombinant polynucleic acid encoding the CFP is designed as a product for off-the-shelf use.

Some of the following embodiments may indicate only NK cell, but NK cell may serve as an exemplary context or feature and may further be generalized for the methods and compositions applicable to T and B cell-specific contexts as well.

One aspect of the disclosure concerns the designing of the CFP that preferentially or predominantly expresses in an NK cell, and is not substantively expressed in a non-NK cell when administered in vivo. Applicants sought to design the CFP to contain specific domains, e.g., a transmembrane domain that oligomerizes or multimerizes with one or more endogenous proteins expressed in an NK cell, whereby the expression and/or function of the encoded CFP is dependent on the oligomerization or multimerization with the one or more endogenous proteins expressed in an NK cell, ensuring expression of the CFP predominantly in NK cells. Therefore, the method for preparing chimeric fusion proteins comprise the steps of (1) screening for domains or subunits for the CFP framework endowing NK cell-specific expression of the CFP as described above; (2) testing for construct for expression and dependency on one or more endogenous NK cell proteins; (3) testing functionality and efficiency of the cell expressing the CFP for its intended use, e.g., tumor cell killing.

Screening for PSR subunit framework: As described above, the design of the receptor comprises at least of one transmembrane domain, and an intracellular signaling domain which enables the enhanced signaling of target cell lysis. In essence a large body of plasma membrane proteins known to be endogenous in NK cells can be screened for novel NK cell-specific co-receptor function in combination with functional enhancements domains, such as intracellular signaling domains. The TM and the ICDs are paired appropriately with extracellular domain, comprising but not limited to one or more domains, e.g., comprising a hinge domain, and one or more antigen binding domain. In some embodiments, antigen binding domains are designed to drive the NK cell specifically to the target cell, e.g., the antigen binding domain binds to an antigenic ligand expressed on a tumor cell. In other embodiments, additional endogenous NK receptor extracellular domains can be included for improved functionality, e.g, degranulation and lysis of the target cell. Methods for screening NK cell receptor subunits as used herein utilize molecular cloning methods known to one of skill in the art. Additional information can be found in The Examples section. In general, functional genomics and reverse engineering is often employed to obtain a genetic sequence encoding a functionally relevant protein, polypeptide or a portion thereof. In some embodiments, primers and probes are constructed for identification, and or isolation of a protein, a polypeptide or a fragment thereof or a nucleic acid fragment encoding the same. In some embodiments, the primer or probe may be tagged for experimental identification. In some embodiments, tagging of a protein or a peptide may be useful in intracellular or extracellular localization.

Potential antibodies are screened for selecting specific antigen binding domains of high affinity. Methods of screening for antibodies or antibody domains are known to one of skill in the art. Specific examples provide further information. Examples of antibodies and fragments thereof include, but are not limited to IgAs, IgDs, IgEs, IgGs, IgMs, Fab fragments, F(ab′)2 fragments, monovalent antibodies, scFv fragments, scRv-Fc fragments, IgNARs, hcIgGs, VHH antibodies, nanobodies, and alphabodies.

Commercially available antibodies can be adapted to generate extracellular domains of a chimeric receptor. Examples of commercially available antibodies include, but are not limited to: anti-HGPRT, clone 13H11.1 (EMD Millipore), anti-RORI (ab135669) (Abcam), anti-MUC1 [EP1024Y](ab45167) (Abcam), anti-MUC16 [X75](ab1107) (Abcam), anti-EGFRvIII [L8A4](Absolute antibody), anti-Mesothelin [EPR2685 (2)](abl34109) (Abcam), HER2 [3B5](ab16901) (Abcam), anti-CEA (LS-C84299-1000) (LifeSpan BioSciences), anti-BCMA (ab5972) (Abcam), anti-Glypican 3 [9C2](abl29381) (Abcam), anti-FAP (ab53066) (Abcam), anti-EphA2 [RM-0051-8F21](ab73254) (Abcam), anti-GD2 (LS-0546315) (LifeSpan BioSciences), anti-CD19 [2E2B6B10](ab31947) (Abcam), anti-CD20 [EP459Y](ab78237) (Abcam), anti-CD30 [EPR4102](abl34080) (Abcam), anti-CD33 [SP266](abl99432) (Abcam), anti-CD123 (ab53698) (Abcam), anti-CD133 (BioLegend), anti-CD123 (1A3H4) ab181789 (Abcam), and anti-CD171 (LI. 1) (Invitrogen antibodies). Techniques for creating antibody fragments, such as scFvs, from known antibodies are routine in the art.

The recombinant polynucleic acid can be generated following molecular biology techniques known to one of skill in the art. The methods include but are not limited to designing primers, generating PCR amplification products, restriction digestion, ligation, cloning, gel purification of cloned product, bacterial propagation of cloned DNA, isolation and purification of cloned plasmid or vector. General guidance can be found in: Molecular Cloning of PCR Products: by Michael Finney, Paul E. Nisson, Ayoub Rashtchian in Current Protocols in Molecular Biology, Volume 56, Issue 1 (First published: 1 Nov. 2001); Recombinational Cloning by Jaehong Park, Joshua LaBaer in Current Protocols in Molecular Biology Volume 74, Issue 1 (First published: 15 May 2006) and others. In some embodiments specific amplification techniques may be used, such as TAS technique (Transcription-based Amplification System), described by Kwoh et al. in 1989; the 3SR technique, which are hereby incorporated by reference. (Self-Sustained Sequence Replication), described by Guatelli et al. in 1990; the NASBA technique (Nucleic Acid Sequence Based Amplification), described by Kievitis et al. in 1991; the SDA technique (Strand Displacement Amplification) (Walker et al., 1992); the TMA technique (Transcription Mediated Amplification).

The recombinant polynucleotide is synthesized by ligating DNA encoding, for example, a first binding domain, a linker, and a second binding domain in the same open reading frame using the molecular cloning techniques well known to one of skill in the art. In some embodiments, one or more polynucleotide sequences are arranged in an expression cassette to be expressed under the influence of same promoter and regulatory elements for generation of a single polypeptide. In some embodiments, a short spacer may be inserted between two adjacent polynucleotide sequences encoding two peptides wherein the spacer may encode a post translational cleavage site. The two polypeptides can be separated after translation by induction of the cleavage at the specific cleavage site. In some embodiments, the construct may be monocistronic or polycistronic. In some embodiments, more than one polypeptides are generated which then reassemble after translation. For example, light chain and heavy chain domains of an antibody or parts thereof can be generated by translation from two independent polynucleotide sequences, which are allowed to freely assemble with each other post-translationally. Alternatively, multiple polypeptide chains containing LC and HC variable domains that bind with each other are transcribed and translated from a single polynucleotide, which is cleaved after translation into respective peptide chains which can then reassemble. The polypeptide having a leader sequence is a preprotein and can have the leader sequence cleaved by the host cell to form the mature form of the polypeptide.

In some embodiments, the polynucleotide construct encodes an N-terminal signal sequence upstream of the polypeptide for secretion of the polypeptides. In some embodiments, the N terminal signal sequence comprises a secretion sequence. The resulting translated protein product having the N-terminal signal sequence for secretion would be secreted by the cell.

In some embodiments the plasmid vector is introduced or incorporated in the cell by known methods of transfection, such as using lipofectamine, or calcium phosphate, or via physical means such as electroporation or nucleofection. In some embodiments the viral vector is introduced or incorporated in the cell by infection, a process commonly known as viral transduction.

In some embodiments, recombinant polynucleic acid is integrated or incorporated in an expression vector. A vector comprises one or more promoters, and other regulatory components, including enhancer binding sequence, initiation and terminal codons, a 5′UTR, a 3′UTR comprising a transcript stabilization element, optional conserved regulatory protein binding sequences and others.

In some embodiments the vectors of use in the application are specifically enhanced for expression. Other exemplary vectors of use throughout the process include phages, cosmids, or artificial chromosomes.

It is understood that any one of the binder domains (extracellular binding domain binding to a target cell such as a cancer cell or a diseased cell or a pathogen) can be designed in combination other domains, e.g., transmembrane domains or intracellular domains described anywhere in the specification.

In some embodiments, the recombinant proteins, for example the CFP, or the inflammatory proteins or coreceptors that are co-expressed, or any associated protein designed to be expressed in a NK cell may be encoded by a recombinant polynucleic acid, wherein the recombinant polynucleic acid is an RNA. In some embodiments, the recombinant polynucleic acid is an mRNA. In some embodiments, the mRNA comprises one or more modifications for enhanced expression and stability. In some embodiments, the mRNA may be circularized. In some embodiments, the modifications may include but are not limited to: replacement of a nucleobase with a base analog, or a modified nucleotide; inserting one or more motifs within the mRNA, and introducing modifications in the 5′- and 3′ UTRs. In some embodiments, the recombinant polynucleic acid may be administered directly in a subject in need thereof.

In some embodiments the complementary binding of cognate peptides with each other can be via chemical binding, such as crosslinking. Chemical crosslinkers can be useful for activating the crosslinking in vitro. There are homo- and heterobifunctional protein crosslinkers that can be commercially available. Examples include BS2G crosslinker (BS²G; Bis[Sulfosuccinimidyl]glutarate) is an amine-reactive, water soluble, homobifunctional protein crosslinker (both binding units at the opposite ends of a spacer arm have the identical reactive groups), or its membrane permeable version, DSG (Disuccinimidyl glutarate;Di(N-succinimidyl) glutarate); BS3 crosslinker (Bis[sulfosuccinimidyl]suberate; Sulfo-DSS; BSSS) or DST crosslinker (Disuccinimidyl tartrate), are among other homobifunctional crosslinkers for peptides; whereas BMPS (N-(B-Maleimidopropyloxy) succinimide ester; MBS crosslinker (m-Maleimidobenzoyl-N-hydroxysuccinimide ester); PDPH crosslinker (3-[2-Pyridyldithio]propionyl hydrazide) provide examples of some heterobifunctional crosslinkers.

Testing of potential chimeric constructs: The above method leads to a design of a multitude potential CFPs, which are tested in a cell line for suitability of further development. The CFP constructs can be cloned in a plasmid vector and transfected in any immortalized cell lines, e.g. Chinese hamster ovarian (CHO) cells, HEK cells, MEF fibroblasts can be used. Co-expression of the CFP with the potential endogenous co-receptor is performed to test coreceptor dependency for CFP expression. In some embodiments, NK cells can be first transformed and immortalized, specifically for use in testing the CFP constructs for NK cell-specific expression. Additionally, (or alternatively) cells are electroporated with mRNA constructs, encoding the CFP to test expression, stability, and other characteristics for suitability in in vivo delivery, and can be used to modify UTRs or other structural aspects to improve mRNA delivery and consequent expression of the encoded polypeptide therein. This is done in addition to testing the validity of the design of the recombinant mRNA construct for NK cell-specific expression as described above, comparing the expression of the construct in presence or absence of the co-receptor expression.

mRNA is obtained by IVT. The template for IVT is a linearized plasmid. The mRNA is capped, either cotranscriptionally or post-transcriptionally, and a PolyA tail is added either enzymatically or by transcription from the template. Typically, the length of the poly A is controlled when encoded by the plasmid, and is less controlled when enzymatically added. An mRNA product comprising an enzymatically added poly A tail may be tailored at best to contain a narrowed range of the number of A-residues. The in vitro transcribed mRNA is thereafter purified. Various modes of mRNA purification can be employed, in some cases the mRNA is purified by more than one method, and more than once, e.g., before and after capping and tailing. In some embodiments, HPLC is used to purify mRNA. In some embodiments, filtration, e.g. reverse filtration or transient flow filtration is used to purify mRNA for large scale purposes. Testing grade small scale mRNA can be purified using commercially available kits.

The recombinant constructs are tested in vitro for efficiency in NK cells, e.g., whether or not expression of the CFP improves an NK cell function. NK cells are electroporated with the CFP constructs, and tested for a functional assay, using NK cells not expressing CFP as a control for the assay. NK cell efficiency are tested using any one or more of the parameters: (i) cytokine release (ii) cell-cell interaction, e.g. engaging target cells such as tumor cells, (iii) degranulation upon contact with target cell, (iv) target cell lysis.

### Pharmaceutical Compositions

Provided herein is a pharmaceutical composition, comprising at least a first therapeutic agent which comprises monocyte or macrophage specific engagers. The monocyte or macrophage specific engagers in the composition may be in the form of peptides or polypeptides or a complex of multiple peptides. The monocyte or macrophage specific engagers may be provided in a composition as purified recombinant proteins. The monocyte or macrophage specific engagers may be provided in a composition as conjugated recombinant proteins, VHH complexes, scFv complexes or nanobodies. The monocyte or macrophage specific engagers may be in the form of a polynucleotide encoding the recombinant monocyte or macrophage specific engagers. In some embodiments, polynucleotide encoding the monocyte or macrophage specific engagers may comprise DNA, mRNA or circRNA or a liposomal composition of any one of these. The liposome is a LNP.

Pharmaceutical compositions can include, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration.

Acceptable carriers, excipients, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

Acceptable carriers are physiologically acceptable to the administered patient and retain the therapeutic properties of the compounds with/in which it is administered. Acceptable carriers and their formulations are generally described in, for example, Remington pharmaceutical Sciences (18^{th} ed. A. Gennaro, Mack Publishing Co., Easton, PA 1990). One example of carrier is physiological saline. A pharmaceutically acceptable carrier is a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Acceptable carriers are compatible with the other ingredients of the formulation and not injurious to a subject to whom it is administered. Nor should an acceptable carrier alter the specific activity of the neoantigens.

In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. Compositions can be formulated to be compatible with a particular route of administration (i.e., systemic or local). Thus, compositions include carriers, diluents, or excipients suitable for administration by various routes.

In some embodiments, a composition can further comprise an acceptable additive in order to improve the stability of immune cells in the composition. Acceptable additives may not alter the specific activity of the immune cells. Examples of acceptable additives include, but are not limited to, a sugar such as mannitol, sorbitol, glucose, xylitol, trehalose, sorbose, sucrose, galactose, dextran, dextrose, fructose, lactose and mixtures thereof. Acceptable additives can be combined with acceptable carriers and/or excipients such as dextrose. Alternatively, examples of acceptable additives include, but are not limited to, a surfactant such as polysorbate 20 or polysorbate 80 to increase stability of the peptide and decrease gelling of the solution. The surfactant can be added to the composition in an amount of 0.01% to 5% of the solution. Addition of such acceptable additives increases the stability and half-life of the composition in storage.

The pharmaceutical composition can be administered, for example, by injection. Compositions for injection include aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, and sodium chloride can be included in the composition. The resulting solutions can be packaged for use as is, or lyophilized; the lyophilized preparation can later be combined with a sterile solution prior to administration. For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as needed. Sterile injectable solutions can be prepared by incorporating an active ingredient in the required amount in an appropriate solvent with one or a combination of ingredients enumerated above, as required, followed by filtered sterilization. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation can be vacuum drying and freeze drying which yields a powder of the active ingredient plus any additional desired ingredient from a previously sterile-filtered solution thereof.

Compositions can be conventionally administered intravenously, such as by injection of a unit dose, for example. For injection, an active ingredient can be in the form of a parenterally acceptable aqueous solution which is substantially pyrogen-free and has suitable pH, isotonicity and stability. One can prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. Additionally, compositions can be administered via aerosolization.

When the compositions are considered for use in medicaments or any of the methods provided herein, it is contemplated that the composition can be substantially free of pyrogens such that the composition will not cause an inflammatory reaction or an unsafe allergic reaction when administered to a human patient. Testing compositions for pyrogens and preparing compositions substantially free of pyrogens are well understood to one or ordinary skill of the art and can be accomplished using commercially available kits.

Acceptable carriers can contain a compound that stabilizes, increases or delays absorption, or increases or delays clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (e.g., Fix (1996) Pharm Res. 13:1760 1764; Samanen (1996) J. Pharm. Pharmacol. 48:119 135; and U.S. Pat. No. 5,391,377).

The compositions can be administered in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots are also variable, but are typified by an initial administration followed by repeated doses at one or more hour intervals by a subsequent injection or other administration. Alternatively, continuous intravenous infusions sufficient to maintain concentrations in the blood are contemplated.

In some embodiments the recombinant polynucleic acid sequence is optimized for expression in human.

### Treatment Methods

Provided herein in one aspect is a method for treating a disease in a subject, comprising administering to the subject the pharmaceutical composition described herein. In one embodiment, the subject is a human subject. In one embodiments, the disease is a cancer.

Cancers include, but are not limited to T cell lymphoma, cutaneous lymphoma, B cell cancer (e.g., multiple myeloma, Waldenstrom's macroglobulinemia), the heavy chain diseases (such as, for example, alpha chain disease, gamma chain disease, and mu chain disease), benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In some aspects, the pharmaceutical composition as described herein comprising a recombinant polynucleic acid and a delivery vehicle can encode any gene of interest that can be expressed in an NK cell, such that the cell can be used to treat a disease that requires, for example an active cytotoxic and cytolytic cell, where the recombinant polynucleic acid is specifically expressed in an NK cell in vivo, which may then target and destroy the disease causing organism or cell, even a “self” cell. NK cells can generally discriminate a diseased cell that is also a “self” cell, from a healthy cell, and target to destroy such “self” diseased cell, but may lose this potential by inhibitory signals in vivo, such as in a tumor environment. Uptake and expression of the recombinant polynucleic acid is designed to recharge such NK cell to attack and destroy the diseased cell, e.g., the cancer cell.

In some embodiments the administration of the off the shelf nucleic acid products may be instantaneous, or may be prepared 1 day, 2 days or 3 days or 4 days or 5 days or 6 days or 7 days or more prior to administration. In some embodiments the recombinant polynucleic acid is prepared for more than a month, more than 6 months, more than a year or several years before use on a subject, and is properly stored for minimum degradation, e.g., at −70° C. The pharmaceutical composition comprising cell, or nucleic acid may be preserved over time from preparation until use in frozen condition. In some embodiments, the pharmaceutical composition may be thawed once. In some embodiments, the pharmaceutical composition may be thawed more than once. In some embodiments, the pharmaceutical composition is stabilized after a freeze-thaw cycle prior administering to the subject. In some embodiments the pharmaceutical composition is tested for final quality control after thawing prior administration.

### EXAMPLES

### Example 1. Generation of NK Cell Specific Receptors for In Vivo Delivery

In this example, recombinant polynucleic acid constructs encoding chimeric fusion protein are designed that can specifically express in an NK cell when administered in vivo. For this purpose, NK cell-specific immune receptors are considered that can exhibit a coreceptor-dependent expression or function specifically in an NK cell, and will not do so in a cell that does not express the co-receptors. A list of such receptors that can be potentially tested for including a domain or fragment thereof in a CFP design that will endow the CFP construct to exhibit NK cell-dependent expression or NK cell dependent function is generated using literature survey. An exemplary partial and non-exhaustive list of such potential immune receptors which can pair with endogenous ITAM motif containing co-receptors are listed in Table 3 below, such that a domain, a portion or a sequence thereof that exhibits such pairing will be incorporated in the CFP construct design discussed above.

TABLE 4
Immune receptors that pair with endogenous ITAM-containing co-
receptors and therefore exhibit NK cell dependent function.

| | Immune | ITAM motif containing |
|---|---|---|
| Immune cell | Receptor | co-receptor |
| Natural Killer Cells; | NKG2D | DAP10 or DAP12 |
| NKT Cells | NKG2C | heterodimer with CD94; |
| | | bind to DAP12 |
| | NKG2E | heterodimer with CD94; |
| | | bind to DAP12 |
| | Ly49D | DAP12 |
| | Ly49H | DAP12 |
| | KIR | DAP12 |
| | NKp46 | CD3zeta or Fcgamma chain |
| | NKp44 | CD3zeta or Fcgamma chain |
| | NKp30 | CD3zeta or Fcgamma chain |

After determination and selection of a sequence of a suitable domain, or fragment thereof from an immune receptor (e.g., as shown in the exemplary Table 3) wherein the sequence is critically responsible for interaction with an endogenous protein in an NK cell such that the expression or function of the protein containing the sequence is dependent on the cell expressing the endogenous protein it binds to, such sequence is incorporated into a CFP test construct. Such that a test construct is a recombinant polynucleic acid, having, other than the sequence as mentioned in the preceding sentence, an extracellular antigen binding domain that is capable of binding to a cancer antigen, e.g., CD5, and suitable intracellular and transmembrane domains, as disclosed throughout this document. The construct is designed using routine molecular cloning technologies.

### Example 2. Assay for Screening Test Constructs

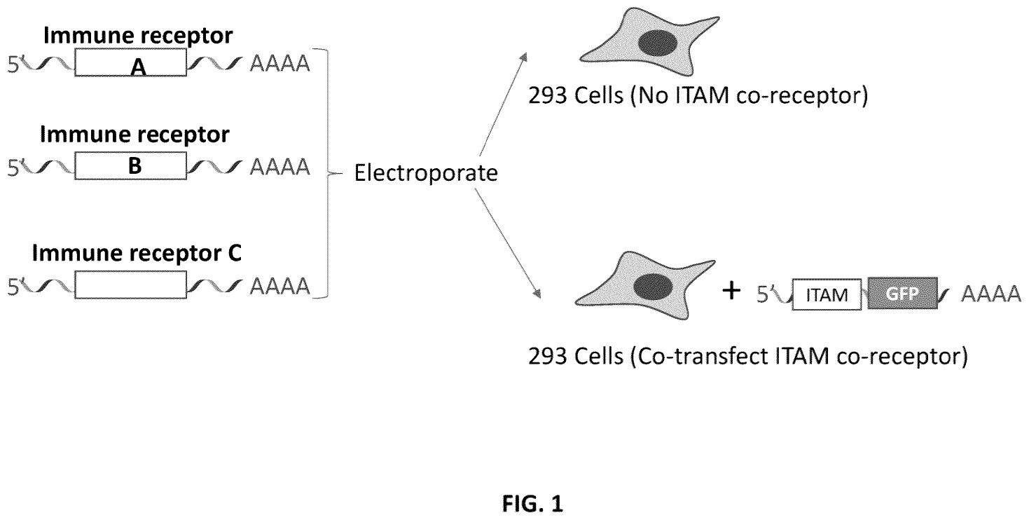

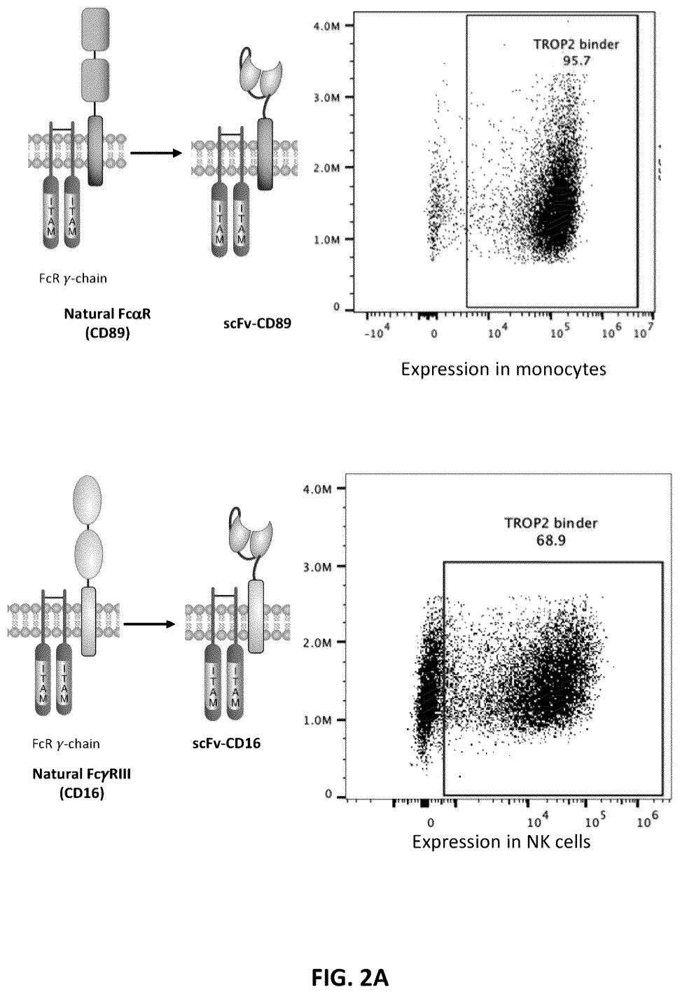

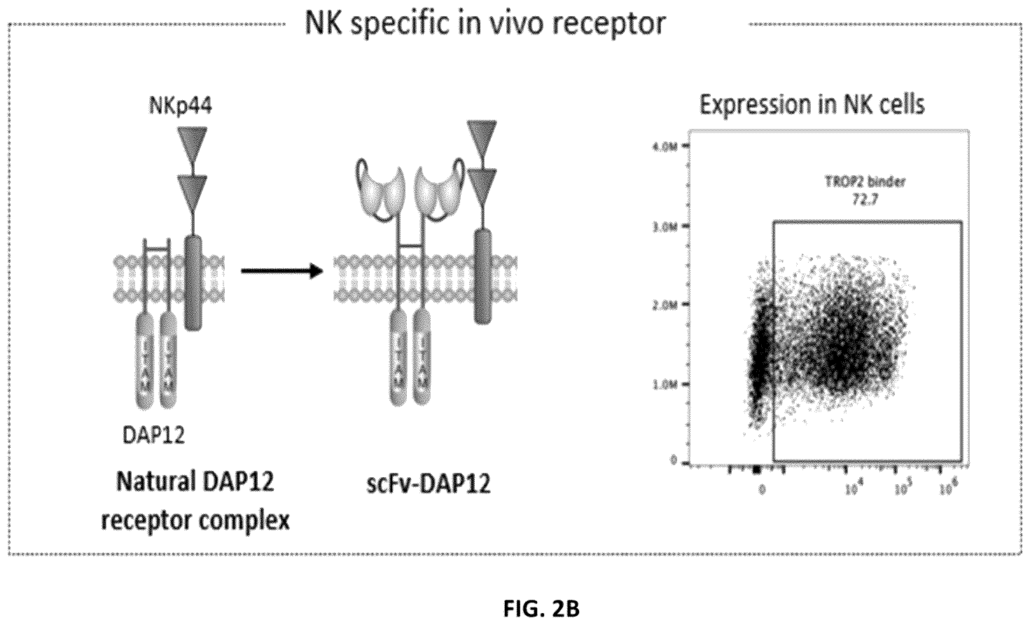

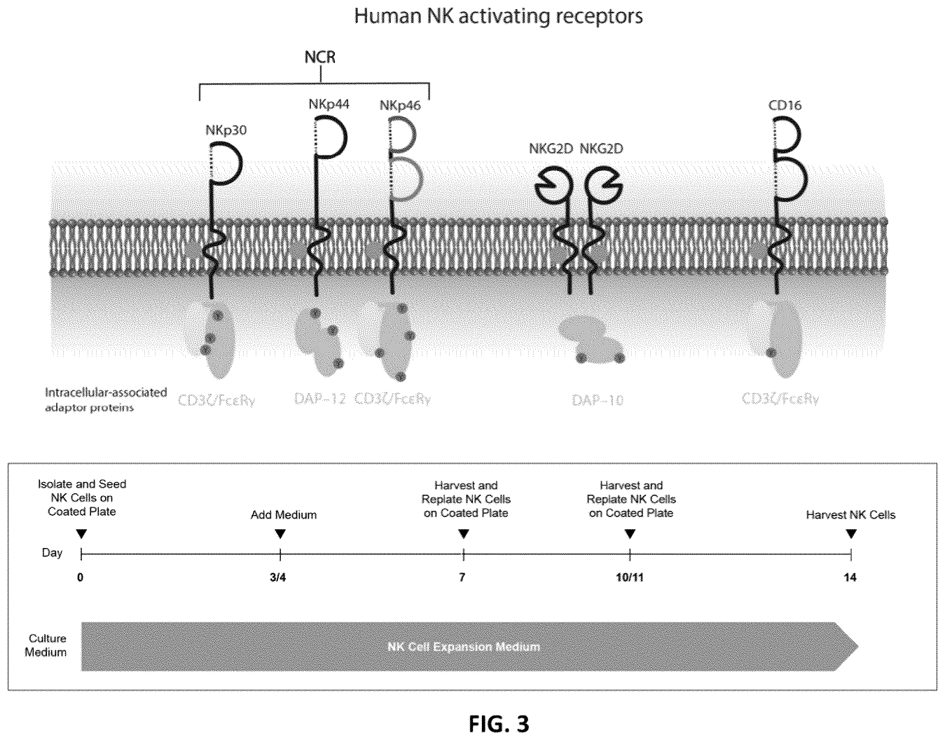

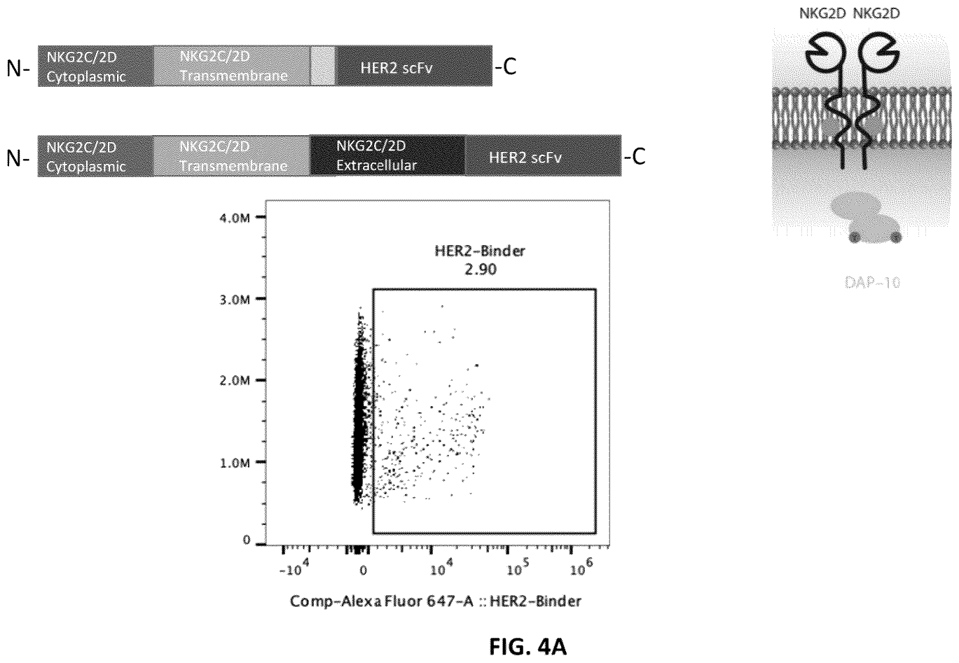

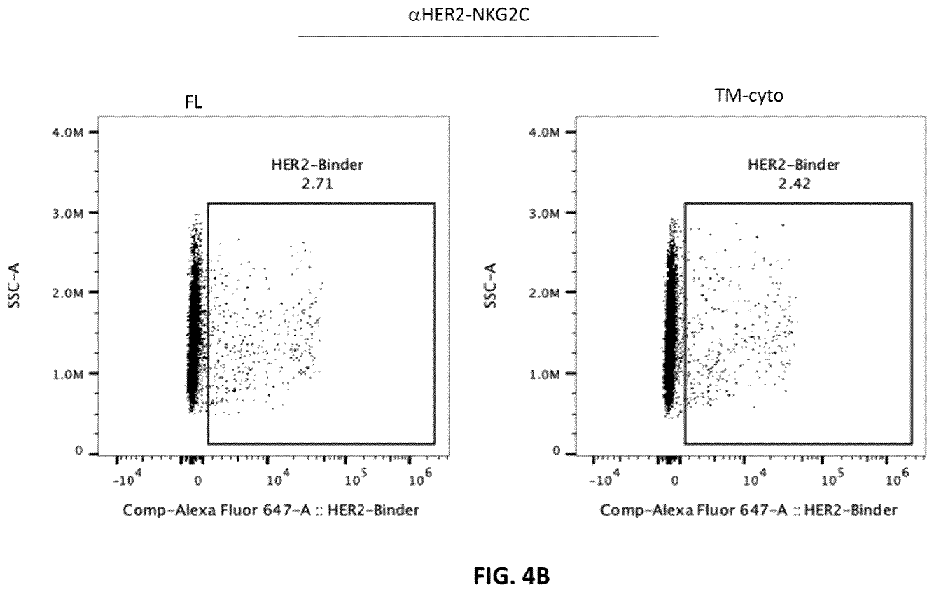

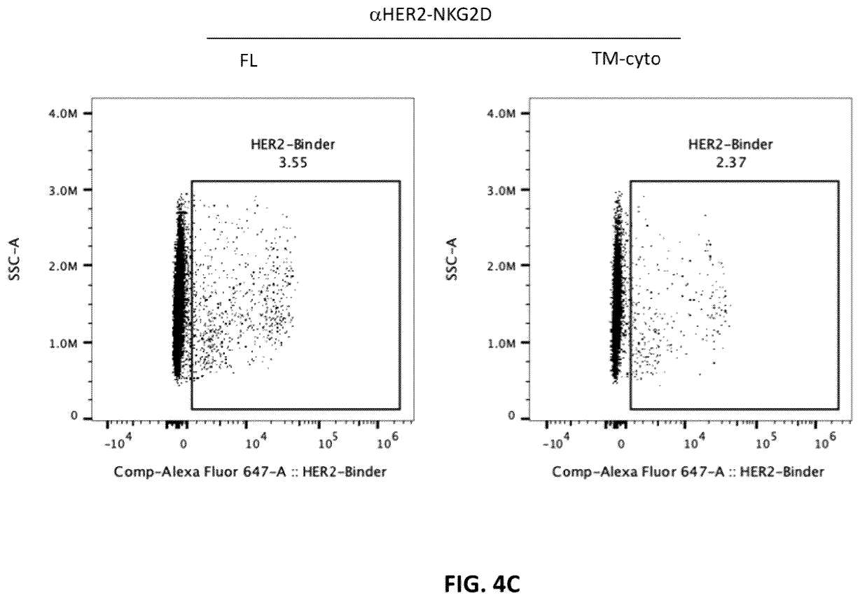

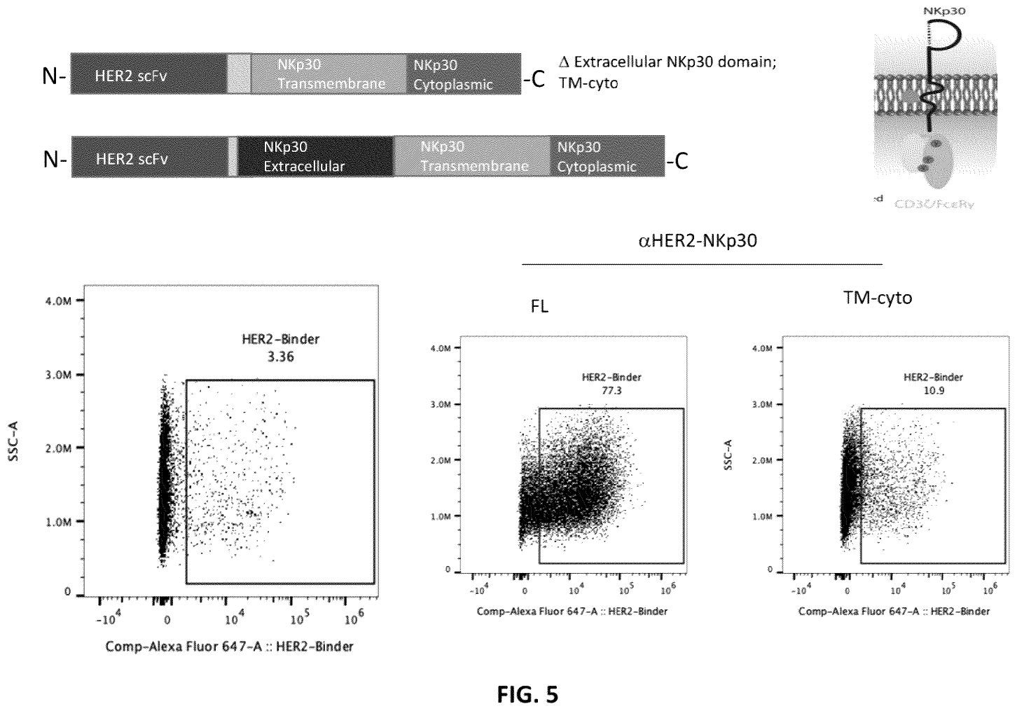

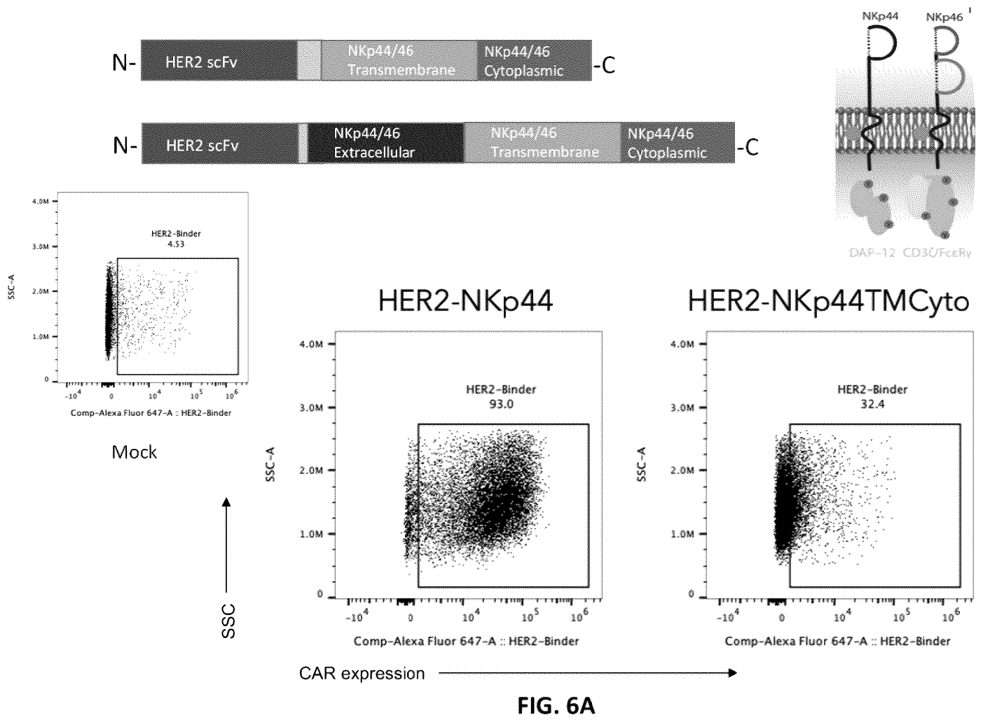

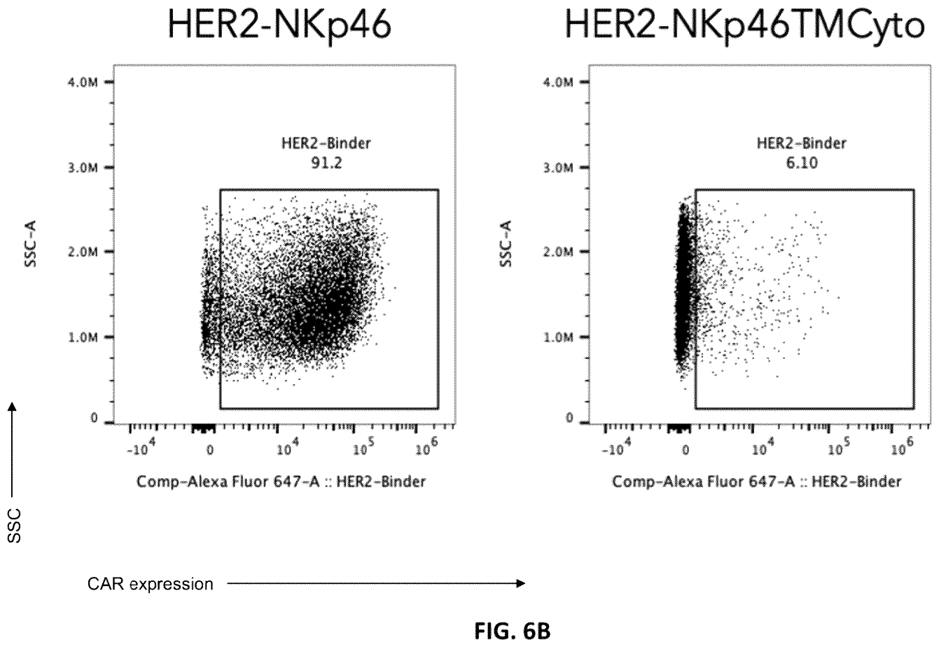

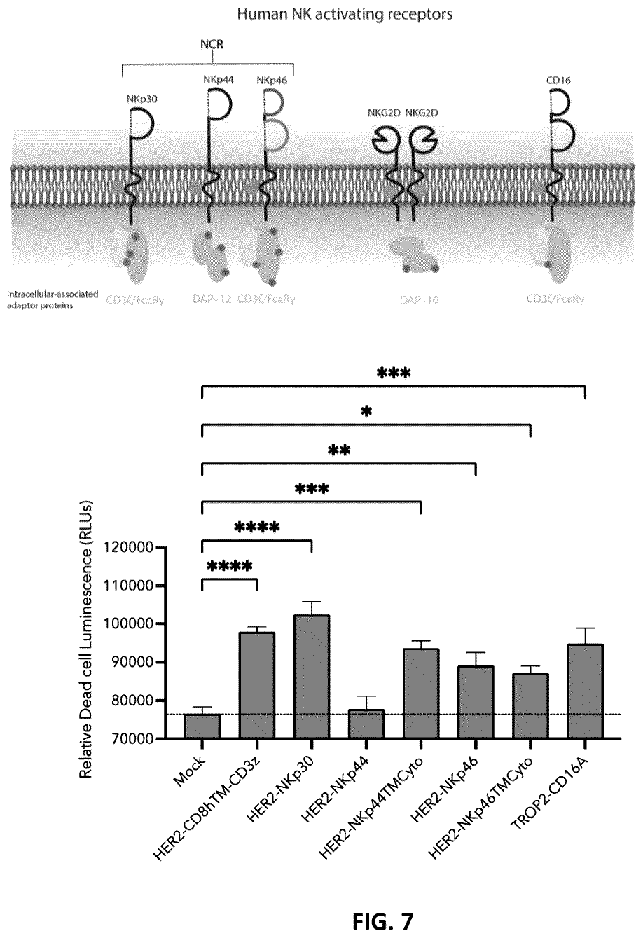

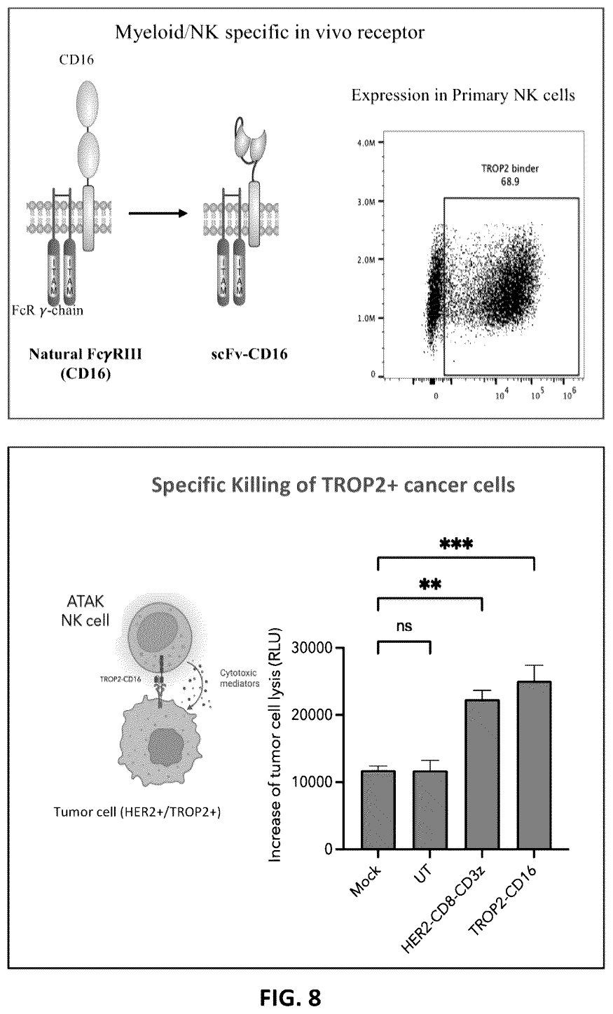

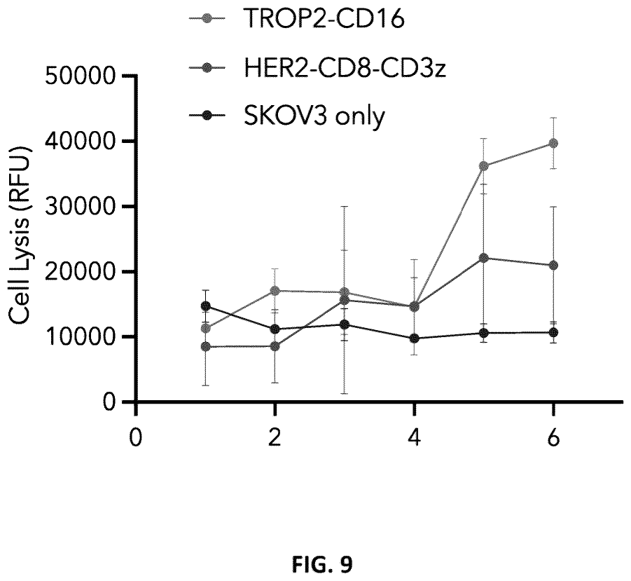

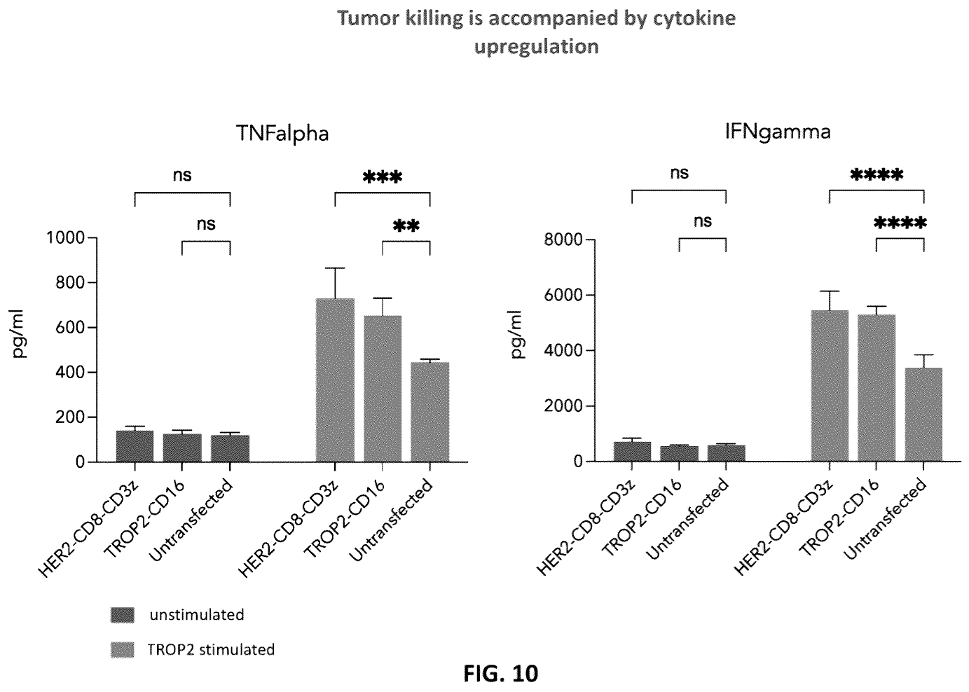

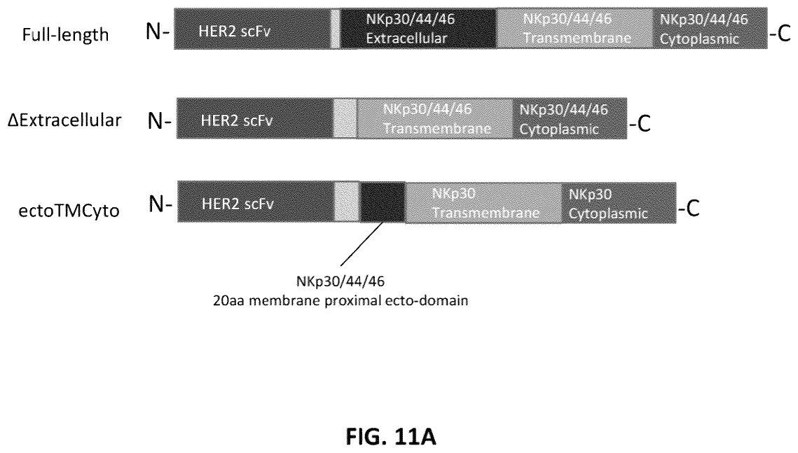

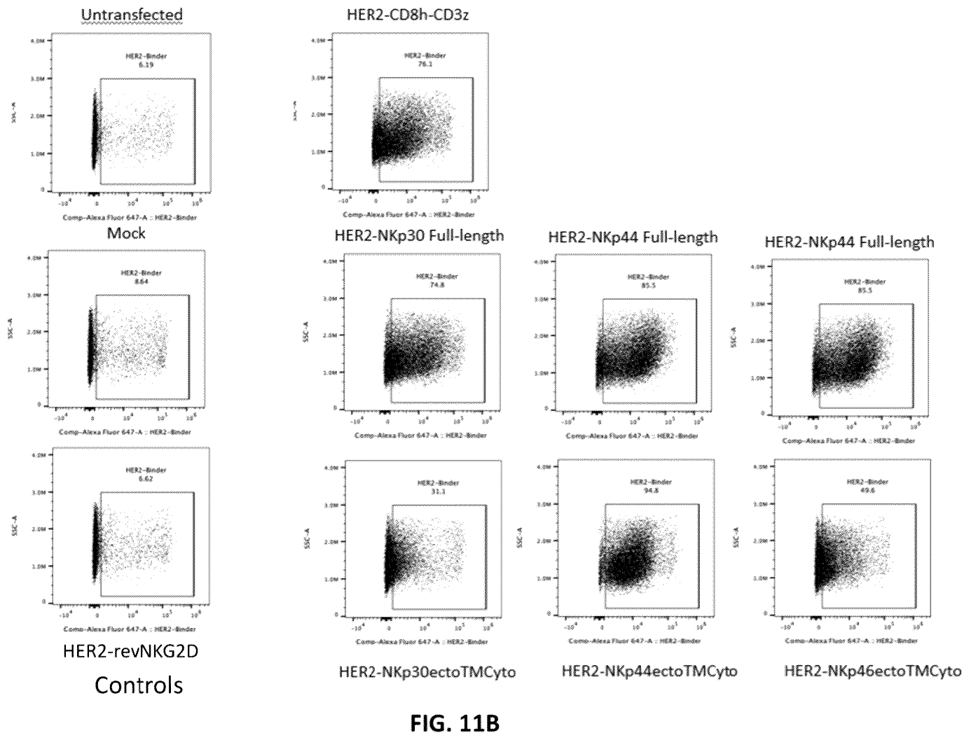

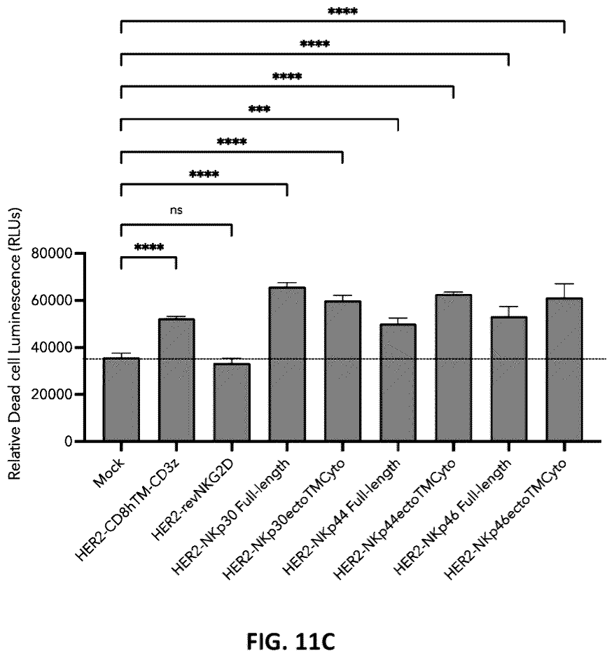

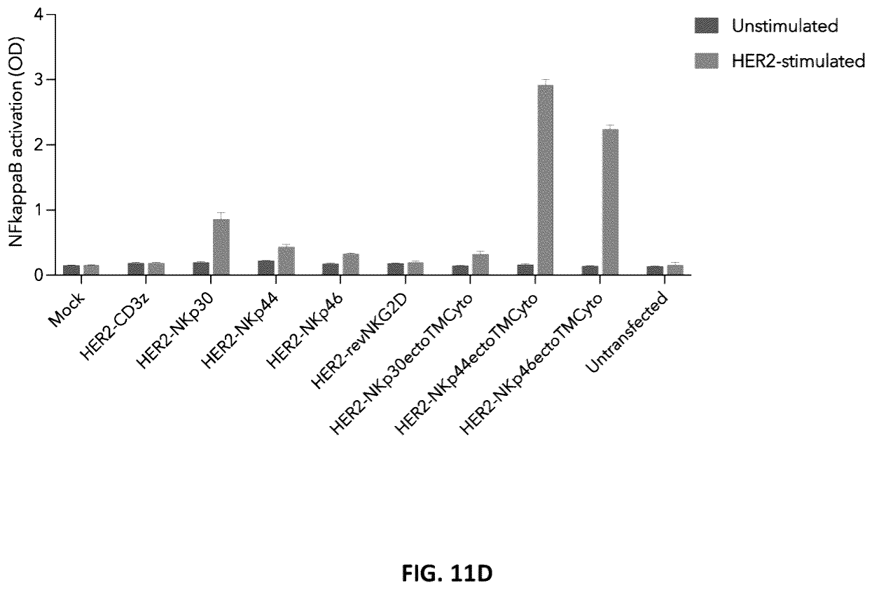

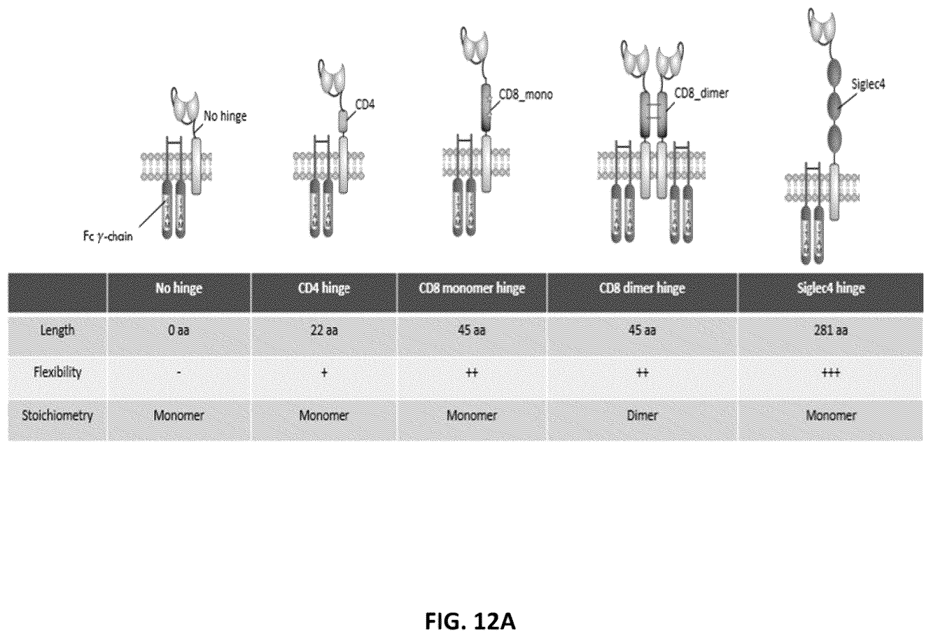

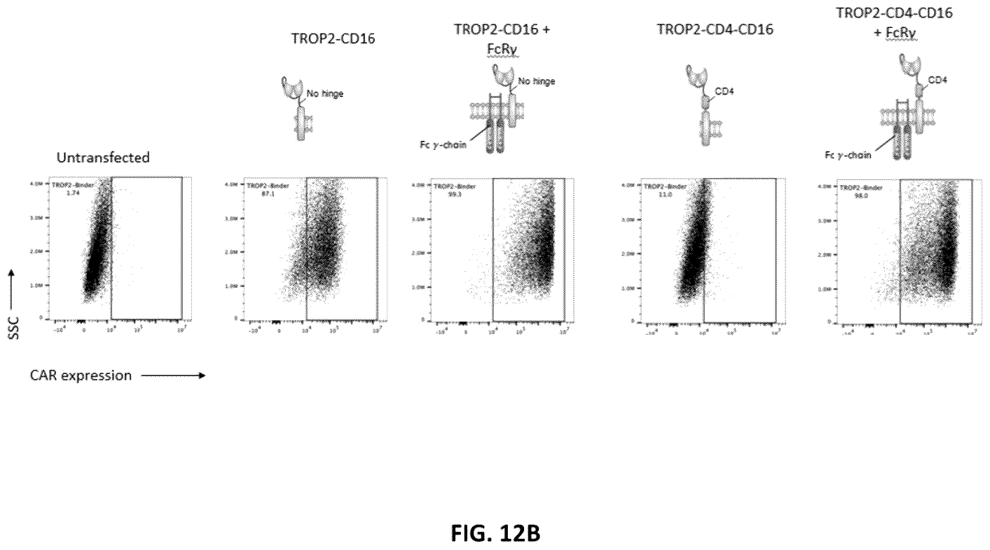

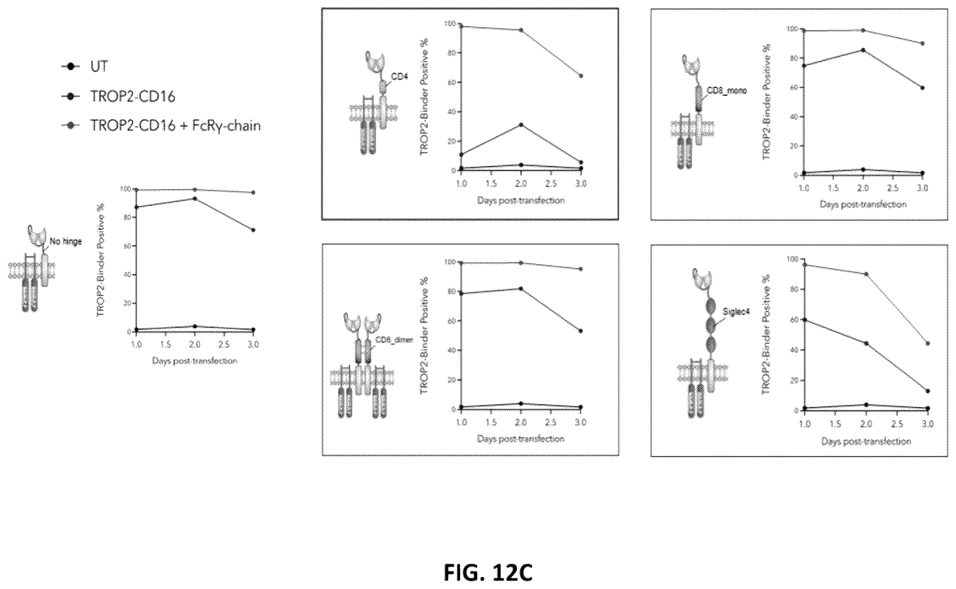

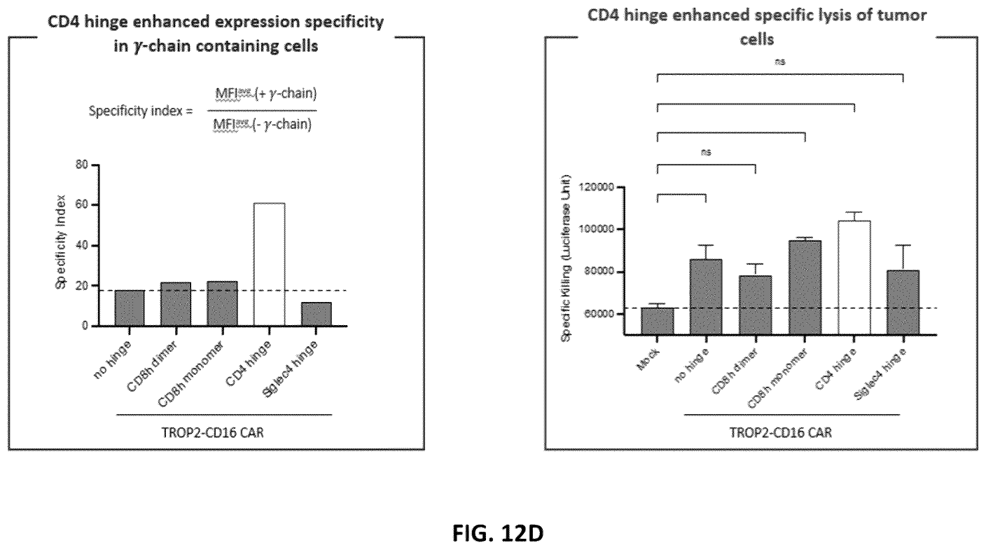

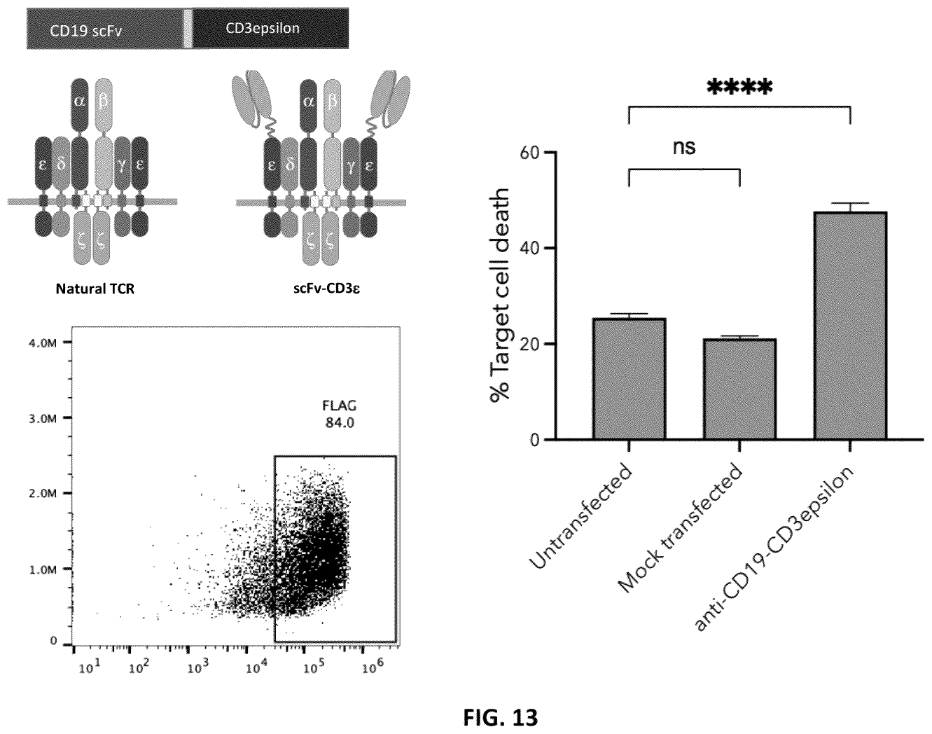

In this example, first, immune receptor constructs are tested for any dependence on the presence of the coreceptor in a cell that does not normally express either the immune receptor or the coreceptor (e.g., an HEK 293 cell) that is known to be endogenously expressed in an NK cell. FIG. 1 shows a schematic diagram of the screening assay for testing the expression dependency of the CFP constructs on ITAM co-receptor. mRNA constructs are generated encoding the different immune receptors, A, B or C as exemplified in the figure. Similarly, GFP-tagged coreceptor mRNA constructs are generated e.g. comprising the coding sequence of the ITAM motif containing co-receptor corresponding to the immune receptor in the same row in Table 3. For each set of immune receptor and coreceptors, HEK 293 cells ae divided into a (i) control group, that are transfected with immune receptor alone, and (ii) an experimental group, transfected with the corresponding ITAM containing coreceptors; and expression of the immune receptors, e.g., A, B or C are assayed by any known immunological methods such as western blot, FACS analysis or ELISA. If expression of the immune receptor is noted in the control set, the expression of the immune receptor is inferred to be not dependent on the corresponding coreceptor, irrespective of the expression in the experimental set, and therefore is rejected. If expression of the immune receptor transfected is absent or below detection level in the control set, but is present in the experimental set, coexpressed with GFP, then the results indicate that the expression of the immune receptor is dependent on the expression of the coreceptor, and is selected for generating a CFP construct using the immune receptor.

Finally, test CFP constructs that are generated as per the method of Example 1, are screened for expression and function in a cell that expresses the co-receptor using the similar method as above.

Exemplary test recombinant construct is cloned in a suitable commercially available vector for in vitro transcription (IVT). An mRNA is generated by IVT, capped and poly-A tailed and purified according to standard protocols and is electroporated into HEK 293 cells, with or without co-transfection of the corresponding ITAM co-receptor having a GFP marker tag. The ITAM-GFP may also be delivered in a cell as an mRNA. Successful expression of the CFP in the cells co-transfected with the coreceptor is verified alongside absence of the same in the cell that does not express the coreceptor.

The screens can be run in parallel in multiwell analysis and designed into high throughput assays as is suitable for faster and efficient readout. Functional assays as described elsewhere in the disclosure are employed to test the efficacy of the recombinant polynucleic acid in potentiating an NK cell for targeted cytotoxicity.

### Example 3. Generation of NK Cell-Specific CFPs

Several NK cell specific constructs are generated and are being tested for expression in NK cells. Each sequence was cloned and expressed. Although DNA sequences are shown, one of skill in the art can readily interpret and obtain the mRNA sequence therefrom. The polynucleic acid sequences are provided in Table 5:

TABLE 5
DNA sequences encoding exemplary NK-specific
CFPs and their domains and component structures

| Construct | | |
|---|---|---|
| name | Domain | Sequences |
| TROP2- | Full length | ATGTGGCTGCAGTCTCTGCTGCTGCTGGGAACAGTGGCCTGTA |
| DAP12 | | GCATCTCTCAAGTGCAGCTGCAGCAGAGCGGCAGCGAGCTGA |
| | | AAAAGCCCGGAGCCAGCGTGAAAGTGTCCTGCAAGGCTTCTG |
| | | GCTACACATTCACCAATTACGGCATGAACTGGGTCAAGCAGG |
| | | CCCCTGGACAGGGCCTGAAGTGGATGGGCTGGATCAACACCT |
| | | ACACCGGCGAACCTACATACACAGATGACTTCAAGGGCAGAT |
| | | TCGCCTTCAGCCTGGACACCAGCGTGTCCACCGCTTATCTGCA |
| | | GATCAGCAGCCTGAAGGCCGACGATACCGCCGTGTACTTTTG |
| | | TGCCCGGGGCGGATTTGGCTCTAGCTACTGGTACTTCGACGTG |
| | | TGGGGCCAGGGCAGCCTGGTGACCGTGTCTAGCGGAGGCGGA |
| | | GGATCAGGTGGCGGTGGATCTGGCGGTGGTGGCTCTGACATC |
| | | CAGCTGACACAGAGCCCATCTAGCCTGAGCGCTAGCGTGGGC |
| | | GACAGAGTGTCTATTACCTGTAAAGCTTCTCAGGACGTGTCCA |
| | | TCGCCGTCGCCTGGTATCAGCAGAAGCCCGGCAAGGCCCCTA |
| | | AGCTGCTGATCTACAGCGCCTCCTACAGATACACCGGCGTGC |
| | | CCGATAGATTCAGCGGAAGCGGCAGCGGAACAGATTTTACCC |
| | | TGACAATCAGCAGCCTGCAGCCTGAGGACTTCGCCGTGTACT |
| | | ACTGCCAGCAACACTACATCACCCCTCTGACCTTCGGCGCCG |
| | | GCACCAAGGTGGAAATCAAGCGGTCAGGCGGCGGAGGAGCG |
| | | GCCGCAGGCAGCCTTCGGCCTGTTCAAGCACAAGCGCAGAGT |
| | | GACTGCTCTTGTAGCACGGTTTCACCTGGCGTATTGGCCGGTA |
| | | TTGTAATGGGGGACCTTGTACTCACGGTTCTCATAGCTCTTGC |
| | | TGTCTATTTTCTCGGACGACTGGTCCCACGGGGACGAGGGGC |
| | | AGCAGAAGCTGCTACACGAAAACAGAGGATTACAGAGACGG |
| | | AGAGTCCCTACCAAGAACTCCAGGGGCAGAGAAGTGATGTCT |
| | | ATTCTGACCTTAACACACAAAGACCATACTATAAATGA (SEQ |
| | | ID NO: 251) |
| | GM-CSF | ATGTGGCTGCAGTCTCTGCTGCTGCTGGGAACAGTGGCCTGTA |
| | signal peptide | GCATCTCT (SEQ ID NO: 252) |
| | TROP2 scFv | CAAGTGCAGCTGCAGCAGAGCGGCAGCGAGCTGAAAAAGCC |
| | | CGGAGCCAGCGTGAAAGTGTCCTGCAAGGCTTCTGGCTACAC |
| | | ATTCACCAATTACGGCATGAACTGGGTCAAGCAGGCCCCTGG |
| | | ACAGGGCCTGAAGTGGATGGGCTGGATCAACACCTACACCGG |
| | | CGAACCTACATACACAGATGACTTCAAGGGCAGATTCGCCTT |
| | | CAGCCTGGACACCAGCGTGTCCACCGCTTATCTGCAGATCAG |
| | | CAGCCTGAAGGCCGACGATACCGCCGTGTACTTTTGTGCCCG |
| | | GGGCGGATTTGGCTCTAGCTACTGGTACTTCGACGTGTGGGG |
| | | CCAGGGCAGCCTGGTGACCGTGTCTAGCGGAGGCGGAGGATC |
| | | AGGTGGCGGTGGATCTGGCGGTGGTGGCTCTGACATCCAGCT |
| | | GACACAGAGCCCATCTAGCCTGAGCGCTAGCGTGGGCGACAG |
| | | AGTGTCTATTACCTGTAAAGCTTCTCAGGACGTGTCCATCGCC |
| | | GTCGCCTGGTATCAGCAGAAGCCCGGCAAGGCCCCTAAGCTG |
| | | CTGATCTACAGCGCCTCCTACAGATACACCGGCGTGCCCGAT |
| | | AGATTCAGCGGAAGCGGCAGCGGAACAGATTTTACCCTGACA |
| | | ATCAGCAGCCTGCAGCCTGAGGACTTCGCCGTGTACTACTGC |
| | | CAGCAACACTACATCACCCCTCTGACCTTCGGCGCCGGCACC |
| | | AAGGTGGAAATCAAGCGG (SEQ ID NO: 253) |
| | Linker | TCAGGCGGCGGAGGAGCGGCCGCAGGCAGC (SEQ ID NO: 254) |
| | DAP12 | CTTCGGCCTGTTCAAGCACAAGCGCAGAGTGACTGCTCTTGTA |
| | | GCACGGTTTCACCTGGCGTATTGGCCGGTATTGTAATGGGGG |
| | | ACCTTGTACTCACGGTTCTCATAGCTCTTGCTGTCTATTTTCTC |
| | | GGACGACTGGTCCCACGGGGACGAGGGGCAGCAGAAGCTGC |
| | | TACACGAAAACAGAGGATTACAGAGACGGAGAGTCCCTACC |
| | | AAGAACTCCAGGGGCAGAGAAGTGATGTCTATTCTGACCTTA |
| | | ACACACAAAGACCATACTATAAATGA (SEQ ID NO: 1) |
| TROP2- | Full length | ATGTGGCTGCAGTCTCTGCTGCTGCTGGGAACAGTGGCCTGTA |
| CD16A | | GCATCTCTCAAGTGCAGCTGCAGCAGAGCGGCAGCGAGCTGA |
| | | AAAAGCCCGGAGCCAGCGTGAAAGTGTCCTGCAAGGCTTCTG |
| | | GCTACACATTCACCAATTACGGCATGAACTGGGTCAAGCAGG |
| | | CCCCTGGACAGGGCCTGAAGTGGATGGGCTGGATCAACACCT |
| | | ACACCGGCGAACCTACATACACAGATGACTTCAAGGGCAGAT |
| | | TCGCCTTCAGCCTGGACACCAGCGTGTCCACCGCTTATCTGCA |
| | | GATCAGCAGCCTGAAGGCCGACGATACCGCCGTGTACTTTTG |
| | | TGCCCGGGGCGGATTTGGCTCTAGCTACTGGTACTTCGACGTG |
| | | TGGGGCCAGGGCAGCCTGGTGACCGTGTCTAGCGGAGGCGGA |
| | | GGATCAGGTGGCGGTGGATCTGGCGGTGGTGGCTCTGACATC |
| | | CAGCTGACACAGAGCCCATCTAGCCTGAGCGCTAGCGTGGGC |
| | | GACAGAGTGTCTATTACCTGTAAAGCTTCTCAGGACGTGTCCA |
| | | TCGCCGTCGCCTGGTATCAGCAGAAGCCCGGCAAGGCCCCTA |
| | | AGCTGCTGATCTACAGCGCCTCCTACAGATACACCGGCGTGC |
| | | CCGATAGATTCAGCGGAAGCGGCAGCGGAACAGATTTTACCC |
| | | TGACAATCAGCAGCCTGCAGCCTGAGGACTTCGCCGTGTACT |
| | | ACTGCCAGCAACACTACATCACCCCTCTGACCTTCGGCGCCG |
| | | GCACCAAGGTGGAAATCAAGCGGTCAGGCGGCGGAGGAGCG |
| | | GCCGCAGGAAGTGGTCTGGCCGTAAGTACCATATCCAGCTTT |
| | | TTTCCGCCAGGATATCAGGTTTCCTTTTGTTTGGTCATGGTACT |
| | | TCTCTTTGCGGTAGACACTGGTCTCTATTTTAGTGTCAAAACT |
| | | AATATACGCTCCTCCACGAGGGATTGGAAGGACCATAAGTTC |
| | | AAATGGAGGAAGGACCCGCAGGACAAATGA (SEQ ID NO: 255) |
| | GM-CSF | ATGTGGCTGCAGTCTCTGCTGCTGCTGGGAACAGTGGCCTGTA |
| | signal peptide | GCATCTCT (SEQ ID NO: 252) |
| | TROP2 scFv | CAAGTGCAGCTGCAGCAGAGCGGCAGCGAGCTGAAAAAGCC |
| | | CGGAGCCAGCGTGAAAGTGTCCTGCAAGGCTTCTGGCTACAC |
| | | ATTCACCAATTACGGCATGAACTGGGTCAAGCAGGCCCCTGG |
| | | ACAGGGCCTGAAGTGGATGGGCTGGATCAACACCTACACCGG |
| | | CGAACCTACATACACAGATGACTTCAAGGGCAGATTCGCCTT |
| | | CAGCCTGGACACCAGCGTGTCCACCGCTTATCTGCAGATCAG |
| | | CAGCCTGAAGGCCGACGATACCGCCGTGTACTTTTGTGCCCG |
| | | GGGCGGATTTGGCTCTAGCTACTGGTACTTCGACGTGTGGGG |
| | | CCAGGGCAGCCTGGTGACCGTGTCTAGCGGAGGCGGAGGATC |
| | | AGGTGGCGGTGGATCTGGCGGTGGTGGCTCTGACATCCAGCT |
| | | GACACAGAGCCCATCTAGCCTGAGCGCTAGCGTGGGCGACAG |
| | | AGTGTCTATTACCTGTAAAGCTTCTCAGGACGTGTCCATCGCC |
| | | GTCGCCTGGTATCAGCAGAAGCCCGGCAAGGCCCCTAAGCTG |
| | | CTGATCTACAGCGCCTCCTACAGATACACCGGCGTGCCCGAT |
| | | AGATTCAGCGGAAGCGGCAGCGGAACAGATTTTACCCTGACA |
| | | ATCAGCAGCCTGCAGCCTGAGGACTTCGCCGTGTACTACTGC |
| | | CAGCAACACTACATCACCCCTCTGACCTTCGGCGCCGGCACC |
| | | AAGGTGGAAATCAAGCGG (SEQ ID NO: 253) |
| | Linker | TCAGGCGGCGGAGGAGCGGCCGCAGGAAGT (SEQ ID NO: 256) |
| | CD16A | GGTCTGGCCGTAAGTACCATATCCAGCTTTTTTCCGCCAGGAT |
| | | ATCAGGTTTCCTTTTGTTTGGTCATGGTACTTCTCTTTGCGGTA |
| | | GACACTGGTCTCTATTTTAGTGTCAAAACTAATATACGCTCCT |
| | | CCACGAGGGATTGGAAGGACCATAAGTTCAAATGGAGGAAG |
| | | GACCCGCAGGACAAATGA (SEQ ID NO: 2) |
| TROP2- | Full length | ATGTGGCTGCAGTCTCTGCTGCTGCTGGGAACAGTGGCCTGTA |
| CD8h- | | GCATCTCTCAAGTGCAGCTGCAGCAGAGCGGCAGCGAGCTGA |
| CD16A | | AAAAGCCCGGAGCCAGCGTGAAAGTGTCCTGCAAGGCTTCTG |
| | | GCTACACATTCACCAATTACGGCATGAACTGGGTCAAGCAGG |
| | | CCCCTGGACAGGGCCTGAAGTGGATGGGCTGGATCAACACCT |
| | | ACACCGGCGAACCTACATACACAGATGACTTCAAGGGCAGAT |
| | | TCGCCTTCAGCCTGGACACCAGCGTGTCCACCGCTTATCTGCA |
| | | GATCAGCAGCCTGAAGGCCGACGATACCGCCGTGTACTTTTG |
| | | TGCCCGGGGCGGATTTGGCTCTAGCTACTGGTACTTCGACGTG |
| | | TGGGGCCAGGGCAGCCTGGTGACCGTGTCTAGCGGAGGCGGA |
| | | GGATCAGGTGGCGGTGGATCTGGCGGTGGTGGCTCTGACATC |
| | | CAGCTGACACAGAGCCCATCTAGCCTGAGCGCTAGCGTGGGC |
| | | GACAGAGTGTCTATTACCTGTAAAGCTTCTCAGGACGTGTCCA |
| | | TCGCCGTCGCCTGGTATCAGCAGAAGCCCGGCAAGGCCCCTA |
| | | AGCTGCTGATCTACAGCGCCTCCTACAGATACACCGGCGTGC |
| | | CCGATAGATTCAGCGGAAGCGGCAGCGGAACAGATTTTACCC |
| | | TGACAATCAGCAGCCTGCAGCCTGAGGACTTCGCCGTGTACT |
| | | ACTGCCAGCAACACTACATCACCCCTCTGACCTTCGGCGCCG |
| | | GCACCAAGGTGGAAATCAAGCGGTCAGGCGGCGGAGGAGCG |
| | | GCCGCAACTACTACTCCAGCTCCAAGGCCTCCCACGCCAGCT |
| | | CCCACTATTGCTTCTCAACCGTTGTCACTGCGACCAGAGGCCT |
| | | GTAGACCTGCAGCTGGAGGCGCTGTTCACACAAGGGGTCTCG |
| | | ATTTTGCGTGTGACGGATATCAGGTTTCCTTTTGTTTGGTCAT |
| | | GGTACTTCTCTTTGCGGTAGACACTGGTCTCTATTTTAGTGTC |
| | | AAAACTAATATACGCTCCTCCACGAGGGATTGGAAGGACCAT |
| | | AAGTTCAAATGGAGGAAGGACCCGCAGGACAAATGA (SEQ ID |
| | | NO: 257) |
| | GM-CSF | ATGTGGCTGCAGTCTCTGCTGCTGCTGGGAACAGTGGCCTGTA |
| | signal peptide | GCATCTCT (SEQ ID NO: 252) |
| | TROP2 scFv | CAAGTGCAGCTGCAGCAGAGCGGCAGCGAGCTGAAAAAGCC |
| | | CGGAGCCAGCGTGAAAGTGTCCTGCAAGGCTTCTGGCTACAC |
| | | ATTCACCAATTACGGCATGAACTGGGTCAAGCAGGCCCCTGG |
| | | ACAGGGCCTGAAGTGGATGGGCTGGATCAACACCTACACCGG |
| | | CGAACCTACATACACAGATGACTTCAAGGGCAGATTCGCCTT |
| | | CAGCCTGGACACCAGCGTGTCCACCGCTTATCTGCAGATCAG |
| | | CAGCCTGAAGGCCGACGATACCGCCGTGTACTTTTGTGCCCG |
| | | GGGCGGATTTGGCTCTAGCTACTGGTACTTCGACGTGTGGGG |
| | | CCAGGGCAGCCTGGTGACCGTGTCTAGCGGAGGCGGAGGATC |
| | | AGGTGGCGGTGGATCTGGCGGTGGTGGCTCTGACATCCAGCT |
| | | GACACAGAGCCCATCTAGCCTGAGCGCTAGCGTGGGCGACAG |
| | | AGTGTCTATTACCTGTAAAGCTTCTCAGGACGTGTCCATCGCC |
| | | GTCGCCTGGTATCAGCAGAAGCCCGGCAAGGCCCCTAAGCTG |
| | | CTGATCTACAGCGCCTCCTACAGATACACCGGCGTGCCCGAT |
| | | AGATTCAGCGGAAGCGGCAGCGGAACAGATTTTACCCTGACA |
| | | ATCAGCAGCCTGCAGCCTGAGGACTTCGCCGTGTACTACTGC |
| | | CAGCAACACTACATCACCCCTCTGACCTTCGGCGCCGGCACC |
| | | AAGGTGGAAATCAAGCGG (SEQ ID NO: 253) |
| | Linker | TCAGGCGGCGGAGGAGCGGCCGCA (SEQ ID NO: 258) |
| | CD8a hinge | ACTACTACTCCAGCTCCAAGGCCTCCCACGCCAGCTCCCACTA |
| | | TTGCTTCTCAACCGTTGTCACTGCGACCAGAGGCCTGTAGACC |
| | | TGCAGCTGGAGGCGCTGTTCACACAAGGGGTCTCGATTTTGC |
| | | GTGTGAC (SEQ ID NO: 3) |
| | CD16A | GGATATCAGGTTTCCTTTTGTTTGGTCATGGTACTTCTCTTTGC |
| | | GGTAGACACTGGTCTCTATTTTAGTGTCAAAACTAATATACGC |
| | | TCCTCCACGAGGGATTGGAAGGACCATAAGTTCAAATGGAGG |
| | | AAGGACCCGCAGGACAAATGA (SEQ ID NO: 259) |
| TROP2- | Full length | ATGTGGCTGCAGTCTCTGCTGCTGCTGGGAACAGTGGCCTGTA |
| CD8hCS | | GCATCTCTCAAGTGCAGCTGCAGCAGAGCGGCAGCGAGCTGA |
| mut- | | AAAAGCCCGGAGCCAGCGTGAAAGTGTCCTGCAAGGCTTCTG |
| CD16A | | GCTACACATTCACCAATTACGGCATGAACTGGGTCAAGCAGG |
| | | CCCCTGGACAGGGCCTGAAGTGGATGGGCTGGATCAACACCT |
| | | ACACCGGCGAACCTACATACACAGATGACTTCAAGGGCAGAT |
| | | TCGCCTTCAGCCTGGACACCAGCGTGTCCACCGCTTATCTGCA |
| | | GATCAGCAGCCTGAAGGCCGACGATACCGCCGTGTACTTTTG |
| | | TGCCCGGGGCGGATTTGGCTCTAGCTACTGGTACTTCGACGTG |
| | | TGGGGCCAGGGCAGCCTGGTGACCGTGTCTAGCGGAGGCGGA |
| | | GGATCAGGTGGCGGTGGATCTGGCGGTGGTGGCTCTGACATC |
| | | CAGCTGACACAGAGCCCATCTAGCCTGAGCGCTAGCGTGGGC |
| | | GACAGAGTGTCTATTACCTGTAAAGCTTCTCAGGACGTGTCCA |
| | | TCGCCGTCGCCTGGTATCAGCAGAAGCCCGGCAAGGCCCCTA |
| | | AGCTGCTGATCTACAGCGCCTCCTACAGATACACCGGCGTGC |
| | | CCGATAGATTCAGCGGAAGCGGCAGCGGAACAGATTTTACCC |
| | | TGACAATCAGCAGCCTGCAGCCTGAGGACTTCGCCGTGTACT |
| | | ACTGCCAGCAACACTACATCACCCCTCTGACCTTCGGCGCCG |
| | | GCACCAAGGTGGAAATCAAGCGGTCAGGCGGCGGAGGAGCG |
| | | GCCGCAACTACTACTCCAGCTCCAAGGCCTCCCACGCCAGCT |
| | | CCCACTATTGCTTCTCAACCGTTGTCACTGCGACCAGAGGCCA |
| | | GCAGACCTGCAGCTGGAGGCGCTGTTCACACAAGGGGTCTCG |
| | | ATTTTGCGAGCGACGGATATCAGGTTTCCTTTTGTTTGGTCAT |
| | | GGTACTTCTCTTTGCGGTAGACACTGGTCTCTATTTTAGTGTC |
| | | AAAACTAATATACGCTCCTCCACGAGGGATTGGAAGGACCAT |
| | | AAGTTCAAATGGAGGAAGGACCCGCAGGACAAATGA (SEQ ID |
| | | NO: 260) |
| | GM-CSF | ATGTGGCTGCAGTCTCTGCTGCTGCTGGGAACAGTGGCCTGTA |
| | signal peptide | GCATCTCT (SEQ ID NO: 252) |
| | TROP2 scFv | CAAGTGCAGCTGCAGCAGAGCGGCAGCGAGCTGAAAAAGCC |
| | | CGGAGCCAGCGTGAAAGTGTCCTGCAAGGCTTCTGGCTACAC |
| | | ATTCACCAATTACGGCATGAACTGGGTCAAGCAGGCCCCTGG |
| | | ACAGGGCCTGAAGTGGATGGGCTGGATCAACACCTACACCGG |
| | | CGAACCTACATACACAGATGACTTCAAGGGCAGATTCGCCTT |
| | | CAGCCTGGACACCAGCGTGTCCACCGCTTATCTGCAGATCAG |
| | | CAGCCTGAAGGCCGACGATACCGCCGTGTACTTTTGTGCCCG |
| | | GGGCGGATTTGGCTCTAGCTACTGGTACTTCGACGTGTGGGG |
| | | CCAGGGCAGCCTGGTGACCGTGTCTAGCGGAGGCGGAGGATC |
| | | AGGTGGCGGTGGATCTGGCGGTGGTGGCTCTGACATCCAGCT |
| | | GACACAGAGCCCATCTAGCCTGAGCGCTAGCGTGGGCGACAG |
| | | AGTGTCTATTACCTGTAAAGCTTCTCAGGACGTGTCCATCGCC |
| | | GTCGCCTGGTATCAGCAGAAGCCCGGCAAGGCCCCTAAGCTG |
| | | CTGATCTACAGCGCCTCCTACAGATACACCGGCGTGCCCGAT |
| | | AGATTCAGCGGAAGCGGCAGCGGAACAGATTTTACCCTGACA |
| | | ATCAGCAGCCTGCAGCCTGAGGACTTCGCCGTGTACTACTGC |
| | | CAGCAACACTACATCACCCCTCTGACCTTCGGCGCCGGCACC |
| | | AAGGTGGAAATCAAGCGG (SEQ ID NO: 253) |
| | Linker | TCAGGCGGCGGAGGAGCGGCCGCA (SEQ ID NO: 258) |
| | CD8aCS | ACTACTACTCCAGCTCCAAGGCCTCCCACGCCAGCTCCCACTA |
| | mut hinge | TTGCTTCTCAACCGTTGTCACTGCGACCAGAGGCCAGCAGAC |
| | | CTGCAGCTGGAGGCGCTGTTCACACAAGGGGTCTCGATTTTG |
| | | CGAGCGAC (SEQ ID NO: 261) |
| | CD16A | GGATATCAGGTTTCCTTTTGTTTGGTCATGGTACTTCTCTTTGC |
| | | GGTAGACACTGGTCTCTATTTTAGTGTCAAAACTAATATACGC |
| | | TCCTCCACGAGGGATTGGAAGGACCATAAGTTCAAATGGAGG |
| | | AAGGACCCGCAGGACAAATGA (SEQ ID NO: 259) |
| TROP2- | Full length | ATGTGGCTGCAGTCTCTGCTGCTGCTGGGAACAGTGGCCTGTA |
| CD4H- | | GCATCTCTCAAGTGCAGCTGCAGCAGAGCGGCAGCGAGCTGA |
| CD16A | | AAAAGCCCGGAGCCAGCGTGAAAGTGTCCTGCAAGGCTTCTG |
| | | GCTACACATTCACCAATTACGGCATGAACTGGGTCAAGCAGG |
| | | CCCCTGGACAGGGCCTGAAGTGGATGGGCTGGATCAACACCT |
| | | ACACCGGCGAACCTACATACACAGATGACTTCAAGGGCAGAT |
| | | TCGCCTTCAGCCTGGACACCAGCGTGTCCACCGCTTATCTGCA |
| | | GATCAGCAGCCTGAAGGCCGACGATACCGCCGTGTACTTTTG |
| | | TGCCCGGGGCGGATTTGGCTCTAGCTACTGGTACTTCGACGTG |
| | | TGGGGCCAGGGCAGCCTGGTGACCGTGTCTAGCGGAGGCGGA |
| | | GGATCAGGTGGCGGTGGATCTGGCGGTGGTGGCTCTGACATC |
| | | CAGCTGACACAGAGCCCATCTAGCCTGAGCGCTAGCGTGGGC |
| | | GACAGAGTGTCTATTACCTGTAAAGCTTCTCAGGACGTGTCCA |
| | | TCGCCGTCGCCTGGTATCAGCAGAAGCCCGGCAAGGCCCCTA |
| | | AGCTGCTGATCTACAGCGCCTCCTACAGATACACCGGCGTGC |
| | | CCGATAGATTCAGCGGAAGCGGCAGCGGAACAGATTTTACCC |
| | | TGACAATCAGCAGCCTGCAGCCTGAGGACTTCGCCGTGTACT |
| | | ACTGCCAGCAACACTACATCACCCCTCTGACCTTCGGCGCCG |
| | | GCACCAAGGTGGAAATCAAGCGGTCAGGCGGCGGAGGAGCG |
| | | GCCGCATCTGGTCAAGTTCTGCTGGAGTCAAACATTAAAGTG |
| | | CTGCCTACTTGGAGCACTCCTGTTCAGCCTGGATATCAGGTTT |
| | | CCTTTTGTTTGGTCATGGTACTTCTCTTTGCGGTAGACACTGGT |
| | | CTCTATTTTAGTGTCAAAACTAATATACGCTCCTCCACGAGGG |
| | | ATTGGAAGGACCATAAGTTCAAATGGAGGAAGGACCCGCAG |
| | | GACAAATGA (SEQ ID NO: 262) |
| | GM-CSF | ATGTGGCTGCAGTCTCTGCTGCTGCTGGGAACAGTGGCCTGTA |
| | Signal | GCATCTCT (SEQ ID NO: 252) |
| | Peptide | |
| | TROP2 scFv | CAAGTGCAGCTGCAGCAGAGCGGCAGCGAGCTGAAAAAGCC |
| | | CGGAGCCAGCGTGAAAGTGTCCTGCAAGGCTTCTGGCTACAC |
| | | ATTCACCAATTACGGCATGAACTGGGTCAAGCAGGCCCCTGG |
| | | ACAGGGCCTGAAGTGGATGGGCTGGATCAACACCTACACCGG |
| | | CGAACCTACATACACAGATGACTTCAAGGGCAGATTCGCCTT |
| | | CAGCCTGGACACCAGCGTGTCCACCGCTTATCTGCAGATCAG |
| | | CAGCCTGAAGGCCGACGATACCGCCGTGTACTTTTGTGCCCG |
| | | GGGCGGATTTGGCTCTAGCTACTGGTACTTCGACGTGTGGGG |
| | | CCAGGGCAGCCTGGTGACCGTGTCTAGCGGAGGCGGAGGATC |
| | | AGGTGGCGGTGGATCTGGCGGTGGTGGCTCTGACATCCAGCT |
| | | GACACAGAGCCCATCTAGCCTGAGCGCTAGCGTGGGCGACAG |
| | | AGTGTCTATTACCTGTAAAGCTTCTCAGGACGTGTCCATCGCC |
| | | GTCGCCTGGTATCAGCAGAAGCCCGGCAAGGCCCCTAAGCTG |
| | | CTGATCTACAGCGCCTCCTACAGATACACCGGCGTGCCCGAT |
| | | AGATTCAGCGGAAGCGGCAGCGGAACAGATTTTACCCTGACA |
| | | ATCAGCAGCCTGCAGCCTGAGGACTTCGCCGTGTACTACTGC |
| | | CAGCAACACTACATCACCCCTCTGACCTTCGGCGCCGGCACC |
| | | AAGGTGGAAATCAAGCGG (SEQ ID NO: 253) |
| | Linker | TCAGGCGGCGGAGGAGCGGCCGCA (SEQ ID NO: 258) |
| | CD4 hinge | TCTGGTCAAGTTCTGCTGGAGTCAAACATTAAAGTGCTGCCTA |
| | | CTTGGAGCACTCCTGTTCAGCCT (SEQ ID NO: 263) |
| | CD16A | GGATATCAGGTTTCCTTTTGTTTGGTCATGGTACTTCTCTTTGC |
| | | GGTAGACACTGGTCTCTATTTTAGTGTCAAAACTAATATACGC |
| | | TCCTCCACGAGGGATTGGAAGGACCATAAGTTCAAATGGAGG |
| | | AAGGACCCGCAGGACAAATGA (SEQ ID NO: 259) |
| TROP2- | Full length | ATGTGGCTGCAGTCTCTGCTGCTGCTGGGAACAGTGGCCTGTA |
| SIGLEC | | GCATCTCTCAAGTGCAGCTGCAGCAGAGCGGCAGCGAGCTGA |
| 4H- | | AAAAGCCCGGAGCCAGCGTGAAAGTGTCCTGCAAGGCTTCTG |
| CD16A | | GCTACACATTCACCAATTACGGCATGAACTGGGTCAAGCAGG |
| | | CCCCTGGACAGGGCCTGAAGTGGATGGGCTGGATCAACACCT |
| | | ACACCGGCGAACCTACATACACAGATGACTTCAAGGGCAGAT |
| | | TCGCCTTCAGCCTGGACACCAGCGTGTCCACCGCTTATCTGCA |
| | | GATCAGCAGCCTGAAGGCCGACGATACCGCCGTGTACTTTTG |
| | | TGCCCGGGGCGGATTTGGCTCTAGCTACTGGTACTTCGACGTG |
| | | TGGGGCCAGGGCAGCCTGGTGACCGTGTCTAGCGGAGGCGGA |
| | | GGATCAGGTGGCGGTGGATCTGGCGGTGGTGGCTCTGACATC |
| | | CAGCTGACACAGAGCCCATCTAGCCTGAGCGCTAGCGTGGGC |
| | | GACAGAGTGTCTATTACCTGTAAAGCTTCTCAGGACGTGTCCA |
| | | TCGCCGTCGCCTGGTATCAGCAGAAGCCCGGCAAGGCCCCTA |
| | | AGCTGCTGATCTACAGCGCCTCCTACAGATACACCGGCGTGC |
| | | CCGATAGATTCAGCGGAAGCGGCAGCGGAACAGATTTTACCC |
| | | TGACAATCAGCAGCCTGCAGCCTGAGGACTTCGCCGTGTACT |
| | | ACTGCCAGCAACACTACATCACCCCTCTGACCTTCGGCGCCG |
| | | GCACCAAGGTGGAAATCAAGCGGTCAGGCGGCGGAGGAGCG |
| | | GCCGCATATCCACCTGTCATAGTTGAAATGAATTCCAGTGTTG |
| | | AGGCTATCGAGGGCAGTCACGTATCACTCCTGTGTGGTGCAG |
| | | ATTCCAATCCACCACCCCTCCTTACATGGATGCGGGATGGAA |
| | | CTGTTCTGAGAGAAGCGGTGGCGGAAAGTTTGCTCCTTGAAT |
| | | TGGAGGAGGTTACTCCCGCCGAGGACGGCGTTTATGCCTGCC |
| | | TGGCCGAGAATGCGTACGGACAAGACAATCGAACGGTCGGTT |
| | | TGAGCGTGATGTACGCGCCTTGGAAACCTACGGTTAACGGCA |
| | | CTATGGTTGCGGTAGAAGGGGAAACGGTATCCATACTCTGTA |
| | | GTACACAATCAAATCCTGATCCCATCCTCACGATCTTTAAAGA |
| | | GAAACAAATCCTTTCCACAGTCATTTATGAGTCTGAGCTTCAG |
| | | CTCGAACTGCCAGCAGTCTCCCCTGAGGATGATGGAGAATAT |
| | | TGGTGCGTTGCCGAAAACCAGTATGGCCAGAGAGCTACAGCG |
| | | TTCAATCTCAGCGTAGAATTTGCTCCAGTTCTCTTGCTGGAGA |
| | | GTCACTGTGCGGCGGCACGGGATACTGTCCAGTGTCTTTGTGT |
| | | AGTGAAAAGCAATCCTGAGCCTTCTGTAGCTTTTGAGTTGCCT |
| | | TCACGCAACGTGACGGTAAATGAGAGCGAACGCGAGTTCGTG |
| | | TATAGTGAGAGAAGCGGATTGGTGCTGACTTCAATCCTCACG |
| | | CTTCGGGGCCAGGCGCAGGCGCCACCTCGCGTGATTTGCACT |
| | | GCTCGGAACCTTTACGGCGCAAAATCCTTGGAGCTGCCGTTTC |
| | | AGGGAGCCCATCGGCTTATGTGGGCTAAGATTGGTCCTGTGG |
| | | GGGCTGGATATCAGGTTTCCTTTTGTTTGGTCATGGTACTTCT |
| | | CTTTGCGGTAGACACTGGTCTCTATTTTAGTGTCAAAACTAAT |
| | | ATACGCTCCTCCACGAGGGATTGGAAGGACCATAAGTTCAAA |
| | | TGGAGGAAGGACCCGCAGGACAAATGA (SEQ ID NO: 264) |
| | GM-CSF | ATGTGGCTGCAGTCTCTGCTGCTGCTGGGAACAGTGGCCTGTA |
| | Signal | GCATCTCT (SEQ ID NO: 252) |
| | Peptide | |
| | TROP2 scFv | CAAGTGCAGCTGCAGCAGAGCGGCAGCGAGCTGAAAAAGCC |
| | | CGGAGCCAGCGTGAAAGTGTCCTGCAAGGCTTCTGGCTACAC |
| | | ATTCACCAATTACGGCATGAACTGGGTCAAGCAGGCCCCTGG |
| | | ACAGGGCCTGAAGTGGATGGGCTGGATCAACACCTACACCGG |
| | | CGAACCTACATACACAGATGACTTCAAGGGCAGATTCGCCTT |
| | | CAGCCTGGACACCAGCGTGTCCACCGCTTATCTGCAGATCAG |
| | | CAGCCTGAAGGCCGACGATACCGCCGTGTACTTTTGTGCCCG |
| | | GGGCGGATTTGGCTCTAGCTACTGGTACTTCGACGTGTGGGG |
| | | CCAGGGCAGCCTGGTGACCGTGTCTAGCGGAGGCGGAGGATC |
| | | AGGTGGCGGTGGATCTGGCGGTGGTGGCTCTGACATCCAGCT |
| | | GACACAGAGCCCATCTAGCCTGAGCGCTAGCGTGGGCGACAG |
| | | AGTGTCTATTACCTGTAAAGCTTCTCAGGACGTGTCCATCGCC |
| | | GTCGCCTGGTATCAGCAGAAGCCCGGCAAGGCCCCTAAGCTG |
| | | CTGATCTACAGCGCCTCCTACAGATACACCGGCGTGCCCGAT |
| | | AGATTCAGCGGAAGCGGCAGCGGAACAGATTTTACCCTGACA |
| | | ATCAGCAGCCTGCAGCCTGAGGACTTCGCCGTGTACTACTGC |
| | | CAGCAACACTACATCACCCCTCTGACCTTCGGCGCCGGCACC |
| | | AAGGTGGAAATCAAGCGG (SEQ ID NO: 253) |
| | Linker | TCAGGCGGCGGAGGAGCGGCCGCA (SEQ ID NO: 258) |
| | SIGLEC4 | TATCCACCTGTCATAGTTGAAATGAATTCCAGTGTTGAGGCTA |
| | Hinge | TCGAGGGCAGTCACGTATCACTCCTGTGTGGTGCAGATTCCA |
| | | ATCCACCACCCCTCCTTACATGGATGCGGGATGGAACTGTTCT |
| | | GAGAGAAGCGGTGGCGGAAAGTTTGCTCCTTGAATTGGAGGA |
| | | GGTTACTCCCGCCGAGGACGGCGTTTATGCCTGCCTGGCCGA |
| | | GAATGCGTACGGACAAGACAATCGAACGGTCGGTTTGAGCGT |
| | | GATGTACGCGCCTTGGAAACCTACGGTTAACGGCACTATGGT |
| | | TGCGGTAGAAGGGGAAACGGTATCCATACTCTGTAGTACACA |
| | | ATCAAATCCTGATCCCATCCTCACGATCTTTAAAGAGAAACA |
| | | AATCCTTTCCACAGTCATTTATGAGTCTGAGCTTCAGCTCGAA |
| | | CTGCCAGCAGTCTCCCCTGAGGATGATGGAGAATATTGGTGC |
| | | GTTGCCGAAAACCAGTATGGCCAGAGAGCTACAGCGTTCAAT |
| | | CTCAGCGTAGAATTTGCTCCAGTTCTCTTGCTGGAGAGTCACT |
| | | GTGCGGCGGCACGGGATACTGTCCAGTGTCTTTGTGTAGTGA |
| | | AAAGCAATCCTGAGCCTTCTGTAGCTTTTGAGTTGCCTTCACG |
| | | CAACGTGACGGTAAATGAGAGCGAACGCGAGTTCGTGTATAG |
| | | TGAGAGAAGCGGATTGGTGCTGACTTCAATCCTCACGCTTCG |
| | | GGGCCAGGCGCAGGCGCCACCTCGCGTGATTTGCACTGCTCG |
| | | GAACCTTTACGGCGCAAAATCCTTGGAGCTGCCGTTTCAGGG |
| | | AGCCCATCGGCTTATGTGGGCTAAGATTGGTCCTGTGGGGGC |
| | | T (SEQ ID NO: 6) |
| | CD16A | GGATATCAGGTTTCCTTTTGTTTGGTCATGGTACTTCTCTTTGC |
| | | GGTAGACACTGGTCTCTATTTTAGTGTCAAAACTAATATACGC |
| | | TCCTCCACGAGGGATTGGAAGGACCATAAGTTCAAATGGAGG |
| | | AAGGACCCGCAGGACAAATGA (SEQ ID NO: 259) |
| HER2- | Full length | ATGTGGCTGCAGTCTCTGCTGCTGCTGGGAACAGTGGCCTGTA |
| CD16A | | GCATCTCTGACATCCAGATGACACAGAGCCCTAGCAGCCTGT |
| | | CTGCCTCTGTGGGCGATAGAGTGACCATCACCTGTAGAGCCA |
| | | GCCAGGATGTGAATACCGCCGTGGCCTGGTATCAGCAGAAGC |
| | | CTGGAAAAGCCCCTAAGCTGCTGATCTACAGCGCCAGCTTTCT |
| | | GTACAGCGGCGTGCCAAGCAGATTCAGCGGCAGCAGATCTGG |
| | | CACCGACTTCACCCTGACCATCTCTAGCCTGCAGCCTGAGGAC |
| | | TTCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATTACTGGGGACAGG |
| | | GCACCCTGGTCACAGTTTCTTCTTCAGGCGGCGGAGGAGCGG |
| | | CCGCAGGAAGTGGTCTGGCCGTAAGTACCATATCCAGCTTTTT |
| | | TCCGCCAGGATATCAGGTTTCCTTTTGTTTGGTCATGGTACTT |
| | | CTCTTTGCGGTAGACACTGGTCTCTATTTTAGTGTCAAAACTA |
| | | ATATACGCTCCTCCACGAGGGATTGGAAGGACCATAAGTTCA |
| | | AATGGAGGAAGGACCCGCAGGACAAATGA (SEQ ID NO: 265) |
| | GM-CSF | ATGTGGCTGCAGTCTCTGCTGCTGCTGGGAACAGTGGCCTGTA |
| | Signal | GCATCTCT (SEQ ID NO: 252) |
| | Peptide | |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATTACTGGGGACAGGGCACCCTG |
| | | GTCACAGTTTCTTCT (SEQ ID NO: 266) |
| | Linker | TCAGGCGGCGGAGGAGCGGCCGCAGGAAGT (SEQ ID NO: 256) |
| | CD16A | GGTCTGGCCGTAAGTACCATATCCAGCTTTTTTCCGCCAGGAT |
| | | ATCAGGTTTCCTTTTGTTTGGTCATGGTACTTCTCTTTGCGGTA |
| | | GACACTGGTCTCTATTTTAGTGTCAAAACTAATATACGCTCCT |
| | | CCACGAGGGATTGGAAGGACCATAAGTTCAAATGGAGGAAG |
| | | GACCCGCAGGACAAATGA (SEQ ID NO: 2) |
| HER2- | Full length | ATGAATAAGCAGCGAGGGACCTTTTCAGAAGTCTCACTCGCT |
| NKG2C | | CAGGATCCTAAAAGACAACAAAGAAAGCCTAAGGGAAATAA |
| | | GTCCAGCATATCAGGTACGGAACAGGAAATTTTTCAAGTCGA |
| | | ACTGAATTTGCAGAACCCTAGCCTGAATCACCAAGGTATCGA |
| | | CAAGATCTATGATTGTCAAGGGCTCCTGCCACCGCCTGAAAA |
| | | GCTTACGGCGGAGGTGCTGGGCATTATTTGTATAGTCCTGATG |
| | | GCAACTGTACTTAAAACTATTGTACTTATCCCGTTTCTGGAAC |
| | | AAAACAATTTTTCTCCGAACACTCGGACACAAAAGGCCCGAC |
| | | ATTGTGGTCACTGTCCAGAAGAGTGGATAACTTACTCTAATA |
| | | GCTGTTACTATATCGGAAAAGAGAGGAGAACGTGGGAAGAA |
| | | AGCTTGCTCGCATGCACTTCCAAAAACTCTTCACTCTTGTCCA |
| | | TTGATAACGAGGAGGAGATGAAATTTCTGGCCTCAATCCTGC |
| | | CATCATCTTGGATAGGCGTATTCCGCAACTCAAGTCATCACCC |
| | | TTGGGTAACTATAAATGGTTTGGCGTTCAAGCACAAGATTAA |
| | | AGACTCTGATAATGCCGAGTTGAACTGCGCTGTTCTTCAGGTG |
| | | AACCGCCTCAAATCTGCCCAGTGCGGAAGTTCTATGATATATC |
| | | ACTGCAAACATAAACTGTCAGGCGGCGGAGGAGCGGCCGCA |
| | | GGAAGTGACATCCAGATGACACAGAGCCCTAGCAGCCTGTCT |
| | | GCCTCTGTGGGCGATAGAGTGACCATCACCTGTAGAGCCAGC |
| | | CAGGATGTGAATACCGCCGTGGCCTGGTATCAGCAGAAGCCT |
| | | GGAAAAGCCCCTAAGCTGCTGATCTACAGCGCCAGCTTTCTG |
| | | TACAGCGGCGTGCCAAGCAGATTCAGCGGCAGCAGATCTGGC |
| | | ACCGACTTCACCCTGACCATCTCTAGCCTGCAGCCTGAGGACT |
| | | TCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATTACTGGGGACAGG |
| | | GCACCCTGGTCACAGTTTCTTCTTGA (SEQ ID NO: 267) |
| | NKG2C | ATGAATAAGCAGCGAGGGACCTTTTCAGAAGTCTCACTCGCT |
| | (cytoplasmic, | CAGGATCCTAAAAGACAACAAAGAAAGCCTAAGGGAAATAA |
| | TM and | GTCCAGCATATCAGGTACGGAACAGGAAATTTTTCAAGTCGA |
| | extracellular) | ACTGAATTTGCAGAACCCTAGCCTGAATCACCAAGGTATCGA |
| | | CAAGATCTATGATTGTCAAGGGCTCCTGCCACCGCCTGAAAA |
| | | GCTTACGGCGGAGGTGCTGGGCATTATTTGTATAGTCCTGATG |
| | | GCAACTGTACTTAAAACTATTGTACTTATCCCGTTTCTGGAAC |
| | | AAAACAATTTTTCTCCGAACACTCGGACACAAAAGGCCCGAC |
| | | ATTGTGGTCACTGTCCAGAAGAGTGGATAACTTACTCTAATA |
| | | GCTGTTACTATATCGGAAAAGAGAGGAGAACGTGGGAAGAA |
| | | AGCTTGCTCGCATGCACTTCCAAAAACTCTTCACTCTTGTCCA |
| | | TTGATAACGAGGAGGAGATGAAATTTCTGGCCTCAATCCTGC |
| | | CATCATCTTGGATAGGCGTATTCCGCAACTCAAGTCATCACCC |
| | | TTGGGTAACTATAAATGGTTTGGCGTTCAAGCACAAGATTAA |
| | | AGACTCTGATAATGCCGAGTTGAACTGCGCTGTTCTTCAGGTG |
| | | AACCGCCTCAAATCTGCCCAGTGCGGAAGTTCTATGATATATC |
| | | ACTGCAAACATAAACTG (SEQ ID NO: 8) |
| | Linker | TCAGGCGGCGGAGGAGCGGCCGCAGGAAGT (SEQ ID NO: 256) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATTACTGGGGACAGGGCACCCTG |
| | | GTCACAGTTTCTTCTTGA (SEQ ID NO: 268) |
| HER2- | Full length | ATGAATAAGCAGCGAGGGACCTTTTCAGAAGTCTCACTCGCT |
| NKG2C | | CAGGATCCTAAAAGACAACAAAGAAAGCCTAAGGGAAATAA |
| TMCyto | | GTCCAGCATATCAGGTACGGAACAGGAAATTTTTCAAGTCGA |
| | | ACTGAATTTGCAGAACCCTAGCCTGAATCACCAAGGTATCGA |
| | | CAAGATCTATGATTGTCAAGGGCTCCTGCCACCGCCTGAAAA |
| | | GCTTACGGCGGAGGTGCTGGGCATTATTTGTATAGTCCTGATG |
| | | GCAACTGTACTTAAAACTATTGTACTTTCAGGCGGCGGAGGA |
| | | GCGGCCGCAGGAAGTGACATCCAGATGACACAGAGCCCTAGC |
| | | AGCCTGTCTGCCTCTGTGGGCGATAGAGTGACCATCACCTGTA |
| | | GAGCCAGCCAGGATGTGAATACCGCCGTGGCCTGGTATCAGC |
| | | AGAAGCCTGGAAAAGCCCCTAAGCTGCTGATCTACAGCGCCA |
| | | GCTTTCTGTACAGCGGCGTGCCAAGCAGATTCAGCGGCAGCA |
| | | GATCTGGCACCGACTTCACCCTGACCATCTCTAGCCTGCAGCC |
| | | TGAGGACTTCGCCACCTACTACTGCCAGCAGCACTACACCAC |
| | | ACCTCCAACCTTTGGCCAGGGCACCAAGGTGGAAATCAAGAG |
| | | AACAGGCAGCACCAGCGGCTCTGGAAAGCCTGGATCTGGCGA |
| | | AGGATCTGAGGTGCAGCTGGTTGAATCTGGCGGAGGACTTGT |
| | | TCAGCCTGGCGGCTCTCTGAGACTGTCTTGTGCCGCCAGCGGC |
| | | TTCAACATCAAGGACACCTACATCCACTGGGTCCGACAGGCC |
| | | CCTGGAAAGGGACTTGAATGGGTCGCCAGAATCTACCCCACC |
| | | AACGGCTACACCAGATACGCCGATAGCGTGAAGGGCAGATTC |
| | | ACCATCAGCGCCGACACCAGCAAGAACACCGCCTACCTGCAG |
| | | ATGAACAGCCTGAGAGCCGAGGACACCGCCGTGTACTACTGT |
| | | TCTAGATGGGGAGGCGACGGCTTCTACGCCATGGATTACTGG |
| | | GGACAGGGCACCCTGGTCACAGTTTCTTCTTGA (SEQ ID NO: |
| | | 269) |
| | NKG2C | ATGAATAAGCAGCGAGGGACCTTTTCAGAAGTCTCACTCGCT |
| | (cytoplasmic | CAGGATCCTAAAAGACAACAAAGAAAGCCTAAGGGAAATAA |
| | and TM) | GTCCAGCATATCAGGTACGGAACAGGAAATTTTTCAAGTCGA |
| | | ACTGAATTTGCAGAACCCTAGCCTGAATCACCAAGGTATCGA |
| | | CAAGATCTATGATTGTCAAGGGCTCCTGCCACCGCCTGAAAA |
| | | GCTTACGGCGGAGGTGCTGGGCATTATTTGTATAGTCCTGATG |
| | | GCAACTGTACTTAAAACTATTGTACTT (SEQ ID NO: 270) |
| | Linker | TCAGGCGGCGGAGGAGCGGCCGCAGGAAGT (SEQ ID NO: 256) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATtacTGGGGACAGGGCACCCTGG |
| | | TCACAGTTTCTTCTTGA (SEQ ID NO: 268) |
| HER2- | Full length | ATGGGGTGGATACGAGGCAGGAGGTCTCGGCACAGCTGGGA |
| NKG2D | | GATGTCAGAGTTTCACAACTACAACCTCGACCTGAAAAAATC |
| | | CGACTTCTCTACCCGATGGCAAAAGCAGCGATGTCCGGTAGT |
| | | GAAGTCAAAATGTCGGGAAAACGCATCTCCGTTTTTTTTTTGC |
| | | TGCTTCATAGCCGTCGCGATGGGCATAAGATTCATCATTATGG |
| | | TGACTATTTGGTCTGCCGTGTTTCTTAATAGCTTGTTCAACCA |
| | | AGAAGTGCAAATACCCCTTACCGAGAGTTACTGTGGTCCGTG |
| | | TCCTAAGAACTGGATATGTTACAAGAACAACTGCTACCAATT |
| | | CTTCGATGAAAGTAAAAACTGGTATGAGTCACAGGCAAGCTG |
| | | CATGAGCCAAAACGCTAGTCTCCTCAAAGTCTACAGTAAAGA |
| | | GGATCAGGACCTGCTGAAACTTGTGAAAAGTTATCACTGGAT |
| | | GGGTTTGGTACACATTCCAACTAACGGTTCCTGGCAATGGGA |
| | | AGATGGATCAATCCTTAGCCCCAATCTCCTCACTATTATAGAA |
| | | ATGCAAAAGGGTGACTGTGCTCTGTACGCGTCCAGCTTTAAG |
| | | GGGTATATCGAGAATTGTAGCACACCCAATACTTACATCTGT |
| | | ATGCAACGAACCGTGTCAGGCGGCGGAGGAGCGGCCGCAGG |
| | | AAGTGACATCCAGATGACACAGAGCCCTAGCAGCCTGTCTGC |
| | | CTCTGTGGGCGATAGAGTGACCATCACCTGTAGAGCCAGCCA |
| | | GGATGTGAATACCGCCGTGGCCTGGTATCAGCAGAAGCCTGG |
| | | AAAAGCCCCTAAGCTGCTGATCTACAGCGCCAGCTTTCTGTAC |
| | | AGCGGCGTGCCAAGCAGATTCAGCGGCAGCAGATCTGGCACC |
| | | GACTTCACCCTGACCATCTCTAGCCTGCAGCCTGAGGACTTCG |
| | | CCACCTACTACTGCCAGCAGCACTACACCACACCTCCAACCTT |
| | | TGGCCAGGGCACCAAGGTGGAAATCAAGAGAACAGGCAGCA |
| | | CCAGCGGCTCTGGAAAGCCTGGATCTGGCGAAGGATCTGAGG |
| | | TGCAGCTGGTTGAATCTGGCGGAGGACTTGTTCAGCCTGGCG |
| | | GCTCTCTGAGACTGTCTTGTGCCGCCAGCGGCTTCAACATCAA |
| | | GGACACCTACATCCACTGGGTCCGACAGGCCCCTGGAAAGGG |
| | | ACTTGAATGGGTCGCCAGAATCTACCCCACCAACGGCTACAC |
| | | CAGATACGCCGATAGCGTGAAGGGCAGATTCACCATCAGCGC |
| | | CGACACCAGCAAGAACACCGCCTACCTGCAGATGAACAGCCT |
| | | GAGAGCCGAGGACACCGCCGTGTACTACTGTTCTAGATGGGG |
| | | AGGCGACGGCTTCTACGCCATGGATtacTGGGGACAGGGCACC |
| | | CTGGTCACAGTTTCTTCTTGA (SEQ ID NO: 271) |
| | NKG2D | ATGGGGTGGATACGAGGCAGGAGGTCTCGGCACAGCTGGGA |
| | (cytoplasmic, | GATGTCAGAGTTTCACAACTACAACCTCGACCTGAAAAAATC |
| | TM and | CGACTTCTCTACCCGATGGCAAAAGCAGCGATGTCCGGTAGT |
| | extracellular) | GAAGTCAAAATGTCGGGAAAACGCATCTCCGTTTTTTTTTTGC |
| | | TGCTTCATAGCCGTCGCGATGGGCATAAGATTCATCATTATGG |
| | | TGACTATTTGGTCTGCCGTGTTTCTTAATAGCTTGTTCAACCA |
| | | AGAAGTGCAAATACCCCTTACCGAGAGTTACTGTGGTCCGTG |
| | | TCCTAAGAACTGGATATGTTACAAGAACAACTGCTACCAATT |
| | | CTTCGATGAAAGTAAAAACTGGTATGAGTCACAGGCAAGCTG |
| | | CATGAGCCAAAACGCTAGTCTCCTCAAAGTCTACAGTAAAGA |
| | | GGATCAGGACCTGCTGAAACTTGTGAAAAGTTATCACTGGAT |
| | | GGGTTTGGTACACATTCCAACTAACGGTTCCTGGCAATGGGA |
| | | AGATGGATCAATCCTTAGCCCCAATCTCCTCACTATTATAGAA |
| | | ATGCAAAAGGGTGACTGTGCTCTGTACGCGTCCAGCTTTAAG |
| | | GGGTATATCGAGAATTGTAGCACACCCAATACTTACATCTGT |
| | | ATGCAACGAACCGTG (SEQ ID NO: 272) |
| | Linker | TCAGGCGGCGGAGGAGCGGCCGCAGGAAGT (SEQ ID NO: 256) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATtacTGGGGACAGGGCACCCTGG |
| | | TCACAGTTTCTTCTTGA (SEQ ID NO: 268) |
| HER2- | Full length | ATGTGGCTGCAGTCTCTGCTGCTGCTGGGAACAGTGGCCTGTA |
| NKp30 | | GCATCTCTGACATCCAGATGACACAGAGCCCTAGCAGCCTGT |
| | | CTGCCTCTGTGGGCGATAGAGTGACCATCACCTGTAGAGCCA |
| | | GCCAGGATGTGAATACCGCCGTGGCCTGGTATCAGCAGAAGC |
| | | CTGGAAAAGCCCCTAAGCTGCTGATCTACAGCGCCAGCTTTCT |
| | | GTACAGCGGCGTGCCAAGCAGATTCAGCGGCAGCAGATCTGG |
| | | CACCGACTTCACCCTGACCATCTCTAGCCTGCAGCCTGAGGAC |
| | | TTCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATtacTGGGGACAGGG |
| | | CACCCTGGTCACAGTTTCTTCTTCAGGCGGCGGAGGAGCGGC |
| | | CGCAGGAAGTCTGTGGGTTAGCCAGCCACCCGAGATTCGAAC |
| | | CCTCGAAGGCAGTAGTGCTTTCTTGCCGTGTAGCTTCAATGCG |
| | | TCCCAAGGGCGACTGGCAATCGGTTCAGTCACATGGTTTCGC |
| | | GACGAAGTTGTACCTGGCAAGGAGGTCAGGAATGGGACACC |
| | | GGAATTTCGCGGCCGACTGGCCCCGTTGGCATCTTCCCGATTT |
| | | CTTCATGATCACCAGGCAGAGCTTCACATTCGCGACGTACGA |
| | | GGACACGACGCAAGCATCTATGTATGTAGAGTTGAAGTTTTG |
| | | GGACTTGGAGTAGGCACAGGGAATGGGACTAGGTTGGTAGTG |
| | | GAAAAGGAGCATCCCCAGTTGGGCGCAGGTACCGTACTTCTT |
| | | CTGCGGGCAGGGTTCTATGCTGTCAGCTTTCTGTCTGTGGCAG |
| | | TTGGGTCCACAGTCTATTACCAGGGTAAGTGTCTCACGTGGA |
| | | AGGGACCACGGCGGCAATTGCCTGCGGTTGTTCCCGCACCCC |
| | | TCCCTCCTCCATGCGGTTCAAGTGCACATCTCCTTCCGCCAGT |
| | | TCCAGGCGGCTGA (SEQ ID NO: 273) |
| | GM-CSF | ATGTGGCTGCAGTCTCTGCTGCTGCTGGGAACAGTGGCCTGTA |
| | Signal | GCATCTCT (SEQ ID NO: 252) |
| | Peptide | |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATTACTGGGGACAGGGCACCCTG |
| | | GTCACAGTTTCTTCT (SEQ ID NO: 266) |
| | LINKER | TCAGGCGGCGGAGGAGCGGCCGCAGGAAGT (SEQ ID NO: 256) |
| | NKP30 | CTGTGGGTTAGCCAGCCACCCGAGATTCGAACCCTCGAAGGC |
| | (extracellular, | AGTAGTGCTTTCTTGCCGTGTAGCTTCAATGCGTCCCAAGGGC |
| | TM, | GACTGGCAATCGGTTCAGTCACATGGTTTCGCGACGAAGTTG |
| | cytoplasmic) | TACCTGGCAAGGAGGTCAGGAATGGGACACCGGAATTTCGCG |
| | | GCCGACTGGCCCCGTTGGCATCTTCCCGATTTCTTCATGATCA |
| | | CCAGGCAGAGCTTCACATTCGCGACGTACGAGGACACGACGC |
| | | AAGCATCTATGTATGTAGAGTTGAAGTTTTGGGACTTGGAGT |
| | | AGGCACAGGGAATGGGACTAGGTTGGTAGTGGAAAAGGAGC |
| | | ATCCCCAGTTGGGCGCAGGTACCGTACTTCTTCTGCGGGCAG |
| | | GGTTCTATGCTGTCAGCTTTCTGTCTGTGGCAGTTGGGTCCAC |
| | | AGTCTATTACCAGGGTAAGTGTCTCACGTGGAAGGGACCACG |
| | | GCGGCAATTGCCTGCGGTTGTTCCCGCACCCCTCCCTCCTCCA |
| | | TGCGGTTCAAGTGCACATCTCCTTCCGCCAGTTCCAGGCGGCT |
| | | GA (SEQ ID NO: 10) |
| HER2- | Full length | ATGTGGCTGCAGTCTCTGCTGCTGCTGGGAACAGTGGCCTGTA |
| NKp30 | | GCATCTCTGACATCCAGATGACACAGAGCCCTAGCAGCCTGT |
| TM Cyto | | CTGCCTCTGTGGGCGATAGAGTGACCATCACCTGTAGAGCCA |
| | | GCCAGGATGTGAATACCGCCGTGGCCTGGTATCAGCAGAAGC |
| | | CTGGAAAAGCCCCTAAGCTGCTGATCTACAGCGCCAGCTTTCT |
| | | GTACAGCGGCGTGCCAAGCAGATTCAGCGGCAGCAGATCTGG |
| | | CACCGACTTCACCCTGACCATCTCTAGCCTGCAGCCTGAGGAC |
| | | TTCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATTACTGGGGACAGG |
| | | GCACCCTGGTCACAGTTTCTTCTTCAGGCGGCGGAGGAGCGG |
| | | CCGCAGGAAGTGCAGGTACCGTACTTCTTCTGCGGGCAGGGT |
| | | TCTATGCTGTCAGCTTTCTGTCTGTGGCAGTTGGGTCCACAGT |
| | | CTATTACCAGGGTAAGTGTCTCACGTGGAAGGGACCACGGCG |
| | | GCAATTGCCTGCGGTTGTTCCCGCACCCCTCCCTCCTCCATGC |
| | | GGTTCAAGTGCACATCTCCTTCCGCCAGTTCCAGGCGGCTGA |
| | | (SEQ ID NO: 274) |
| | GM-CSF | ATGTGGCTGCAGTCTCTGCTGCTGCTGGGAACAGTGGCCTGTA |
| | signal peptide | GCATCTCT (SEQ ID NO: 252) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATtacTGGGGACAGGGCACCCTGG |
| | | TCACAGTTTCTTCT (SEQ ID NO: 266) |
| | Linker | TCAGGCGGCGGAGGAGCGGCCGCAGGAAGT (SEQ ID NO: 256) |
| | NKp30 (TM, | GCAGGTACCGTACTTCTTCTGCGGGCAGGGTTCTATGCTGTCA |
| | cytoplasmic) | GCTTTCTGTCTGTGGCAGTTGGGTCCACAGTCTATTACCAGGG |
| | | TAAGTGTCTCACGTGGAAGGGACCACGGCGGCAATTGCCTGC |
| | | GGTTGTTCCCGCACCCCTCCCTCCTCCATGCGGTTCAAGTGCA |
| | | CATCTCCTTCCGCCAGTTCCAGGCGGCTGA (SEQ ID NO: 11) |
| HER2- | Full length | ATGTGGCTGCAGTCTCTGCTGCTGCTGGGAACAGTGGCCTGTA |
| NKp44 | | GCATCTCTGACATCCAGATGACACAGAGCCCTAGCAGCCTGT |
| | | CTGCCTCTGTGGGCGATAGAGTGACCATCACCTGTAGAGCCA |
| | | GCCAGGATGTGAATACCGCCGTGGCCTGGTATCAGCAGAAGC |
| | | CTGGAAAAGCCCCTAAGCTGCTGATCTACAGCGCCAGCTTTCT |
| | | GTACAGCGGCGTGCCAAGCAGATTCAGCGGCAGCAGATCTGG |
| | | CACCGACTTCACCCTGACCATCTCTAGCCTGCAGCCTGAGGAC |
| | | TTCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATtacTGGGGACAGGG |
| | | CACCCTGGTCACAGTTTCTTCTTCAGGCGGCGGAGGAGCGGC |
| | | CGCAGGAAGTCAGTCAAAAGCACAAGTTCTGCAGAGTGTAGC |
| | | AGGCCAGACACTTACGGTTAGGTGTCAATATCCGCCTACCGG |
| | | CAGTCTGTATGAGAAAAAGGGCTGGTGTAAAGAAGCGTCCGC |
| | | GTTGGTTTGCATTCGGCTCGTAACGAGCTCCAAGCCTCGGACT |
| | | ATGGCATGGACATCACGGTTCACTATATGGGACGACCCTGAT |
| | | GCAGGATTTTTTACTGTCACCATGACGGACCTTCGGGAGGAG |
| | | GATTCCGGCCACTATTGGTGTAGAATCTATAGACCGTCAGAC |
| | | AATTCTGTCAGCAAGAGCGTGCGCTTCTACCTTGTGGTCTCTC |
| | | CGGCTTCCGCCTCCACCCAAACATCTTGGACTCCGAGGGATCT |
| | | CGTGTCATCACAAACCCAGACACAGAGTTGTGTGCCGCCCAC |
| | | GGCGGGAGCAAGACAGGCTCCGGAGAGCCCATCTACAATTCC |
| | | GGTCCCTAGCCAACCACAGAACTCTACCTTGAGGCCCGGACC |
| | | CGCTGCACCCATCGCTTTGGTTCCAGTGTTTTGCGGACTCCTT |
| | | GTTGCCAAGTCACTTGTCCTTTCTGCTCTCCTGGTATGGTGGG |
| | | GCGACATTTGGTGGAAAACGATGATGGAGCTTCGATCCTTGG |
| | | ACACACAGAAGGCGACATGTCATCTCCAACAGGTGACAGACC |
| | | TGCCATGGACTAGTGTGTCAAGTCCCGTCGAGCGCGAAATCC |
| | | TTTATCATACCGTGGCCCGAACCAAAATAAGCGACGATGACG |
| | | ATGAGCACACTCTGTGA (SEQ ID NO: 275) |
| | GM-CSF | ATGTGGCTGCAGTCTCTGCTGCTGCTGGGAACAGTGGCCTGTA |
| | signal peptide | GCATCTCT (SEQ ID NO: 252) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATtacTGGGGACAGGGCACCCTGG |
| | | TCACAGTTTCTTCT (SEQ ID NO: 266) |
| | Linker | TCAGGCGGCGGAGGAGCGGCCGCAGGAAGT (SEQ ID NO: 256) |
| | NKp44 | CAGTCAAAAGCACAAGTTCTGCAGAGTGTAGCAGGCCAGACA |
| | (extracellular, | CTTACGGTTAGGTGTCAATATCCGCCTACCGGCAGTCTGTATG |
| | TM, | AGAAAAAGGGCTGGTGTAAAGAAGCGTCCGCGTTGGTTTGCA |
| | cytoplasmic) | TTCGGCTCGTAACGAGCTCCAAGCCTCGGACTATGGCATGGA |
| | | CATCACGGTTCACTATATGGGACGACCCTGATGCAGGATTTTT |
| | | TACTGTCACCATGACGGACCTTCGGGAGGAGGATTCCGGCCA |
| | | CTATTGGTGTAGAATCTATAGACCGTCAGACAATTCTGTCAGC |
| | | AAGAGCGTGCGCTTCTACCTTGTGGTCTCTCCGGCTTCCGCCT |
| | | CCACCCAAACATCTTGGACTCCGAGGGATCTCGTGTCATCAC |
| | | AAACCCAGACACAGAGTTGTGTGCCGCCCACGGCGGGAGCAA |
| | | GACAGGCTCCGGAGAGCCCATCTACAATTCCGGTCCCTAGCC |
| | | AACCACAGAACTCTACCTTGAGGCCCGGACCCGCTGCACCCA |
| | | TCGCTTTGGTTCCAGTGTTTTGCGGACTCCTTGTTGCCAAGTC |
| | | ACTTGTCCTTTCTGCTCTCCTGGTATGGTGGGGCGACATTTGG |
| | | TGGAAAACGATGATGGAGCTTCGATCCTTGGACACACAGAAG |
| | | GCGACATGTCATCTCCAACAGGTGACAGACCTGCCATGGACT |
| | | AGTGTGTCAAGTCCCGTCGAGCGCGAAATCCTTTATCATACCG |
| | | TGGCCCGAACCAAAATAAGCGACGATGACGATGAGCACACTC |
| | | TGTGA (SEQ ID NO: 13) |
| HER2- | Full length | ATGTGGCTGCAGTCTCTGCTGCTGCTGGGAACAGTGGCCTGTA |
| NKp44 | | GCATCTCTGACATCCAGATGACACAGAGCCCTAGCAGCCTGT |
| TM Cyto | | CTGCCTCTGTGGGCGATAGAGTGACCATCACCTGTAGAGCCA |
| | | GCCAGGATGTGAATACCGCCGTGGCCTGGTATCAGCAGAAGC |
| | | CTGGAAAAGCCCCTAAGCTGCTGATCTACAGCGCCAGCTTTCT |
| | | GTACAGCGGCGTGCCAAGCAGATTCAGCGGCAGCAGATCTGG |
| | | CACCGACTTCACCCTGACCATCTCTAGCCTGCAGCCTGAGGAC |
| | | TTCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATtacTGGGGACAGGG |
| | | CACCCTGGTCACAGTTTCTTCTTCAGGCGGCGGAGGAGCGGC |
| | | CGCAGGAAGTTTGGTTCCAGTGTTTTGCGGACTCCTTGTTGCC |
| | | AAGTCACTTGTCCTTTCTGCTCTCCTGGTATGGTGGGGCGACA |
| | | TTTGGTGGAAAACGATGATGGAGCTTCGATCCTTGGACACAC |
| | | AGAAGGCGACATGTCATCTCCAACAGGTGACAGACCTGCCAT |
| | | GGACTAGTGTGTCAAGTCCCGTCGAGCGCGAAATCCTTTATC |
| | | ATACCGTGGCCCGAACCAAAATAAGCGACGATGACGATGAGC |
| | | ACACTCTGTGA (SEQ ID NO: 276) |
| | GM-CSF | ATGTGGCTGCAGTCTCTGCTGCTGCTGGGAACAGTGGCCTGTA |
| | signal peptide | GCATCTCT (SEQ ID NO: 252) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATtacTGGGGACAGGGCACCCTGG |
| | | TCACAGTTTCTTCT (SEQ ID NO: 266) |
| | Linker | TCAGGCGGCGGAGGAGCGGCCGCAGGAAGT (SEQ ID NO: 256) |
| | NKp44 (TM, | TTGGTTCCAGTGTTTTGCGGACTCCTTGTTGCCAAGTCACTTG |
| | cytoplasmic) | TCCTTTCTGCTCTCCTGGTATGGTGGGGCGACATTTGGTGGAA |
| | | AACGATGATGGAGCTTCGATCCTTGGACACACAGAAGGCGAC |
| | | ATGTCATCTCCAACAGGTGACAGACCTGCCATGGACTAGTGT |
| | | GTCAAGTCCCGTCGAGCGCGAAATCCTTTATCATACCGTGGCC |
| | | CGAACCAAAATAAGCGACGATGACGATGAGCACACTCTGTGA |
| | | (SEQ ID NO: 277) |
| HER2- | Full length | ATGTGGCTGCAGTCTCTGCTGCTGCTGGGAACAGTGGCCTGTA |
| NKp46 | | GCATCTCTGACATCCAGATGACACAGAGCCCTAGCAGCCTGT |
| | | CTGCCTCTGTGGGCGATAGAGTGACCATCACCTGTAGAGCCA |
| | | GCCAGGATGTGAATACCGCCGTGGCCTGGTATCAGCAGAAGC |
| | | CTGGAAAAGCCCCTAAGCTGCTGATCTACAGCGCCAGCTTTCT |
| | | GTACAGCGGCGTGCCAAGCAGATTCAGCGGCAGCAGATCTGG |
| | | CACCGACTTCACCCTGACCATCTCTAGCCTGCAGCCTGAGGAC |
| | | TTCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATTACTGGGGACAGG |
| | | GCACCCTGGTCACAGTTTCTTCTTCAGGCGGCGGAGGAGCGG |
| | | CCGCAGGAAGTCAGCAACAGACGCTGCCCAAACCATTTATTT |
| | | GGGCTGAACCTCACTTTATGGTCCCCAAGGAAAAACAAGTTA |
| | | CAATCTGTTGCCAGGGGAATTACGGAGCTGTGGAGTACCAGC |
| | | TGCATTTCGAGGGATCTCTTTTTGCGGTTGATAGGCCCAAGCC |
| | | GCCCGAGCGGATCAACAAGGTGAAGTTTTACATACCAGATAT |
| | | GAACTCCAGGATGGCGGGACAGTACTCCTGTATCTATCGCGT |
| | | GGGCGAGCTGTGGAGTGAGCCTTCCAATCTGCTTGATCTTGTC |
| | | GTCACCGAGATGTACGATACACCTACGCTGAGCGTTCATCCC |
| | | GGGCCGGAAGTTATTAGTGGCGAAAAGGTCACTTTCTATTGC |
| | | CGCCTTGACACGGCTACGTCAATGTTCCTCTTGCTGAAAGAAG |
| | | GAAGATCTTCCCATGTCCAACGAGGATACGGAAAAGTCCAAG |
| | | CGGAATTTCCCCTGGGACCAGTAACTACCGCTCATAGGGGAA |
| | | CATACCGATGCTTCGGCAGCTACAACAACCACGCTTGGAGTT |
| | | TTCCGTCTGAGCCTGTAAAATTGCTCGTTACCGGAGACATTGA |
| | | GAACACGAGCCTTGCCCCTGAAGATCCGACGTTCCCAGCAGA |
| | | TACATGGGGGACTTATCTGTTGACTACGGAGACAGGACTTCA |
| | | AAAGGACCATGCGTTGTGGGATCATACAGCTCAGAACTTGCT |
| | | CCGCATGGGCCTGGCCTTTCTTGTACTCGTTGCACTCGTTTGG |
| | | TTCCTTGTTGAGGATTGGCTCTCTAGAAAGAGAACTAGAGAA |
| | | CGGGCCTCCAGGGCATCCACGTGGGAAGGCCGCAGACGACTC |
| | | AATACCCAGACCCTGTGA (SEQ ID NO: 278) |
| | GM-CSF | ATGTGGCTGCAGTCTCTGCTGCTGCTGGGAACAGTGGCCTGTA |
| | signal peptide | GCATCTCT (SEQ ID NO: 252) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATTACTGGGGACAGGGCACCCTG |
| | | GTCACAGTTTCTTCT (SEQ ID NO: 266) |
| | Linker | TCAGGCGGCGGAGGAGCGGCCGCAGGAAGT (SEQ ID NO: 256) |
| | NKp46 | CAGCAACAGACGCTGCCCAAACCATTTATTTGGGCTGAACCT |
| | (extracellular, | CACTTTATGGTCCCCAAGGAAAAACAAGTTACAATCTGTTGC |
| | TM, | CAGGGGAATTACGGAGCTGTGGAGTACCAGCTGCATTTCGAG |
| | cytoplasmic) | GGATCTCTTTTTGCGGTTGATAGGCCCAAGCCGCCCGAGCGG |
| | | ATCAACAAGGTGAAGTTTTACATACCAGATATGAACTCCAGG |
| | | ATGGCGGGACAGTACTCCTGTATCTATCGCGTGGGCGAGCTG |
| | | TGGAGTGAGCCTTCCAATCTGCTTGATCTTGTCGTCACCGAGA |
| | | TGTACGATACACCTACGCTGAGCGTTCATCCCGGGCCGGAAG |
| | | TTATTAGTGGCGAAAAGGTCACTTTCTATTGCCGCCTTGACAC |
| | | GGCTACGTCAATGTTCCTCTTGCTGAAAGAAGGAAGATCTTCC |
| | | CATGTCCAACGAGGATACGGAAAAGTCCAAGCGGAATTTCCC |
| | | CTGGGACCAGTAACTACCGCTCATAGGGGAACATACCGATGC |
| | | TTCGGCAGCTACAACAACCACGCTTGGAGTTTTCCGTCTGAGC |
| | | CTGTAAAATTGCTCGTTACCGGAGACATTGAGAACACGAGCC |
| | | TTGCCCCTGAAGATCCGACGTTCCCAGCAGATACATGGGGGA |
| | | CTTATCTGTTGACTACGGAGACAGGACTTCAAAAGGACCATG |
| | | CGTTGTGGGATCATACAGCTCAGAACTTGCTCCGCATGGGCCT |
| | | GGCCTTTCTTGTACTCGTTGCACTCGTTTGGTTCCTTGTTGAGG |
| | | ATTGGCTCTCTAGAAAGAGAACTAGAGAACGGGCCTCCAGGG |
| | | CATCCACGTGGGAAGGCCGCAGACGACTCAATACCCAGACCC |
| | | TGTGA (SEQ ID NO: 16) |
| HER2- | | ATGTGGCTGCAGTCTCTGCTGCTGCTGGGAACAGTGGCCTGTA |
| NKp46T | | GCATCTCTGACATCCAGATGACACAGAGCCCTAGCAGCCTGT |
| MCyto | | CTGCCTCTGTGGGCGATAGAGTGACCATCACCTGTAGAGCCA |
| | | GCCAGGATGTGAATACCGCCGTGGCCTGGTATCAGCAGAAGC |
| | | CTGGAAAAGCCCCTAAGCTGCTGATCTACAGCGCCAGCTTTCT |
| | | GTACAGCGGCGTGCCAAGCAGATTCAGCGGCAGCAGATCTGG |
| | | CACCGACTTCACCCTGACCATCTCTAGCCTGCAGCCTGAGGAC |
| | | TTCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATTACTGGGGACAGG |
| | | GCACCCTGGTCACAGTTTCTTCTTCAGGCGGCGGAGGAGCGG |
| | | CCGCAGGAAGTATGGGCCTGGCCTTTCTTGTACTCGTTGCACT |
| | | CGTTTGGTTCCTTGTTGAGGATTGGCTCTCTAGAAAGAGAACT |
| | | AGAGAACGGGCCTCCAGGGCATCCACGTGGGAAGGCCGCAG |
| | | ACGACTCAATACCCAGACCCTGTGA (SEQ ID NO: 279) |
| | GM-CSF | ATGTGGCTGCAGTCTCTGCTGCTGCTGGGAACAGTGGCCTGTA |
| | signal peptide | GCATCTCT (SEQ ID NO: 252) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATTACTGGGGACAGGGCACCCTG |
| | | GTCACAGTTTCTTCT (SEQ ID NO: 266) |
| | Linker | TCAGGCGGCGGAGGAGCGGCCGCAGGAAGT (SEQ ID NO: 256) |
| | NKp46 (TM, | ATGGGCCTGGCCTTTCTTGTACTCGTTGCACTCGTTTGGTTCCT |
| | cytoplasmic) | TGTTGAGGATTGGCTCTCTAGAAAGAGAACTAGAGAACGGGC |
| | | CTCCAGGGCATCCACGTGGGAAGGCCGCAGACGACTCAATAC |
| | | CCAGACCCTGTGA (SEQ ID NO: 17) |
| HER2- | Full length | ATGTGGCTGCAGTCTCTGCTGCTGCTGGGAACAGTGGCCTGTA |
| revNKG | | GCATCTCTGACATCCAGATGACACAGAGCCCTAGCAGCCTGT |
| 2D | | CTGCCTCTGTGGGCGATAGAGTGACCATCACCTGTAGAGCCA |
| | | GCCAGGATGTGAATACCGCCGTGGCCTGGTATCAGCAGAAGC |
| | | CTGGAAAAGCCCCTAAGCTGCTGATCTACAGCGCCAGCTTTCT |
| | | GTACAGCGGCGTGCCAAGCAGATTCAGCGGCAGCAGATCTGG |
| | | CACCGACTTCACCCTGACCATCTCTAGCCTGCAGCCTGAGGAC |
| | | TTCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATTACTGGGGACAGG |
| | | GCACCCTGGTCACAGTTTCTTCTTCAGGCGGCGGAGGAGCGG |
| | | CCGCAGGATGCTACAGCGAGACCCTGCCCATCCAGGTGGAGC |
| | | AGAACTTCCTGAGCAACCTGTTCGTGGCCAGCTGGATCACCG |
| | | TGATGATCATCTTCAGGATCGGCATGGCCGTGGCCATCTTCTG |
| | | CTGCTTCTTCTTCCCCAGCGCCAACGAGAGGTGCAAGAGCAA |
| | | GGTGGTGCCCTGCAGGCAGAAGCAGTGGAGGACCAGCTTCGA |
| | | CAGCAAGAAGCTGGACCTGAACTACAACCACTTCGAGAGCAT |
| | | GGAGTGGAGCCACAGGAGCAGGAGGGGCAGGATCTGGGGCA |
| | | TGTGA (SEQ ID NO: 280) |
| | GM-CSF | ATGTGGCTGCAGTCTCTGCTGCTGCTGGGAACAGTGGCCTGTA |
| | signal peptide | GCATCTCT (SEQ ID NO: 252) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATtacTGGGGACAGGGCACCCTGG |
| | | TCACAGTTTCTTCT (SEQ ID NO: 266) |
| | Linker | TCAGGCGGCGGAGGAGCGGCCGCAGGA (SEQ ID NO: 281) |
| | NKG2D | TGCTACAGCGAGACCCTGCCCATCCAGGTGGAGCAGAACTTC |
| | (reverse | CTGAGCAACCTGTTCGTGGCCAGCTGGATCACCGTGATGATC |
| | orientation- | ATCTTCAGGATCGGCATGGCCGTGGCCATCTTCTGCTGCTTCT |
| | extracellular, | TCTTCCCCAGCGCCAACGAGAGGTGCAAGAGCAAGGTGGTGC |
| | TM, | CCTGCAGGCAGAAGCAGTGGAGGACCAGCTTCGACAGCAAG |
| | cytoplasmic) | AAGCTGGACCTGAACTACAACCACTTCGAGAGCATGGAGTGG |
| | | AGCCACAGGAGCAGGAGGGGCAGGATCTGGGGCATGTGA |
| | | (SEQ ID NO: 19) |
| HER2- | Full length | ATGTGGCTGCAGTCTCTGCTGCTGCTGGGAACAGTGGCCTGTA |
| NKp30 | | GCATCTCTGACATCCAGATGACACAGAGCCCTAGCAGCCTGT |
| ecto | | CTGCCTCTGTGGGCGATAGAGTGACCATCACCTGTAGAGCCA |
| TMCyto | | GCCAGGATGTGAATACCGCCGTGGCCTGGTATCAGCAGAAGC |
| | | CTGGAAAAGCCCCTAAGCTGCTGATCTACAGCGCCAGCTTTCT |
| | | GTACAGCGGCGTGCCAAGCAGATTCAGCGGCAGCAGATCTGG |
| | | CACCGACTTCACCCTGACCATCTCTAGCCTGCAGCCTGAGGAC |
| | | TTCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATTACTGGGGACAGG |
| | | GCACCCTGGTCACAGTTTCTTCTTCAGGCGGCGGAGGAGCGG |
| | | CCGCAGGAAATGGGACTAGGTTGGTAGTGGAAAAGGAGCAT |
| | | CCCCAGTTGGGCGCAGGTACCGTACTTCTTCTGCGGGCAGGG |
| | | TTCTATGCTGTCAGCTTTCTGTCTGTGGCAGTTGGGTCCACAG |
| | | TCTATTACCAGGGTAAGTGTCTCACGTGGAAGGGACCACGGC |
| | | GGCAATTGCCTGCGGTTGTTCCCGCACCCCTCCCTCCTCCATG |
| | | CGGTTCAAGTGCACATCTCCTTCCGCCAGTTCCAGGCGGCTGA |
| | | (SEQ ID NO: 282) |
| | GM-CSF | ATGTGGCTGCAGTCTCTGCTGCTGCTGGGAACAGTGGCCTGTA |
| | signal peptide | GCATCTCT (SEQ ID NO: 252) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATTACTGGGGACAGGGCACCCTG |
| | | GTCACAGTTTCTTCT (SEQ ID NO: 266) |
| | Linker | TCAGGCGGCGGAGGAGCGGCCGCAGGA (SEQ ID NO: 281) |
| | NKp30 (short | AATGGGACTAGGTTGGTAGTGGAAAAGGAGCATCCCCAGTTG |
| | extracellular | GGCGCAGGTACCGTACTTCTTCTGCGGGCAGGGTTCTATGCTG |
| | (15 aa), TM, | TCAGCTTTCTGTCTGTGGCAGTTGGGTCCACAGTCTATTACCA |
| | cytoplasmic) | GGGTAAGTGTCTCACGTGGAAGGGACCACGGCGGCAATTGCC |
| | | TGCGGTTGTTCCCGCACCCCTCCCTCCTCCATGCGGTTCAAGT |
| | | GCACATCTCCTTCCGCCAGTTCCAGGCGGCTGA (SEQ ID NO: |
| | | 12) |
| HER2- | Full length | ATGTGGCTGCAGTCTCTGCTGCTGCTGGGAACAGTGGCCTGTA |
| NKp44 | | GCATCTCTGACATCCAGATGACACAGAGCCCTAGCAGCCTGT |
| ecto | | CTGCCTCTGTGGGCGATAGAGTGACCATCACCTGTAGAGCCA |
| TMCyto | | GCCAGGATGTGAATACCGCCGTGGCCTGGTATCAGCAGAAGC |
| | | CTGGAAAAGCCCCTAAGCTGCTGATCTACAGCGCCAGCTTTCT |
| | | GTACAGCGGCGTGCCAAGCAGATTCAGCGGCAGCAGATCTGG |
| | | CACCGACTTCACCCTGACCATCTCTAGCCTGCAGCCTGAGGAC |
| | | TTCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATtacTGGGGACAGGG |
| | | CACCCTGGTCACAGTTTCTTCTTCAGGCGGCGGAGGAGCGGC |
| | | CGCAGGAGTCCCTAGCCAACCACAGAACTCTACCTTGAGGCC |
| | | CGGACCCGCTGCACCCATCGCTTTGGTTCCAGTGTTTTGCGGA |
| | | CTCCTTGTTGCCAAGTCACTTGTCCTTTCTGCTCTCCTGGTATG |
| | | GTGGGGCGACATTTGGTGGAAAACGATGATGGAGCTTCGATC |
| | | CTTGGACACACAGAAGGCGACATGTCATCTCCAACAGGTGAC |
| | | AGACCTGCCATGGACTAGTGTGTCAAGTCCCGTCGAGCGCGA |
| | | AATCCTTTATCATACCGTGGCCCGAACCAAAATAAGCGACGA |
| | | TGACGATGAGCACACTCTGTGA (SEQ ID NO: 283) |
| | GM-CSF | ATGTGGCTGCAGTCTCTGCTGCTGCTGGGAACAGTGGCCTGTA |
| | signal peptide | GCATCTCT (SEQ ID NO: 252) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATtacTGGGGACAGGGCACCCTGG |
| | | TCACAGTTTCTTCT (SEQ ID NO: 266) |
| | Linker | TCAGGCGGCGGAGGAGCGGCCGCAGGA (SEQ ID NO: 281) |
| | NKp44 (short | GTCCCTAGCCAACCACAGAACTCTACCTTGAGGCCCGGACCC |
| | extracellular | GCTGCACCCATCGCTTTGGTTCCAGTGTTTTGCGGACTCCTTG |
| | (19 aa), TM, | TTGCCAAGTCACTTGTCCTTTCTGCTCTCCTGGTATGGTGGGG |
| | cytoplasmic) | CGACATTTGGTGGAAAACGATGATGGAGCTTCGATCCTTGGA |
| | | CACACAGAAGGCGACATGTCATCTCCAACAGGTGACAGACCT |
| | | GCCATGGACTAGTGTGTCAAGTCCCGTCGAGCGCGAAATCCT |
| | | TTATCATACCGTGGCCCGAACCAAAATAAGCGACGATGACGA |
| | | TGAGCACACTCTGTGA (SEQ ID NO: 15) |
| HER2- | Full length | ATGTGGCTGCAGTCTCTGCTGCTGCTGGGAACAGTGGCCTGTA |
| NKp46 | | GCATCTCTGACATCCAGATGACACAGAGCCCTAGCAGCCTGT |
| ecto | | CTGCCTCTGTGGGCGATAGAGTGACCATCACCTGTAGAGCCA |
| TMCyto | | GCCAGGATGTGAATACCGCCGTGGCCTGGTATCAGCAGAAGC |
| | | CTGGAAAAGCCCCTAAGCTGCTGATCTACAGCGCCAGCTTTCT |
| | | GTACAGCGGCGTGCCAAGCAGATTCAGCGGCAGCAGATCTGG |
| | | CACCGACTTCACCCTGACCATCTCTAGCCTGCAGCCTGAGGAC |
| | | TTCGCCACCTACTACTGCCAGCAGCACTACACCACACCTCCAA |
| | | CCTTTGGCCAGGGCACCAAGGTGGAAATCAAGAGAACAGGC |
| | | AGCACCAGCGGCTCTGGAAAGCCTGGATCTGGCGAAGGATCT |
| | | GAGGTGCAGCTGGTTGAATCTGGCGGAGGACTTGTTCAGCCT |
| | | GGCGGCTCTCTGAGACTGTCTTGTGCCGCCAGCGGCTTCAACA |
| | | TCAAGGACACCTACATCCACTGGGTCCGACAGGCCCCTGGAA |
| | | AGGGACTTGAATGGGTCGCCAGAATCTACCCCACCAACGGCT |
| | | ACACCAGATACGCCGATAGCGTGAAGGGCAGATTCACCATCA |
| | | GCGCCGACACCAGCAAGAACACCGCCTACCTGCAGATGAACA |
| | | GCCTGAGAGCCGAGGACACCGCCGTGTACTACTGTTCTAGAT |
| | | GGGGAGGCGACGGCTTCTACGCCATGGATtacTGGGGACAGGG |
| | | CACCCTGGTCACAGTTTCTTCTTCAGGCGGCGGAGGAGCGGC |
| | | CGCAGGAGGACTTCAAAAGGACCATGCGTTGTGGGATCATAC |
| | | AGCTCAGAACTTGCTCCGCATGGGCCTGGCCTTTCTTGTACTC |
| | | GTTGCACTCGTTTGGTTCCTTGTTGAGGATTGGCTCTCTAGAA |
| | | AGAGAACTAGAGAACGGGCCTCCAGGGCATCCACGTGGGAA |
| | | GGCCGCAGACGACTCAATACCCAGACCCTGTGA (SEQ ID NO: |
| | | 284) |
| | GM-CSF | ATGTGGCTGCAGTCTCTGCTGCTGCTGGGAACAGTGGCCTGTA |
| | signal peptide | GCATCTCT (SEQ ID NO: 252) |
| | HER2 scFv | GACATCCAGATGACACAGAGCCCTAGCAGCCTGTCTGCCTCT |
| | | GTGGGCGATAGAGTGACCATCACCTGTAGAGCCAGCCAGGAT |
| | | GTGAATACCGCCGTGGCCTGGTATCAGCAGAAGCCTGGAAAA |
| | | GCCCCTAAGCTGCTGATCTACAGCGCCAGCTTTCTGTACAGCG |
| | | GCGTGCCAAGCAGATTCAGCGGCAGCAGATCTGGCACCGACT |
| | | TCACCCTGACCATCTCTAGCCTGCAGCCTGAGGACTTCGCCAC |
| | | CTACTACTGCCAGCAGCACTACACCACACCTCCAACCTTTGGC |
| | | CAGGGCACCAAGGTGGAAATCAAGAGAACAGGCAGCACCAG |
| | | CGGCTCTGGAAAGCCTGGATCTGGCGAAGGATCTGAGGTGCA |
| | | GCTGGTTGAATCTGGCGGAGGACTTGTTCAGCCTGGCGGCTCT |
| | | CTGAGACTGTCTTGTGCCGCCAGCGGCTTCAACATCAAGGAC |
| | | ACCTACATCCACTGGGTCCGACAGGCCCCTGGAAAGGGACTT |
| | | GAATGGGTCGCCAGAATCTACCCCACCAACGGCTACACCAGA |
| | | TACGCCGATAGCGTGAAGGGCAGATTCACCATCAGCGCCGAC |
| | | ACCAGCAAGAACACCGCCTACCTGCAGATGAACAGCCTGAGA |
| | | GCCGAGGACACCGCCGTGTACTACTGTTCTAGATGGGGAGGC |
| | | GACGGCTTCTACGCCATGGATtacTGGGGACAGGGCACCCTGG |
| | | TCACAGTTTCTTCT (SEQ ID NO: 266) |
| | Linker | TCAGGCGGCGGAGGAGCGGCCGCAGGA (SEQ ID NO: 281) |
| | NKp46 (short | GGACTTCAAAAGGACCATGCGTTGTGGGATCATACAGCTCAG |
| | extracellular | AACTTGCTCCGCATGGGCCTGGCCTTTCTTGTACTCGTTGCAC |
| | (18 aa), TM, | TCGTTTGGTTCCTTGTTGAGGATTGGCTCTCTAGAAAGAGAAC |
| | cytoplasmic) | TAGAGAACGGGCCTCCAGGGCATCCACGTGGGAAGGCCGCA |
| | | GACGACTCAATACCCAGACCCTGTGA (SEQ ID NO: 18) |

A general protocol for testing expression and functional characterization of the constructs is provided in a schematic form in FIG. 3 indicating the timeline in days for treatments, expansion and assay. NK cells are isolated from primary human donors, usually leukopak samples. Cells were expand and activated in vitro for 7 days using IMMUJNOCULT NK cell expansion kit. 2×10\hat{ }6 cells were then electroporated (EP) with 20 microgram/ml mRNA encoding a CFP, e.g., a TROP2 expressing CFP in a MaxCyte system using the NK-2 program. Cell surface expression of receptor detected by TROP2-AF647 labeling 24 hours post-EP. The schematic diagrams of each CFP polypeptides are graphically shown in FIG. 2A (Top: for myeloid cell-specific expression, Bottom: myeloid/NK cell-specific expression), FIG. 2B (for NK cell-specific expression). FIG. 13, diagram on the left of the graph shows exemplary CFP structures for T cell-specific expression. For instance, a CFP designed for myeloid cell-specific expression may comprise a CD89 receptor TM domain, that specifically integrates within a membrane protein complex of the FcR-gamma chains endogenously expressed in myeloid cells and intracellular signaling occurs through FcR-gamma intracellular domains, ICDs. A CFP designed for myeloid/NK cell-specific expression may comprise a CD16 receptor TM domain, that specifically integrates within a membrane protein complex of the FcγR-gamma III chain endogenously expressed in myeloid cells and intracellular signaling occurs through FcγR-gamma III chain ICDs. For instance, a CFP designed for NK cell-specific expression, it may comprise a DAP12 receptor TM domain, that specifically integrates within a DAP12 membrane protein complex of the FcR-gamma chains endogenously expressed in myeloid cells and intracellular signaling occurs through FcR-gamma intracellular domains, ICDs. Since DAP12 membrane protein complexes are predominantly found naturally in NK cells and not in all cells, e.g., epithelial cell, the construct would not express on an epithelial cell even if the recombinant nucleic acid is uptaken by other such cell lacking DAP12 membrane protein complexes. For exemplary purposes the extracellular antigen binding domains are HER2 binding domains (HER2 CFP constructs) or TROP2 binding domains (TROP2-CFP constructs), but essentially, the antigen binding domains could be swapped out for any antigen binding domain necessary.

For killing assays, NK cells expressing the CFP were co-cultured with SKOV3-Luc cells, i.e., SKOV3 tumor cells expressing Luciferase, making the cell fluorescent. Decrease in fluorescence indicates killing of SKOV3 cells, and is measured by Promega CytoTox-Glo assay.

Preliminary experiments yielded failure of expression of NKG2C and NKG2D based receptor (FIGS. 4A-4C, data from the same experiment). In subsequent experiments the constructs were redesigned flipping the orientation of the protein.

FIG. 5 shows successful expression of NKp30 constructs in NK cells. FIG. 6A and FIG. 6B show successful expression of NKp44 and NKp46 constructs in NK cells. FIG. 7 shows representative results of successful killing of cancer cells by NK cells expressing the constructs as indicated in the graph.

### Example 4. Further Characterization of CD16 Based NK Cell-Specific CFPs

Upon further characterization of the constructs, it was observed that with the constructs with CD16 TM containing CFP constructs had high efficiency of specific killing of tumor cells, for example, targeting HER2 cancer cells or TROP2 cancer cells: FIG. 7, showing activity of NK cells expressing CFP with CD16 TM and anti-HER2 scFv; FIG. 8, showing activity of NK cells expressing CFP with CD16 TM and anti-TROP2 scFv. In both cases comparison was made with first generation CFPs constructed by this group having CD8TM and CD3z ICDs—and did not have cell specificity for expression, and the CD16 constructs had higher efficiency in comparison.

Additionally a time course study of tumor cell lysis also corroborated in FIG. 9 the finding of higher efficiency similar to of FIGS. 7-8. FIG. 10 shows that Tumor killing is accompanied by cytokine upregulation.

### Example 5. Inclusion of an Extracellular Domain or Portion Thereof from the Protein Contributing the TM Domain Increases Expression of the CFP

In an exemplary study concerning improving expression of the CFP constructs, it was found that inclusion of the extracellular domain of related TM domain augments expression of the CFP, whereas constructs lacking the extracellular domain (designated as a AExtracellular domain construct or a TM-cyto construct, both referring to the same construct) have lower expression (FIG. 5, FIG. 6A-6B). In FIGS. 6A and 6B, HER2-NKp44 and HER2-NKp46 comprise an extracellular domains of NKp44 or NKp46 respectively, full length (FL)). This shows that inclusion of the ectodomain increased the robustness of expression of the In a further study, it was seen that about 18-20 amino acids from the extracellular domain next to the TM domain (referred to as ectodomain, or ectoTMcyto constructs) was capable of inducing the higher expression effect. (FIG. 11A-FIG. 11B). For a construct with NKp30 TM domain, having a full length extracellular portion (e.g. NKp30 full length, FL constructs) improves expression of the CFP in NK cells more than having the short extracellular segment (referred to as NKp30ctoTMcyto); and having NKp30ctoTMcyto improved expression over a nonspecific linker between the TM domain and the extracellular antigen binding domain (e.g., anti-TROP2 scFv or anti-HER scFV) improves expression of the CFP in NK cells. As shown in FIG. 11C, the change in expression level roughly correlated with killing of tumor cells in vivo. Additionally, upon HER2 stimulation and activation of the CFP receptor, the NK cells expressing the constructs with the short extracellular domains exhibited high NF-kappaB activity (FIG. 11D). This indicates potent signaling through the intracellular domain in case of the CFPs that have retained a portion of the extracellular domain of the corresponding TM domain. Without wishing to be bound by a theory, it may be possible that the presence of the short extracellular domain further facilitates activation of the CFP receptor.

### Example 6. Impact of Hinge Region in Expression and Function of the CFP

In an exemplary study the impact of the hinge domain on the expression and function of the CFP was evaluated. FIG. 12A shows an array of hinge domains tested, with the lengths and structural nature of the hinge domains. Data demonstrated in FIGS. 12B-12D on the influence of hinge parameters on exemplary CD16 based CFPs indicate that the hinge region influences CAR expression and function. Longer, flexible hinges enable binding to membrane proximal antigen. Also, different hinges result in different oligomerization state. Additionally, inclusion of CD4 hinge improved Fcγ chain dependent expression in liver cell line, Huh7 (FIG. 12B). Additionally, an assessment of duration of receptor expression post translation was tested, and compared with a construct with no hinge versus CD4 hinge domain or no hinge versus CD8 hinge (FIG. 12C). It was seen that inclusion of a CD4 hinge domain improved Fc-gamma receptor dependent expression in NK cells. Additionally FIG. 12D shows that having the CD4 hinge domain included in the CFP construct increased both expression and tumoricidal activity.

### Example 7. T Cell-Specific Receptor Generation and Characterization

In an exemplary study using an anti-CD19 CFP generated for T cell-specific expression, the construct comprises an scFv that can bind to CD19 antigens, and a CD3a TM domain (FIG. 13, left). The CFP was expressed in T cells and the cells demonstrated potent killing of CD19+ Raji cells, when co-cultured (FIG. 13, right).

Taken together, these studies demonstrate successful expression and functionality of the newly designed CFP constructs for cell type-specific expression in vivo, and can be developed for delivery and in vivo application.

1-139. (canceled)

140. A composition comprising:
a lipid nanoparticle delivery vehicle,
a polynucleic acid comprising a sequence encoding a chimeric fusion protein (CFP), the CFP comprising: (i) an extracellular domain comprising an antigen binding domain that binds to a target antigen; (ii) a transmembrane domain operably linked with the extracellular domain, and wherein the transmembrane domain dimerizes or multimerizes with a protein that naturally expresses on an immune cell, and wherein after administering the composition to a subject, the CFP expresses on the immune cell of the subject, and wherein the immune cell is a natural killer (NK) cell or a B cell,
wherein the polynucleic acid is encapsulated in the lipid delivery vehicle.

141. The composition of claim 140, wherein the immune cell is the natural killer (NK) cell, and wherein the transmembrane domain is a transmembrane domain from a protein selected from a group consisting of CD39, CD56, CD57, CD94, CD159a, CD159c, CD314, CD335, CD336, CD337, DAP12, DAP10, NKG2C, NKG2D, NKG2E, Ly49D, Ly49D, KIR, NKp46, NKp30 and NKp44.

142. The composition of claim 141, wherein the transmembrane domain dimerizes with a domain from an ITAM motif containing co-receptor, wherein the ITAM motif containing co-receptor is selected from the group consisting of DAP10, DAP12 and CD94.

143. The composition of claim 141, wherein the CFP further comprises (iii) an intracellular domain from a protein selected from a group consisting of CD39, CD56, CD57, CD94, CD159a, CD159c, CD314, CD335, CD336, CD337, DAP12, DAP10, NKG2C, NKG2D, NKG2E, Ly49D, Ly49D, NKp46, NKp30 and NKp44.

144. The composition of claim 141, wherein the extracellular domain further comprises at least a portion of an extracellular domain from a protein selected from a group consisting of CD39, CD56, CD57, CD94, CD159a, CD159c, CD314, CD335, CD336, CD337, DAP12, DAP10, NKG2C, NKG2D, NKG2E, Ly49D, Ly49D, KIR, NKp46, NKp30 and NKp44.

145. The composition of claim 141, wherein the CFP is not substantially expressed on a non-NK cell.

146. The composition of claim 141, wherein the NK cell is a cytotoxic or cytolytic NK cell.

147. The composition of claim 141, wherein the CFP comprises the transmembrane domain of NKp30; and an NKp30 intracellular domain.

148. The composition of claim 147, wherein the CFP comprises a sequence that is at least 90% identical to the sequence encoded by SEQ ID NO: 10, SEQ ID NO: 11 or SEQ ID NO:12.

149. The composition of claim 147, wherein the CFP comprises a sequence that is at least 90% identical to the amino acid sequence of amino acids 272-337 of SEQ ID NO: 32.

150. The composition of claim 141, wherein the CFP comprises the transmembrane domain of NKp44; and an NKp44 intracellular domain.

151. The composition of claim 150, wherein the CFP comprises a sequence that is at least 90% identical to the sequence encoded by SEQ ID NO: 13, SEQ ID NO: 15 or SEQ ID NO: 277.

152. The composition of claim 150, wherein the CFP comprises a sequence that is at least 90% identical to amino acid sequence of amino acids 272-355 of SEQ ID NO: 34.

153. The composition of claim 141, wherein the CFP comprises the transmembrane domain of NKp46; and an NKp46 intracellular domain.

154. The composition of claim 153, wherein the CFP comprises a sequence that is at least 90% identical to the sequence encoded by SEQ ID NO: 16, SEQ ID NO: 17 or SEQ ID NO: 18.

155. The composition of claim 153, wherein the CFP comprises a sequence that is at least 90% identical to amino acid sequence of amino acids 272-317 of SEQ ID NO: 36.

156. The composition of claim 141, wherein the target antigen is on a cancer cell, and wherein the target antigen is selected from the group consisting of Thymidine Kinase (TK1), Hypoxanthine-Guanine Phosphoribosyltransferase (HPRT), Receptor Tyrosine Kinase-Like Orphan Receptor 1 (RORI), Mucin-1, Mucin-16 (MUC16), MUC1, Epidermal Growth Factor Receptor vIII (EGFRvIII), Mesothelin, EBNA-1, LEMD1, Phosphatidyl Serine, Carcinoembryonic Antigen (CEA), B-Cell Maturation Antigen (BCMA), Follicular Stimulating Hormone receptor, Fibroblast Activation Protein (FAP), Erythropoietin-Producing Hepatocellular Carcinoma A2 (EphA2), EphB2, a Natural Killer Group 2D (NKG2D) ligand, Disialoganglioside 2 (GD2), CD2, CD3, CD4, CD7, CD8, CD19, CD20, CD22, CD24, CD30, CD33, CD3δ, CD44v6, CD45, CD56, CD79b, CD97, CD117, CD123, CD133, CD138, CD171, CD179a, CD213A2, CD248, CD276, PSCA, CS-1, CLECLI, GD3, PSMA, FLT3, TAG72, EPCAM, IL-1, an integrin receptor, PRSS21, VEGFR2, PDGFRβ, SSEA-4, EGFR, NCAM, prostase, PAP, ELF2M, GM3, TEM7R, CLDN6, TSHR, GPRC5D, ALK, Dsg1, Dsg3, IGLL1, CCR4, CD5, Human Epidermal Growth Factor Receptor 2 (HER2), Glypican 3 (GPC3), and TROP2.

157. The composition of claim 141, wherein the antigen binding domain binds to a T cell lymphoma antigen.

158. The composition of claim 141, wherein the antigen binding domain binds to a target antigen on a B cell surface.

159. The composition of claim 158, wherein the target antigen on a B cell surface is CD19 or CD20 antigen.

160. The composition of claim 141, wherein the polynucleic acid is RNA.

161. The composition of claim 140, wherein the immune cell is the B cell; and wherein after administering the composition to a subject, the CFP is expressed on B cells of the subject, and not substantially expressed on a cell other than a B cell.

162. A polynucleic acid, comprising: (i) a sequence encoding a CFP of claim 141, comprising a sequence that is at least 90% identical to any one from SEQ ID NO: 1, SEQ ID NO: 8, SEQ ID NO: 10, SEQ ID NO: 11, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 270, SEQ ID NO: 272, and SEQ ID NO: 277; or (ii) a sequence that is a reverse complement of (i).

163. A method of treating a disease in a subject in need thereof, the method comprising, administering to the subject a therapeutically effective amount of the composition of claim 141.

164. The method of claim 163, wherein the disease is a cancer, an autoimmune disease, a fibrotic diseases, or an infectious disease.

165. A composition comprising a lipid nanoparticle delivery vehicle, comprising a polynucleic acid comprising a sequence encoding a chimeric fusion protein (CFP), the CFP comprising: (i) an extracellular domain comprising an antigen binding domain that binds to an antigen expressed on a target cell; (ii) a transmembrane domain that dimerizes or multimerizes with a membrane receptor or a member of a membrane receptor complex that naturally expresses on a T cell; and wherein after administering the composition to a subject, the CFP expresses on T cells of the subject and not substantially expressed on a cell other than a T cell.
